SEQUENCE LISTING (69 sequences)

SEQ ID NO: 1 - DNA, 1497 bp, Saccharomyces cerevisiae
  CDS (1)..(1497): GWT1
atggcaacagtacatcagaagaatatgtcgactttaaaacagagaaaagaggactttgtgacagggctcaatggcggttctataacagaaattaacgcagtgacatcaattgctttggtaacttacatatcatggaacttattgaaaaattccaaccttatgcctcctggcatttccagcgtgcaatacataattgattttgcattgaactgggttgctttgcttctatctattactatttatgctagtgaaccataccttctaaacacgctaatactgttaccttgtttgctcgcattcatatatggaaaatttactagctcgagtaaaccttctaatccaatatacaataaaaaaaaaatgattacacagcggttccaactagaaaaaaagccgtatattactgcgtatcgtggtgggatgcttattctgactgctattgccatcttggctgtagattttccaattttcccaaggaggtttgccaaggtggaaacttgggggacatccctgatggatcttggtgtaggatcattcgttttcagtaacggtattgtttcttctagggcactgttgaaaaacctaagcttgaagagtaaacccagcttcttaaaaaatgcatttaatgccttaaaatcaggaggaactctattgttcctaggattgctgaggttgttttttgtaaaaaatttggaatatcaagaacatgtcacagaatatggggttcattggaatttttttatcaccctatcattgttgccacttgtattgacctttattgatcccgtcacaagaatggttccacgctgctcaattgcaatattcatttcatgcatttatgaatggctacttttaaaggacgatcgcactttaaactttttaattttggctgatagaaattgtttcttcagtgctaatagagaaggcatcttctcatttctaggttattgctcgatttttctttggggccaaaacacgggattttacttgttgggaaataaaccaactttaaacaatctttataagccttctacgcaagacgtagttgcagcatcaaagaagtcttcgacttgggactattggacttcagtaaccccattaagtggcctctgtatatggagtacaatttttcttgttatcagccagttggtttttcaataccatccttatagtgtttcaagaaggtttgctaacttaccatatactttgtgggtcattacttataatttactatttttgactgggtactgcttgactgacaaaattttcggtaattcttcggaatattataaagttgccgaatgcttggaatcaatcaactccaatgggttgtttttatttttgttggcaaatgtctctactggtttagtcaatatgtctatggtcacgatagattcttcacccttaaaatcattcctggttttgttggcatactgctcattcatagctgtcatatcggttttcttgtatagaaaaagaatattcattaagctataa

SEQ ID NO: 2 - protein, 498 aa, Saccharomyces cerevisiae
  GWT1
MATVHQKNMSTLKQRKEDFVTGLNGGSITEINAVTSIALVTYISWNLLKNSNLMPPGISSVQYIIDFALNWVALLLSITIYASEPYLLNTLILLPCLLAFIYGKFTSSSKPSNPIYNKKKMITQRFQLEKKPYITAYRGGMLILTAIAILAVDFPIFPRRFAKVETWGTSLMDLGVGSFVFSNGIVSSRALLKNLSLKSKPSFLKNAFNALKSGGTLLFLGLLRLFFVKNLEYQEHVTEYGVHWNFFITLSLLPLVLTFIDPVTRMVPRCSIAIFISCIYEWLLLKDDRTLNFLILADRNCFFSANREGIFSFLGYCSIFLWGQNTGFYLLGNKPTLNNLYKPSTQDVVAASKKSSTWDYWTSVTPLSGLCIWSTIFLVISQLVFQYHPYSVSRRFANLPYTLWVITYNLLFLTGYCLTDKIFGNSSEYYKVAECLESINSNGLFLFLLANVSTGLVNMSMVTIDSSPLKSFLVLLAYCSFIAVISVFLYRKRIFIKL

SEQ ID NO: 3 - DNA, 1458 bp, Candida albicans
  CDS (1)..(1458): CaGWT1
atgtcatcgtctttaaaacaattgaaagaacaatttgtctcagatttgactggtggcacaattgaagaaatttatgctgtaaccagtatagcattatcatcttatttgtcctttagattgttgaaaaagtctcttggtgatttagctttgatttacgactacattcttaatgtgttgacaattctagcatccattactgtttatagcaacagcccttcttatttgcattattttattgttattccatcattagttatatatctagtgaattaccatgttgagaaaccatcttcaccccatagacaaaatgatacaaaagaagataaatcggacgaactattgccgagaaaacaatttataacagcctatcgttctcaaatgttgataattactaatctagctatattagctgttgattttcctattttcccaagaagatttgccaaagtggaaacatggggcacgtcaatgatggatttaggagttgggtcgtttgtgttctccatggggttggctaattctcgacaattgatcaagaaccacaccgacaactacaaatttagttggaagagttatttgaaaacaatcaagcagaactttatcaagtcagtgcctatacttgttttaggagctattcgttttgttagtgttaagcaattggactatcaggaacacgaaacagagtatggaatccattggaattttttcttcacattagggttcttgccaattgtattgggaatattagacccggtgttgaatttggttccacgcttcataataggaattggtatctcaattgcttatgaggtagcgttgaataagactggtttgttgaagttcattttgagcagcgaaaacagacttgaatctctcatcaccatgaataaagaaggtattttttcgtttattggatatctttgtatttttataattggtcagtcttttgggtcatttgttttaacaggctacaaaacaaagaacaacttaataaccattagcaaaattcgtatttcaaaaaaacaacacaagaaagagctgctgctgtttttctcagtcgccactactcagggattatatttggcatgtatcttctatcacttagctttcagtttgttcatcagcaacttatcattcttgcaaccaatttcaagacgattggccaatttcccctacgtcatgtgggtcgtttcgtacaatgctacgtttttattatgttatgacttaattgaaaaatttatcccggggaaccttacttctactgtattggactctattaataacaatggtttatttatcttcttggtcagcaatttattaacagggtttattaacatgtccatcaacactttggaaactagcaataaaatggcagtgattatcttgattggctatagtcttacttggacattgctcgccttatatttggataagaggaagatctacatcaagctttag

SEQ ID NO: 4 - protein, 485 aa, Candida albicans
  CaGWT1
MSSSLKQLKEQFVSDLTGGTIEEIYAVTSIALSSYLSFRLLKKSLGDLALIYDYILNVLTILASITVYSNSPSYLHYFIVIPSLVIYLVNYHVEKPSSPHRQNDTKEDKSDELLPRKQFITAYRSQMLIITNLAILAVDFPIFPRRFAKVETWGTSMMDLGVGSFVFSMGLANSRQLIKNHTDNYKFSWKSYLKTIKQNFIKSVPILVLGAIRFVSVKQLDYQEHETEYGIHWNFFFTLGFLPIVLGILDPVLNLVPRFIIGIGISIAYEVALNKTGLLKFILSSENRLESLITMNKEGIFSFIGYLCIFIIGQSFGSFVLTGYKTKNNLITISKIRISKKQHKKELLLFFSVATTQGLYLACIFYHLAFSLFISNLSFLQPISRRLANFPYVMWVVSYNATFLLCYDLIEKFIPGNLTSTVLDSINNNGLFIFLVSNLLTGFINMSINTLETSNKMAVIILIGYSLTWTLLALYLDKRKIYIKL

SEQ ID NO: 5 - DNA, 1458 bp, Candida albicans
  CDS (1)..(1458): CaGWT1
atgtcatcgtctttaaaacaattgaaagaacaatttgtctcagatttgactggtggcacaattgaagaaatttatgctgtaaccagtatagcattatcatcttatttgtcctttagattgttgaaaaagtctcttggtgatttagctttgatttacgactacattcttaatgtgttgacaattctagcatccattactgtttatagcaacagcccttcttatttgcattattttattgttattccatcattagttatatatctagtgaattaccatgttgagaaaccatcttcaccccatagacaaaatgatacaaaagaagataaatcggacgaactattgccgagaaaacaatttataacagcctatcgttctcaaatgttgataattactaatctagctatattagctgttgattttcctattttcccaagaagatttgccaaagtggaaacatggggcacgtcaatgatggatttaggagttgggtcgtttgtgttctccatggggttggctaattctcgacaattgatcaagaaccacaccgacaattacaaatttagttggaagagttatttgaaaacaatcaagcagaactttatcaagtcagtgcctatacttgttttaggagctattcgttttgttagtgttaagcaattggactatcaggaacacgaaacagagtatggaatccattggaattttttcttcacattagggttcttgccaattgtattgggaatattagacccggtgttgaatttggttccacgcttcataataggaattggtatctcaattggttatgaggtagcgttgaataagactggtttgttgaagttcattttgagcagcgaaaacagacttgaatctctcatcgccatgaataaagaaggtattttttcgtttattggatatctttgtatttttataattggtcagtcttttgggtcatttgttttaacaggctacaaaacaaagaacaacttaataaccattagcaaaattcgtatttcaaaaaaacaacacaagaaagagctgctgctgtttttctcagtcgccactactcagggattatatttggcatgtatcttctatcacttagctttcagtttgttcatcagcaacttatcattcttgcaaccaatttcaagacgattggccaatttcccctacgtcatgtgggtcgtttcgtacaatgctacgtttttattatgttatgacttaattgaaaaatttatcccggggaaccttacttctactgtattggactctattaataacaatggtttatttatcttcttggtcagcaatttattaacagggtttattaacatgtccatcaacactttggaaactagcaataaaatggcagtgattatcttgattggctatagtcttacttggacattgctcgccttatatttggataagaggaagatctacatcaagctttag

SEQ ID NO: 6 - protein, 485 aa, Candida albicans
  CaGWT1
MSSSLKQLKEQFVSDLTGGTIEEIYAVTSIALSSYLSFRLLKKSLGDLALIYDYILNVLTILASITVYSNSPSYLHYFIVIPSLVIYLVNYHVEKPSSPHRQNDTKEDKSDELLPRKQFITAYRSQMLIITNLAILAVDFPIFPRRFAKVETWGTSMMDLGVGSFVFSMGLANSRQLIKNHTDNYKFSWKSYLKTIKQNFIKSVPILVLGAIRFVSVKQLDYQEHETEYGIHWNFFFTLGFLPIVLGILDPVLNLVPRFIIGIGISIGYEVALNKTGLLKFILSSENRLESLIAMNKEGIFSFIGYLCIFIIGQSFGSFVLTGYKTKNNLITISKIRISKKQHKKELLLFFSVATTQGLYLACIFYHLAFSLFISNLSFLQPISRRLANFPYVMWVVSYNATFLLCYDLIEKFIPGNLTSTVLDSINNNGLFIFLVSNLLTGFINMSINTLETSNKMAVIILIGYSLTWTLLALYLDKRKIYIKL

SEQ ID NO: 7 - DNA, 1458 bp, Candida albicans
  homologue of S. cerevisiae GWT1
atgtcatcgtctttaaaacaattgaaagaacaatttgtctcagatttgactggtggcacaattgaagaaatttatgctgtaaccagtatagcattatcatcttatttgtcctttagattgttgaaaaagtctcttggtgatttagctttgatttacgactacattcttaatgtgttgacaattctagcatccattactgtttatagcaacagcccttcttatttgcattattttattgttattccatcattagttatatatctagtgaattaccatgttgagaaaccatcttcaccccatagacaaaatgatacaaaagaagataaatcggacgaactattgccgagaaaacaatttataacagcctatcgttctcaaatgttgataattactaatctagctatattagctgttgattttcctattttcccaagaagatttgccaaagtggaaacatggggcacgtcaatgatggatttaggggttgggtcgtttgtgttctccatggggttggctaattctcgacaattgatcaagaaccacaccgacaactacaaatttagttggaagagttatttgaaaacaatcaagcagaactttatcaagtcagtgcctatacttgttttaggagctattcgttttgttagtgttaagcaattggactatcaggaacacgaaacagagtatggaatccattggaattttttcttcacattagggttcttgccaattgtattgggaatattagacccggtgttgaatttggttccacgcttcataataggaattggtatctcaattggttatgaggtagcgttgaataagactggtttgttgaagttcattttgagcagcgaaaacagacttgaatctctcatcgccatgaataaagaaggtattttttcgtttattggatatctttgtatttttataattggtcagtcttttgggtcatttgttttaacaggctacaaaacaaagaacaacttaataaccattagcaaaattcgtatttcaaaaaaacaacacaagaaagagctgctgctgtttttctcagtcgccactactcagggattatatttggcatgtatcttctatcacttagctttcagtttgttcatcagcaacttatcattcttgcaaccaatttcaagacgattggccaatttcccctacgtcatgtgggtcgtttcgtacaatgctacgtttttattatgttatgacttaattgaaaaatttatcccggggaaccttacttctactgtattggattctattaataacaatggtttatttatcttcttggtcagcaatttattaacagggtttattaacatgtccatcaacactttggaaactagcaataaaatggcagtgattatcttgattggctatagtcttacttggacattgctcgccttatatttggataagaggaagatctacatcaagctttag

SEQ ID NO: 8 - DNA, 33 bp, Artificial Sequence
  Description of Artificial Sequence oligonucleotide primer
gcagtcgactcgatgaggtctttgctaatcttg

SEQ ID NO: 9 - DNA, 33 bp, Artificial Sequence
  Description of Artificial Sequence oligonucleotide primer
gcagaattcgacaccacaaccttgaacgtattg

SEQ ID NO: 10 - DNA, 33 bp, Artificial Sequence
  Description of Artificial Sequence oligonucleotide primer
cccgaattcactgacggtcaaatccaagctact

SEQ ID NO: 11 - DNA, 32 bp, Artificial Sequence
  Description of Artificial Sequence oligonucleotide primer
ggaagcttttataacaacatagcggcagcagc

SEQ ID NO: 12 - DNA, 49 bp, Artificial Sequence
  Description of Artificial Sequence oligonucleotide primer
cccgcggccgcttgatagtaagcttgcttgggccgcatcatgtaattag

SEQ ID NO: 13 - DNA, 33 bp, Artificial Sequence
  Description of Artificial Sequence oligonucleotide primer
cccggtaccaaattaaagccttcgagcctccca

SEQ ID NO: 14 - DNA, 33 bp, Artificial Sequence
  Description of Artificial Sequence oligonucleotide primer
cccggatcctgtttgcagcatgagacttgcata

SEQ ID NO: 15 - DNA, 45 bp, Artificial Sequence
  Description of Artificial Sequence oligonucleotide primer
cccgcggccgccccttccaattcgaaaaccttccccagagcagcc

SEQ ID NO: 16 - DNA, 32 bp, Artificial Sequence
  Description of Artificial Sequence oligonucleotide primer
ggttcgaagccgcaaaaacagaacaacaaatt

SEQ ID NO: 17 - DNA, 32 bp, Artificial Sequence
  Description of Artificial Sequence oligonucleotide primer
ggtctagattgcagtttttcaagaatgcgcca

SEQ ID NO: 18 - DNA, 33 bp, Artificial Sequence
  Description of Artificial Sequence oligonucleotide primer
gggtctagaactgacggtcaaatccaagctact

SEQ ID NO: 19 - DNA, 32 bp, Artificial Sequence; identical to SEQ ID NO: 11
  Description of Artificial Sequence oligonucleotide primer

SEQ ID NO: 20 - protein, 17 aa, Artificial Sequence
  Description of Artificial SequenceA1s1p synthetic peptide
CFTAGTNTVFNDGDKDI

SEQ ID NO: 21 - DNA, 27 bp, Artificial Sequence
  Description of Artificial SequencePCR amplification primer
aaactgttcactgaacaaccaaatctc

SEQ ID NO: 22 - DNA, 27 bp, Artificial Sequence
  Description of Artificial SequencePCR amplification primer
caactgtaccatttgttagacatcact

SEQ ID NO: 23 - DNA, 30 bp, Artificial Sequence
  Description of Artificial SequencePCR amplification primer
aaacagctgggatcgcaataagaagacacg

SEQ ID NO: 24 - DNA, 29 bp, Artificial Sequence
  Description of Artificial SequencePCR amplification primer
aaacagctgatggaaatgtggatggtgtg

SEQ ID NO: 25 - DNA, 60 bp, Artificial Sequence
  Description of Artificial SequencePCR amplification primer
atggcaacagtacatcaggagaatatgtcgactttaaaaccggatccccgtcgtttaaac

SEQ ID NO: 26 - DNA, 60 bp, Artificial Sequence
  Description of Artificial SequencePCR amplification primer
ttatagcttaatgaatattctttttctatacaagaaaaccgaattcgagctcgtttaaac

SEQ ID NO: 27 - DNA, 1380 bp, Schizosaccharomyces pombe
  CDS (1)..(1380): homologue of S. cerevisiae GWT1
atgtcatacaaattggaaaaagaagcatttgtctcaaacctgacgggttcaagttccattgagacatgtggcttgttattaataggaattgcttgcaacgttttgtgggtaaacatgactgcgagaaacatcttacccaaagggaatcttgggtttcttgttgagtttttcatcttttgcttaattccattatttgtcatttacgtttcatcgaaagttggcgttttcactctttgcatagcctcttttttgccttccttcgtccttcatgttataagtccaattaattgggatgtgctgagaagaaaacctggttgttgtcttactaaaaaaaatgaaaatacttttgatcgacgaattgctggagtcacattttatcgttctcaaatgatgttggttactgtcacttgcatcctggccgttgactttacccttttcccgaggagatatgccaaagttgaaacctggggaacatcactgatggatcttggtgttggatctttcatgttttcttcaggtactgtggctggacggaaaaatgacattaaaaaaccaaatgcgtttaaaaatgtattgtggaattctttcatccttttgattttaggatttgcgcgcatgtttttaacgaaaagcatcaattaccaagaacatgtaagcgaatatggcatgcattggaacttttttttcaccctaggtttcatggctcttggcgtatttttttttcgtcgttctttaaaaaaagtctcctattttaatttagcaaccttcattactcttcttcatcattgtttgcttgttttaacccctttccaaaaatgggcactatccgcccccagaacaaatattttggctcagaatagagagggtattgcttctcttcccggatacattgctatttacttttatggaatgtataccggtagtgtagttttggctgatcgacctctaatgtatactagagctgagtcgtggaagcgctttcaacgtctattattcccgctatgcattttgttagtgttgtatcttgtgtctaactttttgtcagttggtgtttctcgccgacttgctaatacgccttatgttgcgaatgttgcctttatcaatatgttttttcttactatatacatacttattgatgcctatttattcccatcttctgtgccatatggaagtcgcgtccccaaactgcttgaagatgccaataataatggcttgttggtgtttttgattgctaacgttttaacaggagtagttaatttatcgttcgacacccttcattctagcaatgcaaaaggcttgacaatcatgactatgtatctttttattatttgctatatggcacattggcttgctcaacacggaattcgttttcgcctttag

SEQ ID NO: 28 - protein, 459 aa, Schizosaccharomyces pombe
  homologue of S. cerevisiae GWT1
MSYKLEKEAFVSNLTGSSSIETCGLLLIGIACNVLWVNMTARNILPKGNLGFLVEFFIFCLIPLFVIYVSSKVGVFTLCIASFLPSFVLHVISPINWDVLRRKPGCCLTKKNENTFDRRIAGVTFYRSQMMLVTVTCILAVDFTLFPRRYAKVETWGTSLMDLGVGSFMFSSGTVAGRKNDIKKPNAFKNVLWNSFILLILGFARMFLTKSINYQEHVSEYGMHWNFFFTLGFMALGVFFFRRSLKKVSYFNLATFITLLHHCLLVLTPFQKWALSAPRTNILAQNREGIASLPGYIAIYFYGMYTGSVVLADRPLMYTRAESWKRFQRLLFPLCILLVLYLVSNFLSVGVSRRLANTPYVANVAFINMFFLTIYILIDAYLFPSSVPYGSRVPKLLEDANNNGLLVFLIANVLTGVVNLSFDTLHSSNAKGLTIMTMYLFIICYMAHWLAQHGIRFRL

SEQ ID NO: 29 - DNA, 35 bp, Artificial Sequence
  Description of Artificial Sequenceprimer based on presumed DNA encoding GWT1 highly conserved region
gcnaargtngaracntggggnacnwsnytnatgga

SEQ ID NO: 30 - DNA, 38 bp, Artificial Sequence
  Description of Artificial Sequenceprimer based on presumed DNA encoding GWT1 highly conserved region
ttccartgnaynccrtaytcngtnacrtgytcytgrta

SEQ ID NO: 31 - DNA, 32 bp, Artificial Sequence
  Description of Artificial Sequenceprimer based on presumed DNA encoding GWT1 highly conserved region
gtraaraaraarttccartgnaynccrtaytc

SEQ ID NO: 32 - DNA, 188 bp, Aspergillus fumigatus
  fragment having homology to S. cerevisiae GWT1
atggatctgggcgttggatcgtttgtcttttcgggcggagtagtatccgctcgctcactactcaagagcaggaccaatggctctaaaaggttgcctcttgccaagaggttgattgcgtcgacgcgacactctattcctctgctcgtcctcggcctgattcggctatacagcgtcaaaggcttggacta

SEQ ID NO: 33 - DNA, 24 bp, Artificial Sequence
  Description of Artificial SequencePCR amplification primer based on amplified fragment
ggagtagtatccgctcgctcacta

SEQ ID NO: 34 - DNA, 25 bp, Artificial Sequence
  Description of Artificial SequencePCR amplification primer based on amplified fragment
gtccaagcctttgacgctgtatagc

SEQ ID NO: 35 - DNA, 25 bp, Artificial Sequence
  Description of Artificial SequencePCR amplification primer outside gene insertion site of library
gggatgtgctgcaaggcgattaagt

SEQ ID NO: 36 - DNA, 26 bp, Artificial Sequence
  Description of Artificial SequencePCR amplification primer outside gene insertion site of library
tttatgcttccggctcgtatgttgtg

SEQ ID NO: 37 - DNA, 25 bp, Artificial Sequence
  Description of Artificial Sequenceprimer corresponding to sequence upstream of initiation codon
aaaggtgcaaatcccgcggcattga

SEQ ID NO: 38 - DNA, 28 bp, Artificial Sequence
  Description of Artificial Sequenceprimer corresponding to sequence downstream of stop codon
agttcactatatatcttcaacacaccac

SEQ ID NO: 39 - DNA, 1576 bp, Aspergillus fumigatus
  amplified fragment of homologue of S. cerevisiae GWT1 from cDNA library
aaggtgcaaatcccgcggcattgagtcaagatggatccagattataaagctcgcaaagaggcctttgtctcaggtcttgcaggaggaagcatcctggaaatcaacgccgtcaccttggttgcttcggtatccgtttttctgtggtcaattctacaatctcgcctatcctttttcacaccctacagcgccgctgcccttctcgttgatttcctgctcaatgtactagctatcttgttcgcaaccactttatactcttcggcgcctcttcttctcaatctccttctaatatctcccgctctgctgatactcctctctacgaaacgtcctcggacccccgtcaaagcgaaacctcctcgccagtccgctagagctgggaaagatgactcgaaacatgcgacagccttgccagagtctctacccattcatccatttctcacgacatatcgcgccgccatgatggttatcacgtgcatcgctatcttggctgtggattttcgcatttttcctcgccgattcgccaaggtagaaaactggggtacatcactcatggatctgggcgttggatcgtttgtcttttcgggcggagtagtatccgctcgctcactactcaagagcaggaccaatggctctaaaaggttgcctcttgccaagaggttgattgcgtcgacgcgacactctattcctctgctcgtcctcggcctgattcggctatacagcgtcaaaggcttggactatgcggagcacgtcaccgagtacggcgtacattggaacttcttctttacattgggtcttttgcctccgttcgtggaggtcttcgacgccttggctacgatcattccgtcatacgaggttctctccgtggggatcgccgtcttgtatcaagttgccctagagtcaacagacttgaaaagctacatcctcgtctcccctcgtgggccaagcttactgtccaagaatcgtgaaggcgtcttctccttctcaggttatctcgcgatttttcttgctggtcgtgcgatcggcattcggataatccctcgcggaacttctttctcaagaagcccagaacaggccaggagacgggtcctgatcagccttggcgtgcaagcgttagtgtggaccactctttttgtgttgaactccacttatgcgatgggatacggagctaatatccctgtctcccgccgcctcgctaacatgccctatgtcctttgggtttcggcgttcaacaccgcgcaactgtttgtgttctgcctgatcgaaacactctgctttcctgcagttcatcggacaacgactcaagagagcgaatctgagcgagtcgattttgctacgagccgaatcatgtcggccttcaataagaacagtctcgcgatctttcttttggccaatcttctgactggagctgtgaatctgagcatctccacaattgatgctaatacagcgcaggccatcgctgttctcattggatattcatccattatcacaggggttgctctagcattgcatcatgccaatatcaaagtacttcctttctagggtatttacgagcaattggtggtgtgttgaagatatatag

SEQ ID NO: 40 - protein, 501 aa, Aspergillus fumigatus
  homologue of S. cerevisiae GWT1
MDPDYKARKEAFVSGLAGGSILEINAVTLVASVSVFLWSILQSRLSFFTPYSAAALLVDFLLNVLAILFATTLYSSAPLLLNLLLISPALLILLSTKRPRTPVKAKPPRQSARAGKDDSKHATALPESLPIHPFLTTYRAAMMVITCIAILAVDFRIFPRRFAKVENWGTSLMDLGVGSFVFSGGVVSARSLLKSRTNGSKRLPLAKRLIASTRHSIPLLVLGLIRLYSVKGLDYAEHVTEYGVHWNFFFTLGLLPPFVEVFDALATIIPSYEVLSVGIAVLYQVALESTDLKSYILVSPRGPSLLSKNREGVFSFSGYLAIFLAGRAIGIRIIPRGTSFSRSPEQARRRVLISLGVQALVWTTLFVLNSTYAMGYGANIPVSRRLANMPYVLWVSAFNTAQLFVFCLIETLCFPAVHRTTTQESESERVDFATSRIMSAFNKNSLAIFLLANLLTGAVNLSISTIDANTAQAIAVLIGYSSIITGVALALHHANIKVLPF

SEQ ID NO: 41 - DNA, 1648 bp, Aspergillus fumigatus
  cloned homologue of S. cerevisiae GWT1 from genomic library
gcaaatcccgcggcattgagtcaagatggatccagattataaagctcgcaaagaggcctttgtctcaggtcttgcaggaggaagcatcctggaaatcaacgccgtcaccttggttgcttcggttcgtgttactatcttattgtggctacttcgcctacattgtttctcgactaaccgagtctctttgcgatcaatcaggtatccgtttttctgtggtcaattctacaatctcgcctatcctttttcacaccctacagcgccgctgcccttctcgttgatttcctgctcaatgtactagctatcttgttcgcaaccactttatactcttcggcgcctcttcttctcaatctccttctaatatctcccgctctgctgatactcctctctacgaaacgtcctcggacccccgtcaaagcgaaacctcctcgccagtccgctagagctgggaaagatgactcgaaacatgcgacagccttgccagagtctctacccattcatccatttctcacgacatatcgcgccgccatgatggttatcacgtgcatcgctatcttggctgtggattttcgcatttttcctcgccgattcgccaaggtagaaaactggggtacatcactcatggatctgggcgttggatcgtttgtcttttcgggcggagtagtatccgctcgctcactactcaagagcaggaccaatggctctaaaaggttgcctcttgccaagaggttgattgcgtcgacgcgacactctattcctctgctcgtcctcggcctgattcggctatacagcgtcaaaggcttggactatgcggagcacgtcaccgagtacggcgtacattggaacttcttctttacattgggtcttttgcctccgttcgtggaggtcttcgacgccttggctacgatcattccgtcatacgaggttctctccgtggggatcgccgtcttgtatcaagttgccctagagtcaacagacttgaaaagctacatcctcgtctcccctcgtgggccaagcttactgtccaagaatcgtgaaggcgtcttctccttctcaggttatctcgcgatttttcttgctggtcgtgcgatcggcattcggataatccctcgcggaacttctttctcaagaagcccagaacaggccaggagacgggtcctgatcagccttggcgtgcaagcgttagtgtggaccactctttttgtgttgaactccacttatgcgatgggatacggagctaatatccctgtctcccgccgcctcgctaacatgccctatgtcctttgggtttcggcgttcaacaccgcgcaactgtttgtgttctgcctgatcgaaacactctgctttcctgcagttcatcggacaacgactcaagagagcgaatctgagcgagtcgattttgctacgagccgaatcatgtcggccttcaataagaacagtctcgcgatctttcttttggccaatcttctgactggagctgtgaatctgagcatctccacaattgatgctaatacagcgcaggccatcgctgttctcattggatattcatccattatcacaggggttgctctagcattgcatcatgccaatatcaaagtacttcctttctagggtatttacgagcaattggtggtgtgttgaagatatatag

SEQ ID NO: 42 - DNA, 27 bp, Artificial Sequence
  Description of Artificial SequencePCR amplification primer
gccataataagctaccgaattgcaatg

SEQ ID NO: 43 - DNA, 26 bp, Artificial Sequence
  Description of Artificial SequencePCR amplification primer
cattaacacccccattgacaaccacg

SEQ ID NO: 44 - DNA, 1869 bp, Cryptococcus neoformans
  amplified fragment showing homology to S. cerevisiae GWT1
ggggattacaagtcggccaaagaggcctttgtctcggataacccaggtgcttctatctggagtatcaacgctgtcagcctggtcgcactggtatgtagctcgttctccgaggggttctgtcatttggagacgcttattaattgggatcgcaggcgacatatgctctctggatcgccttatcgccgtacatccgtcatggactcctgaacaactacctgatctgtgttcttcccctattattcggggtgaccatcttctcaacttcgcctctcgtatttacctcttttttgtccattatttccctcgctttcatcacgaaatcccaaaaatgcttcaaatctgtcagttcgcccgaaaagccaaaaggccaatggctagacgaatcagactccgatgaggaaccagcggaacctgcttctgcagctggatctgcagcagtctcaccagtaaagcttctaccttcccaagtggcgttcgcttcgggatccctattatctcccgatccgacaacatcccccatgtcgccaagtagttcttcagcttcaggacatgaagaccctttggggattatgggcgttaacagacggaggtcgctattagaaggagtttcgcttgatgttccgtcacatatcgactccaaggtcagaatatctcctgttccctacttgaggctcaaaaagtctagggcaacgaaggcgcaatgggtgaaagaaaagggaagattaccatttttgacagtgtaccgagcgcacatgatgctcatgactgttatctgcatcttggcggtagattttgaagtgtttcctagatggcagggcaagtgcgaagattttggtactagtctggtaagctttccttcagccatggtccagtgctcaccgctctacttgccgtagatggacgtgggtgtcgggtcattcgtcttttccctcggtctcgtctccacaaaatctctttctcctccacctccaactcctacgccctcctcgcccgctctcaactctcacatcattcccctcaccccgtccccgttcacttccatcctcatctcgctccgaaaatccatccccatcctcgtcctcggctttatacggttgattatggtcaagggatctgattatcctgagcatgtgacggagtacggcgtgcactggaatttcttcttcaccctcgcattggttcctgtgctcgccgtgggcattcgaccattgacgcagtggcttcgctggagtgtgcttggggtaatcatctctttgctgcatcagctgtggttaacatattatctccaatccatcgtcttctcattcggccggtcaggtatctttctagcaaacaaggaaggcttctcctctcttcctggttatctttccatatttttgatcggcttgtctattggagatcatgttttaaggctcagtttaccaccaagaagagagagggtcgtgtcagaaacaaatgaagagcatgagcagagtcattttgagagaaaaaaattggatttgattatggagttgattggatatagcttaggctggtgggcactcttaggaggctggatttgggccggcggggaggtatccaggcgtttagtaagtggacatctttggtaatattgtacctatactaatccctgcataaaggccaacgctccttatgtattttgggtagcggcatacaataccacctttctcctcggctacctcctccttacccacattattccatctcccacctcttcccaaacatcaccatcgatcttagtgcctcccttgctcgacgctatgaataaaaacggtctcgcgatatttttggcggccaacttgcttacaggactggtgaatgtgagcatgaagacaatgtatgcgccgg

SEQ ID NO: 45 - DNA, 27 bp, Artificial Sequence
  Description of Artificial SequencePCR amplification primer
gtaaaggaaggcgctagaaaagatatg

SEQ ID NO: 46 - DNA, 26 bp, Artificial Sequence
  Description of Artificial SequencePCR amplification primer
ctcatcggagtctgattcgtctagcc

SEQ ID NO: 47 - DNA, 470 bp, Cryptococcus neoformans
  amplified fragment ovelapping SEQ ID NO44
gaaggcgctagaaaagatatggtcttgtcatagcattaaatccccgccataataagctactgaattgcaatgggggattacaagtcggccaaagaggcctttgtctcggataacccaggtgcttctatctggagtatcaacgctgtcagcctggtcgcactggtatgtagctcgttctccgaggggttctgtcatttggagacgcttattaattgggatcgcaggcgacatatgctctctggatcgccttatcgccgtacatccgtcatggactcctgaacaactacctgatctgtgttcttcccctattattcggggtgaccatcttctcaacttcgcctctcgtatttacctcttttttgtccattatttccctcgctttcatcacgaaatcccaaaaatgcttcaaatctgtcagttcgcccgaaaagccaaaaggccaatggctagacgaatcagactccgatgag

SEQ ID NO: 48 - DNA, 37 bp, Artificial Sequence
  Description of Artificial Sequence3′-RACE adaptor-primer
gcccacgcgtcgactagtacttttttttttttttttt

SEQ ID NO: 49 - DNA, 29 bp, Artificial Sequence
  Description of Artificial SequencePCR amplification primer
catcttggcggtagattttgaagtgttcc

SEQ ID NO: 50 - DNA, 20 bp, Artificial Sequence
  Description of Artificial SequencePCR amplification primer
ggccacgcgtcgactagtac

SEQ ID NO: 51 - DNA, 1136 bp, Cryptococcus neoformans
  amplified fragment showing homology to S. cerevisiae GWT1
gcggtagattttgaagtgttccctagatggcagggcaagtgcgaagattttggtactagtctgatggacgtgggtgtcgggtcattcgtcttttccctcggtctcgtctccacaaaatctctttctcctccacctccaactcctacgccctcctcgcccgctctcaactctcacatcattcccctcaccccgtccccgttcacttccatcctcatctcgctccgaaaatccatccccatcctcgtcctcggctttatacggttgattatggtcaagggatctgattatcctgagcatgtgacggagtacggcgtgcactggaatttcttcttcaccctcgcattggttcctgtgctcgccgtgggcattcgaccattgacgcagtggcttcgctggagtgtgcttggggtaatcatctctttgctgcatcagctgtggttaacatattatctccaatccatcgtcttctcattcggccggtcaggtatctttctagcaaacaaggaaggcttctcctctcttcctggttatctttccatatttttgatcggcttgtctattggagatcatgttttaaggctcagtttaccaccaagaagagagagggtcgtgtcagaaacaaatgaagagcatgagcagagtcattttgagagaaaaaaattggatttgattatggagttgattggatatagcttaggctggtgggcactcttaggaggctggatttgggccggcggggaggtatccaggcgtttagccaacgctccttatgtattttgggtagcggcatacaataccacctttctcctcggctacctcctccttacccacattattccatctcccacctcttcccaaacatcaccatcgatcttagtgcctcccttgctcgacgctatgaataaaaacggtctcgcgatatttttggcggccaacttgcttacaggactggtgaatgtgagcatgaagacaatgtatgcgccggcgtggttgtcaatgggggtgttaatgttgtataccttgacaatcagttgtgtagggtggatactgaaaggacggaggatcaagatatagttaaagtgtttaccatgcaggatactgagtatctcggttcaaaaaaaaaaaaaaaaaaaaaa

SEQ ID NO: 52 - DNA, 27 bp, Artificial Sequence
  Description of Artificial SequencePCR amplification primer
gtcttgtcatagcattaaatccccgcc

SEQ ID NO: 53 - DNA, 28 bp, Artificial Sequence
  Description of Artificial SequencePCR amplification primer
gaaccgagatactcagtatcctgcatgg

SEQ ID NO: 54 - DNA, 2045 bp, Cryptococcus neoformans
  full length homologue of S. cerevisiae GWT1
gtcatagcattaaatccccgccataataagctactgaattgcaatgggggattacaagtcggccaaagaggcctttgtctcggataacccaggtgcttctatctggagtatcaacgctgtcagcctggtcgcactggtatgtagctcgttctccgaggggttctgtcatttggagacgcttattaattgggatcgcaggcgacatatgctctctggatcgccttatcgccgtacatccgtcatggactcctgaacaactacctgatctgtgttcttcccctattattcggggtgaccatcttctcaacttcgcctctcgtatttacctcttttttgtccattatttccctcgctttcatcacgaaatcccaaaaatgcttcaaatctgtcagttcgcccgaaaagccaaaaggccaatggctagacgaatcagactccgatgaggaaccagcggaacctgcttctgcagctggatctgcagcagtctcaccagtaaagcttctaccttcccaagtggcgttcgcttcgggatccctattatctcccgatccgacaacatcccccatgtcgccaagtagttcttcagcttcaggacatgaagaccctttggggattatgggcgttaacagacggaggtcgctattagaaggagtttcgcttgatgttccgtcacatatcgactccaaggtcagaatatctcctgttccctacttgaggctcaaaaagtctagggcaacgaaggcgcaatgggtgaaagaaaagggaagattaccatttttgacagtgtaccgagcgcacatgatgctcatgactgttatctgcatcttggcggtagattttgaagtgtttcctagatggcagggcaagtgcgaagattttggtactagtctggtaagctttccttcagccatggtccagtgctcaccgctctacttgccgtagatggacgtgggtgtcgggtcattcgtcttttccctcggtctcgtctccacaaaatctctttctcctccacctccaactcctacgccctcctcgcccgctctcaactctcacatcattcccctcaccccgtccccgttcacttccatcctcatctcgctccgaaaatccatccccatcctcgtcctcggctttatacggttgattatggtcaagggatctgattatcctgagcatgtgacggagtacggcgtgcactggaatttcttcttcaccctcgcattggttcctgtgctcgccgtgggcattcgaccattgacgcagtggcttcgctggagtgtgcttggggtaatcatctctttgctgcatcagctgtggttaacatattatctccaatccatcgtcttctcattcggccggtcaggtatctttctagcaaacaaggaaggcttctcctctcttcctggttatctttccatatttttgatcggcttgtctattggagatcatgttttaaggctcagtttaccaccaagaagagagagggtcgtgtcagaaacaaatgaagagcatgagcagagtcattttgagagaaaaaaattggatttgattatggagttgattggatatagcttaggctggtgggcactcttaggaggctggatttgggccggcggggaggtatccaggcgtttagtaagtggacatctttggtaatattgtacctatactaatccctgcataaaggccaacgctccttatgtattttgggtagcggcatacaataccacctttctcctcggctacctcctccttacccacattattccatctcccacctcttcccaaacatcaccatcgatcttagtgcctcccttgctcgacgctatgaataaaaacggtctcgcgatatttttggcggccaacttgcttacaggactggtgaatgtgagcatgaagacaatgtatgcgccggcgtggttgtcaatgggggtgttaatgttgtataccttgacaatcagttgtgtagggtggatactgaaaggacggaggatcaagatatagttaaagtgtttaccatgcaggatactgagtatctcggttca

SEQ ID NO: 55 - DNA, 23 bp, Artificial Sequence
  Description of Artificial SequencePCR amplification primer predicted to be junction between exons
cagcctggtcgcactggcgacat

SEQ ID NO: 56 - DNA, 25 bp, Artificial Sequence
  Description of Artificial SequencePCR amplification primer predicted to be junction between exons
cataaggagcgttggctaaacgcct

SEQ ID NO: 57 - DNA, 1418 bp, Cryptococcus neoformans
  amplified fragment showing homology to S. cerevisiae GWT1
cagcctggtcgcactggcgacatatgctctctggatcgccttatcgccgtacatccgtcatggactcctgaacaactacctgatctgtgttcttcccctattattcggggtgaccatcttctcaacttcgcctctcgtatttacctcttttttgtccattatttccctcgctttcatcacgaaatcccaaaaatgcttcaaatctgtcagttcgcccgaaaagccaaaaggccaatggctagacgaatcagactccgatgaggaaccagcggaacctgcttctgcagctggatctgcagcagtctcaccagtaaagcttctaccttcccaagtggcgttcgcttcgggatccctattatctcccgatccgacaacatcccccatgtcgccaagtagttcttcagcttcaggacatgaagaccctttggggattatgggcgttaacagacggaggtcgctattagaaggagtttcgcttgatgttccgtcacatatcgactccaaggtcagaatatctcctgttccctacttgaggctcaaaaagtctagggcaacgaaggcgcaatgggtgaaagaaaagggaagattaccatttttgacagtgtaccgagcgcacatgatgctcatgactgttatctgcatcttggcggtagattttgaagtgtttcctagatggcagggcaagtgcgaagattttggtactagtctgatggacgtgggtgtcgggtcattcgtcttttccctcggtctcgtctccacaaaatctctttctcctccacctccaactcctacgccctcctcgcccgctctcaactctcacatcattcccctcaccccgtccccgttcacttccatcctcatctcgctccgaaaatccatccccatcctcgtcctcggctttatacggttgattatggtcaagggatctgattatcctgagcatgtgacggagtacggcgtgcactggaatttcttcttcaccctcgcattggttcctgtgctcgccgtgggcattcgaccattgacgcagtggcttcgctggagtgtgcttggggtaatcatctctttgctgcatcagctgtggttaacatattatctccaatccatcgtcttctcattcggccggtcaggtatctttctagcaaacaaggaaggcttctcctctcttcctggttatctttccatatttttgatcggcttgtctattggagatcatgttttaaggctcagtttaccaccaagaagagagagggtcgtgtcagaaacaaatgaagagcatgagcagagtcattttgagagaaaaaaattggatttgattatggagttgattggatatagcttaggctggtgggcactcttaggaggctggatttgggccggcggggaggtatccaggcgtttagccaacgctccttatg

SEQ ID NO: 58 - DNA, 1797 bp, Cryptococcus neoformans
  CDS (1)..(1797): homologue of S. cerevisiae GWT1
atgggggattacaagtcggccaaagaggcctttgtctcggataacccaggtgcttctatctggagtatcaacgctgtcagcctggtcgcactggcgacatatgctctctggatcgccttatcgccgtacatccgtcatggactcctgaacaactacctgatctgtgttcttcccctattattcggggtgaccatcttctcaacttcgcctctcgtatttacctcttttttgtccattatttccctcgctttcatcacgaaatcccaaaaatgcttcaaatctgtcagttcgcccgaaaagccaaaaggccaatggctagacgaatcagactccgatgaggaaccagcggaacctgcttctgcagctggatctgcagcagtctcaccagtaaagcttctaccttcccaagtggcgttcgcttcgggatccctattatctcccgatccgacaacatcccccatgtcgccaagtagttcttcagcttcaggacatgaagaccctttggggattatgggcgttaacagacggaggtcgctattagaaggagtttcgcttgatgttccgtcacatatcgactccaaggtcagaatatctcctgttccctacttgaggctcaaaaagtctagggcaacgaaggcgcaatgggtgaaagaaaagggaagattaccatttttgacagtgtaccgagcgcacatgatgctcatgactgttatctgcatcttggcggtagattttgaagtgtttcctagatggcagggcaagtgcgaagattttggtactagtctgatggacgtgggtgtcgggtcattcgtcttttccctcggtctcgtctccacaaaatctctttctcctccacctccaactcctacgccctcctcgcccgctctcaactctcacatcattcccctcaccccgtccccgttcacttccatcctcatctcgctccgaaaatccatccccatcctcgtcctcggctttatacggttgattatggtcaagggatctgattatcctgagcatgtgacggagtacggcgtgcactggaatttcttcttcaccctcgcattggttcctgtgctcgccgtgggcattcgaccattgacgcagtggcttcgctggagtgtgcttggggtaatcatctctttgctgcatcagctgtggttaacatattatctccaatccatcgtcttctcattcggccggtcaggtatctttctagcaaacaaggaaggcttctcctctcttcctggttatctttccatatttttgatcggcttgtctattggagatcatgttttaaggctcagtttaccaccaagaagagagagggtcgtgtcagaaacaaatgaagagcatgagcagagtcattttgagagaaaaaaattggatttgattatggagttgattggatatagcttaggctggtgggcactcttaggaggctggatttgggccggcggggaggtatccaggcgtttagccaacgctccttatgtattttgggtagcggcatacaataccacctttctcctcggctacctcctccttacccacattattccatctcccacctcttcccaaacatcaccatcgatcttagtgcctcccttgctcgacgctatgaataaaaacggtctcgcgatatttttggcggccaacttgcttacaggactggtgaatgtgagcatgaagacaatgtatgcgccggcgtggttgtcaatgggggtgttaatgttgtataccttgacaatcagttgtgtagggtggatactgaaaggacggaggatcaagatatag

SEQ ID NO: 59 - protein, 598 aa, Cryptococcus neoformans
  homologue of S. cerevisiae GWT1
MGDYKSAKEAFVSDNPGASIWSINAVSLVALATYALWIALSPYIRHGLLNNYLICVLPLLFGVTIFSTSPLVFTSFLSIISLAFITKSQKCFKSVSSPEKPKGQWLDESDSDEEPAEPASAAGSAAVSPVKLLPSQVAFASGSLLSPDPTTSPMSPSSSSASGHEDPLGIMGVNRRRSLLEGVSLDVPSHIDSKVRISPVPYLRLKKSRATKAQWVKEKGRLPFLTVYRAHMMLMTVICILAVDFEVFPRWQGKCEDFGTSLMDVGVGSFVFSLGLVSTKSLSPPPPTPTPSSPALNSHIIPLTPSPFTSILISLRKSIPILVLGFIRLIMVKGSDYPEHVTEYGVHWNFFFTLALVPVLAVGIRPLTQWLRWSVLGVIISLLHQLWLTYYLQSIVFSFGRSGIFLANKEGFSSLPGYLSIFLIGLSIGDHVLRLSLPPRRERVVSETNEEHEQSHFERKKLDLIMELIGYSLGWWALLGGWIWAGGEVSRRLANAPYVFWVAAYNTTFLLGYLLLTHIIPSPTSSQTSPSILVPPLLDAMNKNGLAIFLAANLLTGLVNVSMKTMYAPAWLSMGVLMLYTLTISCVGWILKGRRIKI

SEQ ID NO: 60 - DNA, 30 bp, Artificial Sequence
  Description of Artificial SequenceR1 and R5 mutant GWT1 gene primer
aaagaattcatggcaacagtacatcagaag

SEQ ID NO: 61 - DNA, 20 bp, Artificial Sequence
  Description of Artificial SequenceR1 and R5 mutant GWT1 gene primer
gggcactgttgaaaaaccta

SEQ ID NO: 62 - DNA, 1428 bp, Saccharomyces cerevisiae
  promoter (1)..(1428): GWT1 promoter region
gttgttcaaaatgggggtaaaattgagacgtcttacttgagcggcatttacgatcattcttattacatcattccaagtaataaagctcttgactccttcaatgatttacctgagattatagatgataatgatggtatagttacagaatttttcattgaacgctgcttgtattatcaaaaattactacacccaatagatttatggtcaaaacccttcctcagcacaatagagtttcaagtttcgtcttcttcaaagttattgcatcatgaattttcttcttccccttttctgaatgttactatcactggattctctggcgtagagctgttacatctgactaaagtattaaatcttctaaaaccaatgggcatcaattatgtagaatacctcaataaatccactgacattctgctaatcaacttagcagctttacccagtatcccgaaaacccatccgttatggtcgaatgaatttagcgatctttttactcagttttgcattaataacaataatgatgatcctggtgataataacagaaaagattttcaaaataattcaatcttgagaaattcgatgaaaaggaaaattgaatatatcaagaaattccactccataccggtagttactccagcatttatttttaaattattgtccgctgcatctggagaaaataatgaaatctttttaaacaatatcaagtggtgtattatctgcccaagaggacacaaggacgattttaaatgtaagataaaaaaaccatactataccagcattagttcagaaaaaaagtaccaaaacaatgatccaaaaatcgacaaaactattcttttgaaaagaaacaattcctcattatcggagcactctatgaaagataccaaaaacgaattattgcagaaaattagagaaactgattctggaagaaaaaagcgtagtgtctcatcgagtatcatggatgtttcttcagagagacaaatgccggatacgaaaaggatcaagttggagtcactgccaaaaaatttcgttcctaaacaaattaaacgaaccacgagttggggcacaataatgtcagaaaatgtgcctacagagcagccgactgcaatttctaatccagaagagatcccaagaactgaggaagtttcacatactcaagttacctatggctccattcaagataagaaacgtactgcctctttagaaaaacctatgagacgacagacaagaaatcagacaaaggaattagattcttgaaatgtagtccgtaattttataagatattcatttacatacgccatctacagcattattcaaatctactcatctatatgtattaccgttttgtatgataatactttccatgacatgctcgcgtgaaaaaacagcatgagaaaaagaggatcgcaataagaagacacgtaaatatctaaataa

SEQ ID NO: 63 - DNA, 133 bp, Saccharomyces cerevisiae
  terminator (1)..(133): GWT1 terminator region
taacacaccatccacatttccatgtagttcgtatacaaaccctaccagtaaaataaaattaactcctatgtgctttaaataaaaattataaaccgcctccaatagttgacgtagtcaggcatgaaagtgctac

SEQ ID NO: 64 - protein, 31 aa, Saccharomyces cerevisiae
  highly conserved GWT1 F-domain
ILAVDFPIFPRRFAKVETWGTSLMDLGVGSF

SEQ ID NO: 65 - protein, 31 aa, Candida albicans
  highly conserved GWT1 F-domain
ILAVDFPIFPRRFAKVETWGTSMMDLGVGSF

SEQ ID NO: 66 - protein, 31 aa, Schizosaccharomyces pombe
  highly conserved GWT1 F-domain
ILAVDFTLFPRRYAKVETWGTSLMDLGVGSF

SEQ ID NO: 67 - protein, 17 aa, Saccharomyces cerevisiae
  highly conserved GWT1 R-domain
YQEHVTEYGVHWNFFIT

SEQ ID NO: 68 - protein, 17 aa, Candida albicans
  highly conserved GWT1 R-domain
YQEHETEYGIHWNFFFT

SEQ ID NO: 69 - protein, 17 aa, Schizosaccharomyces pombe
  highly conserved GWT1 R-domain
YQEHVSEYGMHWNFFFT

(19) United States
(12) Patent Application Publication
(10) Pub. No.: US 2004/0038239 A1
(43) Pub. Date: Feb. 26, 2004

(54) Fungal cell wall synthesis gene

(76) Inventors: Kappei Tsukahara, Ibaraki (JP); Katsura Hata, Ibaraki (JP); Koji Sagane, Ibaraki (JP); Kazutaka Nakamoto, Ibaraki (JP); Mamiko Tsuchiya, Ibaraki (JP); Naoaki Watanabe, Ibaraki (JP); Fuminori Oba, Saitama (JP); Itaru Tsukada, Ibaraki (JP); Norihiro Ueda, Ibaraki (JP); Keigo Tanaka, Ibaraki (JP); Junko Kai, Ibaraki (JP)

Correspondence Address: Kevin L Bastin, Townsend and Townsend and Crew, Two Embarcadero Center 8th Floor, San Francisco, CA, 94111, US

(21) Appl. No.: 10/332,340

(22) PCT Filed: Jul. 6, 2001
(86) PCT No.: PCT/JP01/05899

(30) Foreign Application Priority Data
Jul. 7, 2000 (JP) 2000-206968

(51) Int. Cl. C12Q 1/68; C07H 21/04; C12N 9/06; C12N 1/18; C12N 15/74; C12N 1/16

(52) U.S. Cl. 435/6; 435/69.1; 435/191; 435/254.2; 435/320.1; 536/23.2

(57) ABSTRACT

A reporter system reflecting the transport process that transports GPI-anchored proteins to the cell wall was constructed and compounds inhibiting this process were discovered. Further, genes conferring resistance to the above compounds were identified and methods of screening for compounds that inhibit the activity of the proteins encoded by these genes were developed.

Therefore, through the novel compounds, the present invention showed that antifungal agents having a novel mechanism, i.e. inhibiting the process that transports GPI-anchored proteins to the cell wall, could be achieved.

### TECHNICAL FIELD

The present invention relates to DNAs encoding proteins participating in fungal cell wall synthesis, proteins encoded by the DNAs, methods for examining whether or not a certain compound has an influence on the transport process involved in the transport of GPI-anchored proteins to the cell wall, and antifungal agents having an influence on the transport process involved in the transport of GPI-anchored proteins to the cell wall.

### BACKGROUND ART

In recent years, management of opportunistic infections are gaining importance more than ever due to an increase in the number of elderly people and immunocompromised patients as a result of advanced chemotherapies, etc. Deep-seated mycosis due to Candida, Aspergillus, Cryptococcus, and such, account for a portion of such opportunistic infections, and the proportion is increasing year after year. The fact that opportunistic infections by many avirulent bacteria occur one after another, shows that the problem of infectious diseases will not end as long as there are underlying diseases that diminish the immune functions of patients. Although new strategies for infectious diseases control, including the problem of resistant bacteria, will be one of the crucial issues in the soon-to-come aged society, extremely few effective therapeutic agents exist at present.

Up to now, therapeutic agents for fungal infections were developed based mainly on the strategy of creating novel compounds by chemically modifying known structure. However, due to problems such as the emergence of resistant bacteria, the development of new drugs based on new mechanisms is eagerly anticipated.

Considering such circumstances, the inventors focused on a novel approach in the area of antifungal agents in which the variety of therapeutic agents is still insufficient. Namely, the present inventors concentrated on influencing the onset, progress, and persistence of infections by preventing pathogens from showing pathogenicity. In order to avoid the establishment and progress of infection, the inventors thought that the most effective way would be to inhibit the adhesion onto the host, which is the first step in the establishment of infection, and the subsequent progression of colonization. In addition, a new unprecedented approach, namely, the inhibition of the expression of adhesion factors themselves, was also carried out.

In order to inhibit the expression of adhesion factors, the present inventors directed their attention to the hypothesis that cell wall glycoproteins such as adhesion factors are first GPI (Glycosylphosphatidylinositol)-anchored to the cell membrane, and then transported to the cell wall (FIG. 1). To date, 30 or more cell wall glycoproteins including adhesion ligands have been found to be transported via GPI-anchoring (referred to as GPI-anchored proteins). Hence, it was thought that if this transport step is inhibited, it may be quite possible to inhibit the expression of adhesion factors and major cell wall-constituting proteins at the cell wall (Hamada K et al, Mol. Gen. Genet., 258: 53-59, 1998). GPI-anchored proteins have been reported to be present in Candida, which is a pathogenic fungi (Kapteyn J C et al, Eur. J. Cell Biol., 65:402-407, 1994).

The inventors initiated their research believing that novel antifungal agents that inhibit cell wall synthesis can be produced by inhibiting the process that transports GPI-anchored proteins existing in the cell membrane of a fungus to the cell wall.

### DISCLOSURE OF THE INVENTION

An objective of this invention is to develop antifungal agents showing effects against the onset, progress, and persistence of infections by inhibiting the expression of cell wall glycoproteins, inhibiting the cell wall assembly and also adhesion onto cells, and preventing pathogens from showing pathogenicity.

In order to screen for compounds that inhibit the process that transports GPI-anchored proteins to the cell wall, the present inventors produced a reporter system that uses a fusion protein comprising a reporter enzyme and a transport signal existing in the C-terminus of one of the GPI-anchored proteins, CWP2 (Van Der Vaat J M et al, J. Bacteriol., 177:3104-3110,1995).

When a DNA comprising a secretion signal gene + reporter enzyme gene + CWP2 C-terminus gene (present or absent) was constructed, and the fusion protein was expressed in Saccharomyces cerevisiae (hereinafter, referred to as S. cerevisiae), it was demonstrated that activity of the reporter enzyme is detected in the cell wall when the CWP2 C-terminus is present, and in the culture supernatant when the CWP2 C-terminus is absent. Accordingly, it was predicted that if the process that transports GPI-anchored proteins to the cell wall is inhibited by a test sample, the activity of the reporter enzyme in the cell wall will be diminished, or the activity of the reporter enzyme will be found in the culture supernatant. Thus was initiated the screening for compounds that inhibit the process that transports GPI-anchored proteins to the cell wall using this reporter system.

From the screening using this reporter system, several compounds that inhibit the process that transports GPI-anchored proteins to the cell wall were discovered. A representative example is the compound shown in formula (Ia).

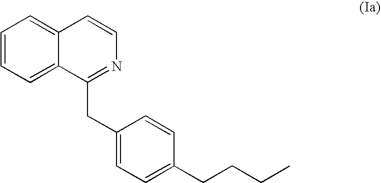

The compound shown in the aforementioned formula (Ia) (hereinafter abbreviated as “compound (Ia)”) inhibits the growth of S. cerevisiae and Candida albicans (hereinafter, referred to as C. albicans), and C. albicans cultured in the presence of the aforementioned compound (Ia) shows a weak ability to adhere onto cells. Thus, the aforementioned compound (Ia) was confirmed to suit the initial objectives of the invention, which was to find a compound that inhibits the adhesion of fungi, due to suppressing the expression of the fungal adhesins, based on the inhibition of transport system of GPI-anchored proteins to the cell wall. Furthermore, observations using a transmission electron microscope confirmed that C. albicans cultured in the presence of the aforementioned compound (Ia) has an abnormality in its cell wall synthesis.

Using the aforementioned compound (Ia), the present inventors proved that antifungal agents based on the mechanism that inhibits the process that transports GPI-anchored proteins to the cell wall, could be achieved.

Furthermore, to specify the target protein on which the aforementioned compound (Ia) acts, the present inventors searched for genes that confer resistance to the aforementioned compound (Ia).

A plasmid library of the S. cerevisiae gene was introduced into S. cerevisiae, and by overexpression, plasmids were collected that showed resistance to the abovementioned compound (Ia) The resistant gene was then cloned, the nucleotide sequence was determined, and the gene was named GWT1 (SEQ ID NO: 1). In S. cerevisiae overexpressing the GWT1 gene product, the aforementioned reporter enzyme that has the C-terminus of a GPI-anchored protein was transported to the cell wall, even in the presence of the aforementioned compound (Ia). Furthermore, observations under a transmission electron microscope confirmed that the cell wall is normal even in the presence of the aforementioned compound (Ia).

Moreover, when point mutations were randomly introduced to the genomic DNA of S. cerevisiae, and mutant strains R1 and R5 showing specific resistance to the aforementioned compound (Ia) were isolated, point mutations involving changes of the 405th codon of the GWT1 gene from GTC to ATC in the R1 mutant strain, and the 140th codon from GGG to AGG in the R5 mutant strain were discovered. Since resistance to the aforementioned compound (Ia) was seen when these mutant GWT1 genes were introduced to a GWT1 gene-disrupted strain, resistance to this compound was found to be explainable by the GWT1 gene alone. Therefore, this suggested that the aforementioned compound (Ia) directly acts on the GWT1 gene product to inhibit the function of the GWT1 protein.

By similar methods, the resistant genes of C. albicans (SEQ ID NOS: 3 and 5) were cloned, the nucleotide sequences were determined, and the genes were named CaGWT1.

Furthermore, a database homology search using GWT1, revealed a homologue (SEQ ID NO: 27) of Schizosaccharomyces pombe (hereinafter, referred to as S. pombe). Furthermore, PCR with primers based on the sequence of the highly conserved region in the proteins encoded by the GWT1 genes of S. cerevisiae, S. pombe, and C. albicans, yielded homologues (SEQ ID NOS: 39 and 41) of Aspergillus fumigatus (hereinafter, referred to as A. fumigatus). Furthermore, by performing PCR based on the sequence discovered from a database homology search with GWT1, revealed homologues (SEQ ID NOS: 54 and 58) of Cryptococcus neoformans (hereinafter, referred to as C. neoformans).

More specifically, this invention relates to the following.

1. A DNA that encodes a protein having an activity to confer resistance to the compound shown in formula (Ia) on a fungus when the DNA is overexpressed in the fungus, wherein the DNA is selected from the group consisting of:

(a) A DNA encoding a protein comprising the amino acid sequence of SEQ ID NO: 2, 4, 6, 28, 40, or 59.

(b) A DNA comprising the nucleotide sequence of SEQ ID NO: 1, 3, 5, 27, 39, 41, 54, or 58.

(c) A DNA that hybridizes under stringent conditions to a DNA comprising the nucleotide sequence of SEQ ID NO: 1, 3, 5, 27, 39, 41, 54, or 58.

(d) A DNA encoding a protein comprising the amino acid sequence of SEQ ID NO: 2, 4, 6, 28, 40, or 59, wherein one or more amino acids have been added, deleted, substituted, and/or inserted.

(e) A DNA that is amplified using SEQ ID NOS: 29 and 31 or SEQ ID NOS: 29 and 30 as primers.

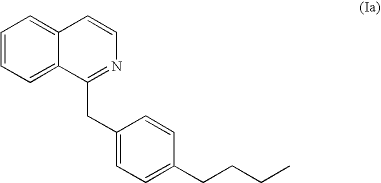

2. A DNA that encodes a protein having an activity to decrease the amount of a GPI-anchored protein in the cell wall of a fungus due to a defect in the function of the DNA, wherein the DNA is selected from the group consisting of:

(a) A DNA encoding a protein comprising the amino acid sequence of SEQ ID NO: 2, 4, 6, 28, 40, or 59,

(b) A DNA comprising the nucleotide sequence of SEQ ID NO: 1, 3, 5, 27, 39, 41, 54, or 58,

(c) A DNA that hybridizes under stringent conditions to a DNA comprising the nucleotide sequence of SEQ ID NO: 1, 3, 5, 27, 39, 41, 54, or 58,

(d) A DNA encoding a protein comprising the amino acid sequence of SEQ ID NO: 2, 4, 6, 28, 40, or 59, wherein one or more amino acids have been added, deleted, substituted, and/or inserted, and

(e) A DNA that is amplified using SEQ ID NOS: 29 and 31 or SEQ ID NOS: 29 and 30 as primers,

and wherein, “stringent conditions” refer to: for example, hybridization in 4×SSC at 65° C., then washing in 0.1×SSC for 1 hour at 65° C.; or in a different method, “stringent conditions” are 4×SSC at 42° C. in 50% formamide; or, hybridization in PerfectHyb™ (TOYOBO) solution for 2.5 hours at 65° C., then washing in (i) 2×SSC, 0.05% SDS solution at 25° C. for 5 minutes, (ii) 2×SSC, 0.05% SDS solution at 25° C. for 15 minutes, and (iii) 0.1×SSC, 0.1% SDS solution at 50° C. for 20 minutes;

a “defect in the DNA function” can occur, when the functional gene product of the DNA is not expressed or when the expression is diminished, for example by inserting a DNA that is irrelevant to the coding region of the DNA, for example a selection marker, using the homologous recombination technique;

and a decrease in the protein derived from the GPI-anchored protein in the fungal cell wall is quantified by using any one of the following methods alone or in combination: (i) a reporter system reflecting the process that transports GPI-anchored proteins to the cell wall, (ii) an ELISA that quantifies a GPI-anchored protein in the cell wall, (iii) measuring the activity of a GPI-anchored protein, such as adhesion onto animal cells, or (4) observing the flocculent, fibrous structure of the outermost layer of the fungal cell by a transmission electron microscope.

3. A protein encoded by the DNA of 1 or 2.

4. A vector into which the DNA of 1 or 2 has been inserted.

5. A transformant harboring the DNA of 1 or 2, or the vector of 4.

6. The transformant of 5 which is a fungus that overexpresses the protein of 3.

7. A fungus, wherein the function of the protein of 3 is defective.

8. A method for producing the protein of 3, which comprises the steps of culturing the transformant of 5, and collecting the expressed protein from the transformant, or from the culture supernatant thereof.

9. An antibody that binds to the protein of 3.

10. A method of screening for a compound having an antifungal action, wherein the method comprises the steps of:

(a) contacting a test sample with the protein of 3;

(b) detecting the binding activity between the protein and the test sample; and

(c) selecting a compound having an activity to bind to the protein.

11. A method of screening for a compound that has an antifungal action, which comprises the steps of:

(a) contacting a test sample with a fungus that is overexpressing the protein of 3;

(b) detecting the amount of transport of a GPI-anchored protein to the cell wall in the fungus; and

(c) selecting a compound that diminishes the amount of transport of the GPI-anchored protein to the cell wall detected in step (b) as compared to the amount of transport detected when the test sample was contacted with a fungus that is not overexpressing the protein of 3,

wherein, a decrease in the amount of GPI-anchored protein transported to the cell wall that results due to the test sample can be detected, for example, by detecting a decrease in growth rate, swelling, or temperature sensitivity of the cell, or by detecting a decrease of the protein derived from the GPI-anchored protein in the cell wall, but preferably, by detecting a decrease in the protein derived from the GPI-anchored protein at the cell wall;

and wherein a decrease of the protein derived from the GPI-anchored protein is quantified by using any one of the following methods alone or in combination: (i) a reporter system reflecting the process that transports GPI-anchored proteins to the cell wall, (ii) an ELISA that quantifies one type of the GPI-anchored protein in the cell wall, (iii) measuring the activity of a GPI-anchored protein such as adhesion to animal cells, and (iv) observing the flocculent, fibrous structure of the outermost layer of a fungal cell by a transmission electron microscope.

12. A compound having an antifungal action that is isolated by the screening of 10 or 11.

13. An antifungal agent, comprising as an active ingredient a compound that inhibits the transport of GPI-anchored proteins to the cell wall of a fungus.

14. An antifungal agent, comprising as an active ingredient the antibody of 9 or the compound of 12.

15. The antifungal agent of 13, comprising as an active ingredient the compound represented by the general formula (I), a salt thereof, or a hydrate thereof, wherein in formula (I):

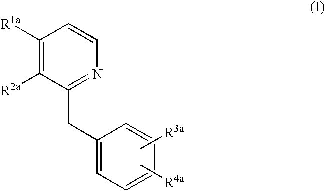

[R^{1a} and R^{2a} are identical to or different from each other and denote individually a hydrogen atom, halogen atom, hydroxyl group, nitro group, cyano group, trifluoromethyl group, trifluoromethoxy group, a substituted or unsubstituted C₁₋₆ alkyl group, C₂₋₆ alkenyl group, C₂₋₆ alkynyl group, a substituted or unsubstituted C₁₋₆ alkoxy group, or a group represented by the formula:

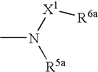

(wherein X¹ stands for a single bond, carbonyl group, or a group represented by the formula —S(O)₂—;

R^{5a} and R^{6a} are identical to or different from each other and denote a hydrogen atom or a substituted or unsubstituted C₁₋₆ alkyl group). Furthermore, R^{1a} and R^{2a} may together form a condensed ring selected from the group consisting of a substituted or unsubstituted benzene ring, a substituted or unsubstituted pyridine ring, a substituted or unsubstituted pyrrole ring, a substituted or unsubstituted thiophene ring, a substituted or unsubstituted furan ring, a substituted or unsubstituted pyridazine ring, a substituted or unsubstituted pyrimidine ring, a substituted or unsubstituted pyrazine ring, a substituted or unsubstituted imidazole ring, a substituted or unsubstituted oxazole ring, a substituted or unsubstituted thiazole ring, a substituted or unsubstituted pyrazole ring, a substituted or unsubstituted isoxazole ring, a substituted or unsubstituted isothiazole ring, a substituted or unsubstituted cyclohexane ring, and a substituted or unsubstituted cyclopentane ring;

R^{3a} and R^{4a} are identical to or different from each other and denote individually a hydrogen atom, halogen atom, hydroxyl group, nitro group, cyano group, carboxyl group, formyl group, hydroxyimino group, trifluoromethyl group, trifluoromethoxy group, C₁₋₆ alkyl group, C₁₋₆ alkoxy group, C₂₋₆ alkenyl group, C₂₋₆ alkynyl group, a group represented by the formula —C(O)NR^{7a}R^{7b} (wherein R^{7a} and R^{7b} are identical to or different from each other and denote individually a hydrogen atom, or a C₁₋₆ alkyl group), the formula —CO₂R^{7a} (wherein R^{7a} has the same meaning as defined above), the formula —S(O)ₙR^{7a} (wherein n stands for an integer of 0 to 2 and R^{7a} has the same meaning as defined above), the formula —S(O)₂NR^{7a}R^{7b} (wherein R^{7a} and R^{7b} have the same meaning as defined above), a group of the formula

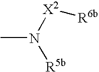

(wherein X² denotes a single bond, carbonyl group, or a group of the formula —S(O)₂—;

R^{5b} and R^{6b} are identical to or different from each other, and denote a hydrogen atom, a substituted or unsubstituted C₁₋₆ alkyl group, or a substituted or unsubstituted C₆₋₁₄ aryl group), or a group of the formula —Z¹—Z²

(wherein Z¹ denotes a single bond, oxygen atom, vinylene group, or ethynylene group;

Z² denotes a single bond, or a C₁₋₆ alkyl group substituted or unsubstituted with 0 to 4 substituents). R^{3a} and R^{4a} may together stand for a methylenedioxy group or 1,2-ethylenedioxy group, alternatively, R^{3a} and R^{4a} may together stand for the formation of a condensed ring selected from a group consisting of a substituted or unsubstituted benzene ring, substituted or unsubstituted pyridine ring, substituted or unsubstituted pyrrole ring, substituted or unsubstituted thiophene ring, substituted or unsubstituted furan ring, substituted or unsubstituted pyridazine ring, substituted or unsubstituted pyrimidine ring, substituted or unsubstituted pyrazine ring, substituted or unsubstituted imidazole ring, substituted or unsubstituted oxazole ring, substituted or unsubstituted thiazole ring, substituted or unsubstituted pyrazole ring, substituted or unsubstituted isoxazole ring, substituted or unsubstituted isothiazole ring, substituted or unsubstituted cyclohexane ring, and substituted or unsubstituted cyclopentane ring, except in cases where both R^{1a} and R^{2a} stand for hydrogen atoms.]

16. The aforementioned antifungal agent of 13, comprising as the active ingredient compound (Ia) of the formula:

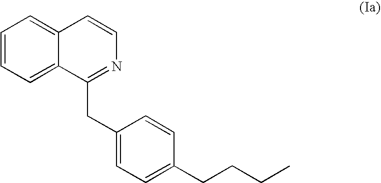

17. A compound represented by the general formula (II), a salt or a hydrate thereof, wherein in formula (II),

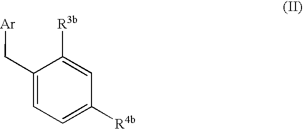

[Ar stands for a substituent selected from a group consisting of the formulae (IIIa) to (IIIf):

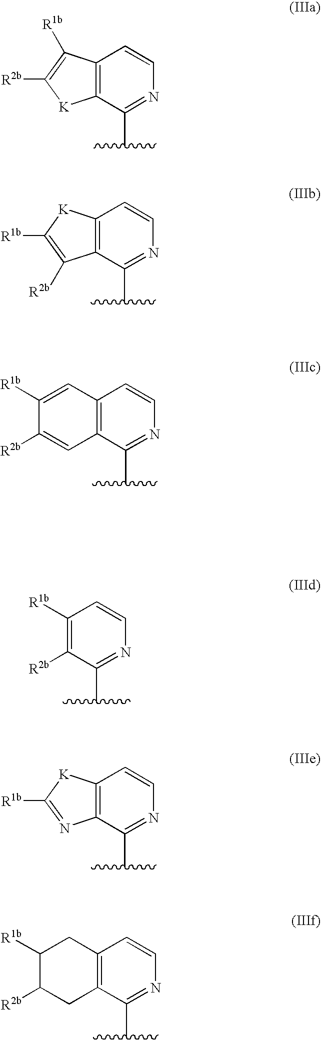

(wherein K denotes a sulfur atom, oxygen atom, or a group represented by the formula —NH—;

R^{1b} and R^{2b} are identical to or different from each other and denote individually a hydrogen atom, halogen atom, hydroxyl group, nitro group, cyano group, trifluoromethyl group, trifluoromethoxy group, a group represented by the formula

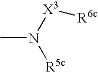

(wherein X³ denotes a single bond, carbonyl group, or a group represented by the formula —S(O)₂—;

R^{5c} and R^{6c} are identical to or different from each other and denote a hydrogen atom, or a substituted or unsubstituted C₁₋₆ alkyl group), or a group represented by the formula —X⁴—R^{8a} (wherein X⁴ denotes a single bond, oxygen atom, or sulfur atom; R^{8a} denotes a C₁₋₆ alkyl group, C₂₋₆ alkenyl group, C₂₋₆ alkynyl group, C₃₋₈ cycloalkyl group, or C₃₋₈ cycloalkenyl group) Alternatively, R^{1b} and R^{2b} may together form a methylenedioxy group, or a 1,2-ethylenedioxy group.);

R^{3b} and R^{4b} are identical to or different from each other and denote individually a hydrogen atom, halogen atom, hydroxyl group, nitro group, cyano group, carboxyl group, formyl group, hydroxyimino group, trifluoromethyl group, trifluoromethoxy group, C₁₋₆ alkyl group, C₁₋₆ alkoxy group, C₂₋₆ alkenyl group, C₂₋₆ alkynyl group, or a group represented by the formula —Z^{1b}—Z^{2b}

(wherein Z^{1b} denotes a single bond, vinylene group, or ethynylene group;

Z^{2b} denotes a single bond, or a C₁₋₆ alkyl group that is substituted or unsubstituted with 0 to 4 substituents); except in cases where (1) Ar stands for the aforementioned formula (IIId) wherein R^{1b} and R^{2b} are both hydrogen atoms, (2) at least one of R^{3b} or R^{4b} denotes a hydrogen atom and the other is a hydrogen atom, methoxy group, hydroxyl group, methyl group, benzyloxy group, or a halogen atom, and Ar stands for the aforementioned formula (IIIc) wherein R^{1b} and R^{2b} both denote hydrogen atoms or methoxy groups, (3) at least one of R^{3b} or R^{4b} denotes a hydrogen atom and the other is a hydrogen atom, hydroxyl group, methoxy group, or benzyloxy group, and Ar stands for the aforementioned formula (IIIc) wherein R^{1b} and R^{2b} both denote hydroxyl groups or benzyloxy groups, or (4) Ar stands for the aforementioned formula (IIId) wherein R^{1b} is a hydrogen atom and R^{2b} is a formyl group, hydroxymethyl group, or methoxycarbonyl group.]

18. The compound of 17, or a salt or hydrate thereof, wherein Ar stands for the formula:

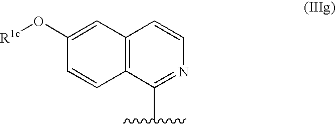

(wherein R^{1c} denotes a hydrogen atom, a substituted or unsubstituted C₁₋₆ alkyl group, or a benzyl group), and excluding the case when R^{3b} denotes a hydrogen atom.

19. A compound represented by the general formula (IIIc2), or a salt or hydrate thereof, wherein in formula (IIIc2),

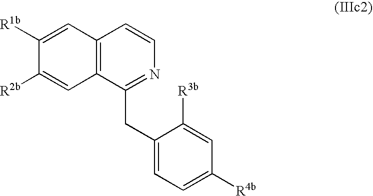

[R^{1b} and R^{2b} have the same meaning as defined above, except in cases wherein (1) R^{1b} denotes a group represented by the formula R^{1c}—O— (wherein R^{1c} has the same meaning as defined above), R^{2b} is a hydrogen atom, and R^{3b} denotes a hydrogen atom, (2) at least one of R^{3b} or R^{4b} denotes a hydrogen atom, and the other is a hydrogen atom, methoxy group, hydroxyl group, methyl group, benzyloxy group, or a halogen atom, and R^{1b} and R^{2b} both denote hydrogen atoms or methoxy groups, or (3) at least one of R^{3b} or R^{4b} denotes a hydrogen atom, and the other is a hydrogen atom, hydroxyl group, methoxy group, or benzyloxy group, and R^{1b} and R^{2b} both denote hydroxyl groups or benzyloxy groups]

20. The antifungal agent of 17, having an antifungal action.

21. The antifungal agent of 15, wherein at least one of R^{3a} and R^{4a} denotes a group represented by the formula —C(O)NR^{7a}R^{7b} (wherein R^{7a} and R^{7b} have the same meaning as defined above), the formula —CO₂R^{7a} (wherein R^{7a} has the same meaning as defined above), the formula —S(O)ₙR^{7a} (wherein n denotes an integer of 0 to 2 and R^{7a} has the same meaning as defined above.), the formula —S(O)₂NR^{7a}R^{7b} (wherein R^{7a} and R^{7b} have the same meaning as defined above), the formula

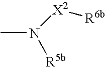

(wherein X², R^{5b}, and R^{6b} have the same meaning as defined above), or a C₁₋₆ alkoxy group substituted or unsubstituted with 0 to 4 substituents, or R^{3a} and R^{4a} together denote a methylenedioxy group, or a 1,2-ethylenedioxy group.

22. The aforementioned antifungal agent of 15, wherein the compound having an antifungal action is (1) 1-benzylisoquinoline, (2) 1-(4-bromobenzyl)isoquinoline, (3) 1-(4-chlorobenzyl)isoquinoline, (4) 1-(4-fluorobenzyl)isoquinoline, (5) 1-(4-iodobenzyl)isoquinoline, (6) 1-(3-methylbenzyl)isoquinoline, (7) 1-(4-methylbenzyl)isoquinoline, (8) 1-(3,4-dimethylbenzyl)isoquinoline, (9) 1-(3-methoxybenzyl)isoquinoline, (10) 1-(4-methoxybenzyl)isoquinoline, (11) 1-(3,4-methylenedioxybenzyl)isoquinoline, (12) 1-(4-benzyloxybenzyl)isoquinoline, (13) 1-(4-cyanobenzyl)isoquinoline, (14) 1-(4-nitrobenzyl)isoquinoline, (15) 1-(4-aminobenzyl)isoquinoline, (16) 1-(4-methoxybenzyl)-6,7-dichloro-isoquinoline, (17) 1-(4-methoxy-2-nitro-benzyl)-isoquinoline, (18) 1-(4-methoxybenzyl)-6,7-methylenedioxy-isoquinoline, (19) 1-(2-amino-4-methoxy-benzyl)isoquinoline, (20) 1-(4-methoxybenzyl)-7-hydroxy-6-methoxy-isoquinoline, (21) 1-(4-benzyloxybenzyl)-6,7-dimethoxy-isoquinoline, (22) 1-(4-methoxybenzyl)6,7-dimethoxy-isoquinoline, (23) 1(4-methoxy-2-nitro-benzyl)-isoquinoline, (24) 3-[4-(1-isoquinolylmethyl)phenoxy]propylcyanide, (25) 1-[4-(2,2,3,3-tetrafluoropropoxy)benzyl]isoquinoline, (26) 1-[4-(2-piperidinoethoxy)benzyl]isoquinoline, (27) 4-(1-isoquinolylmethyl)phenyl(2-morpholinoethyl)ether, (28) 1-[4-(2-methoxyethoxy)benzyl]isoquinoline, (29) N-{2-[4-(1-isoquinolylmethyl)phenoxy]ethyl}-N,N-dimethylamine, (30) 1-[4-(phenethyloxy)benzyl]isoquinoline, (31) 1-{4-[(2-methylallyl)oxy]benzyl}isoquinoline, (32) 1-(4-isobutoxybenzyl)isoquinoline, (33) 1-[4-(2-phenoxyethoxy)benzyl]isoquinoline, (34) methyl 2-[4-(1-isoquinolylmethyl)phenoxy]acetate, (35) 2-[4-(1-isoquinolylmethyl)phenoxy]-1-ethanol, (36) t-butyl N-{2-[4-(1-isoquinolylmethyl)phenoxy]ethyl}carbamate, (37) 1-{4-[3-(tetrahydro-2H-2-pyranyloxy)propoxy]benzyl}isoquinoline, (38) 2-[4-(1-isoquinolylmethyl)phenoxy]-1-ethaneamine, (39) 1-[4-(3-piperidinopropoxy)benzyl]isoquinoline, (40) 3-[4-(1-isoquinolylmethyl)phenoxy]-1-propanol, (41) 1-[4-(2-ethylbutoxy)benzyl]isoquinoline, (42) 4-[4-(1-isoquinolylmethyl)phenoxy]butanoic acid, (43) 1-(4-{3-[(4-benzylpiperazino)sulfonyl]propoxy}benzyl)isoquinoline, (44) 1-(4-{3-[4-(4-chlorophenyl)piperazino]propoxy}benzyl)isoquinoline, (45) 4-(1-isoquinolylmethyl)aniline, (46) N-[4-(1-isoquinolylmethyl)phenyl]butaneamide, (47) N-[4-(1-isoquinolylmethyl)phenyl]propaneamide, (48) N-[4-(1-isoquinolylmethyl)phenyl]-1-ethanesulfonamide, (49) N-[4-(1-isoquinolylmethyl)phenyl]-N-methyl-ethanesulfonamide, (50) N-[4-(1-isoquinolylmethyl)phenyl]-N-methylamine, (51) N-[4-(1-isoquinolylmethyl)phenyl]-N-propylamine, or (52) N-[4-(1-isoquinolylmethyl)phenyl]-N-methyl-N-propylamine.

23. A method for treating a mycotic infection comprising administering a therapeutically effective dose of any one of the antifungal agents of 13 to 22 to a mammal.

The present invention will be described in detail below by explaining the meaning of the terms, symbols, and such mentioned in the present description.

In the present description, the structural formula of the compounds may represent a certain isomer for convenience, however, the present invention includes all geometrical isomers, optical isomers based on asymmetric carbon, stereoisomers, and tautomers that structurally arise from compounds, and mixtures of isomers, and it is not to be construed as being limited to the representation in the formula made for convenience, and may be any one or a mixture of isomers. Therefore, an optically active substance and a racemic substance having an asymmetric carbon atom in the molecule may exist, but in this invention there are no particular limitations and any one of them are included. Furthermore, crystal polymorphism may exist, but similarly there are no limitations, and the crystal form may be any one form or may be a mixture, and may be either an anhydride or a hydrate.

Furthermore, the compounds of the present invention include compounds exhibiting antifungal action after being metabolized, such as after being oxidized, reduced, hydrolyzed, or conjugated in vivo. Furthermore, the present invention includes compounds that produce the compounds of this invention after being metabolized, such as after being oxidized, reduced, and hydrolyzed in vivo.

The “C₁₋₆ alkyl group” in the present description means a straight chain or branched chain alkyl group, wherein the number of carbon ranges from 1 to 6, and specific examples include a methyl group, ethyl group, n-propyl group, i-propyl group, n-butyl group, i-butyl group, tert-butyl group, n-pentyl group, i-pentyl group, neopentyl group, n-hexyl group, 1-methylpropyl group, 1,2-dimethylpropyl group, 2-ethylpropyl group, 1-methyl-2-ethylpropyl group, 1-ethyl-2-methylpropyl group, 1,1,2-trimethylpropyl group, 1-methylbutyl group, 2-methylbutyl group, 1,1-dimethylbutyl group, 2,2-dimethylbutyl group, 2-ethylbutyl group, 1,3-dimethylbutyl group, 2-methylpentyl group, 3-methylpentyl group, and so on.

The “C₂₋₆ alkenyl group” in the present description means a straight chain or branched chain alkenyl group, wherein the number of carbon ranges from 2 to 6, and specific examples include a vinyl group, allyl group, 1-propenyl group, isopropenyl group, 1-butene-1-yl group, 1-butene-2-yl group, 1-butene-3-yl group, 2-butene-1-yl group, 2-butene-2-yl group, and so on.

The “C₂₋₆ alkynyl group” in the present description means a straight chain or branched chain alkynyl group, wherein the number of carbon ranges from 2 to 6, and specific examples include an ethynyl group, 1-propynyl group, 2-propynyl group, butynyl group, pentynyl group, hexynyl group, and so on.

The “C₁₋₆ alkoxy group” in the present description means an oxy group to which “C₁₋₆ alkyl group” defined above is bound, and specific examples include a methoxy group, ethoxy group, n-propoxy group, i-propoxy group, n-butoxy group, i-butoxy group, sec-butoxy group, t-butoxy group, n-pentyloxy group, i-pentyloxy group, sec-pentyloxy group, t-pentyloxy group, neopentyloxy group, 1-methylbutoxy group, 2-methylbutoxy group, 1,1-dimethylpropoxy group, 1,2-dimethylpropoxy group, n-hexyloxy group, i-hexyloxy group, 1-methylpentyloxy group, 2-methylpentyloxy group, 3-methylpentyloxy group, 1,1-dimethylbutoxy group, 1,2-dimethylbutoxy group, 2,2-dimethylbutoxy group, 1,3-dimethylbutoxy group, 2,3-dimethylbutoxy group, 3,3-dimethylbutoxy group, 1-ethylbutoxy group, 2-ethylbutoxy group, 1,1,2-trimethylpropoxy group, 1,2,2-trimethylpropoxy group, 1-ethyl-1-methylpropoxy group, 1-ethyl-2-methylpropoxy group, and so on.

The “C₆₋₁₄ aryl group” in the present description refers to an aromatic ring group, wherein the number of carbon ranges from 6 to 14, and specific examples include a phenyl group, 1-naphthyl group, 2-naphthyl group, as-indacenyl group, s-indacenyl group, acenaphthylenyl group, and so on.

The “halogen atom” of the present description means a fluorine atom, chlorine atom, bromine atom, and iodine atom.

“Substituted or unsubstituted” in the present description means “the substitutable site may have an arbitrary combination of one or more substituents” and specifically the substituents are, for example, a hydrogen atom, halogen, nitro group, cyano group, hydroxyl group, mercapto group, hydroxyalkyl group, carboxyl group, C₁₋₆ alkoxycarbonyl group, C₂-₇ acylamino group, C₁₋₆ alkylamino group, pyridyl group, C₁₋₆ alkylsulfinyl group, C₁₋₆ alkylsulfonyl group, C₁₋₆ alkylsulfamoyl group, C₁₋₆ alkylsulfinamoyl group, C₁-6 alkylsulfenamoyl group, tetrahydropyranyl group, C₁₋₆ alkylcarbamoyl group, or the formula —X⁴—R^{8a} (wherein X⁴ denotes a single bond, oxygen atom, or sulfur atom; R^{8a} denotes a C₁₋₆ alkyl group, C₂₋₆ alkenyl group, C₂₋₆ alkynyl group, C₆₋₁₄ aryl group, C₃₋₈ cycloalkyl group, or C₃₋₈ cycloalkenyl group), and so on.

“May be substituted with 0 to 4 substituents” has the same meaning as “the substitutable site may have an arbitrary combination of 1 to 4 substituents” and the substituents have the same meaning as defined above.

“Salt” in the present invention refers to a pharmaceutically acceptable salt, and there are no particular limitations as long as the salt has formed an addition salt with a compound of this invention, and a preferred example is a haloid acid salt such as hydrofluoride, hydrochloride, hydrobromide, and hydroiodide; an inorganic acid salt such as a sulfate, nitrate, perchlorate, phosphate, carbonate, and bicarbonate, an organic carboxylate such as an acetate, oxalate, maleate, tartrate, and fumarate; an organic sulfonate such as a methanesulfonate, trifluoromethanesulfonate, ethanesulfonate, benzenesulfonate, toluenesulfonate, and camphorsulfonate; an amino acid salt such as an aspartate, and glutamate; salts with an amine such as a trimethylamin, triethylamine, procaine, pyridine, and phenethylbenzylamine; alkali metal salts such as sodium, and potassium; alkaline earth metal salts such as magnesium and calcium; and so on.

Herein below, the following will be disclosed: 1. A method for obtaining DNAs encoding proteins participating in cell wall synthesis, 2. a method for examining whether or not a test sample influences the process that transports GPI-anchored proteins to the cell wall, and 3. a method for obtaining the aforementioned compound (Ia) of the present invention.

### 1. A method for Obtaining DNAs Encoding Proteins Participating in Fungal Cell Wall Synthesis

Hereinafter, (1) a method for obtaining a DNA encoding a protein for acquiring resistance to the aforementioned compound (Ia) by overexpression in fungi; (2) a method for obtaining a DNA that hybridizes under stringent conditions with the DNA of SEQ ID NO: 1, SEQ ID NO: 3, or SEQ ID NO: 5; (3) a method for obtaining a DNA that encodes a protein that participates in fungal cell wall synthesis, based on a homology search; and (4) a method for obtaining a fungus that overexpressed or lacked the protein for acquiring resistance to the aforementioned compound (Ia), will be described.

(1). A Method for Obtaining a DNA Encoding a Protein for Acquiring Resistance to the Aforementioned Compound (Ia) by Overexpression of the DNA in a Fungus

Herein, “fungus” means a fungus belonging to Division Zygomycota, Ascomycota, Basidiomycota, and Deuteromycota. Preferable is a pathogenic fungus, Mucor, Saccharomyces, Candida, Cryptococcus, Trichosporon, Malassezia, Aspergillus, Trichophyton, Microsporum, Sporothrix, Blastmyces, Coccidioides, Paracoccidioides, Penicillinium, or Fusarium, and more preferable is C. albicans, C. glabrata, C. neoformans, or A. fumigatus. S. cerevisiae and S. pombe, for which genetic analyses are easy, are also preferred strains.

A plasmid library of a fungal gene is introduced into a fungus. The plasmid library of S. cerevisiae and S. pombe can be obtained from ATCC (Information for ATCC Number: 37323), and the plasmid library of C. albicans can be produced by the method according to Navaro-Garcia, F. et al, Mol. Cell. Biol., 15: 2197-2206, 1995. The obtained plasmid library is introduced to the fungi by the method according to Gietz, D. et al, Nucl. Acids Res. 20: 1425, 1992. Alternatively, a kit such as YEASTMAKER™ Yeast Transformation System (Clontech) may be used.

The Fungus to which the plasmid library is introduced is cultured in the presence of the aforementioned compound (Ia) Specifically, an agar medium containing the aforementioned compound (Ia) at a concentration of 1.56 to 25 μg/ml, preferably 1.56 to 6.25 μg/ml, and more preferably 3.125 μg/ml, is inoculated with the fungus into which a plasmid library has been introduced, is cultured for an appropriate length of time, at 30° C. to 42° C. for 2 to 5 days, or preferably at 37° C. for 3 days. The colony formed upon proliferation is further cultured in a medium containing the aforementioned compound (Ia), and the plasmid is purified from the proliferated fungal cells. Purification of the plasmid can be performed by the method according to METHODS IN ENZYMOLOGY, Vol. 194: 169-182 (1991), for example.

Preferably, the nucleotide sequence of the obtained plasmid is determined directly, but if necessary, cloning into an appropriate vector, for example pBluescript II, and pUC19 suitable for nucleotide sequence determination, is done to determine the nucleotide sequence. A nucleotide sequence can be determined for example by the method accompanying the ABI377 System (PE applied Biosystems) manual.

In the Examples of the present invention, all 27 of the independently obtained colonies of S. cerevisiae, and 28 colonies out of 30 colonies of C. albicans contained the DNAs of this invention. Only one gene that confers resistance to the aforementioned compound (Ia) exists in these fungi and this can be obtained by the abovementioned method.

(2). A Method for Obtaining a DNA That Hybridizes Under Stringent Conditions to the DNA of SEQ ID NO: 1, SEQ ID NO: 3, or SEQ ID NO: 5

An example of a method for obtaining a DNA encoding a protein participating in fungal cell wall synthesis according to the present invention comprises designing a primer from the information of the nucleotide sequence of SEQ ID NO: 1 using the genomic DNA of S. cerevisiae as a template, or designing a primer from the information of the nucleotide sequence of SEQ ID NO: 3 or SEQ ID NO: 5 using the genomic DNA of C. albicans as the template, then performing PCR, and cloning the amplified DNA into an appropriate vector, such as pBlueScript. The primer is designed as necessary according to the region to be amplified, and the length is preferably 15 bp or more, more preferably 20 bp or more, and in some cases sequences necessary for subsequent DNA construction, such as restriction enzyme sites, may be added. The conditions for PCR can be determined appropriately according to factors such as the length of primer, the length of the region to be amplified, and the amount of template DNA to be used. For example, a DNA encoding a protein participating in cell wall synthesis in a fungus can be obtained using 200 ng of the genomic DNA of C. albicans as a template, and SEQ ID NO: 21 and SEQ ID NO: 22 as primers under conditions of 94° C. for 4 minutes→(94° C. for 30 seconds→68° C. for 5 minutes)×35 cycles→72° C. for 4 minutes.

The DNA obtained by PCR may be used as a probe for obtaining other types of fungal DNA showing homology to the DNA encoding the protein participating in cell wall synthesis. Specifically, for example, to obtain a homologous gene of C. albicans encoding the protein participating in S. cerevisiae cell wall synthesis, DNA that hybridizes under stringent conditions can be cloned from the genomic library or cDNA library of C. albicans, using the genomic DNA of S. cerevisiae as a template, and using DNA that is obtained by PCR as a probe. Herein, stringent conditions refer to hybridization in 4×SSC at 65° C., then washing in 0.1×SSC at 65° C. for 1 hour, for example. Furthermore, in another the stringent conditions are 4×SSC at 42° C. in 50% formamide. Alternatively, conditions such as hybridization in the PerfectHyb™ (TOYOBO) solution at 65° C. for 2.5 hours, then washing in 1). 2×SSC, 0.05% SDS solution at 25° C. for 5 minutes, 2). 2×SSC, 0.05% SDS solution at 25° C. for 15 minutes, and 3). 0.1×SSC, 0.1% SDS solution at 50° C. for 20 minutes, are also allowed.

The Examples of this invention demonstrate from Southern Blot analysis that there is only one gene in C. albicans that hybridizes with the DNA of SEQ ID NO: 1, and shows the cloning of this gene. From the above-mentioned method, DNA that hybridizes with SEQ ID NO: 1 or SEQ ID NO: 3 can be obtained.

(3). A Method for Obtaining a DNA That Encodes a Protein That Participates in Fungal Cell Wall Synthesis, Based on a Homology Search

The present invention revealed the GWT1 homologues of S. cerevisiae, C. albicans, S. pombe, A. fumigatus, and C. neoformans. The region conserved among these genes is considered to be important for GWT1 gene products to exhibit their function, and may very well be conserved in other fungi.

Therefore, a DNA encoding a protein participating in fungal cell wall synthesis can be obtained by either carrying out hybridization upon constructing a probe based on the amino acid sequence of the conserved region, or by performing PCR by designing primers based on the sequence. The PCR primer may be of any sequence as long as it is designed to encode the conserved region, but is preferably SEQ ID NOS: 29 and 31 or preferably SEQ ID NOS: 29 and 30.

Furthermore, as another method, a DNA encoding a protein participating in fungal cell wall synthesis can be obtained by carrying out PCR with cDNA or genomic DNA upon finding a nucleotide sequence showing homology to GWT1 from gene fragments registered in databases, and then designing primers based on that nucleotide sequence.

Examples of PCR methods for obtaining a full-length gene based on the obtained sequence are techniques such as 3′-RACE, 5′-RACE, and inverse PCR, and it is also possible to select by hybridization a clone containing neighboring sequences. A full-length gene can be obtained by combining these techniques.

(4). A Method for Obtaining a Fungus That Overexpresses or Lacks a Protein for Acquiring Resistance to the Aforementioned Compound (Ia)

A Fungus, preferably S. cerevisiae, that overexpresses a protein for acquiring resistance to the aforementioned compound (Ia) of this invention can be obtained by the method of inserting an expression vector expressing the protein into a particular position on the fungal chromosome, for example an expression vector in which the DNA of SEQ ID NO: 1 is connected downstream of a promoter, which can forcibly express the protein in fungi, preferably the promoter of budding yeast enolase gene (ENO1). The insertion method can be performed, for example, by the steps of, inserting a desired sequence into the multicloning site of pRS304 (Sikorski R S et al, Genetics. 122(1): 19-27, 1989), constructing a vector for integration, and introducing the vector into the fungus. One can refer to METHODS IN ENZYMOLOGY Vol.194: 281-301 (1991) for details.

Furthermore, an overexpressed strain of C. albicans can be obtained by incorporating the gene of SEQ ID NO: 3 or SEQ ID NO: 5 into an expression vector for C. albicans, such as pCARS1 and pRM1 (Pla J et al, Yeast 12: 1677-1702, 1996), and then transforming C. albicans (Sanglard D et al, Antimicrobiol. Agents Chemother. 40: 2300-2305, 1996).

Fungi of this invention lacking a gene for acquiring resistance against the aforementioned compound (Ia), preferably S. cerevisiae, can be obtained by the following methods, but is not to be construed as being limited thereto.

PCR amplification is carried out using a marker gene, preferably his5 gene of S. pombe, as a template, and using primers that are designed so that PCR products that contain the gene to be deleted (30 bp or more, or preferably 40 bp or more). In the case of S. cerevisiae, the genetic sequence of SEQ ID NO: 1, positioned on both ends can be obtained. The PCR products can be purified and introduced into fungi, then cultured in a selection medium corresponding to the marker gene, for example, his⁻ for his5, to obtain the deletion strain.

Furthermore, the deletion strain of C. albicans is obtained by the usual method using a hisG-URA3-hisG cassette (Fonzi W A et al, Genetics 134: 717-728,1993) based on the nucleotide sequence information of SEQ ID NO: 3 or SEQ ID NO: 5.

### 2. A Method for Examining Whether or not the Test Sample Influences the Process That Transports GPI-Anchored Proteins to the Cell Wall

Whether or not the test sample inhibits the process that transports GPI-anchored proteins to the cell wall, or whether or not the test sample inhibits the expression of the GPI-anchored protein in the fungal surface can be examined by (1) a method using a reporter enzyme, (2) a method using an antibody that reacts with the surface glycoprotein of the fungal cell wall, (3) a method for examining the adhesion ability towards animal cells, and (4) a method for observing fungi using an optical microscope or an electron microscope.

By using the methods of (1) to (4) described below, preferably the methods of (1) to (4) in combination, the test sample is judged to inhibit the process that transports GPI-anchored proteins to the cell wall, or the expression of the GPI-anchored proteins at the fungal surface. Furthermore, it is judged that the test sample influences the process that transports GPI-anchored proteins to the cell wall when the degree of inhibition diminishes or the inhibition is no longer seen when the protein encoded by the DNA of the present invention is overexpressed in fungi.

Hereinafter, the methods of (1) to (4) will be described.

(1). A Method Using a Reporter Enzyme

The process that transports GPI-anchored proteins to the cell wall can be quantified by a tracer experiment such as labeling a GPI-anchored protein with a radioactive isotope, then upon fractionation of the fungal cell wall fraction, immunoprecipitating with an antibody against a GPI-anchored protein. Alternatively, the quantification can be more readily done by expressing the C-terminal sequence considered to function as a transport signal, which is commonly observed among GPI-anchored proteins, as a fusion protein with an easily measurable enzyme (reporter enzyme), fractionating the fungal cell wall fraction, and then using a reporter system that measures the enzyme activity of each fraction (Van Berkel M A A et al, FEBS Letters, 349: 135-138, 1994). Hereinafter, a method using the reporter enzyme will be explained, but the present invention is not to be construed as being limited thereto.

First, the reporter gene is constructed and is introduced into a fungus. The reporter gene is constructed by linking a promoter sequence that functions in fungi, followed by DNAs that respectively encode a signal sequence, a reporter enzyme, and a GPI-anchored protein C-terminal sequence so that the reading frames match. Examples of the promoter sequences are those of promoters such as GAL10, and ENO1. Examples of signal sequences are those of α-factor, invertase, lysozyme, and such. Examples of reporter enzymes are β-lactamase, lysozyme, alkaline phosphatase, β-galactosidase, and such. Green Fluorescence Protein (GFP), which can be detected easily, can be used, even though it does not have enzyme activity. Examples of GPI-anchored protein C-terminal sequences are α-agglutinin C-terminal sequence, CWP2 C-terminal sequence, and such. Furthermore, it is preferable to insert an appropriate selection marker such as LEU2, and URA3 into the vector containing the constructed reporter gene.

The constructed reporter gene is inserted into a fungus by an appropriate method, such as the lithium acetate method (Gietz D et al, Nucl. Acids Res. 20: 1425, 1992), and cultured, if necessary by a method suitable for the selection marker, such as Leu⁻ medium for LEU2, and Ura⁻ medium for URA3, and then fungi into which the DNA has been introduced are selected.

Whether or not a test sample influences the process that transports GPI-anchored proteins to the cell wall is examined by the following method.

The reporter gene-introduced fungi are cultured under appropriate conditions, for example at 30° C. for 48 hours, in the presence of a test sample. After culturing, the culture supernatant is centrifuged, and the reporter enzyme activity of the culture supernatant fraction is measured. The remaining cell fraction is washed, then the cell wall components are separated by an appropriate method, such as degrading the cell wall glucan with glucanase, and then measuring the reporter enzyme activity of the cell wall fraction and the cytoplasmic fraction. The assay can be simply carried out by determining the amount of reporter enzyme in the cell fraction by centrifuging, then without washing the cells, determining the amount of reporter enzyme derived from the culture supernatant fraction that remains in the cell fraction by proportional calculation, and subtracting this from the amount of reporter enzyme of the cell fraction.

If an activity to increase the reporter enzyme activity within the culture supernatant fraction (activity per cell), or an activity to decrease the reporter enzyme activity in the cell wall fraction (activity per cell) is confirmed in the test sample, the test sample is judged to have influenced the process that transports GPI-anchored proteins to the cell wall.

(2). A Method Using an Antibody That Reacts With the Surface Glycoprotein of a Fungal Cell Wall

Whether or not the test sample influences the expression of the GPI-anchored protein at the fungal surface layer can be detected by quantifying a GPI-anchored protein in the fungal cell wall using an antibody that reacts with the protein.

For example, as the antibody, the antigenic determinant is predicted from the amino acid sequence of a GPI-anchored protein, for example, α-agglutinin, Cwp2p, and Als1p (Chen M H et al, J. Biol. Chem., 270:26168-26177, 1995; Van Der Vaat J M et al, J. Bacteriol., 177:3104-3110,1995; Hoyer L L et al, Mol. Microbiol., 15:39-54, 1995), the peptide of that region is synthesized, this is bound to an antigenic substance, such as a carrier protein, and then polyclonal antibodies can be obtained by immunizing a rabbit and such, or a monoclonal antibody can be obtained by immunizing a mouse and such. Furthermore, a house rabbit polyclonal antibody against the Als1p peptide is preferable.

In an alternative method, a monoclonal antibody against a GPI-anchored protein may be obtained by immunizing a mouse and such with a fungus, preferably a fungus overexpressing the GPI-anchored protein, such as α-agglutinin, Cwp2p, and Als1p, and in some cases, by immunizing with the partially purified GPI-anchored protein , and selecting the clone yielded as a result of the fusion by ELISA, Western blot analysis, and such.

Whether or not the test sample influences the process that transports GPI-anchored proteins to the cell wall, and diminishes the amount of the protein derived from the GPI-anchored protein in the cell wall can be examined by the following method.

A fungus is cultured in the presence of a test sample under appropriate conditions, such as 30° C., for 48 hours. The cultured fungus is collected by centrifugation and the cells are disrupted, preferably using glass beads. The washed, disrupted cells are preferably subjected to centrifugal extraction with SDS, then the precipitate is washed. After the extraction, the disrupted cells are treated with an enzyme that degrades glucan, preferably glucanase, and the centrifuged supernatant thereof is the GPI-anchored protein sample.

The anti-Als1p peptide antibody is coated onto a 96-well plate by incubating at 4° C. overnight. After washing with a washing solution, preferably PBS containing 0.05% Tween 20 (PBST), blocking is carried out with a reagent that blocks the non-specific adsorption sites of the 96-well plate, preferably a protein such as BSA, and gelatin, more preferably BlockAce. After washing again with a washing solution, preferably PBST, in some cases, after adding an appropriately diluted GPI-anchored protein sample, the reaction is carried out for an appropriate length of time, such as 2 hours at room temperature. After washing with a washing solution, preferably with PBST, an antibody against the enzyme-labeled C. albicans, preferably HRP-labeled anti-Candida antibody, is reacted for an appropriate length of time, such as 2 hours at room temperature. The method for labeling may be enzyme labeling or radioactive isotope labeling. After washing with a washing solution, preferably PBST, the amount of Als1p in the GPI-anchored protein sample is calculated by a method appropriate for the type of label, i.e. for an enzyme label, adding a substrate solution, and then upon stopping the reaction, measuring the absorbance at 490 nm.

(3). A Method for Examining the Adhesion Ability Towards Animal Cells

Whether or not the test sample influences expression of a GPI-anchored protein on the fungal surface can be examined by measuring the activity of the GPI-anchored protein in the fungal cell wall, preferably by measuring the adhesion ability of fungi to animal cells, and such. Besides Als1p, Hwp1p, and such participating in adhesion to animal cells, α-agglutinin participating in mating, Flo1p participating in yeast aggregation, and such are known as GPI-anchored proteins. Hereinafter, examination methods that use the adhesion ability of fungi to animal cells will be explained in detail, but this invention is not to be construed as being limited thereto.

As the fungus, a fungus having an adhesion ability towards cells is used, and preferably, the fungus is C. albicans. For mammalian cells, cells that adhere to the fungus are used, and preferably, are intestinal epithelial cells. The mammalian cells are cultured and are immobilized by an appropriate method such as ethanol immobilization. The test sample and the fungi, which have been incubated for an appropriate length of time, such as 48 hours at 30° C., are inoculated, then after culturing for a certain length of time, for example 1 hour at 30° C., the culture supernatant is removed, washed with a buffer, and is superposed onto an agar media, such as Sabouraud Dextrose Agar Medium (Difco). After culturing at 30° C. overnight, the number of colonies is counted, and the adhesion rate is calculated.

If activity to lower the number of colonies formed by adhesion of fungi to cells is observed in a test sample compared to that of fungi that are not treated with the compound, the test sample is judged to have influenced the process that transports GPI-anchored proteins to the cell wall.

(4). A Method for Observing Fungi Using an Electron Microscope or an Optical Microscope

Whether or not a test sample influences the expression of the GPI-anchored proteins in the fungal surface can be examined by observing the structure of the fungal cell wall using an electron microscope.

In the presence of a test sample, a fungus such as C. albicans is cultured for a certain length of time, for example, 48 hours at 30° C., and the ultrafine morphological structure is observed with a transmission electron microscope. Herein, observation with a transmission electron microscope can be carried out, for example by the method according to the Electron Microscope Chart Manual (Medical Publishing Center). The flocculent fibrous structure of the outermost layer of the fungal cell that has a high electron density and is observable by transmission electron microscope image, is considered to be a surface glycoprotein layer having GPI-anchored proteins as its constituents, and is not influenced by other existing antifungal agents. When this flocculent fibrous structure of the outermost layer of a fungal cell, which has a high electron density, disappears leaving a slight layer with a high electron density, compared to that in the untreated cells, the test sample is judged to have influenced the process that transports GPI-anchored proteins to the cell wall.

When images, in which fungal cells are largely swollen and budding (division) is inhibited, are observed under a transmission electron microscope in addition to an optical microscope, the test sample is judged to have an influence on the cell wall.

The compounds of the present invention represented by the formula (I)

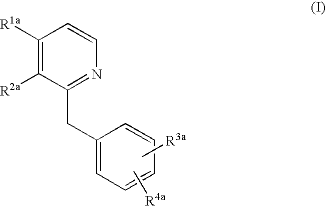

(wherein the symbols have the same meaning as defined above) can be synthesized by utilizing conventional organic chemical reactions and such that have been known to date. For example, it can be synthesized by the following methods.

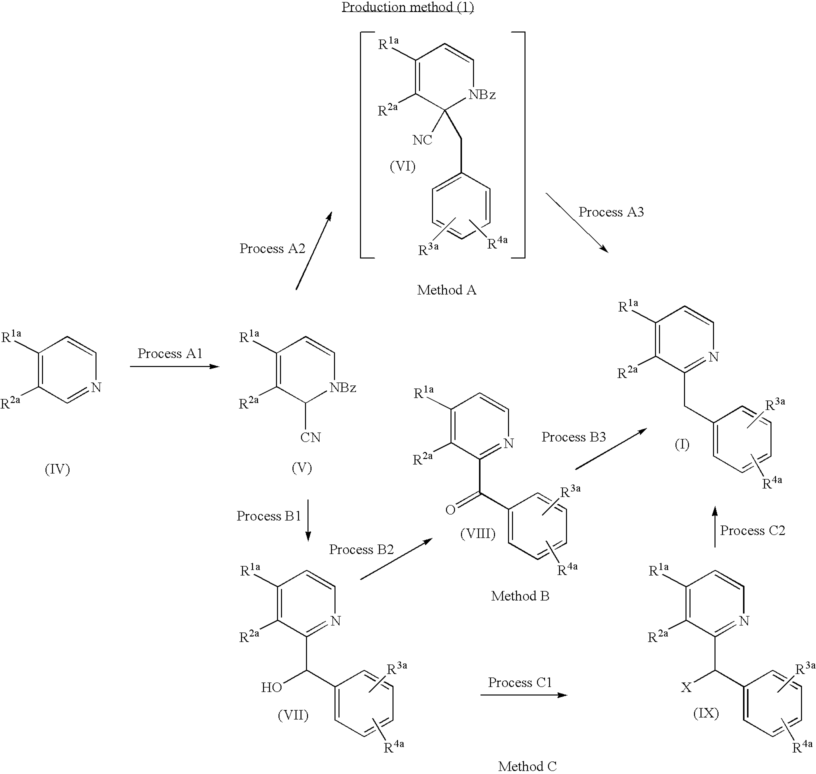

In the above formulae, X is a leaving group such as a halogen group and acyl group. R^{3c} has the same meaning as R^{3a}. Other symbols in the formulae have the same meaning as defined above.

Process A1

A reaction for producing the Reissert compound (V). The compound can be produced based on the reaction conditions according to the literature, such as Org. Synth., VI, 115(1988); Heterocycles, 36(11), 2489(1993); J. Chem. Soc. (C), 666(1969); or J. Heterocycl. Chem., 29(5), 1165(1992). Specifically, the reagents used are, for example, a combination of benzoyl chloride and potassium cyanide.

Process A2

A process for alkylation. The compound (VI) can be produced by reacting the compound (V) with a substituted benzyl halide derivative, a substituted benzylmethanesulfonate derivative, or such in the presence of a base. Specific examples of the base include sodium hydride, sodium hydroxide.

Process A3

A process for hydrolysis reaction. The compound (I) can be produced by hydrolysis of the compound (VI) in the presence of a base.

Method A is a method for producing the compound (I) via Process A1, Process A2, and Process A3.

Process B1

A process for conversion of the compound (V) to the compound (VII) The compound (VII) can be produced by reacting the compound (V) with a substituted benzaldehyde in the presence of a base and a phase-transfer catalyst. Examples of the base include sodium hydroxide and potassium hydroxide. Examples of the phase-transfer catalyst include triethylbenzylammonium chloride.

Process B2

A process for oxidation of the alcohol to the ketone. The ketone derivative (VIII) can be produced by using an oxidizing agent and a condition conventionally used for the oxidation reaction of an alcohol to a ketone. Specifically, the oxidizing agent is, for example, manganese dioxide, chromium dioxide, or benzoquinone.

Process B3

A process for reduction of the ketone to the methylene. The methylene derivative (I) can be produced by using a conventionally used combination of reducing agents for the reduction reaction of the ketone derivative (VIII) to the methylene derivative (I). Examples of the combination of the reducing agents include hydrazine hydrate and sodium hydroxide or potassium hydroxide, triethylsilane and boron trifluoride, and trifluoromethanesulfonic acid.

Method B is a method for producing the compound (I) via Process A1, Process B1, Process B2, and Process B3.

Process C1

A process for halogenation or acylation of the hydroxyl group. The compound (IX) can be produced by reacting a halogenating agent or an acylating agent with the compound (VII). Examples of the halogenating agent include thionyl chloride, concentrated hydrochloric acid, and phosphorus tribromide. Furthermore, examples of the acylating agent include acid halides such as acetyl chloride and acid anhydrides such as acetic anhydride.

Process C2

A process for reductive elimination reaction of the halogen group or the acyl group. The compound (I) can be produced by hydroelimination of the compound (IX), for example, by using a catalyst.

Examples of the catalyst include palladium-carbon.

Method C is a method for producing the compound (I) via Process A1, Process B1, Process C1, and Process C2.

### Production Method (2)

The compound of the present invention represented by the formula (I) can also be synthesized by the following method.

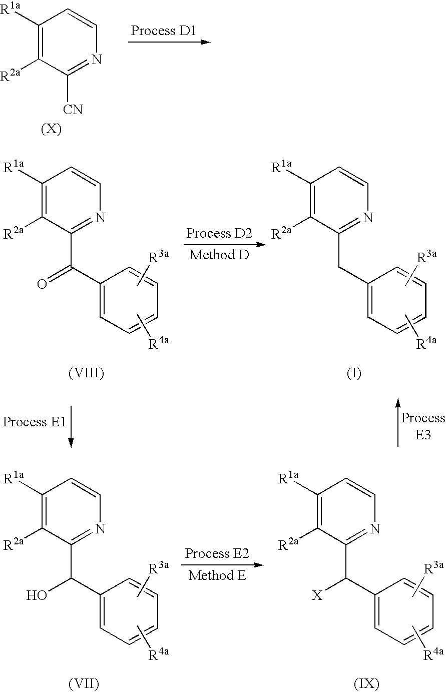

In the formula, X is a leaving group such as a halogen group and acyl group. Other symbols in the formulae have the same meaning as defined above.

Process D1

A process for a Grignard reaction and a subsequent acid hydrolysis reaction. The compound (VIII) can be produced by reacting the compound (X) with a substituted or unsubstituted phenyl Grignard reagent, followed by hydrolysis in the presence of an acid.

Process D2

The methylene derivative (I) can be produced from the ketone derivative (VIII) by conditions similar to that of Process B3.

Method D is a method for producing the compound (I) via Process D1 and Process D2.

Process E1

A process for the reduction reaction from the ketone to the alcohol. The compound (VII) can be produced from the compound (VIII) using a reducing agent and conditions conventionally used for the reduction reaction of a ketone to an alcohol. Specific examples of the reducing agent include sodium borohydride and lithium aluminum hydride.

Process E2

Under conditions similar to that of Process C1, the halogenated or acylated derivative (IX) can be produced from the alcohol derivative (VII).

Process E3

Under conditions for reductive elimination reaction similar to that of Process C2, the compound (I) can be produced from the compound (IX).

Method E is a method for producing the compound (I) via Process D1, Process E1, Process E2, and Process E3.

### Production Method (3)

The compound of the present invention represented by the formula (I) can also be synthesized by the following method.

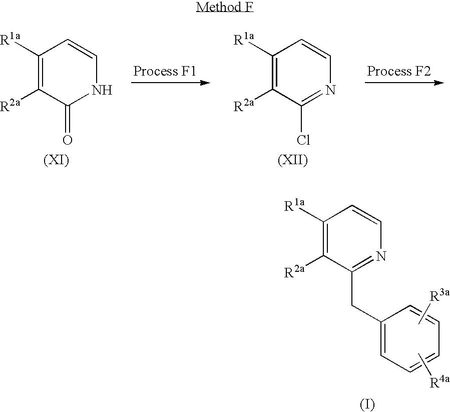

The symbols in the formulae have the same meaning as defined above.

Process F1

A process for the chlorination reaction. The compound (XII) can be produced by reacting the compound (XI) with a chlorinating agent. Examples of the chlorinating agent include phosphorus oxychloride and thionyl chloride.

Process F2

A process for the coupling reaction with a Grignard reagent. The compound (I) can be produced by reacting the compound (XII) with a substituted or unsubstituted benzyl Grignard reagent in the presence of a catalyst, based on the reaction conditions according to the literature, such as Arch. Pharm, 314, 156(1981). Examples of the catalyst include [1,1′-bis(diphenylphosphino)ferrocene]dichloro nickel(II).

Method F is a method for producing the compound (I) via Process F1 and Process F2.

### Production Method (4)

The compound of the present invention of the formula (I) wherein R^{1a} and R^{2a} together form a condensed ring such as a benzene ring, pyridine ring, pyrrole ring, thiophene ring, furan ring, cyclohexane ring, or cyclopentane ring, can be synthesized by the following method.

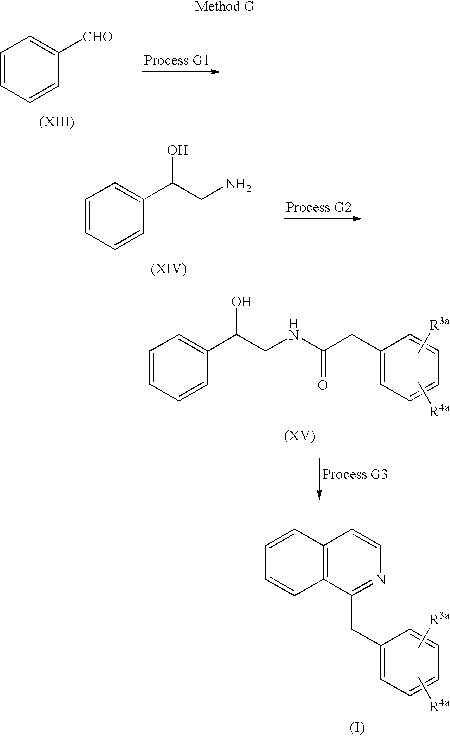

The symbols in the formulae have the same meaning as defined above.

The production method in which the isoquinoline ring is formed is shown below as an example.

Process G1

A process for the condensation reaction and the subsequent reduction reaction. The compound (XIV) can be produced by a condensation reaction between the substituted or unsubstituted benzaldehyde derivative (XIII) and nitromethane, followed by reduction of the nitro group. Examples of the reagent used for the reduction of the nitro group include a combination of palladium-carbon and ammonium formate, and lithium aluminum hydride.

Process G2

An amide bond formation reaction. The compound (XV) can be produced by reacting the compound (XIV) and a substituted or unsubstituted phenylacetyl chloride with a coupling reagent for an amide bond formation reaction. Examples of the coupling reagent include a combination of N,N′-dicyclohexylcarbodiimide and N-hydroxysuccinimide, a combination of N,N′-dicyclohexylcarbodiimide and N-hydroxybenzotriazole, and 1,1′-carbonyldiimidazole.

Process G3

A process for the cyclization reaction. The compound (XV) can be produced based on the reaction conditions according to the literature, such as Organic Reaction, 6, 74(1951); J. Hetetocyclic Chem., 30, 1581(1993). Examples of the reagent for this reaction include phosphorus oxychloride and polyphosphoric acid.

Method G is a method for producing the compound (I) via Process G1, Process G2, and Process G3.

### Production Method (5-1)

Replacement of the substituent R^{3a} or R^{4a} of the compound (I) synthesized by the aforementioned production method (5-1) Replacement of the substituent with an amino group, amide group, sulfonamide group, etc.

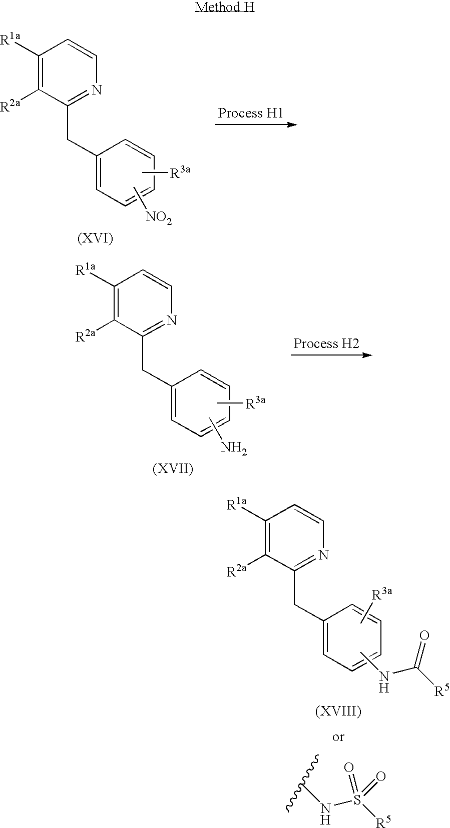

The symbols in the formulae have the same meaning as defined above.

Process H1

A reduction reaction of the nitro group. The compound (XVII) can be produced by reducing the compound (XVI) with a conventionally used method for reduction of a nitro group. Examples of the reduction method are catalytic hydrogenation reduction by palladium-carbon, or palladium hydroxide, and reduction by iron-ammonium chloride, iron-hydrochloric acid, iron-acetic acid, etc.

Process H2

A process for the acylation or sulfonylation reaction. The compound (XVIII) can be produced by treating the compound (XVII) with an acid chloride or acid anhydride.

Method H is a method for producing the compound (XVIII) via Process H1 and Process H2.

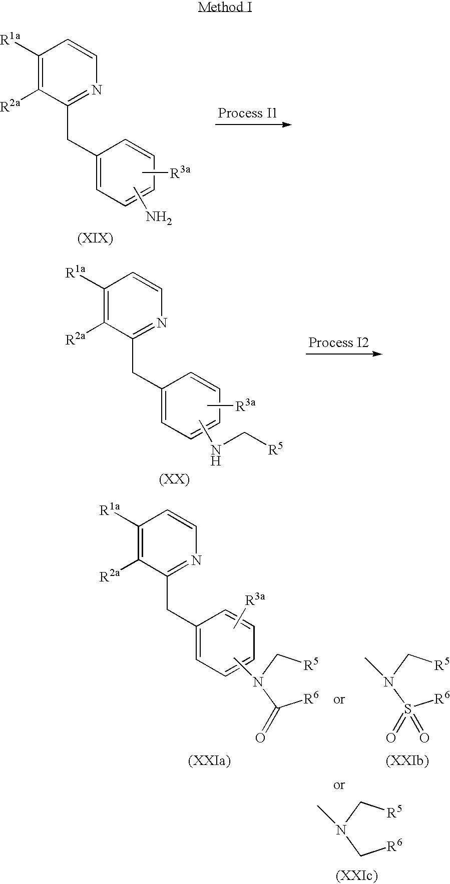

The symbols in the formulae have the same meaning as defined above.

Process I1

A process for the reductive amination reaction. The compound (XX) can be produced from the compound (XIX) and a substituted or unsubstituted aldehyde based on the reaction conditions according to the literature, such as J. Am. Chem. Soc., 93, 2897(1971); Comprehensive Organic Synthese, 8, 25(1991); Tetrahedron, 40, 1783(1984); and Tetrahedron, 41, 5307(1985). Examples of the reductive amination reagent include sodium triacetoxyhydroborate, sodium cyanotrihydroborate, borane-pyridine complex, and palladium-carbon/hydrogen.

Process I2

A process for the acylation, sulfonylation, or reductive amination reaction. The compound (XXIa) or the compound (XXIb) can be produced from the compound (XX) using an acid chloride or an acid anhydride. The compound (XXIc) can be produced by carrying out a reductive amination reaction similarly to that of Process I1.

Method I is a method for producing the compound (XXIa), the compound (XXIb), or the compound (XXIc) via Process I1 and Process I2.

### Production Method (5-2)

Replacement of the Substituent R^{3a} or R^{4a} of the Compound (I) Synthesized by the Aforementioned Production Method

(5-2) Replacement of the Substituent With a Hydroxyl Group, Alkoxy Group, etc.

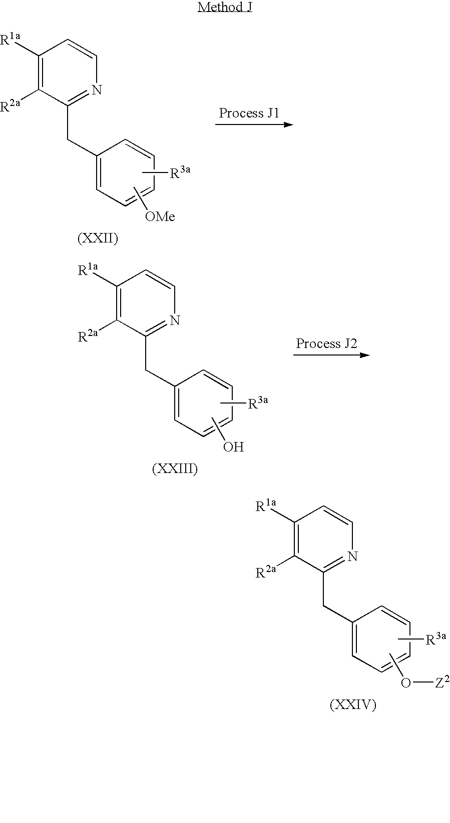

The symbols in the formulae have the same meaning as defined above.

Process J1

The compound (XXIII) can be produced from the compound (XXII) by a demethylation reaction based on the reaction conditions according to the literature, such as Bull. Chem. Soc. Jpn., 44, 1986(1971); Org. Synth., Collect. Vol. V, 412(1073); J. Am. Chem. Soc., 78, 1380 (1956); or J. Org. Chem., 42, 2761 (1977). Examples of the reagent used for the demethylation reaction include 47% aqueous hydrobromic acid solution, boron tribromide, pyridine hydrochloride, and iodotrimethylsilane.

Process J2

A process for the alkylation reaction. The compound (XXIV) can be produced by reacting the compound (XXIII) with a substituted or unsubstituted alkyl halide, a substituted or unsubstituted alkylmethane sulfonate, or such in the presence of a base.

Method J is a method for producing the compound (XXIV) via Process J1 and Process J2.

### Production Method (5-3)

Replacement of the Substituent R^{3a} or R^{4a} of the Compound (I) Synthesized by the Aforementioned Production Method

(5-3) Replacement of the Substituent With a Vinylene Group, an Ethynylene Group, Alkyl Group, etc.

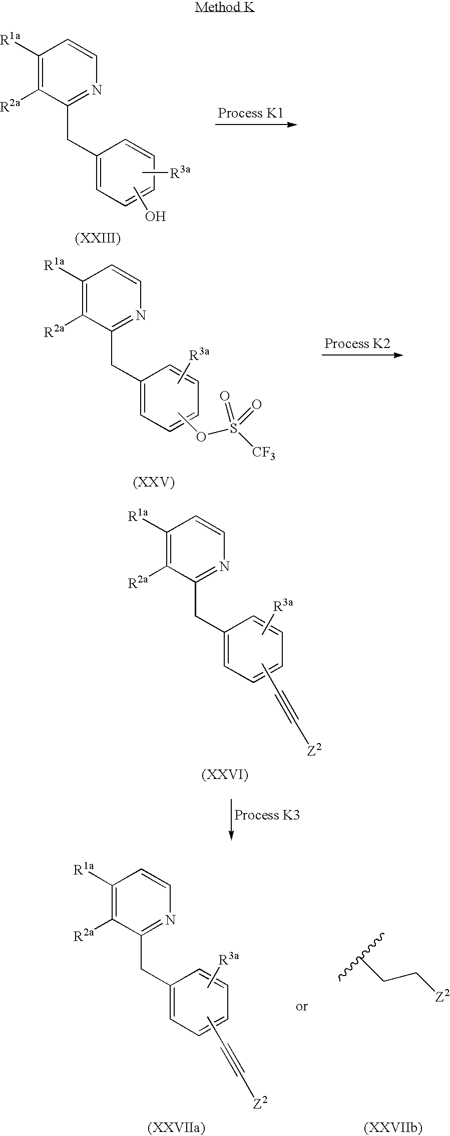

The symbols in the formulae have the same meaning as defined above.

Process K1

A process for the triflation reaction. The compound (XXV) can be produced by reacting the compound (XXIII) with trifluoromethane sulfonic acid anhydride in the presence of a base.

Process K2

A process for the coupling reaction with an alkyne. The compound (XXVI) can be produced by coupling the compound (XXV) with an alkyne derivative in the presence of a palladium phosphine complex, copper iodide, and a base. Examples of reagents that produce the palladium phosphine complex in the reaction system include a combination of palladium-carbon and triphenylphosphine, tetrakistriphenylphosphine palladium (0) and triphenylphosphine, dichlorobistriphenylphosphine palladium (II), palladium (II) acetate and tri(o-tolyl)phosphine, and palladium(II) acetate and 1,1′-bis (diphenylphosphino) ferrocene. Examples of the base include triethylamine, piperidine, pyridine, and potassium carbonate. Depending on the reaction, lithium chloride may be used.

Process K3

A process for the reduction reaction of the unsaturated hydrocarbon. The compound (XXVIIa) or the compound (XXVIIb) can be produced from the compound (XXVI), for example, by catalytic hydrogenation using a catalyst. Examples of the catalyst include palladium-carbon, palladium hydroxide, platinum oxide, and palladium-carbon-calcium carbonate.

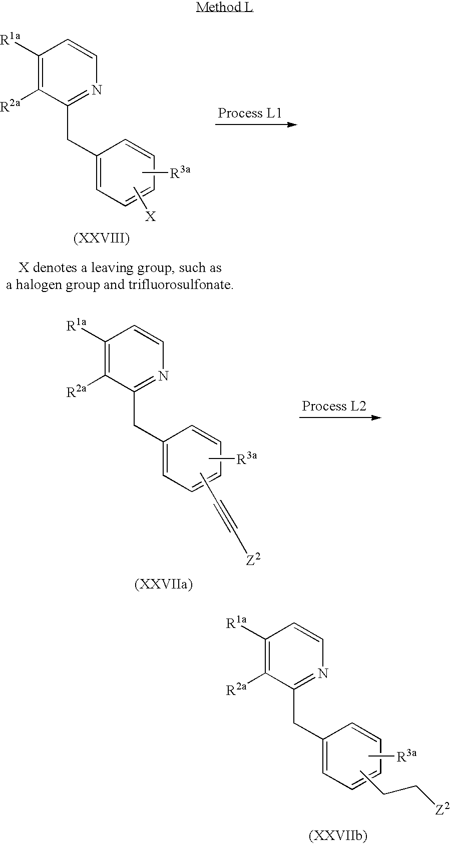

The symbols in the formulae have the same meaning as defined above.

Process L1

A process of the coupling reaction (Heck Reaction) with the alkene. The compound (XXVIIa) can be produced from the compound (XXVIII) using a catalyst (e.g. palladium complex and its ligand), based on the reaction conditions according to the literature, such as J. Org. Chem. 37, 2320 (1972); Org. Reactions., 27, 345 (1982); Comprehensive Organic Synthesis, Vol. 4, 833 (1991); Palladium Reagents and Catalysts, 125 (1995); Chem. Commun., 1287(1984); Tetrahedron Lett, 26, 2667 (1985); and Tetrahedron Lett, 31, 2463 (1990). Examples of the combination of the catalysts used for this reaction (palladium complex and its ligand) include palladium (II) acetate and 1,1′-bis(diphenylphosphino)ferrocene, and palladium (II) acetate and tri(o-tolyl)phosphine. Examples of the tertiary base include triethylamine, diisopropylethylamine, and 1,8-diazabicyclo[5.4.0]-7-undecene. X of the compound (XXVIII) denotes a leaving group, such as a halogen group and trifluoromethanesulfonyloxy group.

Process L2

The compound (XXVIIb) can be produced from the compound (XXVIIa) according to the conditions for a reduction reaction of an unsaturated hydrocarbon, similar to that of process K3.

Method L is a method for producing the compound (XXVIIa) by Process L1, followed by producing the compound (XXVIIb) by Process L2.

Various isomers of the compounds represented by the formula (I) of the present invention can be purified and isolated using ordinary separation techniques (for example, recrystallization, chromatography, and so on).

Compounds of the present invention or salts thereof, or hydrates thereof can be administered as they are to mammals (preferably humans) They can also be formulated by a conventional method into tablets, powders, fine granules, granules, coated tablets, capsules, syrups, troches, inhalants, suppositories, injections, ointments, eye ointments, eye drops, nasal drops, ear drops, cataplasms, lotions, and such, then administered. For the pharmaceutical formulation, ordinarily used auxiliary agents for pharmaceutical formulation (for example, fillers, binders, lubricants, coloring agents, flavoring agents, and as necessary, stabilizers, emulsifiers, absorbefacient, surfactants, pH regulators, antiseptics, antioxidants, etc.) can be used. The pharmatical formulation can be prepared by an ordinary method by combining components that are generally used as ingredients for pharmaceutical preparations. For example, oral preparations can be produced by combining the compounds of the present invention or a pharmaceutically acceptable salt thereof with fillers, and as necessary, binders, disintegrators, lubricants, coloring agents, flavoring agents, and such, and formulating the mixture into powders, fine granules, granules, tablets, coated tablets, capsules, and such by usual methods. Examples of these components include animal fat and vegetable oil such as soybean oil, beef tallow, and synthetic glyceride; hydrocarbons such as liquid paraffin, squalene, and solid paraffin; ester oils such as octyldodecyl myristate and isopropyl myristate; higher alcohols such as cetostearyl alcohol and behenyl alcohol; silicone resin; silicone oil; surfactants such as polyoxyethylene fatty acid ester, sorbitan fatty acid ester, glycerol fatty acid ester, polyoxyethylene sorbitan fatty acid ester, polyoxyethylene hardened castor oil, and polyoxyethylene polyoxypropylene block copolymer; water-soluble macromolecules such as hydroxyethyl cellulose, polyacrylic acid, carboxyvinyl polymer, polyethylene glycol, polyvinyl pyrrolidone, and methyl cellulose; lower alcohols such as ethanol and isopropanol; polyhydric alcohols such as glycerol, propylene glycol, dipropylene glycol, and sorbitol; sugars such as glucose and sucrose; inorganic powder such as silicic acid anhydride, magnesium aluminum silicate, and aluminum silicate; purified water, etc. Examples of fillers include lactose, corn starch, refinedwhite sugar, glucose, mannitol, sorbitol, crystalline cellulose, and silicon dioxide. Examples of binders include polyvinyl alcohol, polyvinyl ether, methyl cellulose, ethyl cellulose, gum arabic, tragacanth, gelatin, shellac, hydroxypropylmethyl cellulose, hydroxypropyl cellulose, polyvinyl pyrrolidone, polypropyleneglycol polyoxyethylene block polymer, and meglumine. Examples of disintegrators include starch, agar, powdered gelatin, crystalline cellulose, calcium carbonate, sodium hydrogencarbonate, calcium citrate, dextrin, pectin, and calcium carboxymethylcellulose. Examples of lubricants include magnesium stearate, talc, polyethyleneglycol, silica, and hardened vegetable oil. Examples of coloring agents are those accepted for addition to medicaments. Examples of flavoring agents include cocoa powder, l-menthol, aromatic dispersant, mint oil, borneol, and cinnamon powder. The use of sugar coating and other appropriate coating as necessary is of course permissible for these tablets and granules. Furthermore, liquid preparations such as syrups and injections can be prepared using conventional methods by adding pH regulators, solubilizers, isotonizing agents, and such, and as necessary, solubilizing adjuvants, stabilizers, and such to the compounds of this invention or pharmaceutically acceptable salts thereof. The method for producing external preparations is not limited and can be produced by a conventional method. That is, base materials used for formulation can be selected from various materials ordinarily used for medicaments, quasi-drugs, cosmetics, and such. Specifically, the base materials to be used are, for example, animal fat and vegetable oil, mineral oil, ester oil, waxes, higher alcohols, fatty acids, silicone oil, surfactants, phospholipids, alcohols, polyhydric alcohols, water soluble macromolecules, clay minerals, and purified water. As necessary, pH regulators, antioxidants, chelating agents, antiseptic and antifungal agents, coloring matters, fragrances, and such may be added, but the base materials of the external preparations of the present invention are not to be construed as being limited thereto. Furthermore, as necessary, components such as those that have a differentiation induction effect, blood flow accelerants, fungicides, antiphlogistic agents, cell activators, vitamins, amino acids, humectants, and keratolytic agents can be combined. The above-mentioned base materials is added to an amount that leads to the concentration usually used for external preparations.

When the compounds of this invention or salts thereof, or hydrates thereof, is administered, there are no particular limitations on their form, and they can be administered orally or parenterally by a conventionally used method. They can be formulated into as dosage forms such as tablets, powder, fine granules, capsules, syrups, troches, inhalents, suppositories, injections, ointments, eye ointments, eye drops, nasal drops, ear drops, cataplasms, and lotions. The dose of the pharmaceutical compositions of this invention can be selected appropriately depending on the degree of the symptom, age, sex, weight, the dosage form, the type of salt, the specific type of disease, and such.

A curative dose of the antifungal agent of this invention is administered to a patient. Herein, “curative dose” refers to the amount of the pharmaceutical agent that yields the desired pharmacological result and is effective for recovery or relief from the symptoms of a patient to be treated. The dose differs markedly depending on the weight of the patient, type of disease, degree of symptom, age of the patient, sex, sensitivity towards the agent, and such. Usually, the daily dose for an adult is approximately 0.03 to 1000 mg, preferably 0.1 to 500 mg, more preferably 0.1 to 100 mg, and is administered once to several times per day, or once to several times per several days. The dose for injections is normally, approximately 1 to 3000 μg/kg, and is preferably approximately 3 to 1000 μg/kg.

### BRIEF DESCRIPTION OF THE DRAWINGS

FIG. 1 is a schematic diagram of the process that transports GPI-anchored proteins to the cell wall. A GPI (Glycosylphosphatidylinositol)-anchored protein is first anchored to GPI, and then transported to the cell wall.

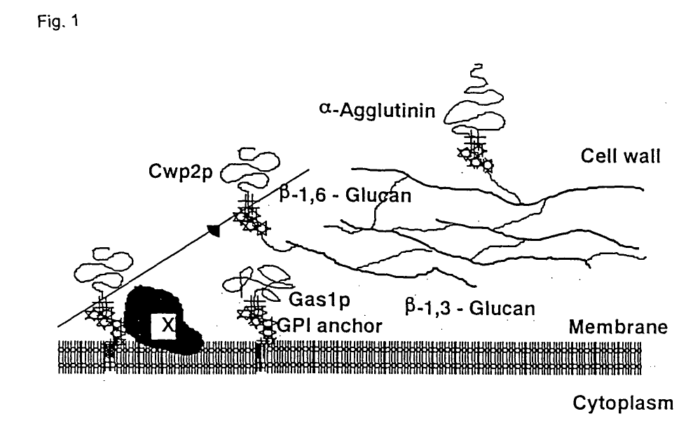

FIG. 2 is a graph showing the activity of the aforementioned compound (Ia) in the S. cerevisiae reporter system. In the presence of the aforementioned compound (Ia) at a concentration of 0.39 to 1.56 μg/ml, cephalosporinase activity increased in the culture supernatant fraction and decreased in the cell wall fraction, and at a concentration of 3.13 μg/ml or more, growth inhibition was observed.

FIG. 3 is a graph showing the effect of the aforementioned compound (Ia) on the adhesion of C. albicans to animal cells. Even at a concentration of 1.56 μg/ml in which growth inhibition cannot be observed, adhesion of C. albicans to animal cells was inhibited to about a half.

FIG. 4 is a graph showing the effect of the aforementioned compound (Ia) on the amount of the Als1p antigen of C. albicans. In the presence of the aforementioned compound (Ia) at a concentration of 0.1 to 0.39 μg/ml, the amount of the Als1p antigen increased in the culture supernatant fraction and the amount of the antigen decreased in the cell wall fraction.

FIG. 5 is a photograph showing the Southern Blot analysis of the C. albicans gene using the GWT1 gene as a probe. A single band was observed at 6.5 kb with EcoRI, at 4.0 kb with HindIII, at 2.0 kb with EcoRI-HindIII, and at 2.5 kb with EcoRI-PstI, and the homologue of the resistant gene to the aforementioned compound (Ia) in C. albicans was expected to exist as a single gene.

FIG. 6 is a graph showing the activity of the aforementioned compound (Ia) in S. cerevisiae that overexpressed the GWT1 gene product. In S. cerevisiae CW63 strain (“W/T” in the Figure), even at the concentration of the aforementioned compound (Ia) (0.39 to 1.56 μg/ml) in which cephalosporinase activity in the culture supernatant fraction is increased, and activity in the cell wall fraction is decreased, such an effect was not observed in S. cerevisiae CW63/GWT1 strain, and in S. cerevisiae CW63 strain, even at the concentration of the aforementioned (>3.13 μg/ml) in which growth is inhibited, growth inhibition was not observed in S. cerevisiae CW63/GWT1 strain (“O/E” in the Figure).

FIG. 7 is a diagram in which the highly conserved regions in the proteins encoded by the GWT1 genes of S. cerevisiae, S. pombe, and C. albicans are aligned.

### BEST MODE FOR CARRYING OUT THE INVENTION

### Example A

The present invention is specifically illustrated below with reference to Examples, but it is not to be construed as being limited thereto.

### Example A1

### Construction of the Reporter Gene and Introduction Thereof into S. cerevisiae

(1). Construction of the Reporter Gene Where Lysozyme is the Reporter Enzyme

A lysozyme gene comprising a promoter sequence was amplified by PCR using pESH plasmid comprising the ENO1 promoter + secretion signal + the lysozyme gene (Ichikawa K et al, Biosci. Biotech. Biochem., 57 (10), 1686-1690, 1993) as template, and the oligonucleotides of SEQ ID NO: 8 and SEQ ID NO: 9 as primers, and this was subcloned into the SalI-EcoRI site of pCR-Script SK(+) (a). Furthermore, a CWP2 gene was amplified by PCR using S. cerevisiae chromosomal DNA as template, and the oligonucleotides of SEQ ID NO: 10 and SEQ ID NO: 11 as primers, and this was subcloned into the EcoRI-HindIII site of pUC19 (b). Similarly, CYC1 terminator was amplified by PCR using pYES2 (INVITROGEN) as a template, and the oligonucleotides of SEQ ID NO: 12 and SEQ ID NO: 13 as primers, and this was subcloned into the newly introduced NotI-KpnI site of pUC19 (c).

Next, the lysozyme gene excised with SalI-EcoRI (a), and the CWP2 gene excised with EcoRI-HindIII (b) were inserted into the SalI-HindIII cleavage site of pESH. Finally, pRLW63T was produced by excising a gene comprising the ENO1 promoter + secretion signal + lysozyme gene + CWP2 gene using BamHI-HindIII, inserting this into a pRS306 integration vector (Sikorski R S et al, Genetics. 122(1): 19-27, 1989), and then inserting the CYC1 terminator excised with HindIII-KpnI (c) into the HindIII-KpnI cleavage site.

(2). Construction of the Reporter Gene Where Cephalosporinase is the Reporter Enzyme

DNA comprising a promoter sequence and secretion signal portion was amplified by PCR using the abovementioned pESH as template, the ENO1 promoter C-terminus + secretion signal portion (d) as template, and the oligonucleotides of SEQ ID NO: 14 and SEQ ID NO: 15 as primers, and this was subcloned into the BamHI-NotI site newly introduced into pUC19 (d). Furthermore, a cephalosporinase gene was amplified by PCR using Citrobacter freundii chromosomal DNA as template, and the oligonucleotides of SEQ ID NO: 16 and SEQ ID NO: 17 as primers, and this was subcloned into the NspV-XbaI site newly introduced into pUC19 (e). Similarly, the CWP2 gene was amplified by PCR using the S. cerevisiae chromosomal DNA as template, and the oligonucleotides of SEQ ID NO: 18 and SEQ ID NO: 19 as primers, and this was subcloned into the XbaI-HindIII site of pUC19 (f).

After producing the full length ENO1 promoter + secretion signal portion by inserting the BamHI-SalI fragment of pESH into the BamHI-SalI cleavage site of a plasmid into which (d) has been inserted, the cephalosporinase gene excised with NspV-XbaI, and the CWP2 gene excised with XbaI-HindIII were inserted into the NspV-HindIII cleavage site. Next, pRCW63T was produced by excising with EcoRI-HindIII, inserting this fragment into the abovementioned pRS306, and then inserting the CYC1 terminator into the HindIII-KpnI cleavage site.

(3). Introduction of the Reporter Gene Into S. cerevisiae

S. cerevisiae G2-10 strain was cultured by shaking in 10 ml of YPD medium at 30° C., then the cells were collected at the late logarithmic growth phase (2-5×10⁷ cells/ml). After washing with sterilized water, the above mentioned pRLW63T and pRCW63T were introduced by lithium acetate method that uses YEASTMAKER™ Yeast Transformation System (Clontech) (according to the YEASTMAKER™ Yeast, Transformation System User Manual). pRLW63T and pRCW63T in which the URA3 gene was cleaved with EcoRV and ApaI, respectively, were used. After culturing in SD(Ura⁻) medium at 30° C. for 3 days, the grown colonies were cultured in YPD medium.

When the localizations of lysozyme and cephalosporinase activities were confirmed, both activities were mainly localized in the cell wall, and the C-terminal sequence of CWP2 was confirmed to function as a transport signal to the cell wall.

### Example A2

### Screening of Pharmaceutical Agents by the S. cerevisiae Reporter System

Since sensitivity of the enzyme reaction is better with cephalosporinase compared to lysozyme, S. cerevisiae introduced with pRCW63T (S. cerevisiae CW63 strain) was used for the screening of compounds.

After stationary cultivation in YPD liquid medium at 30° C. for 48 hours, the yeast cell culture was diluted 100 times with YPD liquid medium (3-5×10⁵ cells/ml) and 75 μl/well aliquots thereof were inoculated into a V-bottomed 96-well plate containing 25 μl/well of a diluted test sample, and this was subjected to stationary cultivation at 30° C. for 48 hours. After centrifuging the plate, 25 μl of the supernatant was sampled and placed in a flat-bottomed 96-well plate, and this was used as the culture supernatant fraction.

The precipitated cells were suspended, and 75 μl/well aliquots of Zymolyase (Seikagaku Corporation) solution prepared with 2.4 M sorbitol were added and were allowed to react at 30° C. for 1 hour. After centrifuging the plate, 10 μl of the supernatant was sampled and placed in a flat-bottomed 96-well plate, 15 μl of phosphate buffer was added, and this was used as the cell wall fraction.

The cephalosporinase activities in the medium and in the cell wall fraction were measured by adding 200 μM of nitrocefin solution to a pooled sample, and after a certain period of time, stopping the reaction with citric acid buffer, and then measuring the absorbance at 490 nm.

Furthermore, fungal growth in the presence of the test sample was determined by visual observation.

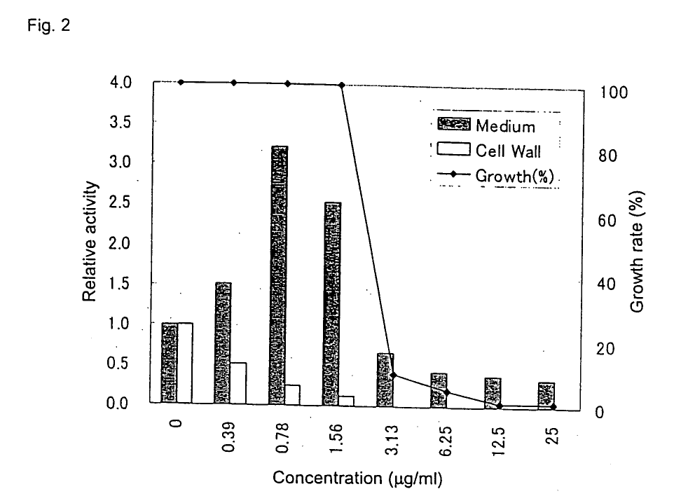

FIG. 2 showed that in the presence of the aforementioned compound (Ia) at a concentration of 0.39 to 1.56 μg/ml, cephalosporinase activity increases in the culture supernatant fraction, and the activity decreases in the cell wall fraction. In this manner, a compound that increases the cephalosporinase activity in the culture supernatant fraction, and in addition decreases the cephalosporinase activity in the cell wall fraction was considered to be a compound that inhibits the process that transports GPI-anchored proteins to the cell wall.

### Example A3

### Screening of Pharmaceutical Agents Using the Adhesion of Candida to Animal Cells

Three-milliliter aliquots of IEC-18 cells (1×10⁵ cells/ml in D-MEM medium (Nissui Pharmaceutical) containing 10% fetal calf serum and 2 mM glutamine) were placed in each well of a 6-well multi-well plate. The plate was incubated in a carbon dioxide gas incubator at 37° C. for 3 days, the culture supernatant was removed, and ethanol immobilization was carried out.

C. albicans cultured in Sabouraud Dextrose Liquid Medium containing various concentrations of the test sample at 30° C. for 48 hours was adjusted to 4×10² cells/ml, and 1 ml was inoculated into each well of the plate in which the immobilized IEC-18 cells were cultured. After cultivation at 30° C. for 1 hour, the culture supernatant was removed, washed with PBS, and then 2 ml of Sabouraud Dextrose Agar Medium (Difco) was superposed. After cultivation at 30° C. overnight, the number of colonies (CFU) that had grown was counted and the adhesion rate was calculated.

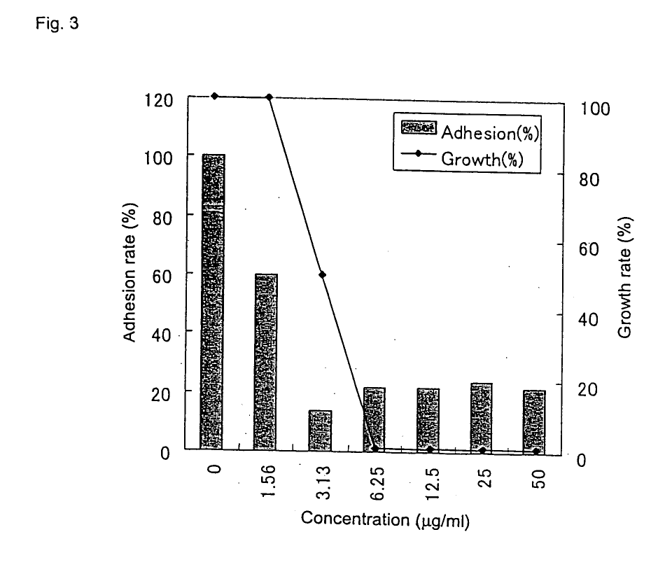

FIG. 3 shows that even at a concentration of 1.56 μg/ml of the aforementioned compound (Ia), in which growth inhibition cannot be observed, adhesion of C. albicans to animal cells was inhibited to about a half. Compared to untreated C. albicans, a test sample that diminished CFU that adhered to cells was considered as a compound that inhibits the adhesion of C. albicans to animal cells.

### Example A4

### Screening of Pharmaceutical Agents Using the Amount of the GPI-Anchored Protein Quantified by ELISA

(1). Production of Anti-Als1p Peptide Antibody

A house rabbit was immunized with the synthetic peptide of SEQ ID NO: 20 which was conjugated with KLH. The obtained antisera was affinity-purified, and the IgG fraction was used as the anti-Als1p peptide antibody.

(2). Screening of Pharmaceutical Agents by ELISA Using Anti-Als1p Peptide Antibody

C. albicans was cultured in Sabouraud Dextrose Liquid Medium (5 ml) containing various concentrations of the test sample at 30° C. for 48 hours, and the cells were collected by centrifugation, washed, and then suspended in 300 μl of Tris-HCl buffer. The suspended cells were transferred to a microtube containing glass beads, and were disrupted by repeating 10 cycles of stirring for 1 minute and cooling on ice for 1 minute. The disrupted cells that were washed were extracted with 2% SDS at 95° C. for 10 minutes, centrifuged, and then the precipitate was washed 5 times with phosphate buffer. To this precipitate, 0.5 ml of 5 μg/ml Zymolyase solution was added, reacted at 37° C. for 1 hour, and the centrifuged supernatant was used as the GPI-anchored protein sample.

A 96-well plate was coated with 50 μL of anti-Als1p peptide antibody (40 μg/ml) at 4° C. overnight. After washing 5 times with PBS containing 0.05% Tween 20 (PBST), blocking was carried out with 25% BlockAce at room temperature for 2 hours. After washing 3 times with PBST, 50 μl of the 2-fold serially diluted GPI-anchored protein sample was reacted at room temperature for 2 hours. After washing 5 times with PBST, 100 μl of 1000-fold diluted HRP-labeled anti-Candida antibody (ViroStat) was reacted at room temperature for 2 hours, then upon washing 5 times with PBST, 75 μl of substrate solution was added. After the reaction was stopped, absorbance at 490 nm was measured.

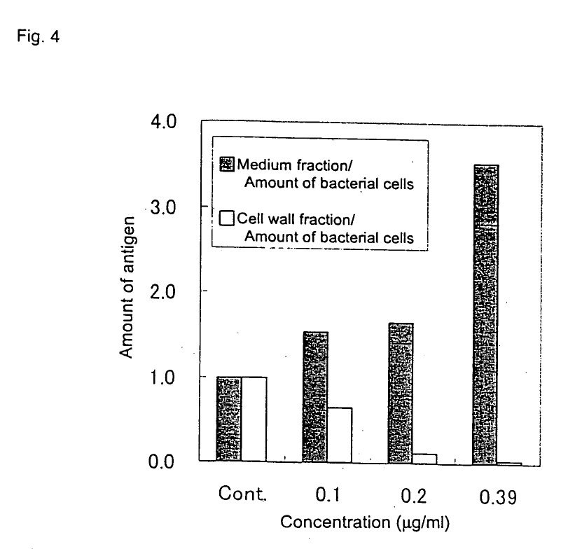

FIG. 4 shows that in the presence of the aforementioned compound (Ia) at a concentration of 0.1 to 0.39 μg/ml, the amount of Als1p antigen increases in the culture supernatant fraction, and the amount of antigen decreases in the cell wall fraction. In this manner, a compound that increased the amount of Als1p in the culture supernatant, or decreased the amount of Als1p in the cell wall fraction, as quantified by ELISA, compared to the amount of Als1p in C. albicans untreated with the compound, was considered to be a compound that inhibits the process that transports GPI-anchored proteins to the cell wall in C. albicans.

### Example A5

### Observation of the Cell Wall of C. albicans Cultured in the Presence of a Test Sample by an Electron Microscope

C. albicans which was cultured in Sabouraud Dextrose Liquid Medium (5 ml) containing various concentrations of the test agent at 30° C. for 48 hours, then centrifuged, and collected, was immobilized by potassium permanganate immobilization method, and the transmission electron microscope image thereof was observed.

The flocculent fibrous structure with high electron density was observed in the outermost layer of the cell, and was considered to be the surface layer glycoprotein layer having the GPI-anchored protein as its constituent. This flocculent fibrous structure was not influenced by other existing antifungal agents.

In C. albicans cultured in the presence of the aforementioned compound (Ia), the flocculent fibrous structure of the outermost layer of the cell having high electron density disappeared leaving a small amount of the layer with high electron density, compared to that in untreated cells. In this manner, when the flocculent fibrous structure of the outermost layer of the fungal cell having high electron density disappeared, the test sample was considered to be the compound influencing the process that transports GPI-anchored proteins to the cell wall.

### Example A6

### Screening of the Resistant Gene to the Aforementioned Compound (Ia) of S. cerevisiae

The plasmid library of the S. cerevisiae gene was obtained from ATCC (Information for ATCC Number: 37323).

S. cerevisiae G2-10 strain was cultured while shaking in 10 ml of YPD medium at 30° C., and cells were collected at the late logarithmic growth phase (1-2×10⁷ cells/ml). After washing the cells with sterilized water, the plasmid library of the S. cerevisiae gene was introduced by the lithium acetate method that uses YEASTMAKER™ Yeast Transformation System (Clontech) (according to YEASTMAKER™ Yeast Transformation System User Manual), and this was spread onto a SD(Leu⁻) plate, and approximately 80,000 colonies were obtained. The colonies were collected and diluted, and were spread onto a SD(Leu⁻) plate containing the aforementioned compound (Ia) at a concentration of 1.56 μg/ml and 3:125 μg/ml so that there were 570,000 colonies per plate. Subsequently, the resistant clone was obtained by incubation at 37° C. for 72 hours.

When 27 clones were picked and plasmids were collected by the method according to METHODS IN ENZYMOLOGY, Vol. 194: 169-182 (1991), and the inserts were analyzed, all 27 contained the same fragment.

As a result of determining the nucleotide sequence using the ABI377 system (PE Applied Biosystems), the DNA of SEQ ID NO: 1 was found to be the DNA that confers resistance to the aforementioned compound (Ia), and was named GWT1.

### Example A7

### Southern Blot Analysis of a C. albicans Homologue of the S. cerevisiae GWT1 Gene

A sample was prepared by treating 25 μg of the C. albicans genomic DNA with EcoRI (TaKaRa), HindIII (TaKaRa), BamHI (TOYOBO), or PstI (New England Biolabs) (including a combination of 2 types of enzymes) for 16 hours, then concentrating by ethanol precipitation, and dissolving in 25 μl of sterilized water. Twenty-five micrograms of genomic DNA digested with restriction enzymes was separated by 0.75% agarose gel electrophoresis method, and was transferred to a nylon membrane (GeneScreen PLUS/NEN).

A probe was produced by labeling 20 ng of the approximately 1.5 kb DNA fragment of SEQ ID NO: 1 with alpha33P-dCTP by the random primer method, and was purified using a GeneQuant column (Amersham-Pharmacia).

Hybridization was carried out by soaking the membrane in 10 ml of PerfectHyb™ (TOYOBO) solution, preincubating at 65° C. for 1 hour, then adding the labeled probe mentioned above, and incubating at 65° C. for 2.5 hours. Washing was carried out with 1). 2×SSC, 0.05% SDS solution at 25° C. for 5 minutes, 2). 2×SSC, 0.05% SDS solution at 25° C. for 15 minutes, and 3). 0.1×SSC, 0.1% SDS solution at 50° C. for 20 minutes. The washed membrane was wrapped with Saran Wrap, and contacted with an Imaging Plate (FUJI) for 12 hours at room temperature, the image that was transferred to the Imaging Plate was captured using BAS2000 (FUJI), and the image was analyzed.

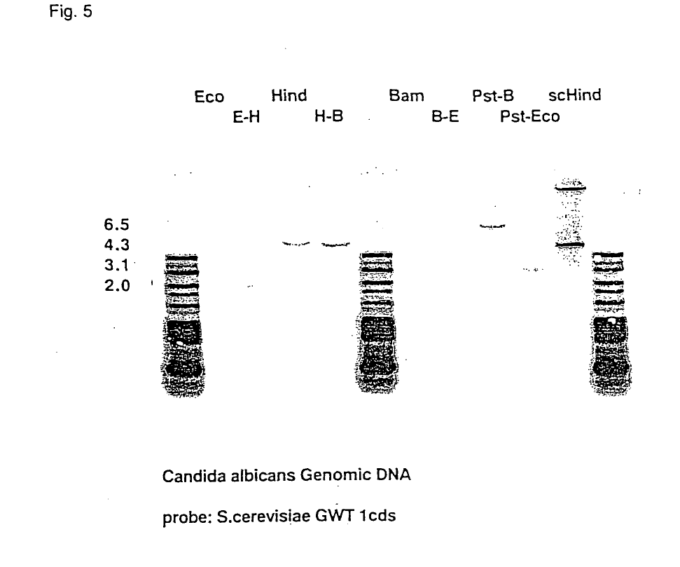

As a result, single bands were observed at 6.5 kb with EcoRI, 4.0 kb with HindIII, 2.0 kb with EcoRI-HindIII, and 2.5 kb with EcoRI-PstI (FIG. 5), and the homologue of the resistant gene to the aforementioned compound (Ia) of C. albicans was expected to exist as a single gene.

### Example A8

### Screening of the Resistant Gene to the Aforementioned Compound (Ia) of C. albicans

The genomic library of C. albicans was produced by the method according to Navaro-Garcia F et al, Mol. Cell. Biol., 15: 2197-2206, 1995. Specifically, the genomic DNA of C. albicans was partially digested with Sau3AI, then DNA fragments around 3 to 5 were collected, and these were inserted into the BamHI site of YEp352 shuttle vector.

S. cerevisiae G2-10 strain was cultured by shaking in 10 ml of YPD medium at 30° C., and cells were collected at the late logarithmic growth phase (2-5×10⁷ cells/ml). After washing the cells with sterilized water, a genomic library of the C. albicans was introduced by the lithium acetate method that uses YEASTMAKER™ Yeast Transformation System (Clontech) (according to YEASTMAKER™ Yeast Transformation System User Manual), and this was spread onto a SD(Ura⁻) plate, and approximately 25,000 colonies were obtained. The colonies were collected and diluted, and were spread onto a SD plate containing the aforementioned compound (Ia) at a concentration of 1.56 μg/ml so that there were 500,000 colonies per plate. Subsequently, the resistant clones were obtained by incubation at 30° C. for 6 hours, and then transferred to 37° C. and incubated for 66 hours.

When 30 clones were picked and plasmids were collected by the method according to METHODS IN ENZYMOLOGY, Vol. 194: 169-182 (1991) and the inserts were analyzed, 28 out of 30 contained the same fragment.

As a result of determining the nucleotide sequence using the ABI377 system (PE Applied Biosystems), the DNA of SEQ ID NO: 3 was found to be the DNA that confers resistance to the aforementioned compound (Ia).

### Example A9

### Cloning of a Homologue of the Resistant Gene to the Aforementioned Compound (Ia) From the Clinical Isolate of C. albicans

PCR amplification was carried out using as template a genomic DNA that was purified from a clinical isolate of C. albicans that is stored by the inventors, and SEQ ID NO: 21 and SEQ ID NO: 22 as primers. A DNA fragment of approximately 1.6 kb was amplified from all three of the independent PCR samples, the amplified fragments were purified, subcloned into a pT7-Blue vector (Novagen), and the nucleotide sequence was determined, and thereby, the DNA sequence of SEQ ID NO: 5 was discovered. The sequence was different at three positions as compared to the DNA of Example A7 (SEQ ID NO: 3).

Furthermore, in the nucleotide sequence of the C. albicans gene determined at Stanford University Sequence Center (http://sequence-www.stanford.edu/), a homologue of the DNA of Example A7 was found (SEQ ID NO: 7), and the sequence was different at four positions as compared to the DNA of Example A7 (SEQ ID NO: 3).

### Example A10

### Construction of S. cerevisiae Overexpressing the GWT1 Gene Product

PCR amplification was carried out using a plasmid purified from the resistant clone to the aforementioned compound (Ia) obtained in Example A6 as a template, and SEQ ID NO: 23 and SEQ ID NO: 24 as primers. A PCR product cleaved with PvuII was inserted into the SalI-HindIII cleavage site of pRLW63T produced in Example A1. The entire insert was excised with BamHI-KpnI, and was inserted into the MCS (multi-cloning site) of pRS304 (Sikorski R S et al, Genetics. 122(1): 19-27, 1989) to produce a vector for integration.

S. cerevisiae CW63 strain having a cephalosporinase gene as the reporter gene was cultured by the method according to Example A1, TRP1 of the integration vector was cleaved with EcoRV, and then transformation was carried out by the method of Example A1. GWT1-overexpressed strain (S. cerevisiae CW63/GWT1 strain) was obtained by culturing in SD(Trp⁻) medium at 30° C. for 3 days.

Other than showing resistance to the aforementioned compound (Ia), GWT1-overexpressed strain is not different from the wild type strain, and was sensitive towards other antifungal agents, cycloheximide, benomyl, and amphotericin B.

### Example A11

### Construction of S. cerevisiae Mutant Lacking the GWT1 Gene

His5 cassette containing the GWT1 sequence on both ends was amplified by PCR using the his5 gene of S. pombe (Longtine M S et al, Yeast, 14: 953-961, 1998) as template and SEQ ID NO: 25 and SEQ ID NO: 26 as primers.

S. cerevisiae G2-10 was cultured and the cells were collected by the method according to Example A1, and the abovementioned PCR product was transformed by the method according to Example A1. A GWT1-deficient strain was obtained by cultivation in SD(His⁻) medium at 30° C. for 5 to 7 days.

Although the GWT1-deficient strain shows very slow growth, it was suggested that the growth is not influenced by the aforementioned compound (Ia), and the GWT1 gene product is the target of the compound. Furthermore, the GWT1-deficient strain indicated the following characteristics: it cannot grow at high temperatures; the cells are swollen; and in the observation by a transmission electron microscope, the flocculent fibrous structure of the outermost layer of the fungal cell having high electron density had disappeared.

### Example A12

### Activity of the Aforementioned Compound (Ia) in S. cerevisiae Overexpressing the GWT1 Gene Product

Using S. cerevisiae CW63 strain and GWT1 gene introduced S. cerevisiae CW63/GWT1, activity of the aforementioned compound (Ia) was examined by a method according to the method described in Example A2.

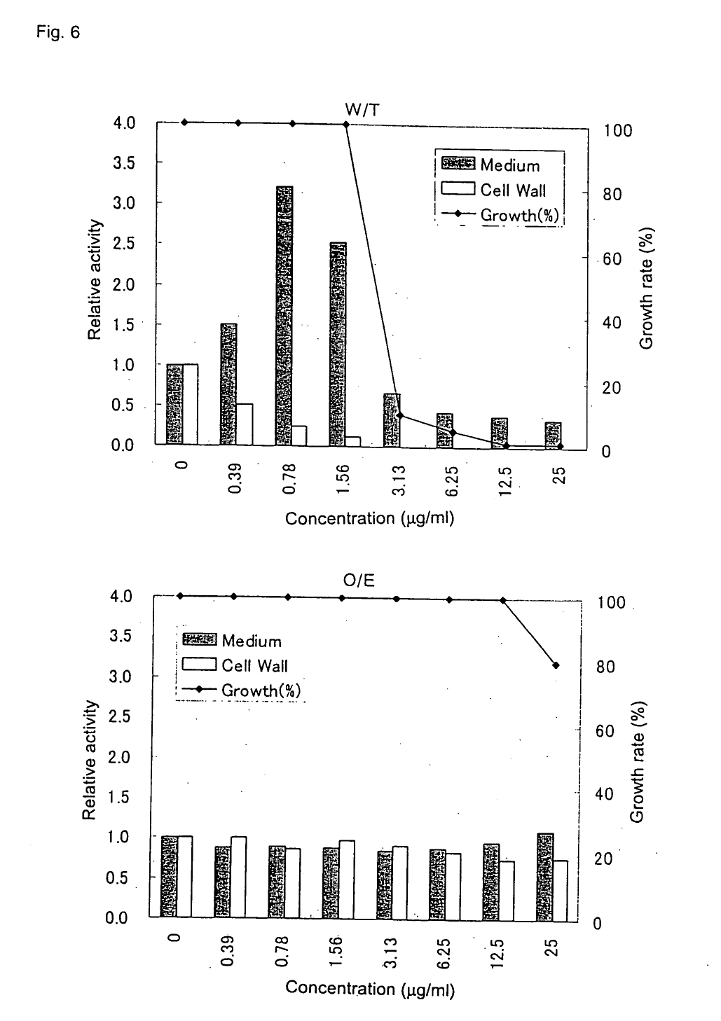

As a result, even at a concentration (0.39 to 1.56 μg/ml) of the aforementioned compound (Ia) at which cephalosporinase activity in the culture supernatant fraction is increased, and the activity in the cell wall fraction is decreased in S. cerevisiae CW63 strain, no influence was observed in the S. cerevisiae CW63/GWT1 strain, and even at a concentration (>3.13 μg/ml) of the aforementioned compound (Ia) at which growth is inhibited in S. cerevisiae CW63 strain, growth inhibition was not observed in the S. cerevisiae CW63/GWT1 strain (FIG. 6).

### Example A13

### Synthesis of (4-Butylphenyl)(1-isoquinolyl)ketone

Under a nitrogen atmosphere, 1-bromo-4-butylbenzene (2.29 ml, 13.0 mmol) was added to a mixed solution of magnesium (338 mg, 13.9 mmol) and tetrahydrofuran (6.5 ml), and as an initiator, catalytic amount of 1,2-dibromoethane was added, and this was stirred under reflux for 10 minutes. The solution was cooled to 0° C., a tetrahydrofuran solution of 1-isoquinolinecarbonitrile (1.0 g, 6.49 mmol) was added, and was stirred for another 1 hour at room temperature, and at 70° C. for 3 hours. Subsequently, the solution was cooled again to 0° C., concentrated hydrochloric acid (2.56 ml) and methanol (11 ml) were added, and then refluxed for 2 hours. The concentrated residue was dissolved in 5 N sodium hydroxide and toluene, and was filtered through celite. The toluene layer of the filtrate was divided, washed with water, dried over magnesium sulfate, and concentrated. The residue was purified by silica gel column chromatography to give 1.72 g of the title compound.

¹H-NMR (CDCl₃) δ (ppm): 0.93 (3H, t), 1.32-1.43 (2H, m), 1.58-1.66 (2H, m), 2.68 (2H, t), 7.28 (2H, d), 7.61 (1H, td), 7.74 (1H, td), 7.80 (1H, d), 7.87 (2H, d), 7.92 (1H, d), 8.20 (1H, d), 8.60 (1H, d).

### Example A14

### Synthesis of {1-(4-Butylbenzyl)isoquinoline}, the Aforementioned Compound of the Formula (Ia)

The compound of Example A13 (1.72 g, 5.95 mmol), hydrazine monohydrate (836 mg, 16.7 mmol), and potassium hydroxide (769 mg, 13.7 mmol) were added to diethylene glycol (8.5 ml), and were stirred at 80° C. for 1 hour, at 160° C. for 3 and a half hours, and at 200° C. for 1 hour. Upon cooling to room temperature, ice water was added and extracted with ethyl acetate. This was washed with water, then dried over magnesium sulfate, and concentrated. The residue was purified by silica gel column chromatography to give 914 mg of the aforementioned compound of the formula (Ia).

¹H-NMR (CDCl₃) δ (ppm): 0.89 (3H, t), 1.26-1.36 (2H, m), 1.50-1.59 (2H, m), 2.53 (2H, t), 4.64 (2H, s), 7.06 (2H, d), 7.19 (2H, d), 7.53 (1H, td), 7.56 (1H, d), 7.64 (1H, td), 7.81 (1H, d), 8.18 (1H, dd,), 8.50 (1H, d).

### Example A15

### Another Method for Producing {1-(4-Butylbenzyl)isoquinoline}, the Aforementioned Compound of the Formula (Ia)

To a dimethylformamide (1.8 ml) solution of 60% sodium hydride (16 mg, 0.40 mmol), a dimethylformamide (3.6 ml) solution of 1-cyano-2-benzoyl-1,2-dihydroisoquinoline (100 mg, 0.38 mmol) synthesized according to the literature of Org. Synth., VI, 115 (1988), and 4-n-butylbenzylchloride (70 mg, 0.38 mmol) was added dropwise under nitrogen atmosphere at −16° C., and was further stirred at room temperature for 30 minutes. Water was added, this was concentrated, and toluene and water were added to this residue. The toluene layer was washed with water, dried over potassium carbonate, and concentrated. To an ethanol (1.6 ml) solution of the residue, 50% aqueous sodium hydroxide solution (0.63 ml) was added, and this was refluxed for 2 hours. After concentration, toluene and water were added. The toluene layer was washed with water, then dried over calcium carbonate, and then concentrated. The residue was purified by silica gel column chromatography to give 18 mg of the aforementioned compound of the formula (Ia).

### Example A16

### Cloning of the C. albicans Homologue of the S. cerevisiae GWT1 Gene

The C. albicans genomic DNA (25 μg) treated with HindIII (TaKaRa) for 16 hours was separated by 0.75% agarose gel electrophoresis method, and the DNA fragments ranging in size from approximately 3.5 to 4.5 kb were recovered from the gel. The recovered DNA fragments were inserted into the HindIII site of the pKF3 vector (TaKaRa), and a Candida genomic library was produced.

Using the produced library, approximately 10,000 colonies were displayed on an LB/Ampicillin plate, colony lifting was performed using a Colony/Plaque Screen (NEN) membrane, and then this was subjected to hybridization. A probe was produced by labeling 20 ng of the approximately 1.5 kb DNA fragment of SEQ ID NO: 1 with alpha ³³P-dCTP by the random primer method, and purifying using a GeneQuant column (Amersham-Pharmacia).

Hybridization was carried out by pre-incubating the membrane in a PerfectHyb™ (TOYOBO) solution at 65° C. for 1 hour, then adding the labeled probe mentioned above, and incubating further at 65° C. for 2.5 hours. Washing was carried out with (i) 2×SSC, 0.05% SDS solution at 25° C. for 5 minutes, (ii) 2×SSC, 0.05% SDS solution at 25° C. for 15 minutes, and (iii) 0.1×SSC, 0.1% SDS solution at 50° C. for 20 minutes. The washed membrane was wrapped with Saran Wrap, contacted with an X-RAY FILM (KONICA) for 24 hours at room temperature, and then developed. The E. coli colonies corresponding to the exposed spots were isolated, and were subjected to secondary screening. Approximately 200 of the isolated colonies were displayed on each LB/Ampicillin plate, colony lifting was performed in a similar manner to primary screening, which was followed by hybridization. The conditions for hybridization were the same as the conditions for primary screening.

As a result, a single colony of E. coli that reacts strongly with the probe was isolated. Plasmids were collected from this colony, and when the contained sequence was determined, a novel sequence having the same sequence as that revealed in Example A9 (SEQ ID NO: 5) was found (the sequence of Candida GWT1), and was presumed to be a C. albicans homologue.

### Example A17

### The S. Pombe Homologue of the S. cerevisiae GWT1 Gene

S. Pombe genes that show homology to the S. cerevisiae GWT1 gene (SEQ ID NO: 27, and the amino acid sequence of the gene product thereof: SEQ ID NO: 28) were found from a database search, and were considered to be the S. Pombe homologues of GWT1.

### Example A18

### Cloning of the Aspergillus fumigatus Homologue of the S. cerevisiae GWT1 Gene

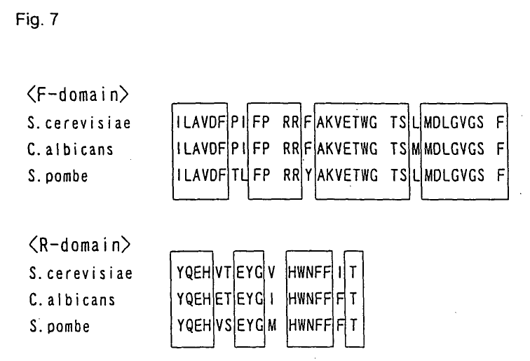

By genetic sequence analysis, the inventors discovered two highly conserved regions in the protein encoded by the GWT1 genes of S. cerevisiae, S. pombe, and C. albicans (FIG. 7). Based on the presumed DNA that encodes the amino acid sequence of this conserved region, primers of SEQ ID NO: 29, SEQ ID NO: 30, and SEQ ID NO: 31 were designed. PCR amplification was carried out using 1 ∥l of the library purchased from STRATAGENE (Aspergillus fumigatus cDNA library: #937053) as a template, and using primers of SEQ ID NO: 29 and SEQ ID NO: 31. Furthermore, as a result of carrying out nested-PCR using 1 μg of this amplified sample as a template, and using primers of SEQ ID NO: 29 and SEQ ID NO: 30, amplification of a single fragment of approximately 250 bp was confirmed. When the sequence of this fragment was determined, a novel sequence having homology to the GWT1 gene of S. cerevisiae, shown in SEQ ID NO: 32, was obtained, and this was presumed to be the homologue of A. fumigatus.

To obtain a full length cDNA, primers of SEQ ID NO: 33 and SEQ ID NO: 34 were designed based on the sequence of the amplified fragment. Furthermore, primers outside the gene insertion site of the library, SEQ ID NO: 35 and SEQ ID NO: 36, were designed. As a result of performing PCR using the A. fumigatus cDNA library as a template, and the primer set of SEQ ID NO: 33 and SEQ ID NO: 35, or the primer set of SEQ ID NO: 34 and SEQ ID NO: 36, amplification of a DNA fragment of approximately 1 kb was confirmed (by both primer sets). As a result of determining the nucleotide sequences of these fragments, a novel sequence that is highly homologous to the GWT1 genes of S. cerevisiae shown in SEQ ID NO: 1 was obtained. Since the sequence is highly homologous to the GWT1 genes of S. cerevisiae, S. pombe, and C. albicans throughout the entire gene, this sequence was strongly suggested to be a homologue of A. fumigatus.

To clone the entire homologue of A. fumigatus, the primer shown in SEQ ID NO: 37 that corresponds to the sequence upstream of the initiation codon, and the primer of SEQ ID NO: 38 that corresponds to the sequence downstream of the stop codon were newly designed based on the obtained sequence. As a result of performing 35 cycles of PCR using the A. fumigatus cDNA library (STRATAGENE) and the A. fumigatus genomic library (STRATAGENE) as templates, and primers of SEQ ID NO: 37 and SEQ ID NO: 38, a single amplified fragment of approximately 1.6 kb was detected from both templates. As a result of determining the nucleotide sequence of this fragment by Direct-Sequencing, the nucleotide sequence shown in SEQ ID NO: 39 was found from the cDNA library, and was suggested to encode a protein comprising 501 amino acids shown in SEQ ID NO: 40. Furthermore, the nucleotide sequence of SEQ ID NO: 41 was found from the genomic library, and was found to have an intron comprising 77 base pairs in one position.

### Example A19

### Cloning of the Cryptococcus Homologue of the S. cerevisiae GWT1 Gene

1). Database Search

As a result of database searching for genes showing homology to the S. cerevisiae GWT1 gene, the sequence of 502042C05.x1 was found from the server of the Genome Center at Stanford University (http://baggage.stanford.edu/cgi-misc/cneoformans/). Furthermore, the sequence of b6e06cn.f1 was found from the server at Oklahoma University, U.S.A (http://www.genome.ou.edu/cneo_blast.html).

2). PCR Using Genomic DNA as Template

The primer of SEQ ID NO: 42 was constructed based on the sequence of 502042C05.x1, and the primer of SEQ ID NO: 43 was constructed based on the sequence of b6e06cn.f1. When PCR amplification was carried out using the genomic DNA of Cryptococcus (Cryptococcus neoformans) as a template, and using the primer of SEQ ID NO: 42, and the primer of SEQ ID NO: 43, an amplified fragment of approximately 2 kb was detected. When the nucleotide sequence of this fragment was determined, a novel sequence showing homology to the GWT1 gene of S. cerevisiae, shown in SEQ ID NO: 44, was obtained.

In order to obtain the sequence upstream of the initiation codon of the Cryptococcus GWT1 gene, the primer of SEQ ID NO: 45 was designed based on the sequence of 502042C05.x1, and the primer of SEQ ID NO: 46 was designed based on the sequence of SEQ ID NO: 44. When PCR amplification was carried out using the genomic DNA of Cryptococcus as a template, and using the primer of SEQ ID NO: 45, and the primer of SEQ ID NO: 46, an amplified fragment of approximately 500 bp was detected. When the nucleotide sequence of this fragment was determined, the sequence of SEQ ID NO: 47 was obtained, and this was found to overlap with SEQ ID NO: 44.

3). 3′-RACE

To obtain the 3′-terminal sequence of the Cryptococcus GWT1 gene, 3′-RACE was carried out. Reverse transcription was carried out by priming with the adaptor-primer of SEQ ID NO: 48, which is based on 16 μg of total RNA extracted from Cryptococcus, and by using SuperScript II Reverse Transcriptase (GIBCO/BRL), and a single stranded cDNA, which is to become the template for the RT-PCR that follows, was produced. As a result of performing 35 cycles of PCR using the single stranded cDNA as a template, and the primers of SEQ ID NO: 49 and SEQ ID NO: 50, an amplified fragment of approximately 1.2 kb was detected. When the nucleotide sequence of this fragment was analyzed by the Direct-Sequencing method, the novel sequence shown in SEQ ID NO: 51 showing homology to the S. cerevisiae GWT1 gene was obtained.

4). PCR of a Full Length Genomic DNA

Using the primer of SEQ ID NO: 52 that was designed based on SEQ ID NO: 47, and the primer of SEQ ID NO: 53 that was designed based on SEQ ID NO: 51, 35 cycles of PCR was carried out on three independent preparations with the genomic DNA of Cryptococcus as template. As a result, an amplified fragment of approximately 2 kb was detected from all three of the independent tubes, and therefore, each of them were individually subjected to Direct-Sequencing, and their entire nucleotide sequences were determined. As a result, the three independent sequences completely matched, and a sequence comprising the full length GWT1 gene homologue of Cryptococcus shown in SEQ ID NO: 54 was obtained.

5). Determination of the cDNA Sequence

Comparison of the sequence of the Cryptococcus GWT1 gene derived from the genome shown in SEQ ID NO: 54 with cDNA sequence 51 obtained by 3′-RACE suggested the presence introns at two positions. Furthermore, since the open reading frame following the ATG initiation codon is not continuous, the presence of another intron was suggested. Therefore, the cDNA structure was predicted from the presumed amino acid sequence and the splicing donor/acceptor sequence, and the primers of SEQ ID NO: 55 and SEQ ID NO: 56 were designed at the position predicted to be the junction between exons. As a result of performing 35 cycles of PCR using the single stranded cDNA derived from Cryptococcus as template with the above-mentioned primers, an amplified fragment of approximately 1.4 kb was confirmed. As a result of determining the nucleotide sequence by subjecting the fragment to Direct-Sequencing, the sequence of SEQ ID NO: 57 was obtained, and by comparing with SEQ, ID NO: 54, the cDNA sequence of the GWT1 gene of Cryptococcus was suggested to have the structure of SEQ ID NO: 58. Since the sequence shows high homology at certain regions with the GWT1 genes of S. cerevisiae, S. pombe, C. albicans, and A. fumigatus, this sequence was strongly suggested to be a homologue of Cryptococcus.

### Example A20

### Genetic Mutation That Confers Resistance to the Aforementioned Compound of the Formula (Ia)

S. cerevisiae LW63 strain having a lysozyme gene as the reporter gene due to introduction of pRLW63T was treated with ethyl methanesulfonate, then by culturing in a SD medium containing the aforementioned compound of the formula (Ia) at concentrations of 1.56, 3.13, and 6.25 μg/ml at 37° C. for 3 days, five resistant mutant strains (R1 to R5) were obtained. Among them, the R1 mutant strain and the R5 mutant strain were found to have acquired a specific resistant characteristic to the aforementioned compound of the formula (Ia) due to a mutation of a single gene. To confirm whether or not these two mutant strains have mutations on the GWT1 gene, genomic DNAs were extracted from both mutant strains, and the nucleotide sequence of the GWT1 gene portion was determined. As a result, in the R1 mutant strain, guanine at position 1213 had been mutated to adenine. Furthermore, in the R5 mutant strain, guanine at position 418 had been mutated to adenine. Therefore, it was elucidated that in the R1 mutant strain, the 405th amino acid, isoleucine, had been changed to valine, and in the R5 mutant strain, the 140th amino acid, glycine, had been changed to arginine.

Next, to confirm whether or not these mutations are the cause of the acquisition of the specific resistant characteristic to the aforementioned compound of the formula (Ia), the mutant GWT1 gene (R1 or R5) was isolated using the genomic DNAs derived from both mutant strains as templates and the primers of SEQ ID NOS: 60 and 61. Simultaneously, the GWT1 promoter region (SEQ ID NO: 62) and the terminator region (SEQ ID NO: 63) were isolated, the GWT1 gene promoter, mutant GWT1 gene ORF, and the GWT1 gene terminator were inserted into the pRS316 vector, and plasmids that express a single copy of the mutant GWT1 gene were constructed (pRS316GWT1-R1, pRS316GWT1-R5). This was introduced to a diploid strain (WDG1) in which only a single copy of the GWT1 gene is disrupted. Spores were formed by culturing the colonies on a sporulation medium, and a clone in which the GWT1 gene on the chromosome is disrupted and also harbors the abovementioned plasmid was obtained by performing a tetrad analysis. When this was cultured in a medium containing the aforementioned compound of the formula (Ia), resistance to the aforementioned compound of the formula (Ia) was seen, similarly to the original R1 mutant strain and R5 mutant strain. From the above, it was elucidated that the specific resistant characteristic to the aforementioned compound of the formula (Ia) is conferred by a point mutation accompanying an amino acid mutation, that occurred on the GWT1 gene, and this compound was strongly suggested to inhibit the function of the GWT1 protein by directly binding to the protein.

### Example B

The compounds of this invention can be produced, for example, by the method of the Examples below. However, the Examples are for illustration purpose only and the compounds of this invention are not to be construed as being limited to those prepared in the following specific examples under any circumstances.

### Example B1

### 1-(Chloromethyl)-4-n-butylbenzene

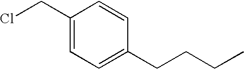

Thionyl chloride (2.5 ml, 34 mmol) was added to a solution of 4-n-butylbenzyl alcohol (2.0 g, 12 mmol) in ether (25 ml), and this mixture was stirred at room temperature for 3 hours. After concentration of the mixture, excess thionyl chloride was removed by azeotropic distillation with benzene to give the title compound (2.3 g). This compound was used in the following reaction without purification.

### Example B2

### 1-(4-Butylbenzylisoquinoline

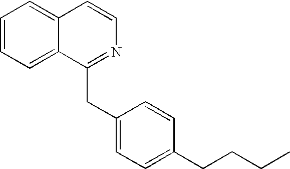

A solution of 1-cyano-2-benzoyl-1,2-dihydroisoquinoline (100 mg, 0.38 mmol), which was synthesized according to Org. Synth., VI, 115 (1988), and 4-n-butylbenzyl chloride (70 mg, 0.38 mmol) in dimethylformamide (3.6 ml) was added dropwise to a solution of 60% sodium hydride (16 mg, 0.40 mmol) in dimethylformamide (1.8 ml) under nitrogen atmosphere at −16° C., and this mixture was stirred at room temperature for 30 minutes. Water was added, the mixture was concentrated under reduced pressure, and toluene and water were added to the residue. The toluene layer was washed with water, dried over potassium carbonate, then concentrated under reduced pressure. A 50% aqueous sodium hydroxide solution (0.63 ml) was added to a solution of the residue in ethanol (1.6 ml) This mixture was heated under reflux for 2 hours and concentrated, and then toluene and water were added. The toluene layer was washed with water, dried over calcium carbonate, and then concentrated under reduced pressure. The residue was purified by silica gel column chromatography to give the title compound (18 mg).

¹H-NMR (CDCl₃) δ (ppm): 0.89 (3H, t), 1.26-1.36 (2H, m), 1.50-1.59 (2H, m), 2.53 (2H, t), 4.64 (2H, s), 7.06 (2H, d), 7.19 (2H, d), 7.53 (1H, td), 7.56 (1H, d), 7.64 (1H, td), 7.81 (1H, d), 8.18 (1H, dd), 8.50 (1H, d).

### Example B3

### (4-Butylphenyl)(1-isoquinolyl)ketone

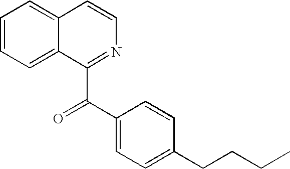

1-Bromo-4-butylbenzene (2.29 ml, 13 mmol) and a catalytic amount of 1,2-dibromoethane as an initiator were added to a mixed solution of magnesium (338 mg, 14 mmol) and tetrahydrofuran (6.5 ml) under nitrogen atmosphere, and this mixture was stirred under reflux for 10 minutes. The mixture was cooled to 0° C., a solution of 1-isoquinolinecarbonitrile (1.0 g, 6.5 mmol) in tetrahydrofuran was added, and this mixture was stirred at room temperature for 1 hour, then at 70° C. for 3 hours. Thereafter, the mixture was cooled again to 0° C., concentrated hydrochloric acid (2.6 ml) and methanol (11 ml) were added, and this mixture was heated under reflux for 2 hours. After the mixture was concentrated, the residue was dissolved in 5 N sodium hydroxide and toluene, and was filtered through celite. The toluene layer of the filtrate was separated, washed with water, dried over anhydrous magnesium sulfate, and then concentrated under reduced pressure. The residue was purified by silica gel column chromatography to give the title compound (1.7 g).

¹H-NMR (CDCl₃) δ (ppm): 0.93 (3H, t), 1.32-1.43 (2H, m), 1.58-1.66 (2H, m), 2.68 (2H, t), 7.28 (2H, d), 7.61 (1H, td), 7.74 (1H, td), 7.80 (1H, d), 7.87 (2H, d), 7.92 (1H, d), 8.20 (1H, d), 8.60 (1H, d).

### Example B4

### Alternative Method for the Production of 1-(4-Butylbenzyl)isoquinoline

The compound of Example B3 (1.7 g, 6.0 mmol), hydrazine monohydrate (836 mg, 17 mmol), and potassium hydroxide (769 mg, 14 mmol) were added to diethylene glycol (8.5 ml), and this mixture was stirred at 80° C. for 1 hour, at 160° C. for 3.5 hours, then at 200° C. for 1 hour. The mixture was cooled to room temperature, ice water was added, and this was extracted with ethyl acetate. The extract was washed with water, dried over anhydrous magnesium sulfate, and concentrated under reduced pressure. The residue was purified by silica gel column chromatography to give the title compound (914 mg).

¹H-NMR (CDCl₃) δ (ppm): 0.89 (3H, t), 1.26-1.36 (2H, m), 1.50-1.59 (2H, m), 2.53 (2H, t), 4.64 (2H, s), 7.06 (2H, d), 7.19 (2H, d), 7.53 (1H, td), 7.56 (1H, d), 7.64 (1H, td), 7.81 (1H, d), 8.18 (1H, dd), 8.50 (1H, d).

### Example B5

### 1-(4-Ethylbenzyl)isoquinoline

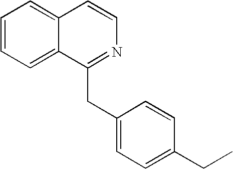

Using p-ethylbenzyl chloride, the title compound was obtained in the same manner as in Example B2.

¹H-NMR (CDCl₃) δ (ppm): 1.18 (3H, t), 2.57 (2H, q), 4.64 (2H, s), 7.08 (2H, d), 7.20 (2H, d), 7.50-7.55 (2H, m), 7.61-7.65 (1H, m), 7.80 (1H, d), 8.16-8.18 (1H, m), 8.49 (1H, d).

### Example B6

### (4-Propylphenyl)methanol

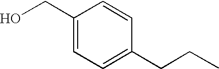

A solution of sodium borohydride (2.9 g, 76 mmol) and concentrated sulfuric acid in ether (prepared by adding 2.0 ml of concentrated sulfuric acid to 4.0 ml of ether) was added dropwise to a solution of p-n-propylbenzoic acid (5.0 g, 32 mmol) in tetrahydrofuran (20 ml) cooled to 0° C. keeping the temperature of the reaction system below 20° C., and then this mixture was stirred at room temperature for 3 hours. After the mixture was cooled on ice, methanol and 1 N sodium hydroxide were added, and this mixture was extracted with ethyl acetate. The ethyl acetate layer was washed with saturated brine, dried over anhydrous magnesium sulfate, and then concentrated under reduced pressure to give the title compound (4.33 g). This compound was used in the following reaction without purification.

### Example B7

### 1-(Chloromethyl)-4-propylbenzene

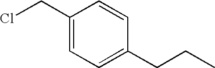

The title compound was obtained by treating the compound of Example B6 in the same manner as in Example B1. This compound was used in the following reaction without further purification.

### Example B8

### 1-(4-Propylbenzyl)isoquinoline

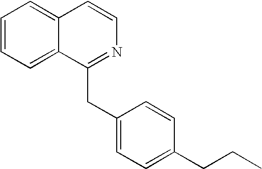

The title compound was obtained by treating the compound of Example B7 in the same manner as in Example B2.

¹H-NMR (CDCl₃) δ (ppm): 0.90 (3H, t), 1.55-1.61 (2H, m), 2.51 (2H, t), 4.64 (2H, s), 7.06 (2H, d), 7.19 (2H, d), 7.51-7.55 (2H, m), 7.61-7.65 (1H, m), 7.81 (1H, d), 8.17 (1H, dd), 8.49 (1H, d).

### Example B9

### (4-Pentylphenyl)methanol

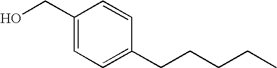

The title compound was obtained by reducing 4-n-amylbenzoic acid in the same manner as in Example B6.

### Example B10

### 1-(Chloromethyl)-4-pentylbenzene

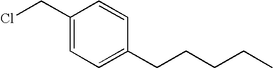

The title compound was obtained by treating the compound of Example B9 in the same manner as in Example B1. This compound was used in the following reaction without further purification.

### Example B11

### 1-(4-Pentylbenzyl)isoquinoline

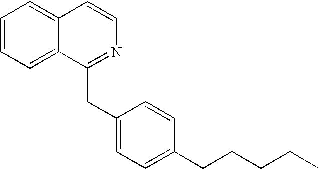

The title compound was obtained by treating the compound of Example B10 in the same manner as in Example B2.

¹H-NMR (CDCl₃) δ (ppm): 0.86 (3H, t), 1.26-1.33 (4H, m), 1.52-1.59 (2H, m), 2.52 (2H, t), 4.64 (2H, s), 7.06 (2H, d), 7.18 (2H, d), 7.50-7.55 (2H, m), 7.61-7.65 (1H, m), 7.80 (1H, d), 8.17(1H, dd), 8.49 (1H, d).

### Example B12

### (4-Hexylphenyl)methanol

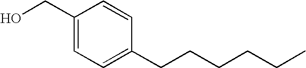

The title compound was obtained by reducing 4-n-hexylbenzoic acid in the same manner as in Example B6. This compound was used in the following reaction without further purification.

### Example B13

### 1-(Chloromethyl)-4-hexylbenzene

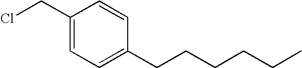

The title compound was obtained by treating the compound of Example B12 in the same manner as in Example B1. This compound was used in the following reaction without further purification.

### Example B14

### 1-(4-Hexylbenzyl)isoquinoline

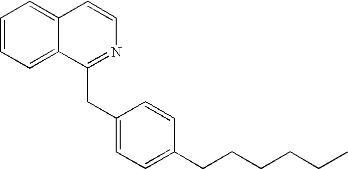

The title compound was obtained by treating the compound of Example B13 in the same manner as in Example B2.

¹H-NMR (CDCl₃) δ (ppm): 0.86 (3H, t), 1.26-1.31 (6H, m), 1.51-1.58 (2H, m), 2.52 (2H, t), 4.63 (2H, s), 7.06 (2H, d), 7.18 (2H, d), 7.50-7.55 (2H, m), 7.61-7.65 (1H, m), 7.80 (1H, d), 8.17 (1H, dd), 8.49 (1H, d).

### Example B15

### 1-(4-Isopropylbenzyl)isoquinoline

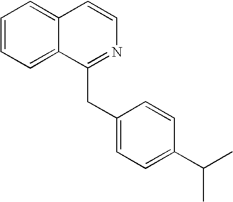

The title compound was obtained by treating p-isopropylbenzyl chloride in the same manner as in Example B2.

¹H-NMR (CDCl₃) δ (ppm): 1.19 (6H, d), 2.80-2.87 (1H, m), 4.64 (2H, s), 7.11 (2H, d), 7.21 (2H, d), 7.51-7.56 (2H, m), 7.61-7.65 (1H, m), 7.81 (1H, d), 8.19 (1H, dd), 8.50 (1H, d).

### Example B16

### 1-[4-(tert-Butyl)benzyl]isoquinoline

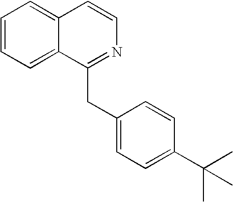

The title compound was obtained by treating 4-tert-butylbenzyl chloride in the same manner as in Example B2.

¹H-NMR (CDCl₃) δ (ppm): 1.26 (9H, s), 4.64 (2H, s), 7.22 (2H, d), 7.27 (2H, d), 7.52-7.56 (2H, m), 7.62-7.66 (1H, m), 7.81 (1H, d), 8.19 (1H, dd), 8.50 (1H, d).

### Example B17

### (4-Isobutylphenyl)methanol

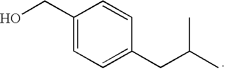

The title compound was obtained by reducing 4-isobutylbenzoic acid in the same manner as in Example B6. This was used in the following reaction without further purification.

### Example B18

### 1-(Chloromethyl)-4-isobutylbenzene

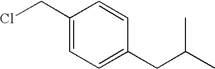

The title compound was obtained by treating the compound of Example B17 in the same manner as in Example B1. This was used in the following reaction without further purification.

### Example B19

### 1-(4-Isobutylbenzyl)isoquinoline

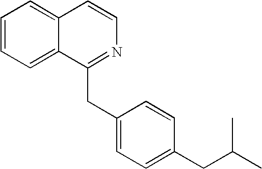

The title compound was obtained by treating the compound of Example B18 in the same manner as in Example B2.

¹H-NMR (CDCl₃) δ (ppm): 0.86 (6H, d), 1.75-1.83 (1H, m), 2.39 (2H, d), 4.66 (2H, s), 7.02 (2H, d), 7.18 (2H, d), 7.52-7.58 (2H, m), 7.63-7.67 (1H, m), 7.82 (1H, d), 8.18 (1H, d), 8.50 (1H, d).

### Example B20

### 1-(Chloromethyl)-4-(trifluoromethyl)benzene

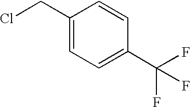

The title compound was obtained by treating 4-trifluoromethylbenzyl alcohol in the same manner as in Example B1. This was used in the following reaction without further purification.

### Example B21

### 1-[4-(Trifluoromethyl)benzyl]isoquinoline

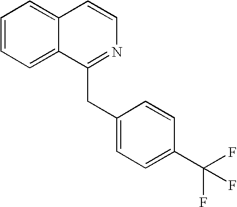

The title compound was obtained by treating the compound of Example B20 in the same manner as in Example B2.

¹H-NMR (CDCl₃) δ (ppm): 4.73 (2H, s), 7.39 (2H, d), 7.51 (2H, d), 7.54-7.60 (2H, m), 7.65-7.69 (1H, m), 7.84 (1H, d), 8.09-8.10 (1H, m), 8.51 (1H, d).

### Example B22

### 1-(Chloromethyl)-4-(trifluoromethoxy)benzene

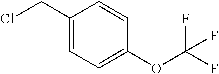

The title compound was obtained by treating 4-trifluoromethoxybenzyl alcohol in the same manner as in Example B1. This was used in the following reaction without further purification.

### Example B23

### 1-[4-(Trifluoromethoxy)benzyl]isoquinoline

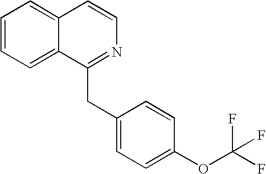

The title compound was obtained by treating the compound of Example B22 in the same manner as in Example B2.

¹H-NMR (CDCl₃) δ (ppm): 4.67 (2H, s), 7.10 (2H, d) 7.27 (2H, d), 7.54-7.59 (2H, m), 7.64-7.68 (1H, m), 7.84 (1H, d), 8.11 (1H, dd), 8.50 (1H, d).

### Example B24

### 1-(Chloromethyl)-2-iodobenzene

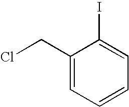

Methanesulfonyl chloride (2.0 ml, 29 mmol) and triethylamine (3.6 ml, 26 mmol) were added to a solution of o-iodobenzyl alcohol (5.0 g, 21 mmol) in methylene chloride (50 ml) cooled to 0° C., and the mixture was stirred at that temperature for 19 hours. A 5% aqueous sodium hydrogencarbonate solution was added, and the resulting mixture was extracted with methylene chloride. The methylene chloride layer was dried over anhydrous magnesium sulfate and concentrated under reduced pressure to give the title compound (5.34 g).

### Example B25

### 1-(2-Iodobenzyl)isoquinoline

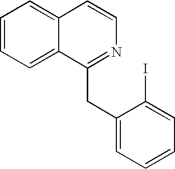

The title compound was obtained by treating the compound of Example B24 in the same manner as in Example B2.

¹H-NMR (CDCl₃) δ (ppm): 4.74 (2H, s), 6.81-6.84 (1H, m), 6.87-6.92 (1H, m), 7.11-7.15 (1H, m), 7.55-7.57 (1H, m), 7.60 (1H, d), 7.64-7.68 (1H, m), 7.83-7.86 (1H, m), 7.89-7.91 (1H, m), 8.00-8.02 (1H, m), 8.50 (1H, d).

### Example B26

### 1-[2-(2-Phenyl-1-ethynyl)benzyl]isoquinoline

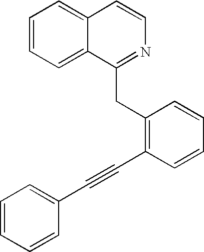

A solution of tetrakis(triphenylphosphine)palladium (58 mg, 0.05 mmol) and ethynylbenzene (204 mg, 2.0 mmol) in pyrrolidine (1.5 ml) was added to a solution of the compound of Example B25 (345 mg, 1.07 mmol) in pyrrolidine (1.5 ml) under nitrogen atmosphere, and the mixture was stirred at 80° C. for 3 hours. The mixture was cooled to room temperature, diluted with ethyl acetate, washed with a saturated aqueous ammonium chloride solution, dried over anhydrous magnesium sulfate, and then concentrated under reduced pressure. The residue was purified by silica gel chromatography to give the title compound (280 mg).

¹H-NMR (CDCl₃) δ (ppm): 4.95 (2H, s), 6.98-7.06 (2H, m), 7.10-7.21 (2H, m), 7.31-7.35 (3H, m), 7.48-7.51 (3H, m), 7.57-7.65 (2H, m), 7.82 (1H, d), 8.25 (1H, d), 8.52 (1H, d).

### Example B27

### 1-(2-Phenylethylbenzyl)isoquinoline

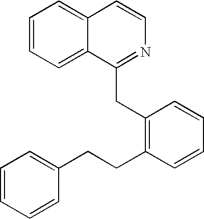

Palladium-carbon (10%, 230 mg) was added to a solution of the compound of Example B26 (280 mg, 0.88 mmol) in tetrahydrofuran (30 ml), and this mixture was stirred at room temperature under hydrogen atmosphere (1 atm) for 3 hours. The catalyst was removed by filtration and the obtained filtrate was concentrated under reduced pressure. The residue was purified by silica gel chromatography to give the title compound (162 mg).

¹H-NMR (CDCl₃) δ (ppm): 2.90-2.94 (2H, m), 3.07-3.10 (2H, m), 4.67 (2H, s), 6.80 (1H, d), 7.02-7.06 (1H, m), 7.15-7.30 (7H, m), 7.49-7.53 (1H, m), 7.58 (1H, d), 7.64-7.68 (1H, m), 7.84 (1H, d), 7.95 (1H, d), 8.50 (1H, d).

### Example B28

### 1-{2-[4-(Tetrahydro-2H-2-pyranyloxy)-1-butynyl]benzyl}-isoquinoline

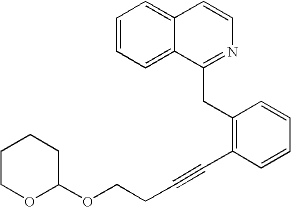

A solution of tetrakis(triphenylphosphine)palladium (58 mg, 0.05 mmol) and 2-(3-butynyloxy)-tetrahydro-2H-pyran (208 mg, 2.0 mmol) in pyrrolidine (1.5 ml) was added to a solution of the compound of Example B25 (345 mg, 1.07 mmol) in pyrrolidine (1.5 ml) under nitrogen atmosphere, and this mixture was stirred for four days at room temperature, and for another 30 minutes at 80° C. The mixture was cooled to room temperature, diluted with ethyl acetate, washed with a saturated aqueous ammonium chloride solution, dried over anhydrous magnesium sulfate, and then concentrated under reduced pressure. The residue was purified by silica gel chromatography to give the title compound (277 mg).

¹H-NMR (CDCl₃) δ (ppm): 1.42-1.60 (4H, m), 1.64-1.68 (1H, m), 1.75-1.81 (1H, m), 2.76-2.80 (2H, m), 3.46-3.51 (1H, m), 3.60-3.66 (1H, m), 3.85-3.95 (2H, m), 4.64-4.66 (1H, m), 4.85 (2H, s), 6.95-6.98 (1H, m), 7.05-7.13 (2H, m), 7.44-7.46 (1H, m), 7.49-7.53 (1H, m), 7.56 (1H, d), 7.60-7.65 (1H, m), 7.80-7.82 (1H, m), 8.15-8.18 (1H, m), 8.49-8.51 (1H, m).

### Example B29

### 4-[2-(1-Isoquinolylmethyl)phenyl]-3-butyn-1-ol

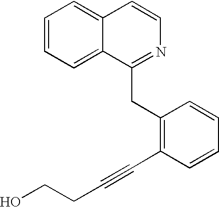

After the compound of Example B28 (200 mg, 0.54 mmol) was cooled to 0° C., a hydrochloric acid-methanol solution (10%, 5 ml) was added, and this mixture was stirred for 15 minutes. A saturated aqueous sodium hydrogencarbonate solution was added, and this mixture was extracted with ethyl acetate. The ethyl acetate layer was dried over anhydrous magnesium sulfate and concentrated under reduced pressure. The residue was purified by silica gel chromatography to give the title compound (86 mg).

¹H-NMR (CDCl₃) δ ppm): 2.72 (2H, t), 3.53-3.60 (1H, brs), 3.85 (2H, t), 4.85 (2H, s), 7.12-7.15 (2H, m), 7.22-7.24 (1H, m), 7.42-7.44 (1H, m), 7.55-7.59 (2H, m), 7.63-7.67 (1H, m), 7.81 (1H, d), 8.30 (1H, m), 8.46 (1H, m).

### Example B30

### 4-[2-(1-Isoquinolylmethyl)phenyl]-1-butanol

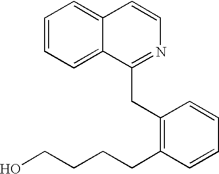

Palladium-carbon (10%, 10 mg) was added to a solution of the compound of Example B29 (44 mg, 0.15 mmol) in tetrahydrofuran (5 ml), and this mixture was stirred at room temperature under hydrogen atmosphere (1 atm) for 1 hour. After the catalyst was removed by filtration, the filtrate was concentrated under reduced pressure. The residue was purified by silica gel chromatography to give the title compound (18 mg).

¹H-NMR (CDCl₃) δ (ppm): 1.61-1.75 (4H, m), 2.33 (1H, brs), 2.77 (2H, t), 3.67 (2H, t), 4.70 (2H, s), 6.91 (1H, d), 7.02-7.06 (1H, m), 7.12-7.16 (1H, m), 7.19-7.21 (1H, m), 7.50-7.55 (1H, m), 7.57 (1H, d), 7.63-7.67 (1H, d), 7.83 (1H, d), 8.09 (1H, d), 8.47 (1H, d).

### Example 31

### 1-Bromo-2-(chloromethyl)benzene

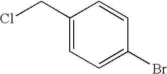

The title compound was obtained by treating p-bromobenzyl alcohol in the same manner as in Example B1.

### Example B32

### 1-(4-Bromobenzyl)isoquinoline 2490

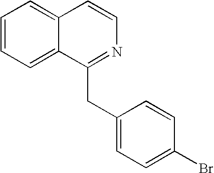

The title compound was obtained by treating the compound of Example B31 in the same manner as in Example B2.

¹H-NMR (CDCl₃) δ (ppm): 4.6 1(2H, s), 7.14-7.16 (2H, m), 7.35-7.39 (2H, m), 7.52-7.58 (2H, m), 7.63-7.67 (1H, m), 7.82 (1H, d), 8.07-8.10 (1H, m), 8.49 (1H, d).

### Example B33

### Ethyl (E)-3-[4-(Isoquinolylmethyl)phenyl]-2-propanoate

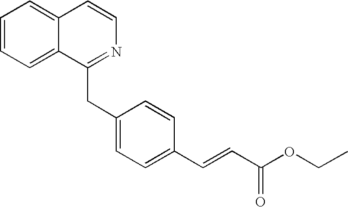

Tris(2-methylphenyl)phosphine (20 mg, 0.067 mmol), palladium(II) acetate (7.5 mg, 0.034 mmol), and triethylamine (70 μl, 0.50 mmol) were added to a solution of the compound of Example B32 (100 mg, 0.34 mmol) and vinyl propionate (73 μl, 0.67 mmol) in dimethylformamide (1.0 ml) under nitrogen atmosphere, and this mixture was stirred at 100° C. for 4 hours. After the mixture was cooled to room temperature, water was added, and this mixture was extracted with ethyl acetate. The organic layer was washed with water, dried over anhydrous magnesium sulfate, and then concentrated under reduced pressure. The residue was purified by silica gel column chromatography to give the title compound (74 mg).

¹H-NMR (CDCl₃) δ (ppm): 1.32 (3H, t), 4.24 (2H, q), 4.69 (2H, s), 6.36 (1H, d), 7.29 (2H, d), 7.42 (2H, d), 7.53-7.67 (4H, m), 7.83 (1H, d), 8.11-8.13 (1H, m), 8.50 (1H, d).

### Example B34

### Ethyl 3-[4-(1-Isoquinolylmethyl)phenyl]propanoate

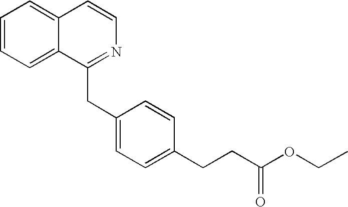

Palladium-carbon (10%, 20 mg) was added to a solution of the compound of Example B33 (71 mg, 0.22 mmol) in methanol (5.0 ml), and this reaction mixture was stirred at room temperature under hydrogen atmosphere at atmospheric pressure for 5 hours. After the catalyst was removed from the reaction mixture by filtration, the filtrate was concentrated under reduced pressure. The residue was purified by silica gel column chromatography to give the title compound (52 mg).

¹H-NMR (CDCl₃) δ (ppm): 1.20 (3H, t), 2.56 (2H, t), 2.88 (2H, t), 4.09 (2H, q), 4.64 (2H, s), 7.09 (2H, d), 7.20 (2H, d), 7.51-7.57 (2H, m), 7.62-7.66 (1H, m), 7.82 (1H, d), 8.15 (1H, dd), 8.50 (1H, d).

### Example B35

### 3-[4-(1-Isoquinolylmethyl)phenyl]-1-propanol

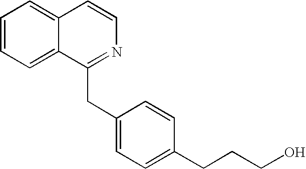

Lithium aluminum hydride (6 mg, 0.16 mmol) was added to tetrahydrofuran (1.0 ml) cooled to 0° C. under nitrogen atmosphere. A solution of the compound of Example B34 (46 mg, 0.14 mmol) in tetrahydrofuran (1.0 ml) was further added, and this reaction mixture was stirred at that temperature for 3 hours. A mixed solution of methanol and water (9:1, 1.0 ml) was added to the reaction mixture, a saturated aqueous ammonium chloride solution was further added, then this mixture was extracted with chloroform. The organic layer was dried over anhydrous magnesium sulfate and concentrated under reduced pressure. The residue was purified by silica gel column chromatography to give the title compound (22 mg).

¹H-NMR (CDCl₃) δ (ppm): 1.30-1.35 (1H, brs), 1.81-1.88 (2H, m), 2.64 (2H, t), 3.62-3.65 (2H, m), 4.64 (2H, s), 7.09 (2H, d), 7.20 (2H, d), 7.51-7.57 (2H, m), 7.62-7.66 (1H, m), 7.81 (1H; d), 8.16-8.18 (1H, m), 8.49 (1H, d).

### Example 36

### 1-Isoquinolyl(4-methoxyphenyl)ketone

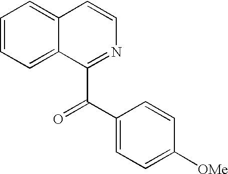

4-Bromoanisol (15.3 ml, 122 mmol) and a catalytic amount of 1,2-dibromoethane as an initiator were added to a mixed solution of magnesium (3059 mg, 125.8 mmol) and tetrahydrofuran (20 ml) under nitrogen atmosphere, and this reaction mixture was stirred while heating under reflux for 45 minutes. The mixture was cooled to 0° C., a solution of 1-isoquinolinecarbonitrile (10.78 g, 69.9 mmol) in tetrahydrofuran (30 ml) was added dropwise thereto, and this reaction mixture was stirred at room temperature for 2 hours. The reaction mixture was cooled on ice, concentrated hydrochloric acid (24 ml) and methanol (120 ml) were added, and this mixture was heated under reflux for 1.5 hours. After cooling on ice, the mixture was adjusted to pH 8 by adding aqueous sodium hydroxide, extracted with ether, washed with saturated brine, dried over anhydrous magnesium sulfate, and then concentrated under reduced pressure. The residue was purified by silica gel column chromatography to give the title compound (15.87 g).

¹H-NMR (CDCl₃) δ (ppm): 3.88 (3H, s), 6.95 (2H, d), 7.61 (1H, dd), 7.74 (1H, dd), 7.76 (1H, d), 7.85 (2H, d), 8.17 (1H, dd), 8.60 (1H, d).

### Example B37

### 1-Isoquinolyl(4-methoxyphenyl)methanol

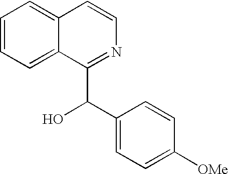

Sodium borohydride (1855 mg) was added to an ice-cooled solution of the compound of Example B36 (8608 mg) in ethanol (170 ml), and this mixture was stirred at room temperature for 35 minutes. Sodium borohydride (957 mg) was further added, and this reaction mixture was stirred at 40° C. for 40 minutes. The reaction mixture was concentrated under reduced pressure, water was added, and this mixture was extracted with ether. The organic layer was washed with water and saturated brine, dried over anhydrous magnesium sulfate, and then concentrated under reduced pressure. The obtained title compound (7881 mg) was used in the following reaction without further purification.

¹H-NMR (DMSO-d6) δ (ppm): 3.66 (3H, s), 6.30-6.32 (1H, brs), 6.81 (2H, d), 7.28 (2H, d), 7.54 (1H, dd), 7.68 (1H, dd), 7.76 (1H, d), 7.94 (1H, d), 8.37 (1H, d), 8.47 (1H, d).

The proton of the hydroxyl group was not observed in the NMR spectrum.

### Example B38

### 1-Isoquinolyl(4-methoxyphenyl)methyl Acetate

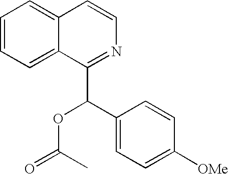

Acetic anhydride (20 ml) was added to a solution of the compound of Example B37 (7881 mg) in pyridine (100 ml), and this reaction mixture was stirred at 50° C. for 4 hours. The reaction mixture was concentrated under reduced pressure and subjected to azeotropic distillation with toluene. The residue was purified by silica gel column chromatography to give the title compound (8.79 g).

¹H-NMR (CDCl₃) δ (ppm): 2.22 (3H, s), 3.76 (3H, s), 6.84 (2H, d), 7.39 (2H, d), 7.54 (1H, dd), 7.56 (1H, s), 7.60 (1H, d), 7.64 (1H, dd), 7,82 (1H, d), 8.19 (1H, d), 8.57 (1H, d).

### Example B39

### 1-(4-Methoxybenzyl)isoquinoline

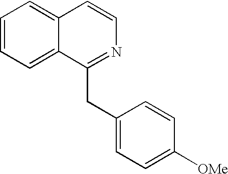

Palladium-carbon (10%, 4.0 g) was added to a solution of the compound of Example B38 (8.79 g) in methanol (150 ml), and this mixture was stirred at room temperature under hydrogen atmosphere at atmospheric pressure for 5.5 hours. The catalyst was removed by filtration through celite, and the filtrate was concentrated under reduced pressure. The residue was purified by silica gel column chromatography to give the title compound (4.48 g).

¹H-NMR (CDCl₃) δ (ppm): 3.74 (3H, s), 4.61 (2H, s), 6.79 (2H, d), 7.21 (2H, d), 7.53 (1H, dd), 7.56 (1H, d), 7.63 (1H, dd), 7.80 (1H, d), 8.16 (1H, d), 8.49 (1H, d).

### Example B40

### 4-(1-Isoquinolylmethyl)phenol

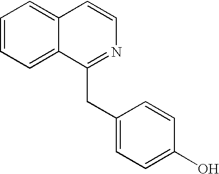

An aqueous hydrobromic acid solution (47%, 40 ml) was added to the compound of Example B39 (2185 mg), and this reaction mixture was heated under reflux for 14 hours. The reaction mixture was cooled to room temperature, further cooled on ice, neutralized with a 50% aqueous sodium hydroxide solution, and extracted with ethyl acetate. The ethyl acetate layer was washed with water, dried over anhydrous magnesium sulfate, and then concentrated under reduced pressure. The obtained powder was washed with petroleum ether to give the title compound (1822 mg).

¹H-NMR (DMSO-d6) δ (ppm): 4.48 (2H, s), 6.61 (2H, d), 7.07 (2H, d), 7.60 (1H, dd), 7.68 (1H, d), 7.71 (1H, dd), 7.92 (1H, d), 8.27 (1H, d), 8.41 (1H, d), 9.19 (1H, brs).

### Example B41

### 4-(1-Isoquinolylmethyl)phenyl Trifluoromethanesulfonate

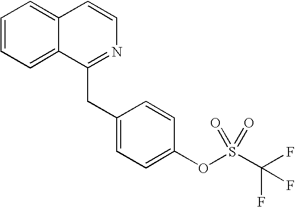

Trifluoromethanesulfonic anhydride (0.55 ml) was added dropwise to an ice-cold solution of the compound of Example B40 (513 mg) in pyridine (10 ml), and this reaction mixture was stirred at that temperature for 45 minutes. After ice was added, the reaction mixture was extracted with ether. The organic layer was washed with water and saturated brine, dried over anhydrous magnesium sulfate, and then concentrated under reduced pressure. The residue was purified by silica gel column chromatography to give the title compound (546 mg).

¹H-NMR (CDCl₃) δ (ppm): 4.69 (2H, s), 7.16 (2H, d), 7.35 (2H, d), 7.57 (1H, dd), 7,60 (1H, d), 7.68 (1H, dd), 7.85 (1H, d), 8.09 (1H, d), 8.50 (1H, d).

### Example B42

### 1-[4-(2-Phenyl-1-ethynyl)benzyl]isoquinoline

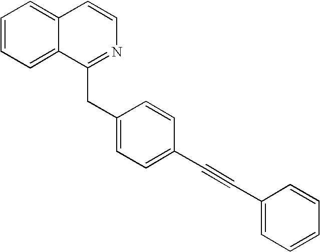

Phenylacetylene (53 μl), palladium acetate (9 mg), 1,1′-bis(diphenylphosphino)ferrocene (67 mg), copper(I) iodide (3 mg), lithium chloride (20 mg), and triethylamine (50 μl) were added to a solution of the compound of Example B41 (88 mg) in N, N-dimethylformamide (2.0 ml) that had been degassed and placed under nitrogen, and this mixture was stirred at 80° C. for 8 hours. After cooling the mixture to room temperature, water was added, and this mixture was extracted with ethyl acetate. The organic layer was washed with water and saturated brine, dried over anhydrous magnesium sulfate, and then concentrated under reduced pressure. The residue was purified by silica gel column chromatography to give the title compound (53 mg).

¹H-NMR (CDCl₃) 6 (ppm): 4.69 (2H, s), 7.12-7.32 (3H, m), 7.25 (2H, d), 7.42 (2H, d), 7.43-7.52 (2H, m), 7.54 (1H, dd), 7.58 (1H, d), 7.65 (1H, dd), 7.83 (1H, d), 8.10 (1H, d), 8.51 (1H, d).

### Example B43

### 1-(4-Phenethylbenzyl)isoquinoline

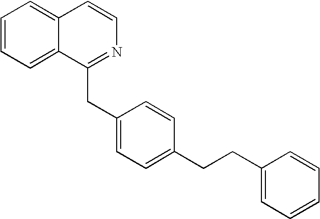

Palladium-carbon catalyst (10%, 20 mg) was added to a solution of the compound of Example B42 (45 mg) in tetrahydrofuran (2 ml), and this mixture was stirred at room temperature under hydrogen atmosphere at atmospheric pressure for 2 hours. The catalyst was removed by filtration through celite, and the filtrate was concentrated under reduced pressure. The residue was purified by silica gel column chromatography to give the title compound (23 mg).

¹H-NMR (CDCl₃) δ (ppm): 2.78-2.90 (4H, m), 4.64 (2H, s), 7.07 (2H, d), 7.10-7.20 (5H, m), 7.22 (2H, d), 7.53 (1H, dd), 7.55 (1H, d), 7.63 (1H, dd), 7.80 (1H, d), 8.15 (1H, d), 8.49 (1H, d).

### Example B44

### 1-[4-(4-Phenyl-1-butynyl)benzyl]isoquinoline

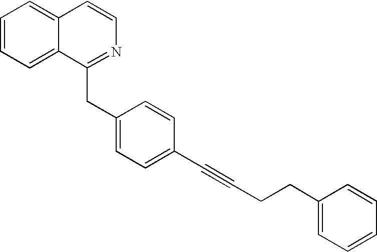

The title compound was obtained by treating the compound of Example B41 and 4-phenyl-1-butyne in the same manner as in Example B42.

¹H-NMR (CDCl₃) δ (ppm): 2.65 (2H, t), 2.88 (2H, t), 4.68 (2H, s), 7.12-7.40 (9H, m), 7.50-7.70 (3H, m), 7.80-7.88 (1H, m), 8.00-8.10 (1H, m), 8.48-8.51 (1H, m).

### Example B45

### 1-[4-(4-Phenyl-1-butyl)benzyl]isoquinoline

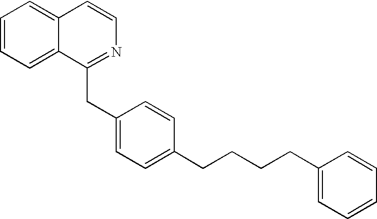

The title compound was obtained by treating the compound of Example B44 in the same manner as in Example B43.

¹H-NMR (CDCl₃) δ (ppm): 1.55-1.80 (4H, m), 2.50-2.65 (4H, m), 4.68 (2H, s), 7.00-7.30 (9H, m), 7.52 (1H, dd), 7.56 (1H, d), 7.63 (1H, dd), 7.81 (1H, d), 8.15 (1H, d), 8.50 (1H, d).

### Example 46

### 1-{4-[4-(Tetrahydro-2H-2-pyranyloxy)-1-butynyl]benzyl}-isoquinoline

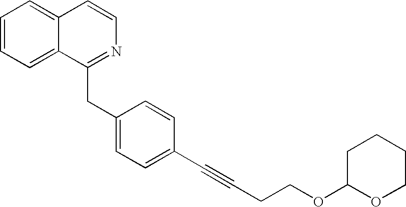

The title compound was obtained by treating the compound of Example B41 and 2-(3-butynyloxy)tetrahydro-2H-pyran in the same manner as in Example B42.

¹H-NMR (CDCl₃) δ (ppm): 1.48-1.90 (6H, m), 2.67 (2H, t), 3.49-3.55 (1H, m), 3.60 (1H, dd), 3.65-3.94 (2H, m), 4.66 (2H, s), 4.65-4.70 (1H, m), 7.14-7.20 (2H, m), 7.23-7.30 (2H, m), 7.53 (1H, dd), 7.58 (1H, d), 7.65 (1H, dd), 7.82 (1H, d), 8.10 (1H, d), 8.49 (1H, d).

### Example B47

### 4-[4-(1-Isoquinolylmethyl)phenyl]-3-butyn-1-ol

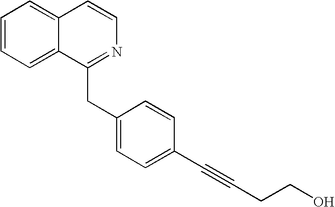

The compound of Example B46 (1048 mg) was dissolved in a 10% hydrochloric acid-methanol solution (50 ml), and this reaction mixture was stirred at room temperature for 1.5 hours. The reaction mixture was cooled on ice, a saturated aqueous sodium hydrogencarbonate solution was added, and the resulting mixture was extracted with ethyl acetate. The organic layer was washed with water and saturated brine, dried over anhydrous magnesium sulfate, and then concentrated under reduced pressure. The residue was purified by silica gel column chromatography to give the title compound (666 mg).

¹H-NMR (CDCl₃) δ (ppm): 2.65 (2H, t), 3.77 (2H, t), 4.65 (2H, s), 7.18 (2H, d), 7.29 (2H, d), 7.52 (1H, dd), 7.57 (1H, d), 7.64 (1H, dd), 7.81 (1H, d), 8.07 (1H, d), 8.49 (1H, d).

The proton of the hydroxyl group was not observed in the NMR spectrum.

### Example B48

### 4-[4-(1-Isoquinolylmethyl)phenyl]-1-butanol

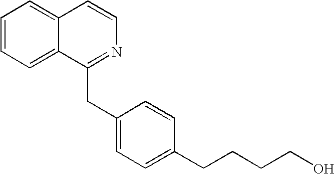

The title compound was obtained by treating the compound of Example B47 in the same manner as in Example B43.

¹H-NMR (CDCl₃) δ (ppm): 1.50-1.70 (4H, m), 2.57 (2H, t), 3.62 (2H, t), 4.64 (2H, s), 7.06 (2H, d), 7.18 (2H, d), 7.53 (1H, dd), 7.55 (1H, d), 7.63 (1H, dd), 7.80 (1H, d), 8.16 (1H, d), 8.49 (1H, d).

The proton of the hydroxyl group was not observed in the NMR spectrum.

### Example 49

### 1-[4-(3-Cyclopentyl-1-propynyl)benzyl]isoquinoline

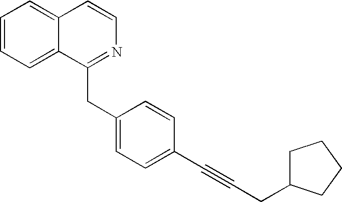

The title compound was obtained by treating the compound of Example B41 and 3-cyclopentyl-1-propyne in the same manner as in

### Example B42.

¹H-NMR (CDCl₃) δ (ppm): 1.25-1.35 (2H, m), 1.45-1.70 (6H, m), 1.75-1.85 (2H, m), 2.05-2.13 (1H, m), 4.65 (2H, s), 7.17 (2H, d), 7.27 (2H, d), 7.51 (1H, dd), 7.56 (1H, d), 7.64 (1H, dd), 7.81 (1H, d), 8.08 (1H, d), 8.49 (1H, d).

### Example B50

### 1-[4-(3-Cyclopentylpropyl)benzyl]isoquinoline

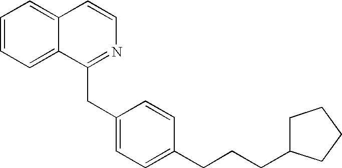

The title compound was obtained by treating the compound of Example B49 in the same manner as in Example B43.

¹H-NMR (CDCl₃) δ (ppm): 1.25-1.74 (13H, m), 2.49-2.54 (2H, m), 4.64 (2H, s), 7.06 (2H, d), 7.18 (2H, d), 7.53 (1H, dd), 7.55 (1H, d), 7.63 (1H, dd), 7.80 (1H, d), 8.17 (1H, d), 8.49 (1H, d).

### Example B51

### 4-[4-(1-Isoquinolylmethyl)phenyl]-2-methyl-3-butyn-2-ol

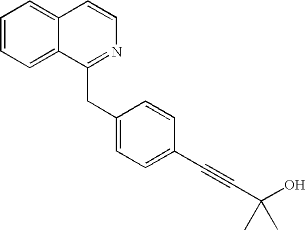

The title compound was obtained by treating the compound of Example B41 and 2-methyl-3-butyn-2-ol in the same manner as in Example B42.

¹H-NMR (DMSO-d6) δ (ppm): 1.35 (1H, s), 1.40 (6H, s), 4.62 (2H, s), 7.20-7.30 (4H, m), 7.61 (1H, dd), 7.71 (1H, d), 7.69-7.76 (1H, m), 7.95 (1H, d), 8.26 (1H, d), 8.42 (1H, d).

### Example B52

### 4-[4-(1-Isoquinolylmethyl)phenyl]-2-methyl-2-butanol

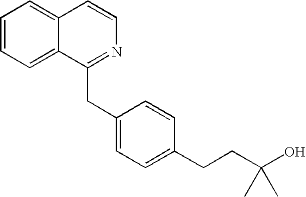

The title compound was obtained by treating the compound of Example B51 in the same manner as in Example B43.

¹H-NMR (CDCl₃) δ (ppm): 1.25 (6H, s), 1.70-1.77 (2H, m), 2.60-2.67 (2H, m), 4.64 (2H, s), 7.08 (2H, d), 7.19 (2H, d), 7.53 (1H, dd), 7.55 (1H, d), 7.63 (1H, dd), 7.80 (1H, d), 8.16 (1H, d), 8.49 (1H, d).

The proton of the hydroxyl group was not observed in the NMR spectrum.

### Example B53

### 1-[4-(3-Methoxy-1-propynyl)benzyl]isoquinoline

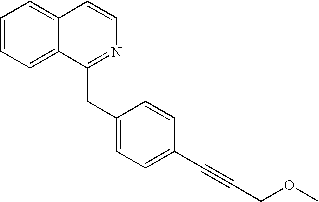

The title compound was obtained by treating the compound of Example B41 and methylpropargyl ether in the same manner as in Example B42.

¹H-NMR (CDCl₃) δ (ppm): 3.42 (3H, s), 4.29 (2H, s), 4.66 (2H, s), 7.21 (2H, d), 7.34 (2H, d), 7.54 (1H, dd), 7.58 (1H, d), 7.65 (1H, dd) 7.82 (1H, d), 8.10 (1H, d) 8.49 (1H, d).

### Example B54

### 1-[4-(3-Methoxypropyl)benzyl]isoquinoline

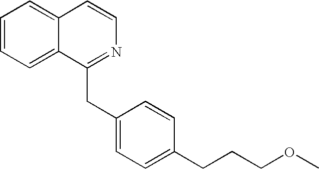

The title compound was obtained by treating the compound of Example B53 in the same manner as in Example B43.

¹H-NMR (CDCl₃) δ (ppm): 1.78-1.87 (2H, m), 2.06 (2H, t), 3.31 (3H, s), 3.35 (2H, t), 4.64 (2H, s), 7.07 (2H, d), 7.22 (2H, d), 7.53 (1H, dd), 7.55 (1H, d), 7.64 (1H, dd), 7.81 (1H, d), 8.17 (1H, d), 8.49 (1H, d).

### Example B55

### 1-{4-[2-(2-Pyridyl)-1-ethynyl]benzyl}isoquinoline

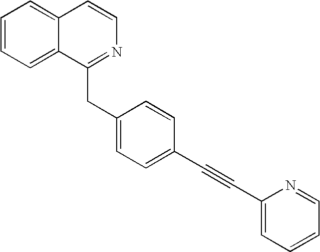

The title compound was obtained by treating the compound of Example B41 and 2-ethynylpyridine in the same manner as in Example B42.

¹H-NMR (CDCl₃) δ (ppm): 4.71 (2H, s), 7.20-7.25 (2H, m), 7.29 (2H, d), 7.48-7.53 (1H, m), 7.51 (2H, d), 7.57 (1H, dd), 7.61 (1H, d), 7.67 (1H, dd), 7.85 (1H, d), 8.13 (1H, d), 8.53 (1H, d), 8.59-8.63 (1H, m).

### Example B56

### 1-{4-[2-(2-Pyridyl)ethyl]benzyl}isoquinoline

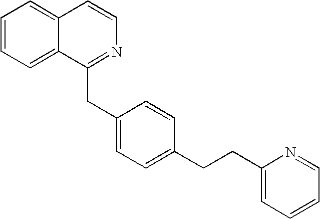

The title compound was obtained by treating the compound of Example B55 in the same manner as in Example B43.

¹H-NMR (CDCl₃) δ (ppm): 2.94-3.06 (4H, m), 4.64 (2H, s), 7.04 (1H, d), 7.09 (1H, dd), 7.09 (2H, d), 7.18 (2H, d), 7.53 (1H, ddd), 7.54 (1H, dd), 7.55 (1H, d), 7.64 (1H, d) 7.81 (1H, d), 8.15 (1H, d), 8.49 (1H, d), 8.53 (1H, dd).

### Example B57

### 1-{4-[2-(3-Pyridyl)-1-ethynyl]benzyl}isoquinoline

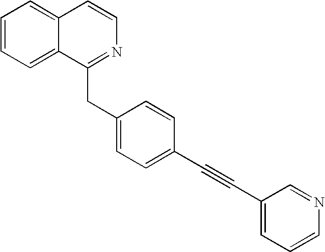

The title compound was obtained by treating the compound of Example B41 and 3-ethynylpyridine in the same manner as in Example B42. ¹H-NMR (CDCl₃) δ (ppm): 4.69 (2H, s), 7.27 (2H, d), 7.31 (1H, dd), 7.43 (2H, d), 7.55 (1H, dd), 7.59 (1H, d), 7.66 (1H, dd), 7.82 (1H, ddd), 7.83 (1H, d), 8.10 (1H, d), 8.51 (1H, d), 8.60 (1H, dd), 8.77 (1H, d).

### Example B58

### 1-{4-[2-(3-Pyridyl)ethyl]benzyl}isoquinoline

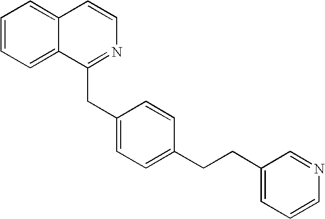

The title compound was obtained by treating the compound of Example B57 in the same manner as in Example B43.

¹H-NMR (CDCl₃) δ (ppm): 2.80-2.90 (4H, m), 4.65 (2H, s), 7.04 (2H, d), 7.15 (1H, dd), 7.19 (2H, d), 7.39 (1H, dd), 7.54 (1H, dd), 7.56 (1H, d), 7.64 (1H, dd), 7.81 (1H, d), 8.15 (1H, d), 8.40 (1H, d), 8.42 (1H, d), 8.49 (1H, d).

### Example B59

### N-(2-Propynyl)acetamide

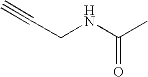

Pyridine (16.3 ml) and acetic anhydride (10.4 ml) were added to an ice-cooled solution of propargylamine (3023 mg) in methylene chloride (30 ml), and this reaction mixture was stirred at room temperature for l hour. The reaction mixture was poured on ice, extracted with ethyl acetate, washed successively with 1 N hydrochloric acid, a saturated aqueous sodium hydrogencarbonate solution, and saturated brine, dried over anhydrous magnesium sulfate, and then filtered through silica gel. The filtrate was concentrated under reduced pressure to give the title compound (743 mg). The obtained compound was used in the following reaction without further purification.

¹H-NMR (DMSO-d6) δ (ppm): 1.79 (3H, s), 3.07 (1H, t), 3.81 (2H, d), 8.25 (1H, brs).

### Example B60

### N-{3-[4-(1-Isoquinolylmethyl)phenyl]-2-propynyl}acetamide

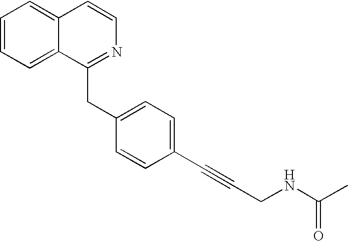

The title compound was obtained by treating the compound of Example B41 and the compound of Example B59 in the same manner as in Example B42.

¹H-NMR (DMSO-d6) δ (ppm): 1.79 (3H, s), 4.04 (2H, s), 4.61 (2H, s), 7.45-7.68 (4H, m), 7.68-7.75 (2H, m), 7.90-8.00 (1H, m), 8.25-8.38 (2H, m), 8.40-8.45 (1H, m).

### Example B61

### N-{3-[4-(1-Isoquinolylmethyl)phenyl]propyl}acetamide

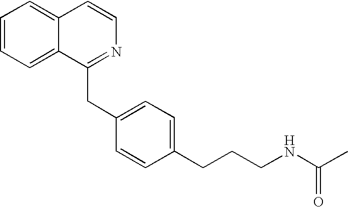

The title compound was obtained by treating the compound of Example B60 in the same manner as in Example B43.

¹H-NMR (CDCl₃) δ (ppm): 1.95 (3H, s), 1.74-1.84 (2H, m), 2.55 (2H, t), 3.25 (2H, dt), 4.68 (2H, s), 7.10 (2H, d), 7.18 (2H, d), 7.20-7.28 (1H, m), 7.50-7.58 (2H, m), 7.60-7.68 (1H, m), 7.75-7.85 (1H, m), 8.10-8.16 (1H, m), 8.45-8.50 (1H, m).

### Example B62

### N-(2-Propynyl)methanesulfonamide

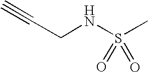

Triethylamine (9.77 ml) was added to an ice-cooled solution of propargylamine (3023 mg) in methylene chloride (30 ml). After dropwise addition of methanesulfonyl chloride (5.19 ml), the reaction mixture was stirred for 3 hours at that temperature, warmed to room temperature, and further stirred for 2 hours. Ice was added to the reaction mixture, the resulting mixture was extracted with ethyl acetate, washed with saturated brine, dried over anhydrous magnesium sulfate, and concentrated under reduced pressure. The residue was dissolved in methanol (120 ml), potassium carbonate (11.7 g) was added, and this reaction mixture was stirred at room temperature for 3 hours. The reaction mixture was concentrated under reduced pressure, neutralized with dilute hydrochloric acid while cooling on ice, and then extracted with ethyl acetate. The extract was washed with saturated brine, dried over anhydrous magnesium sulfate, and then concentrated under reduced pressure. The residue was purified by silica gel column chromatography to give the title compound (6.67 g).

¹H-NMR (CDCl₃) δ (ppm): 2.39 (1H, t), 3.10 (3H, s), 3.99 (2H, dd), 4.60 (1H, brs).

### Example B63

### N-{3-[4-(1-Isoquinolylmethyl)phenyl]-2-propynyl}-methanesulfonamide

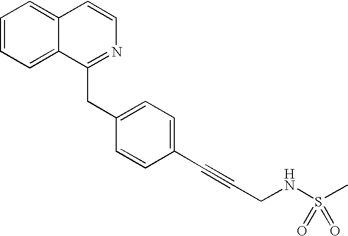

The title compound was obtained by treating the compound of Example B41 and the compound of Example B62 in the same manner as in Example B42.

¹H-NMR (DMSO-d6) δ (ppm): 2.97 (3H, s), 4.00 (2H, d), 4.63 (2H, s), 7.25-7.37 (4H, m), 7.57 (1H, t), 7.62 (1H, dd), 7.71 (1H, d), 7.73 (1H, dd), 7.94 (1H, d), 8.28 (1H, d), 8.42 (1H, d).

### Example B64

### N-{3-[4-(1-Isoquinolylmethyl)phenyl]propyl}methanesulfonamide

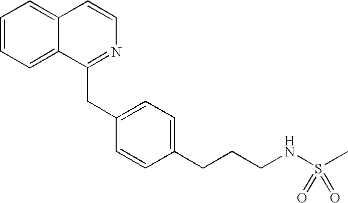

The title compound was obtained by treating the compound of Example B63 in the same manner as in Example B43.

¹H-NMR (CDCl₃) δ (ppm): 1.80-1.90 (2H, m), 2.62 (2H, t), 2.89 (3H, s), 3.11 (2H, dt), 4.25 (1H, brs), 4.64 (2H, s), 7.05 (2H, d) 7.20 (2H, d), 7.50 (1H, dd), 7.56 (1H, d), 7.63 (1H, dd), 7.81 (1H, d), 8.15 (1H, d), 8.49 (1H, d).

### Example B65

### 1-{4-[3-(Ethylsulfanyl)-1-propynyl]benzyl}isoquinoline

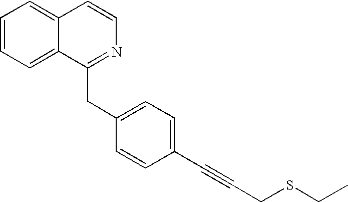

The title compound was obtained by treating the compound of Example B41 and propargyl ethyl sulfide in the same manner as in Example B42.

¹H-NMR (CDCl₃) δ (ppm): 1.30 (3H, t), 2.73 (2H, q), 3.47 (2H, s), 4.67 (2H, s), 7.20-7.32 (4H, m), 7.52 (1H, dd), 7.57 (1H, d), 7.64 (1H, dd), 7.81 (1H, d), 8.08 (1H, d), 8.49 (1H, d).

### Example B66

### t-Butyl N-(Propynyl)carbamate

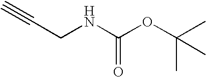

A solution of di-t butyl-dicarbonate (10.84 g) in tetrahydrofuran (20 ml) was added dropwise to an ice-cooled solution of propargylamine (3040 mg) in tetrahydrofuran (20 ml), the temperature of the mixture was gradually raised to room temperature, and the reaction mixture was stirred for 20 hours. After water was added, the reaction mixture was extracted with ethyl acetate, washed with saturated brine, dried over anhydrous magnesium sulfate, and then concentrated under reduced pressure to give the title compound (9.34 g). The obtained compound was used in the following reaction without further purification.

¹H-NMR (DMSO-d6) δ (ppm): 1.36 (9H, s), 3.04 (1H, t), 3.62-3.70 (2H, m), 7.20-7.30 (1H, m).

### Example B67

### tert-Butyl N-{3-[4-(1-Isoquinolylmethyl)phenyl]-2-propynyl}-carbamate

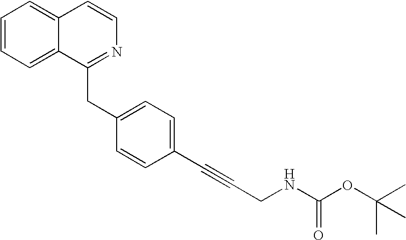

The title compound was obtained by treating the compound of Example B41 and the compound of Example B66 in the same manner as in Example B42.

¹H-NMR (CDCl₃) δ (ppm): 1.45 (9H, s), 4.06-4.13 (2H, m), 4.66 (2H, s), 7.19 (2H, d), 7.20-7.28 (1H, m), 7.29 (2H, d), 7.52 (1H, dd), 7.57 (1H, d), 7.65 (1H, dd), 7.82 (1H, d), 8.08 (1H, d), 8.49 (1H, d).

### Example B68

### tert-Butyl N-{3-[4-(1-Isoquinolylmethyl)phenyl]propyl}carbamate

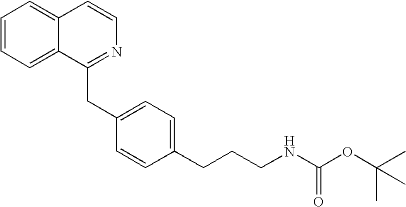

The title compound was obtained by treating the compound of Example B67 in the same manner as in Example B43.

¹H-NMR (CDCl₃) δ (ppm): 1.43 (9H, s), 1.70-1.81 (2H, m), 2.54-2.60 (2H, m), 3.01-3.20 (2H, m), 4.47-4.57 (1H, m), 4.65 (2H, s), 7.07 (2H, d), 7.21 (2H, d), 7.55 (1H, dd), 7.57 (1H, d), 7.65 (1H, dd), 7.83 (1H, d), 8.18 (1H, d), 8.51 (1H, d).

### Example B69

### 3-[4-(1-Isoquinolylmethyl)phenyl]-2-propyn-1-amine

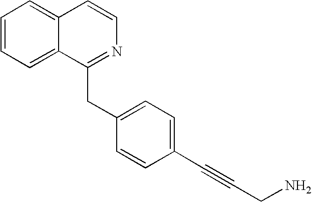

Trifluoroacetic acid (0.3 ml) was added to an ice-cooled solution of the compound of Example B67 (4 mg) in methylene chloride (0.6 ml), and the reaction mixture was stirred at that temperature for 1 hour. After a saturated aqueous sodium hydrogencarbonate solution was added, the reaction mixture was extracted with ethyl acetate, dried over anhydrous magnesium sulfate, and then concentrated under reduced pressure. The residue was purified by silica gel column chromatography to give the title compound (4 mg).

¹H-NMR (CDCl₃) δ (ppm): 3.60-3.68 (2H, m), 4.66 (2H, s), 7.19 (2H, d), 7.29 (2H, d), 7.53 (1H, dd), 7.56 (1H, d), 7.63 (1H, dd), 7.82 (1H, d), 8.10 (1H, d), 8.49 (1H, d).

The amine proton was not observed in the NMR spectrum.

### Example B70

### 3-[4-(1-Isoquinolylmethyl)phenyl]-1-propanamine

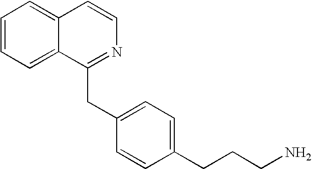

The title compound was obtained by treating the compound of Example B68 in the same manner as in Example B69.

¹H-NMR (CDCl₃) δ (ppm): 1.20-1.30 (2H, m), 1.78-1.88 (2H, m), 2.45-2.52 (2H, m), 2.73-2.81 (2H, m), 4.55 (2H, s), 6.94 (2H, d), 7.08 (2H, d), 7.50 (1H, dd), 7.51 (1H, d), 7.61 (1H, dd), 7.76 (1H, d), 8.10 (1H, d), 8.38 (1H, d).

### Example B71

### N-Methyl-N-(2-propynyl)acetamide

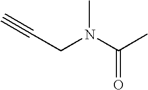

The title compound was obtained by treating N-methyl-N-(2-propynyl)amine in the same manner as in Example B59.

¹H-NMR (CDCl₃) δ (ppm): 2.11 (2.1H, s), 2.17 (0.9H, s), 2.21 (0.7H, t), 2.31 (0.3H, t), 3.00 (0.9H, s), 3.08 (2.1H, s), 4.04 (0.6H, d), 4.23 (1.4H, d).

The obtained compound contained a 7:3 mixture of geometrical isomers of the amide.

### Example B72

### N-{3-[4-(1-Isoquinolylmethyl)phenyl]-2-propynyl}-N-methyl-acetamaide

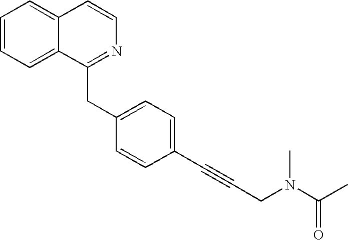

The title compound was obtained by treating the compound of Example B41 and the compound of Example B71 in the same manner as in Example B42.

¹H-NMR (CDCl₃) δ (ppm): 2.10 (1.8H, s), 2.11 (1.2H, s), 3.01 (1.2H, s), 3.10 (1.8H, s), 4.21 (1.2H, s), 4.41 (0.8H, s), 4.67 (2H, s), 7.18-7.23 (2H, m), 7.29-7.32 (2H, m), 7.53 (1H, dd), 7.58 (1H, d), 7.65 (1H, dd), 7.82 (1H, d), 8.09 (1H, d), 8.49 (1H, d).

The obtained compound contained a 3:2 mixture of geometrical isomers of the amide.

### Example B73

### N-{3-[4-(1-Isoquinolylmethyl)phenyl]propyl}-N1-methylacetamide

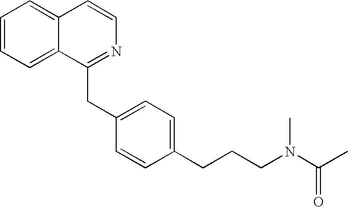

The title compound was obtained by treating the compound of Example B72 in the same manner as in Example B43.

¹H-NMR (CDCl₃) δ (ppm): 1.70-1.90 (2H, m), 1.89 (1.5H, s), 2.03 (1.5H, s), 2.50-2.59 (2H, m), 2.88 (1.5H, s), 2.91 (1.5H, s), 3.20-3.25 (1H, m), 3.36-3.40 (1H, m), 4.66 (2H, s), 7.03-7.10 (2H, m), 7.18-7.30 (2H, m), 7.53 (1H, dd), 7.58 (1H, d), 7.66 (1H, dd), 7.82 (1H, d), 8.17 (1H, d), 8.50 (1H, d).

The obtained compounds contained a 1:1 mixture of geometrical isomers of the amide.

### Example B74

### N-Methyl-N-(2-propynyl)methanesulfonamide

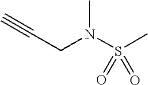

Triethylamine (6.55 ml) was added to an ice-cooled solution of N-methyl-N-(2-propynyl)amine (2603 mg) in methylene chloride (25 ml). Methanesulfonyl chloride (3.50 ml) was further added dropwise, the reaction mixture was stirred at that temperature for 1 hour, and then stirred further at room temperature for 2 hours. After ice was added, the reaction mixture was extracted with ethyl acetate, washed successively with 1 N hydrochloric acid, a saturated aqueous sodium hydrogencarbonate solution, and saturated brine, dried over anhydrous magnesium sulfate, and then filtered through silica gel. The filtrate was concentrated under reduced pressure to give the title compound (4522 mg). The obtained compound was used in the following reaction without further purification.

¹H-NMR (CDCl₃) δ (ppm): 2.41 (1H, t), 2.93 (3H, s), 2.96 (3H, s), 4.09 (2H, d).

### Example B75

### N-{3-[4-(1-Isoquinolylmethyl)phenyl]-2-propynyl}-N-methyl Methanesulfonamide

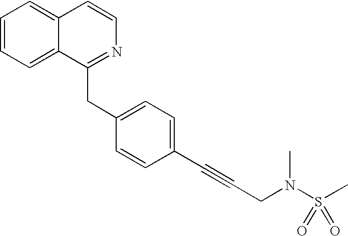

The title compound was obtained by treating the compound of Example B41 and the compound of Example B74 in the same manner as in Example B42.

¹H-NMR (CDCl₃) δ (ppm): 2.95 (3H, s), 2.97 (3H, s), 4.26 (2H, s), 4.68 (2H, s), 7.24 (2H, d), 7.31 (2H, d), 7.55 (1H, dd), 7.59 (1H, d), 7.66 (1H, dd), 7.83 (1H, d), 8.10 (1H, d), 8.49 (1H, d).

### Example B76

### N-{3-[4-(1-Isoquinolylmethyl)phenyl]propyl}-N-methyl Methanesulfonamide

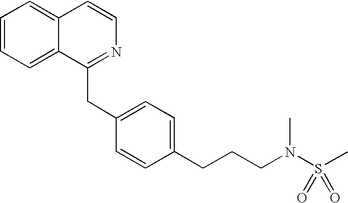

Treating the compound of Example B75 in the same manner as in Example B43, the obtained residue was separated and purified by LC-MS [eluent: an acetonitrile solution containing 0.1% trifluoroacetic acid: an aqueous solution containing 0.1% trifluoroacetic acid=1:99 to 100:0/20-minute cycle, flow rate: 20 ml/minute, column: YMC Combiprep ODS-AM, 20 mm Φ×50 mm (long)] to give the title compound.

MS m/z (ESI:MH⁺): 369.2.

### Example B77

### 5-[4-(1-Isoquinolylmethyl)phenyl]-4-pentyn-2-ol

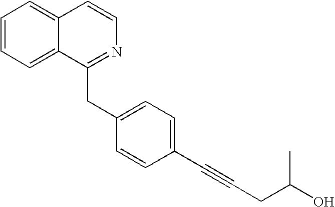

The title compound was obtained by treating the compound of Example B41 and 4-pentyn-2-ol in the same manner as in Example B42.

¹H-NMR (CDCl₃) δ (ppm): 1.27 (3H, t), 2.38-2.62 (2H, m), 3.95-4.03 (1H, m), 4.65 (2H, s), 7.19 (2H, d), 7.29 (2H, d), 7.52 (1H, dd), 7.57 (1H, d), 7.64 (1H, dd), 7.81 (1H, d), 8.08 (1H, d), 8.48 (1H, d).

The proton of the hydroxyl group was not observed in the NMR spectrum.

### Example B78

### 5-[4-(1-Isoquinolylmethyl)phenyl]-2-pentanol

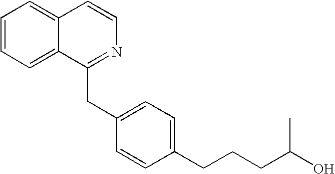

Treating the compound of Example B77 in the same manner as in Example B43, the obtained residue was separated and purified by LC-MS [eluent: an acetonitrile solution containing 0.1% trifluoroacetic acid: an aqueous solution containing 0.1% trifluoroacetic acid=1:99 to 100:0/20-minute cycle, flow rate: 20 ml/minute, column: YMC Combiprep ODS-AM, 20 mm Φ×50 mm (long)] to give the title compound.

MS m/z(ESI:MH⁺): 306.2.

### Example B79

### 3-Butylphenol

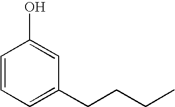

The title compound was obtained by treating 1-butyl-3-methoxybenzene in the same manner as in Example B40.

¹H-NMR (CDCl₃) δ (ppm): 0.94 (3H, t), 1.30-1.55 (2H, m), 1.55-1.62 (2H, m), 2.56 (2H, t), 4.76 (1H, brs), 6.63 (1H, dd), 6.66 (1H, d), 6.75 (1H, d), 7.12 (1H, dd).

### Example B80

### 1-Butyl-3-(methoxymethoxy)benzene

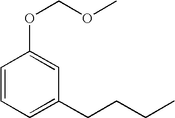

A 60% suspension of sodium hydride dispersed in mineral oil (102 mg) was added to an ice-cooled solution of the compound of Example B79 (318 mg) in dimethylformamide (5 ml), and the reaction mixture was stirred at room temperature for 30 minutes. The mixture was cooled again on ice, chloromethyl methyl ether (0.18 ml) was added, and this reaction mixture was stirred at room temperature for 12 hours. After water was added, the reaction mixture was extracted with ethyl acetate, washed with a saturated aqueous sodium hydrogencarbonate solution and saturated brine, dried over anhydrous magnesium sulfate, and then filtered through silica gel. The filtrate was concentrated under reduced pressure to give the title compound (341 mg). The obtained compound was used in the following reaction without further purification.

¹H-NMR (CDCl₃) δ (ppm): 0.94 (3H, t), 1.30-1.42 (2H, m), 1.55-2.04 (2H, m), 2.58 (2H, t), 3.49 (3H, s), 5.17 (2H, s), 6.80-6.87 (3H, m), 7.18 (1H, dd).

### Example B81

### 4-Butyl-2-(methoxymethoxy)benzaldehyde

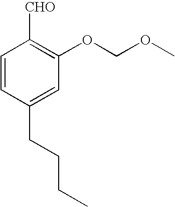

A solution of t-butyl lithium in pentane (1.51 M, 10.6 ml) was added dropwise to a solution of the compound of Example B80 (2396 mg) in petroleum ether cooled to −20° C., and this reaction mixture was stirred at a temperature in the range of −10° C. to 0° C. for 1.5 hours. The reaction mixture was cooled to −70° C., anhydrous ether (17 ml) and dimethylformamide (1.91 ml) were added, and the resulting mixture was stirred at that temperature for 3 hours, then stirred for another 1 hour at room temperature. The reaction mixture was cooled on ice, a saturated aqueous ammonium chloride solution was added, and the mixture was extracted with ethyl acetate. The extract was washed with saturated brine, dried over anhydrous magnesium sulfate, and then concentrated under reduced pressure. The residue was purified by silica gel column chromatography to give the title compound (1821 mg).

¹H-NMR (CDCl₃) δ (ppm): 0.94 (3H, t), 1.32-1.42 (2H, m), 1.57-1.65 (2H, m), 2.64 (2H, t), 3.54 (3H, s), 5.29 (2H, s), 6.91 (1H, d), 7.01 (1H, s), 7.76 (1H, d), 10.44 (1H, s).

### Example B82

### [4-Butyl-2-(methoxymethoxy)phenyl](1-isoquinolyl)methanol

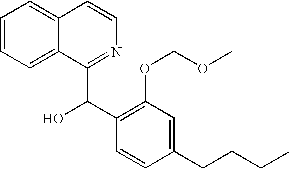

An aqueous sodium hydroxide solution (50%, 1.4 ml) was added to a solution of 1-cyano-benzoyl-1,2-dihydroisoquinoline (815 mg), which was synthesized according to Org. Synth., IV, 155 (1988), the compound of Example B81 (869 mg), and triethylbenzylammonium chloride (7 mg) in methylene chloride (1.6 ml), and the reaction mixture was subjected to ultrasonication in a water bath for 10 minutes. After methylene chloride (8.3 ml) and ethanol (4.4 ml) were added, the reaction mixture was further subjected to ultrasonication in a water bath for 85 minutes. Water was added and the resulting reaction mixture was extracted with methylene chloride. The extract was dried over anhydrous magnesium sulfate, then concentrated under reduced pressure. The residue was purified by silica gel column chromatography to give the title compound (1144 mg).

¹H-NMR (DMSO-d6) δ (ppm): 0.86 (3H, t), 1.22-1.31 (2H, m), 1.44-1.52 (2H, m), 2.44-2.51 (2H, m), 3.16 (3H, s), 5.10 (1H, d), 5.12 (1H, d), 6.72 (1H, s), 6.75 (1H, d), 6.84 (1H, s), 7.21 (1H, d), 7.61 (1H, dd), 7.72 (1H, dd), 7.74 (1H, d), 7.95 (1H, d), 8.31 (1H, d), 8.42 (1H, d).

The proton of the hydroxyl group was not observed in the NMR spectrum.

### Example B83

### [4-Butyl-2-(methoxymethoxy)phenyl](1-isoquinolyl)methyl Acetate

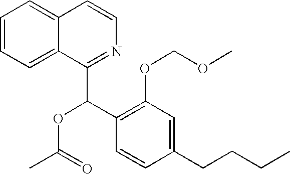

The title compound was obtained by treating the compound of Example B82 in the same manner as in Example B38.

¹H-NMR (CDCl₃) δ (ppm): 0.90 (3H, t), 1.28-1.40 (2H, m), 1.50-1.60 (2H, m), 2.22 (3H, s), 2.54 (2H, t), 3.41 (3H, s), 5.22 (1H, d), 5.26 (1H, d), 6.77 (1H, d), 6.94 (1H, s), 7.29 (1H, d), 7.55 (1H, dd), 7.58 (1H, d), 7.70 (1H, dd), 7.81 (1H, d), 8.05 (1H, s), 8.35 (1H, d), 8.55 (1H, d).

### Example B84

### 1-[4-Butyl-2-(methoxymethoxy)benzyl]isoquinoline

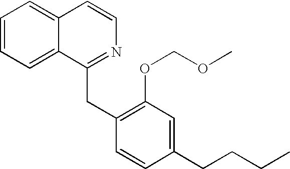

The title compound was obtained by treating the compound of Example B83 in the same manner as in Example B39.

¹H-NMR (CDCl₃) δ (ppm): 0.89 (3H, t), 1.28-1.37 (2H, m), 1.50-1.58 (2H, m), 2.53 (2H, t), 3.46 (3H, s), 4.65 (2H, s), 5.24 (2H, s), 6.66 (1H, dd), 6.89 (1H, d), 6.92 (1H, d), 7.51 (1H, dd), 7.53 (1H, d), 7.62 (1H, dd), 7.79 (1H, d), 8.23 (1H, d), 8.47 (1H, d).

### Example B85

### 5-Butyl-2-(1-isoquinolylmethyl)phenol

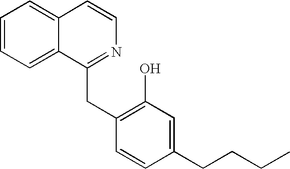

5 N hydrochloric acid (1.0 ml) was added to a solution of the compound of Example B84 (88 mg) in methanol (1.5 ml), and this reaction mixture was stirred at room temperature for 14 hours. The reaction mixture was neutralized with a 5 N aqueous sodium hydroxide solution, adjusted to pH 6.8 with phosphate buffer, and extracted with ethyl acetate. The extract was dried over anhydrous magnesium sulfate and concentrated under reduced pressure to give the title compound (44 mg).

¹H-NMR (CDCl₃) δ (ppm): 0.89 (3H, t), 1.23-1.37 (2H, m), 1.48-1.60 (2H, m), 2.51 (2H, t), 4.56 (2H, s), 6.65 (1H, dd), 6.82 (1H, d), 7.21,(H, d), 7.55 (1H, d), 7.68 (1H, dd), 7.72 (1H, dd), 7.82 (1H, d), 8.35 (1H, d), 8.44 (1H, d).

The proton of the hydroxyl group was not observed in the NMR spectrum.

### Example B86

### N-{3-[4-(1-Isoquinolylmethyl)phenyl]-2-propynyl}-N,N-dimethylamine

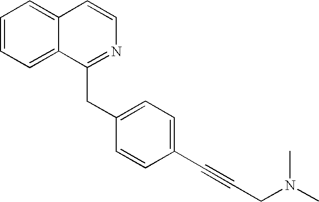

The title compound was obtained by treating the compound of Example B41 and 1-dimethylamino-2-propyne in the same manner as in Example B42.

¹H-NMR (CDCl₃) δ (ppm): 2.04 (3H, s), 2.34 (3H, s), 3.47 (2H, s), 4.66 (2H, s), 7.20 (2H, d), 7.32 (2H, d), 7.53 (1H, dd), 7.56 (1H, d), 7.65 (1H, dd), 7.82 (1H, d), 8.10 (1H, d), 8.50 (1H, d).

### Example B87

### 1-{4-[3-(Tetrahydro-2H-2-pyranyloxy)-1-propynyl]benzyl}isoquinoline

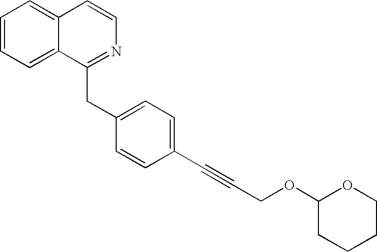

The title compound was obtained by treating the compound of Example B41 and tetrahydro-2-(2-propynyloxy)-2H-pyran in the same manner as in Example B42.

¹H-NMR (CDCl₃) δ (ppm): 1.45-1.85 (6H, m), 3.50-3.60 (1H, m), 3.84-3.90 (1H, m), 4.42 (1H, d), 4.48 (1H, d), 4.66 (2H, 8), 4.87 (1H, dd), 7.15-7.21 (2H, m), 7.33-7.36 (2H, m), 7.50-7.70 (3H, m), 7.81-7.86 (1H, m), 8.07-8.10 (1H, m), 8.48-8.51 (1H, m).

### Example B88

### 3-[4-(1-Isoquinolylmethyl)phenyl]-2-propyn-1-ol

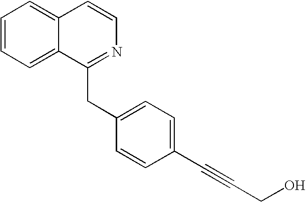

The title compound was obtained by treating the compound of Example B87 in the same manner as in Example B47.

¹H-NMR (CDCl₃) δ (ppm): 1.20-1.30 (1H, m), 4.46 (2H, s), 4.67 (2H, s), 7.23 (2H, d), 7.31 (2H, d), 7.53 (1H, dd), 7.58 (1H, d), 7.65 (1H, dd), 7.83 (1H, d), 8.09 (1H, d), 8.49 (1H, d).

### Example B89

### N,N-Dimethyl-4-pentynamide

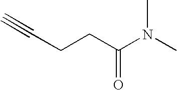

Dimethylamine (2 M solution in tetrahydrofuran, 8.53 ml), triethylamine (2.59 ml), and 1-(3-dimethylaminopropyl)-3-ethylcarbodiimide (3221 mg), were added to a solution of 4-pentynoic acid (552 mg) in methylene chloride (150 ml) and this reaction mixture was stirred at room temperature for 24 hours. The reaction mixture was washed successively with 1 N hydrochloric acid, a saturated aqueous sodium hydrogencarbonate solution, water, and saturated brine, dried over anhydrous magnesium sulfate, then concentrated under reduced pressure to give the title compound (129 mg). The obtained compound was used in the following reaction without further purification.

¹H-NMR (CDCl₃) δ (ppm): 1.96-1.99 (1H, m), 2.50-2.60 (4H, m), 2.96 (3H, s), 3.02 (3H, s).

### Example B90

### N,N-dimethyl-5-[4-(1-isoquinolylmethyl)phenyl]-4-pentynamide

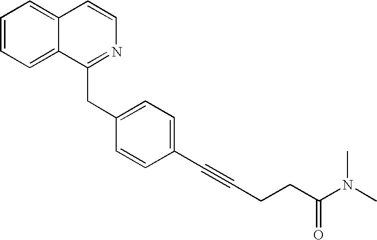

The title compound was obtained by treating the compound of Example B41 and the compound of Example B89 in the same manner as in Example B42.

¹H-NMR (CDCl₃) δ (ppm): 2.59-2.64 (2H, m), 2.71-2.75 (2H, m), 2.96 (3H, s), 3.03 (3H, s) 4.66 (2H, s), 7.18 (2H, d), 7.28 (2H, d), 7.43-7.70 (3H, m), 7.90 (1H, d), 8.09 (1H, d), 8.50 (1H, d).

### Example B91

### 1-Methyl-2-propynyltetrahydro-2H-2-pyranyl Ether

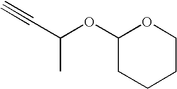

3,4-Dihydro-2H-pyran (7.15 ml) and pyridinium p-toluenesulfonate (2187 mg) were added to a solution of 3-butyn-2-ol (3051 mg) in dichloromethane (150 ml), and this reaction mixture was stirred at room temperature for 29 hours.

The reaction mixture was washed successively with a saturated aqueous sodium hydrogencarbonate solution, water, and saturated brine, dried over anhydrous magnesium sulfate, and then concentrated under reduced pressure. The residue was purified by silica gel column chromatography to give the title compound (4698 mg).

¹H-NMR (CDCl₃) δ (ppm): 1.45 (1.05H, d), 1.48 (1.95H, d), 1.50-1.90 (6H, m), 2.37 (0.65H, d), 2.43 (0.35H, d), 3.50-3.60 (1.3H, m), 3.80-3.86 (0.7H, m), 4.4-3-4.50 (0.35H, m), 4.52-4.60 (0.65H, m), 4.77 (0.35H, t), 4.94 (0.65H, t).

### Example B92

### 1-{4-[3-(Tetrahydro-2H-2-pyranyloxy)-1-butynyl]benzyl)isoquinoline

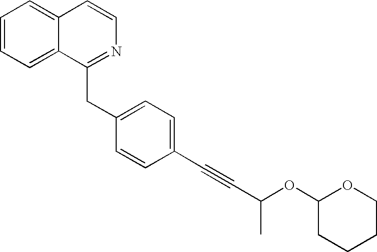

The title compound was obtained by treating the compound of Example B41 and the compound of Example B91 in the same manner as in Example B42.

¹H-NMR (CDCl₃) δ (ppm): 1.40-1.80 (6H, m), 1.49 (1.05H, d), 1.52 (1.95H, d), 3.49-3.60 (1H, m), 3.80-3.88 (0.65H, m), 3.99-4.06 (0.35H, m), 4.65 (2H, s), 4.74 (1H, q), 4.83 (0.35H, t), 4.97 (0.65H, t), 7.18-7.22 (2H, m), 7.32 (2H, d), 7.54 (1H, dd), 7.57 (1H, d), 7.64 (1H, dd), 7.82 (1H, d), 8.08 (1H, d), 8.49 (1H, d).

### Example B93

### 4-[4-(1-Isoquinolylmethyl)phenyl]-3-butyn-2-ol

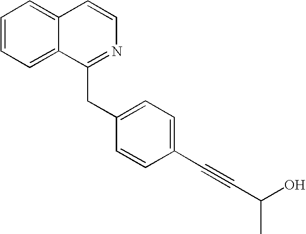

The title compound was obtained by treating the compound of Example B92 in the same manner as in Example B47.

¹H-NMR (CDCl₃) δ (ppm): 1.53 (3H, d), 2.15 (1H, brs), 4.68 (2H, s), 4.72 (1H, q), 7.21 (2H, d), 7.31 (2H, d), 7.54 (1H, dd), 7.59 (1H, d), 7.66 (1H, dd), 7.84 (1H, d), 8.10 (1H, d), 8.51 (1H, d).

### Example B94

### 4-[4-(1-Isoquinolylmethyl)phenyl]-2-butanol

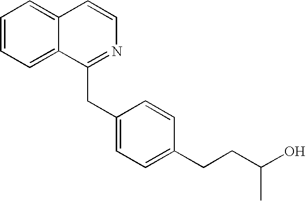

Treating the compound of Example B93 in the same manner as in Example B43, the obtained residue was separated and purified by LC-MS [eluent: an acetonitrile solution containing 0.1% trifluoroacetic acid: an aqueous solution containing 0.1% trifluoroacetic acid=1:99 to 100:0/20-minute cycle, flow rate: 20 ml/minute, column: YMC Combiprep ODS-AM, 20 mm Φ×50 mm (long)] to give the title compound.

MS m/z(ESI:MH⁺): 292.2.

### Example B95

### 2-Methyl-4-pentyn-2-ol

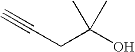

Lithium acetylide-ethylenediamine complex was added gradually to a mixed solution of isobutylene oxide (1889 mg) in tetrahydrofuran (13ml) and dimethylsulfoxide (20 ml) cooled to 0° C., and this reaction mixture was stirred at 0° C. for 5 hours. After water was added, the reaction mixture was extracted with ethyl acetate, washed with saturated brine, dried over anhydrous magnesium sulfate, and then filtered through silica gel. The filtrate was concentrated under reduced pressure to give the title compound (3316 mg). This was used in the following reaction without further purification.

¹H-NMR (CDCl₃) δ (ppm): 1.33 (6H, s), 2.09 (1H, t), 2.38 (2H, t).

The proton of the hydroxyl group was not observed in the NMR spectrum.

### Example B96

### 5-[4-(1-Isoquinolylmethyl)phenyl]-2-methyl-4-pentyn-2-ol

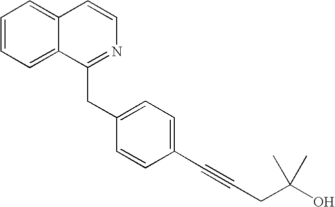

The title compound was obtained by treating the compound of Example B41 and the compound of Example B95 in the same manner as in Example B42.

¹H-NMR (DMSO-d6) δ (ppm): 1.18 (6H, s), 2.28 (1H, s), 2.42 (2H, s), 4.62 (2H, s), 7.10-7.30 (4H, m), 7.62 (1H, dd), 7.71 (1H, d), 7.72 (1H, dd), 7.94 (1H, d), 8.27 (1H, d), 8.42 (1H, d).

### Example B97

### 5-[4-(1-Isoquinolylmethyl)phenyl]-2-methyl-2-pentanol

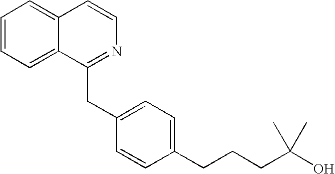

Treating the compound of Example B96 in the same manner as in Example B43, the obtained residue was separated and purified by LC-MS [eluent: an acetonitrile solution containing 0.1% trifluoroacetic acid: an aqueous solution containing 0.1% trifluoroacetic acid=1:99 to 100:0/20-minute cycle, flow rate: 20 ml/minute, column: YMC Combiprep ODS-AM, 20 mm Φ×50 mm (long)] to give the title compound.

MS m/z (ESI:MH⁺): 320.2.

### Example B98

### 4-Benzyloxy-2-(methoxymethoxy)benzaldehyde

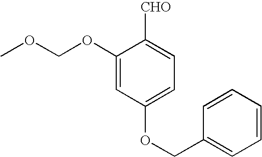

N,N-diisopropylethylamine (1.98 ml) and chloromethyl methyl ether (0.76 ml) were added to a solution of 4-benzyloxy-2-hydroxybenzaldehyde (2071 mg) in tetrahydrofuran (30 ml), and this reaction mixture was stirred and heated under reflux for 19 hours. N,N-diisopropylethylamine (2.7 ml) and chloromethyl methyl ether (1.04 ml) were further added, and the resulting mixture was stirred and heated under reflux for another 10 hours. After water was added, the reaction mixture was extracted with ethyl acetate, washed with a saturated aqueous ammonium chloride solution and saturated brine, dried over anhydrous magnesium sulfate, then filtered through silica gel and alumina. The filtrate was concentrated under reduced pressure to give the title compound (2470 mg). This compound was used in the following reaction without further purification.

¹H-NMR (CDCl₃) δ (ppm): 3.52 (3H, s), 5.12 (2H, s), 5.27 (2H, s), 6.68 (1H, dd), 6.80 (1H, d), 7.33-7.45 (5H, m), 7.82 (1H, d), 10.33 (1H, s).

### Example B99

### [4-(Benzyloxy)-2-(methoxymethoxy)phenyl](1-isoquinolyl)methanol

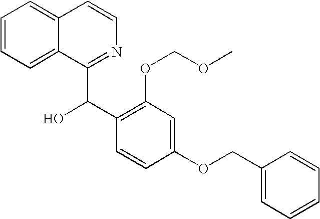

The title compound was obtained by treating the compound of Example B98 in the same manner as in Example B82.

¹H-NMR (DMSO-d6) δ (ppm): 3.16 (3H, s), 5.01 (2H, s), 5.1 (11H, d), 5.14 (1H, d), 6.59 (1H, dd), 6.66-6.70 (2H, m), 7.18 (1H, d), 7.31 (1H, d), 7.34-7.42 (4H, m), 7.61 (1H, dd), 7.71 (1H, d), 7.75 (1H, d), 7.95 (1H, d), 8.28 (1H, d), 8.43 (1H, d).

The proton of the-hydroxyl group was not observed in the NMR spectrum.

### Example B100

### [4-(Benzyloxy)-2-(methoxymethoxy)phenyl](1-isoquinolyl)methyl Acetate

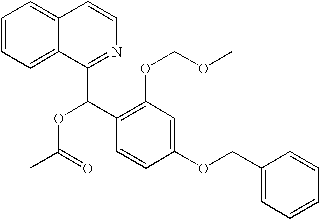

The title compound was obtained by treating the compound of Example B99 in the same manner as in Example B38.

¹H-NMR (CDCl₃) δ (ppm): 2.21 (3H, s), 3.42 (3H, s), 4.98 (1H, d), 5.00 (1H, d), 5.21-5.27 (2H, m), 6.54 (1H, dd), 6.81 (1H, d), 7.25 (1H, d), 7.30-7.41 (5H, m), 7.53 (1H, dd), 7.57 (1H, d), 7.63 (1H, dd), 7.80 (1H, d), 8.00 (1H, s), 8.29 (1H, d), 8.55 (1H, d).

### Example B101

### 4-(1-Isoquinolylmethyl)-3-(methoxymethoxy)phenol

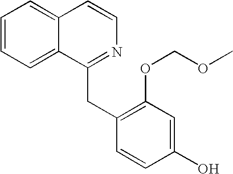

The title compound was obtained by treating the compound of Example B100 in the same manner as in Example B39.

¹H-NMR (DMSO-d6) δ (ppm): 3.36 (3H, s), 4.44 (2H, s), 5.17 (2H, s), 6.22 (1H, d), 6.52 (1H, s), 6.67 (1H, d), 7.57-7.76 (3H, m), 7.92 (1H, d), 8.22 (1H, d), 8.37 (1H, d), 9.24 (1H, brs).

### Example B102

### 4-(1-Isoquinolylmethyl)-3-(methoxymethoxy)phenyl Trifluoromethanesulfonate

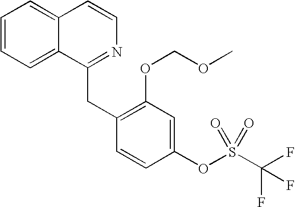

The title compound was obtained by treating the compound of Example B101 in the same manner as in Example B41.

¹H-NMR (CDCl₃) δ (ppm): 3.43 (3H, s), 4.65 (2H, s), 5.24 (2H, s), 6.77 (1H, dd), 7.04 (1H, d), 7.07 (1H, d), 7.54-7.61 (2H, m), 7.67 (1H, dd), 7.84 (1H, d), 8.16 (1H, d), 8.47 (1H, d).

### Example B103

### 1-{2-(Methoxymethoxy)-[4-(tetrahydro-2H-2-pyranyloxy)-1-butynyl]benzyl}isoquinoline

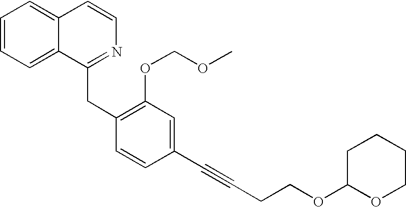

The title compound was obtained by treating the compound of Example B102 and 2-(3-butynyloxy)tetrahydro-2H-pyran in the same manner as in Example B42.

¹H-NMR (CDCl₃) δ (ppm): 1.51-1.90 (6H, m), 2.68 (2H, t), 3.50 (3H, s), 3.49-3.55 (1H, m), 3.58-3.65 (1H, m), 3.84-3.94 (2H, m), 4.63-4.68 (1H, m), 4.65 (2H, s), 5.23 (2H, s), 6.76 (1H, dd), 7.04 (1H, d), 7.07 (1H, d), 7.49-7.69 (3H, m), 7.81 (1H, d), 8.14 (1H, d), 8.47 (1H, d).

### Example B104

### 5-(4-Hydroxy-1-butynyl)-2-(1-isoquinolylmethyl)phenol

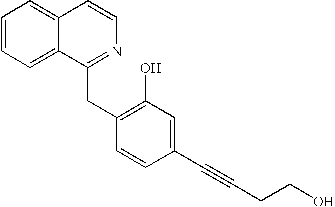

The title compound was obtained by treating the compound of Example B103 in the same manner as in Example B85.

¹H-NMR (CDCl₃) δ (ppm): 1.80 (1H, brs), 2.66 (2H, t), 3.73-3.82 (2H, m), 4.58 (2H, s), 6.87 (1H, d), 7.04 (1H, s), 7.23 (1H, d), 7.60 (1H, d), 7.69-7.78 (2H, m), 7.86 (1H, d), 8.37 (1H, d), 8.42 (1H, d).

The proton of the phenolic hydroxyl group was not observed in the NMR spectrum.

### Example B105

### 1-(t-Butyl)-1,1-dimethylsilyl {4-[4-(1-Isoquinolylmethyl)-phenyl]-2-methyl-3-butynyl}ether

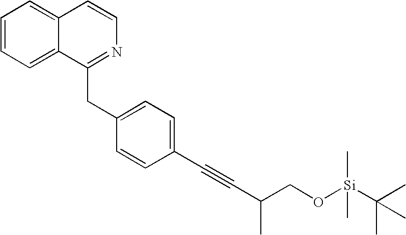

Triphenylphosphine (18.37 g) was added to an ice-cooled solution of carbon tetrabromide (11.19 g) in methylene chloride (60 ml), and this reaction mixture was stirred at that temperature for 1 hour. A solution of 3-{[1-(t-butyl)-1,1-dimethylsilyl]oxy}-2-methylpropanal, which was synthesized according to Tetrahedron Lett., 4347 (1979), in methylene chloride (14 ml) was added dropwise, and the resulting reaction mixture was further stirred for 1 hour. The reaction mixture was diluted with methylene chloride, washed successively with saturated aqueous sodium hydrogencarbonate solution, saturated an aqueous ammonium chloride solution and saturated brine, dried over magnesium sulfate, and then concentrated under reduced pressure. Ether was added to this residue, insoluble material was separated by filtration, and the filtrate was concentrated under reduced pressure. The residue was purified by silica gel column chromatography to give t-butyl[(4,4-dibromo-2-methyl-3-butenyl)oxy]-dimethylsilane (2385 mg).

Next, a 2.47 M n-butyl lithium solution in hexane (3.15 ml) was added dropwise to a solution of t-butyl[(4,4-dibromo-2-methyl-3-butenyl)oxy]dimethylsilane (1326 mg) in tetrahydrofuran (10 ml) cooled to −70° C., and this mixture was stirred at that temperature for 1 hour. A saturated aqueous ammonium chloride solution was further added, and the resulting mixture was warmed to room temperature. After water was added, the reaction mixture was extracted with ether. The ether layer was washed with saturated brine, dried over anhydrous magnesium sulfate, then filtered through silica gel. The filtrate was concentrated under reduced pressure. The obtained residue and the compound of Example B41 were treated in the same manner as in Example B42 to obtain the title compound.

¹H-NMR (CDCl₃) δ (ppm): 0.07 (6H, s), 0.90 (9H, s), 1.18 (3H, d), 2.70-2.80 (1H, m), 3.47 (1H, dd), 3.70 (1H, dd), 4.65 (2H, s), 7.16 (2H, d), 7.27 (2H, d), 7.51 (1H, dd), 7.56 (1H, d), 7.64 (1H, dd), 7.81 (1H, d), 8.07 (1H, d), 8.49 (1H, d).

### Example B106

### 4-[4-(1-Isoquinolylmethyl)phenyl]-2-methyl-3-butyn-1-ol

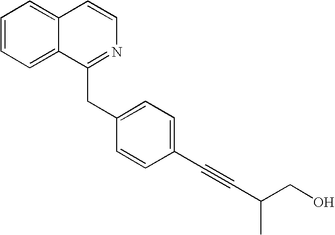

The title compound was obtained by treating the compound of Example B105 in the same manner as in Example B47.

¹H-NMR (DMSO-d6) δ (ppm): 1.11 (3H, d), 2.60-2.70 (1H, m), 3.28 (1H, d), 3.44 (1H, d), 4.58 (2H, s), 4.85-4.90 (1H, m), 7.23 (4H, s), 7.61 (1H, dd), 7.70 (1H, d), 7.71 (1H, dd), 7.93 (1H, d), 8.25 (1H, d), 8.42 (1H, d).

### Example B107

### 1-([1-(t-Butyl)-1,1-dimethylsilyl]oxy}-3-butyn-2-ol

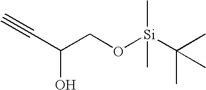

Ethynyl magnesium bromide in tetrahydrofuran (0.5 M, 90 ml) was added to anhydrous tetrahydrofuran (20 ml) cooled to −78° C. under nitrogen atmosphere. A solution of t-butyldimethylsiloxyacetaldehyde (6000 mg) in tetrahydrofuran (30 ml) was added dropwise, and thee resulting mixture was stirred at −78° C. for 45 minutes, warmed to room temperature, stirred for 1 hour 40 minutes, then cooled on ice. After a saturated aqueous ammonium chloride solution was added, the reaction mixture was extracted with ether, washed with water and saturated brine, dried over anhydrous magnesium sulfate, and then filtered through silica gel. The filtrate was concentrated under reduced pressure to give the title compound (8.55 g). This compound was used in the following reaction without further purification.

¹H-NMR (CDCl₃) δ (ppm): 0.08 (6H, s), 0.91 (9H, s), 2.43 (1H, d), 2.60-2.66 (1H, m), 3.65-3.70 (1H, m), 3.73-3.81 (1H, m), 4.38-4.42 (1H, m).

### Example B108

### 1-{[1-(t-Butyl)-1,1-dimethylsilyl]oxy}methyl)-2-propynyl Acetate

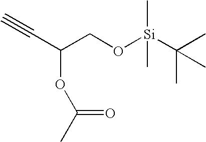

The title compound was obtained by treating the compound of Example B107 in the same manner as in Example B38.

¹H-NMR (CDCl₃) δ (ppm): 0.08 (6H, s), 0.90 (9H, s), 2.11 (3H, s), 2.44 (1H, d), 3.80-3.88 (2H, m), 5.41-5.55 (1H, m).

### Example B109

### 4-[4-(1-Isoquinolylmethyl)phenyl]-3-butyn-1,2-diol

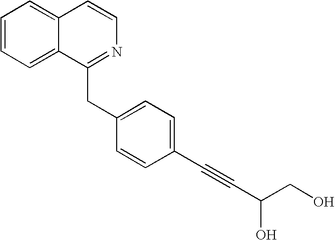

The compound of Example B41 and the compound of Example B108 were treated in the same manner as in Example B42 to give the coupling product. The title compound was obtained by deprotecting the hydroxyl protecting group of the coupling product in the same manner as in Example B47.

¹H-NMR (DMSO-d6) δ (ppm): 3.40-3.45 (1H, m), 3.70-3.82 (1H, m), 4.30-4.35 (1H, m) 4.63 (2H, s) 4.90 (1H, t), 5.46 (1H, d), 7.25-7;30 (4H, m), 7.62 (1H, dd), 7.71 (1H, d), 7.73 (1H, dd), 7.94 (1H, d), 8.28 (1H, d), 8.43 (1H, d).

### Example B110

### 1-{4-[2-(2,2-Dimethyl-1,3-dioxolan-4-yl)-1-ethynyl]benzyl}isoquinoline

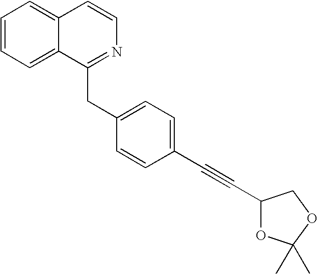

2,2-Dimethoxypropane (0.36 ml), 10-camphorsulfonic acid (43 mg), and molecular sieves (4 Å) were added to a solution of the compound of Example B109 (34 mg) in dimethylformamide (2 ml), and this reaction mixture was stirred at 75° C. for 9 hours. After an saturated aqueous sodium carbonate solution was added, the reaction mixture was extracted with ethyl acetate, washed with water, dried over anhydrous magnesium sulfate, and then concentrated under reduced pressure. The residue was purified by silica gel column chromatography to give the title compound (14 mg).

¹H-NMR (CDCl₃) δ (ppm): 1.40 (3H, s), 1.50 (3H, s), 3.97 (1H, dd), 4.21 (1H, dd), 4.66 (2H, s), 4.91 (1H, dd), 7.19 (2H, d), 7.32 (2H, d), 7.52 (1H, dd), 7.65-7.78 (2H, m), 8.08 (1H, d), 8.09 (1H, d), 8.49 (1H, d).

### Example B111

### t-Butyl{[2-(1-ethoxyethoxy)-3-butynyl]oxy}dimethylsilane

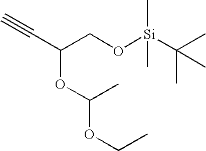

Ethyl vinyl ether (1.21 ml) and pyridinium p-toluenesulfonate (317 mg) were added to a solution of 1-{[1-(t-butyl)-1,1-dimethylsilyl]oxy}-3-butyn-2-ol (1687 mg) in methylene chloride (90 ml), and this mixture was stirred at room temperature for 1 hour. The methylene chloride layer was washed with a saturated aqueous sodium hydrogencarbonate solution and saturated brine, dried over anhydrous magnesium sulfate, then concentrated under reduced pressure to give the title compound (1962 mg). This compound was used in the following reaction without further purification.

¹H-NMR (DMSO-d6) δ (ppm): 0.00 (6H, s), 0.81 (9H, s), 1.01-1.07 (3H, m), 1.10-1.20 (1H, m), 1.18 (3H, d), 3.35-3.63 (4H, m), 4.18-4.27 (1H, m), 4.74 (0.5H, q), 4.81 (0.5H, q).

### Example B112

### 1-{4-[4-{[1-(t-Butyl)-1,1-dimethylsilyl]oxy}-3-(1-ethoxyethoxy)-1-butynyl]benzyl}isoquinoline

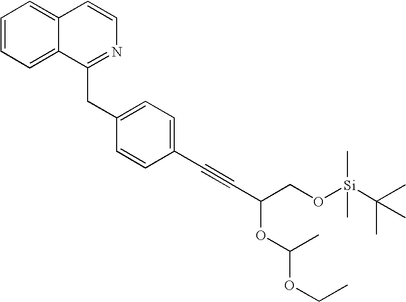

The title compound was obtained by treating the compound of Example B41 and the compound of Example B111 in the same manner as in Example B42.

¹H-NMR (DMSO-d6) δ (ppm): 0.00 (6H, s), 0.80 (9H, s), 1.01-1.05 (3H, m), 1.19 (3H, d), 3.39-3.70 (4H, m), 4.41 (0.5H, t), 4.48 (0.5H, t), 4.59 (2H, s), 4.79 (0.5H, q), 4.87 (0.5H, q), 7.20-7.30 (4H, m), 7.58 (1H, dd), 7.68 (1H, d), 7.69 (1H, dd), 7.91 (1H, d), 8.24 (1H, d), 8.38 (1H, d).

### Example B113

### 1-{[1-(t-Butyl)-1,1-dimethylsilyl]oxy}4-[4-(1-isoquinolylmethyl)phenyl]-3-butyn-2-ol

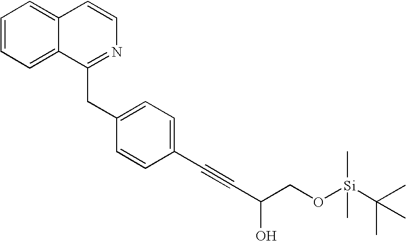

Pyridinium p-toluenesulfonate (486 mg) was added to a solution of the compound of Example B112 (474 mg) in methanol (15 ml), and this reaction mixture was stirred at room temperature for 24 hours. After ethyl acetate was added, the reaction mixture was washed with a saturated aqueous sodium hydrogencarbonate solution and saturated brine, dried over anhydrous magnesium sulfate, and then concentrated under reduced pressure. The residue was purified by silica gel column chromatography to give the title compound (265 mg).

¹H-NMR (DMSO-d6) δ (ppm): 0.01 (6H, s), 0.82 (9H, s), 3.55-3.62 (2H, m), 4.30-4.39 (1H, m), 4.61 (2H, s), 5.51 (1H, d), 7.20-7.27 (4H, m), 7.50-7.63 (1H, m), 7.67-7.74 (2H, m), 7.92 (1H, d), 8.27 (1H, d), 8.41 (1H, d).

### Example B114

### 1-(t-Butyl)-1,1-dimethylsilyl{2-fluoro-4-[4-(1-isoquinolylmethyl)phenyl]-3-butynyl}Ether

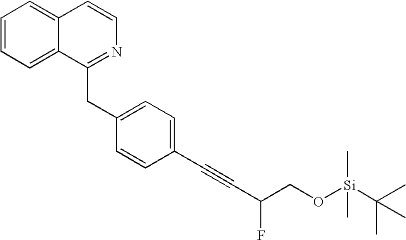

A solution of the compound of Example B113 (116 mg) in methylene chloride (2 ml) was added dropwise to a solution of (diethylamino)sulfur trifluoride (44 μl) in methylene chloride (2 ml) cooled to −78° C. under nitrogen atmosphere. Upon stirring for 15 minutes, the reaction mixture was stirred at room temperature for another 8 hours. A saturated aqueous sodium hydrogencarbonate solution was added, the resulting reaction mixture was extracted with methylene chloride. The methylene chloride layer was washed with water, dried over anhydrous magnesium sulfate, and then concentrated under reduced pressure. The residue was purified by silica gel column chromatography to give the title compound (42 mg).

¹H-NMR (CDCl₃) δ (ppm): 0.10 (6H, s), 0.91 (9H, s), 3.83-4.00 (2H, m), 4.67 (2H, s), 5.17 (1H, ddd), 7.22 (2H, d), 7.34 (2H, d), 7.53 (1H, dd), 7.58 (1H, d), 7.65 (1H, dd), 7.83 (1H, d), 8.08 (1H, d), 8.50 (1H, d).

### Example B115

### 2-Fluoro-4-[4-(1-isoquinolylmethyl)phenyl]-3-butyn-1-ol

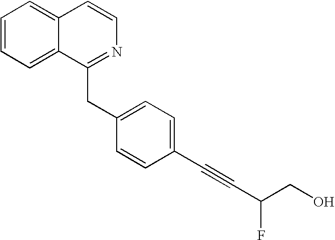

The title compound was obtained by treating the compound of Example B114 in the same manner as in Example B47.

¹H-NMR (CDCl₃) δ (ppm): 1.31 (1H, brs), 3.77-3.95 (2H, m), 4.67 (2H, s), 5.35 (1H, ddd), 7.22 (2H, d), 7.35 (2H, d), 7.53 (1H, dd), 7.58 (1H, d), 7.65 (1H, dd), 7.83 (1H, d), 8.07 (1H, d), 8.50 (1H, d).

### Example B116

### 1-(t-Butyl)-1,1-dimethylsilyl{6-[4-(1-isoquinolylmethyl)-phenyl]-5-hexynyl}Ether

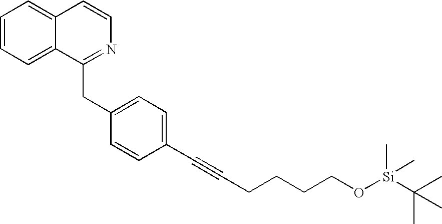

The title compound was obtained by treating the compound of Example B41 and t-butyl(5-hexynyloxy)dimethylsilane in the same manner as in Example B42.

¹H-NMR (CDCl₃) δ (ppm): 0.04 (6H, s), 0.88 (9H, s), 1.55-1.70 (4H, m), 2.39 (2H, t), 3.64 (2H, t), 4.65 (2H, s), 7.17 (2H, d), 7.27 (2H, d), 7.51 (1H, dd), 7.55 (1H, d), 7.64 (1H, dd), 7.82 (1H, d), 8.08 (1H, d), 8.49 (1H, d):

### Example B117

### 6-[4-(1-Isoquinolylmethyl)phenyl]-5-hexyn-1-ol

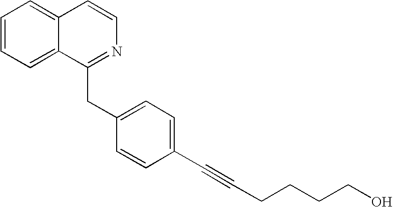

The title compound was obtained by treating the compound of Example B116 in the same manner as in Example B47.

¹H-NMR (CDCl₃) δ (ppm): 1.60-1.80 (4H, m), 2.42 (2H, t), 3.69 (2H, t), 4.65 (2H, s), 7.17 (2H, d), 7.27 (2H, d), 7.52 (1H, dd), 7.57 (1H, d), 7.64 (1H, dd), 7.81 (1H, d), 8.08 (1H, d), 8.49 (1H, d).

The proton of the hydroxyl group was not observed in the NMR spectrum.

### Example B118

### 6-[4-(1-Isoquinolylmethyl)phenyl]-1-hexanol

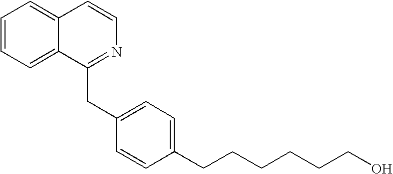

Treating the compound of Example B117 in the same manner as in Example B43, the obtained residue was separated and purified by LC-MS [eluent: an acetonitrile solution containing 0.1% trifluoroacetic acid: an aqueous solution containing 0.1% trifluoroacetic acid=1:99 to 100:0/20-minute cycle, flow rate: 20 ml/minute, column: YMC Combiprep ODS-AM, 20 mm Φ×50 mm (long)] to give the title compound.

MS m/z(ESI:MH⁺): 320.2.

### Example B119

### 2-(4-Pentynyloxy)tetrahydro-2H-pyran

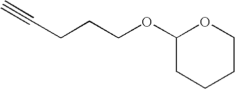

The title compound was obtained by treating 4-pentyn-1-ol in the same manner as in Example B91.

¹H-NMR (CDCl₃) δ (ppm): 1.50-1.90 (8H, m), 1.95 (1H, t), 2.30-2.35 (2H, m), 3.46-3.54 (2H, m), 3.80-3.90 (2H, m), 4.60 (1H, dd).

### Example B120

### 1-{4-[5-(Tetrahydro-2H-2-pyranyloxy)-1-pentynyl]benzyl}isoquinoline

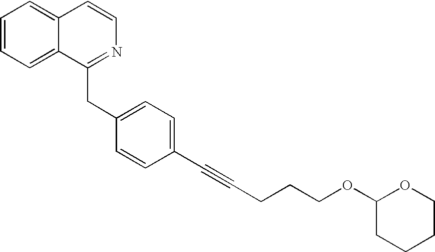

The title compound was obtained by treating the compound of Example B41 and the compound of Example B119 in the same manner as in Example B42.

¹H-NMR (CDCl₃) δ (ppm): 1.49-1.90 (8H, m), 2.49 (2H, t), 3.47-3.54 (2H, m), 3.82-3.90 (2H, m), 4.60 (1H, dd), 4.65 (2H, s), 7.17 (2H, d), 7.27 (2H, d), 7.52 (1H, dd), 7.58 (1H, d), 7.64 (1H, dd), 7.82 (1H, d), 8.09 (1H, d), 8.49 (1H, d).

### Example B121

### 5-[4-(1-Isoquinolylmethyl)phenyl]-4-pentyn-1-ol

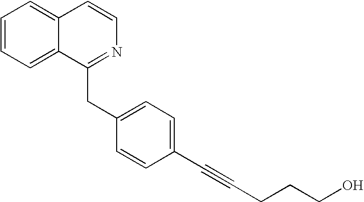

The title compound was obtained by treating the compound of Example B120 in the same manner as in Example B47.

¹H-NMR (CDCl₃) δ (ppm): 1.80-1.88 (2H, m), 2.51 (2H, t), 3.80 (2H, t), 4.65 (2H, s), 7.18 (2H, d), 7.29 (2H, d), 7.52 (1H, dd), 7.58 (1H, d), 7.65 (1H, dd), 7.82 (1H, d), 8.09 (1H, d), 8.49 (1H, d).

The proton of the hydroxyl group was not observed in the NMR spectrum.

### Example B122

### 5-[4-(1-Isoquinolylmethyl)phenyl]-4-pentynylcyanide

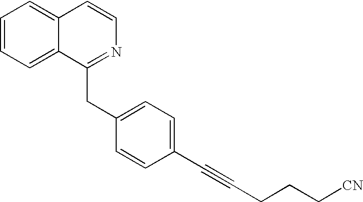

The title compound was obtained by treating the compound of Example B41 and 5-cyano-1-pentyne in the same manner as in Example B42.

¹H-NMR (CDCl₃) δ (ppm): 1.85-1.98 (2H, m), 2.40-2.60 (4H, m), 4.66 (2H, s), 7.20 (2H, d), 7.28 (2H, d), 7.53 (1H, dd), 7.58 (1H, d), 7.65 (1H, dd), 7.83 (1H, d), 8.09 (1H, d), 8.50 (1H, d).

### Example B123

### 1-[4-(3-Methyl-1-butynyl)benzyl]isoquinoline

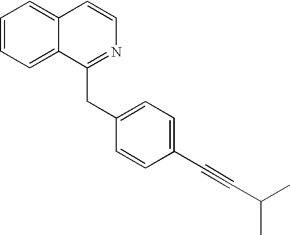

The title compound was obtained by treating the compound of Example B41 and 3-methyl-1-butyne in the same manner as in Example B42.

¹H-NMR (CDCl₃) δ (ppm): 1.23 (6H, d), 2.70-2.78 (1H, m), 4.65 (2H, s), 7.18 (2H, d), 7.28 (2H, d), 7.51 (1H, dd), 7.58 (1H, d), 7.64 (1H, dd), 7.82 (1H, d), 8.08 (1H, d), 8.50 (1H, d).

### Example B124

### 1-[4-(5-Methyl-1-hexynyl)benzyl]isoquinoline

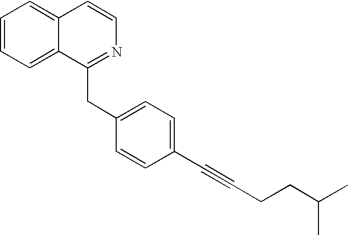

The title compound was obtained by treating the compound of Example B41 and 5-methyl-1-hexyne in the same manner as in Example B42.

¹H-NMR (CDCl₃) δ (ppm): 0.91 (6H, d), 1.47 (2H, dt), 1.68-1.77 (1H, m), 2.37 (2H, t), 4.65 (2H, s), 7.17 (2H, d), 7.28 (2H, d), 7.52 (1H, dd), 7.57 (1H, d), 7.64 (1H, dd), 7.81 (1H, d), 8.09 (1H, d), 8.49 (1H, d).

### Example B125

### 4-Pentynamide

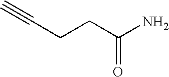

1-Ethoxycarbonyl-2-ethoxy-1,2-dihydroquinoline (6775 mg) and ammonium hydrogencarbonate (5905 mg) were added to a solution of 4-pentynoic acid (2446 mg) in chloroform (75 ml), and this reaction mixture was stirred at room temperature for 17.5 hours. The reaction mixture was filtered through celite and concentrated under reduced pressure. The residue was purified by silica gel column chromatography to give the title compound (249 mg).

¹H-NMR (DMSO-d6) δ (ppm): 2.21 (2H, t), 2.29-2.33 (2H, m), 2.73 (1H, t), 6.78-6.88 (1H, m), 7.28-7.38 (1H, m).

### Example B126

### 5-[4-(1-Isoquinolylmethyl)phenyl]-4-pentynamide

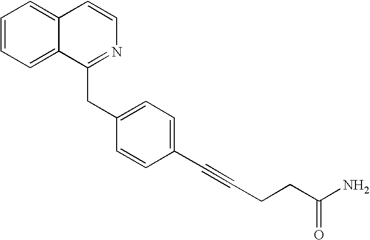

The title compound was obtained by treating the compound of Example B41 and the compound of Example B125 in the same manner as in Example B42.

¹H-NMR (DMSO-d6) δ (ppm): 2.51 (2H, t), 2.85 (2H, t), 3.70 (2H, brs), 4.59 (2H, s), 7.05 (2H, d), 7.23 (2H, d), 7.61 (1H, dd), 7.70 (1H, d), 7.72 (1H, dd), 7.94 (1H, d), 8.30 (1H, d), 8.43 (1H, d).

### Example B127

### t-Butyl 4-Pentynoate

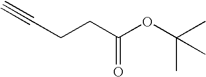

Benzyltriethylammonium chloride (5.92 g), potassium carbonate (93.4 g), and t-butyl bromide (143 ml) were added to a solution of 4-pentynoic acid (2550 mg) in N,N-dimethylacetamide (230 ml), and this reaction mixture was stirred at 55° C. for 24 hours. After water was added, the reaction mixture was extracted with ethyl acetate, washed with water, dried over anhydrous magnesium chloride, and then filtered through silica gel. The filtrate was concentrated under reduced pressure to give the title compound (2.10 g) This compound was used in the following reaction without further purification.

¹H-NMR (CDCl₃) δ (ppm): 1.46 (9H, s), 1.96-1.97 (1H, m), 2.45-2.47 (4H, m).

### Example B128

### t-Butyl 5-[4-(1-Isoquinolylmethyl)phenyl]-4-pentynoate

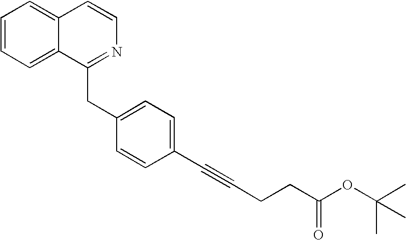

The title compound was obtained by treating the compound of Example B41 and the compound of Example B127 in the same manner as in Example B42.

¹H-NMR (CDCl₃) δ (ppm): 1.45 (9H, s), 2.49 (2H, t), 2.64 (2H, t), 4.64 (2H, s), 7.21 (2H, d), 7.26 (2H, d), 7.52 (1H, dd), 7.57 (1H, d), 7.64 (1H, dd), 7.82 (1H, d), 8.09 (1H, d), 8.49 (1H, d).

### Example B129

### 5-[4-(1-Isoquinolylmethyl)phenyl]-4-pentynoic Acid

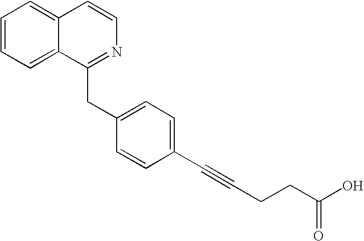

Treating the compound of Example B128 in the same manner as in Example B69, the obtained residue was separated and purified by LC-MS [eluent: an acetonitrile solution containing 0.1% trifluoroacetic acid: an aqueous solution containing 0.1% trifluoroacetic acid=1:99 to 100:0/20-minute cycle, flow rate: 20 ml/minute, column: YMC Combiprep ODS-AM, 20 mm Φ×50 mm (long)] to give the title compound.

MS m/z(ESI:MH⁺): 316.1.

The following compounds were synthesized as follows. That is, the title compound was obtained by reacting the compound of Example B41 with various reactants described below, according to Example B33. The various reactants are acrylamide, N,N-dimethylacrylamide, t-butyl acrylate, and methyl vinyl sulfone. Furthermore, the coupling product obtained in this manner was subjected to either the reduction according to Example B39 or the deprotection of t-butyl ester according to Example B40, or both. The resulting product was purified by silica gel column chromatography or by LC-MS [eluent: an acetonitrile solution containing 0.1% trifluoroacetic acid: an aqueous solution containing 0.1% trifluoroacetic acid=1:99 to 100:0/20-minute cycle, flow rate: 20 ml/minute, column: YMC Combiprep ODS-AM, 20 mm Φ×50 mm (long)].

### Example B130

### (E)-3-[4-(1-Isoquinolylmethyl)phenyl]-2-propenamide

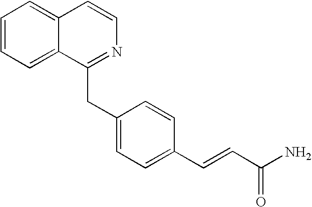

MS m/z(ESI:MH⁺): 289.3.

### Example B131

### 3-[4-(1-Isoquinolylmethyl)phenyl]-2-propanamide

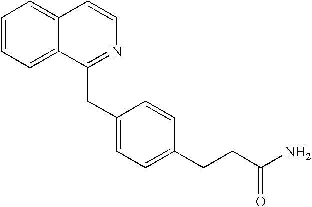

MS m/z(ESI:MH⁺): 291.2.

### Example B132

### N,N-Dimethyl-(E)-3-[4-(1-isoquinolylmethyl)phenyl]-2-propenamide

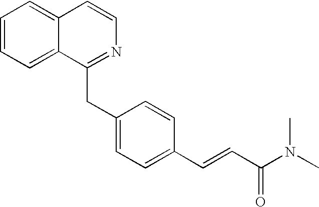

MS m/z(ESI:MH⁺): 317.3.

### Example B133

### N,N-Dimethyl-3-[4-(1-isoquinolylmethyl)phenyl]propanamide

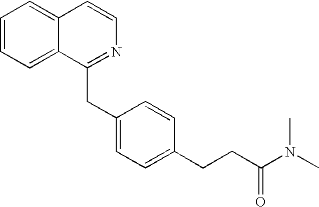

MS m/z(ESI:MH⁺): 319.1.

### Example B134

### t-Butyl (E)-3-[4-(1-isoquinolylmethyl)phenyl]-2-propenoate

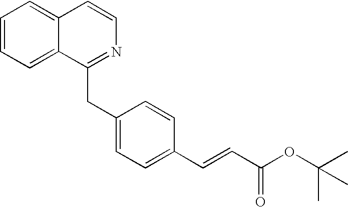

¹H-NMR (CDCl₃) δ (ppm): 1.51 (9H, s), 4.68 (2H, s), 6.28 (1H, d), 7.27 (2H, d), 7.39 (2H, d), 7.49-7.60 (3H, m), 7.65 (1H, dd), 7.82 (1H, d), 8.11 (1H, d), 8.50 (1H, d).

### Example B135

### (E)-3-[4-(1-Isoquinolylmethyl)phenyl]-2-propenoic Acid

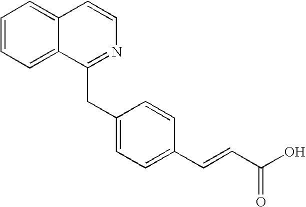

MS m/z(ESI:MH⁺): 290.2.

### Example B136

### t-Butyl 3-[4-(1-Isoquinolylmethyl)phenyl]propanoate

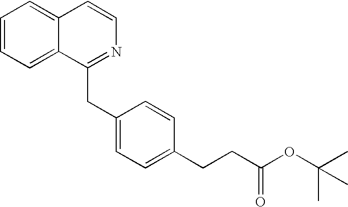

¹H-NMR (CDCl₃) δ (ppm): 1.37 (9H, s), 2.47 (2H, t), 2.83 (2H, t), 4.64 (2H, s), 7.07 (2H, d), 7.19 (2H, d), 7.52 (1H, dd), 7.56 (1H, d), 7.63 (1H, dd), 7.81 (1H, d), 8.14 (1H, d), 8.49 (1H, d).

### Example B137

### 3-[4-(1-Isoquinolylmethyl)phenyl]propanoic Acid

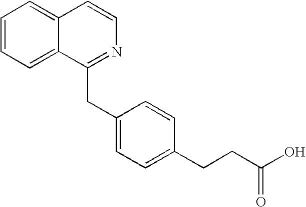

MS m/z(ESI:MH⁺): 292.1.

### Example B138

### (E)-2-[4-(1-Isoquinolylmethyl)phenyl]-1-ethenyl Methylsulfone

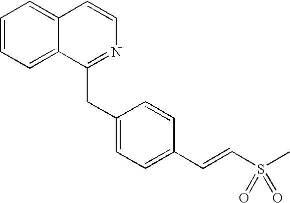

MS m/z(ESI:MH⁺): 324.1.

### Example B139

### 1-{4-[2-(Methylsulfonyl)ethyl]benzyl}isoquinoline

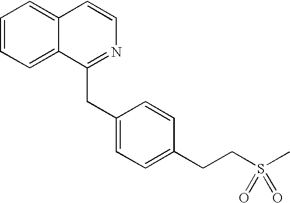

MS m/z(ESI:MH⁺): 326.1.

### Example B140

### 2-Benzoyl-6,7-dimethoxy-1,2-dihydro-1-isoquinolinecarbonitrile

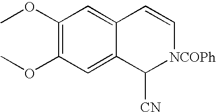

An aqueous potassium cyanide (1.0 g, 16 mmol) solution (2.3 ml) and benzoyl chloride (1.1 ml, 9.5 mmol) were added to a solution of 6,7-dimethoxyisoquinoline (1.0 g, 5.3 mmol), which was synthesized according to Tetrahedron, 37 (23), 3977 (1981), in methylene chloride (6.0 ml), and this reaction mixture was stirred while heating under reflux for 2 hours. The reaction mixture was cooled to room temperature, filtered through celite, and washed with methylene chloride and water. After the obtained filtrate was separated, the methylene chloride layer was washed successively with water, 2 N hydrochloric acid, water, and 2 N sodium hydroxide, dried over anhydrous magnesium sulfate, and then concentrated under reduced pressure. The residue was purified by silica gel column chromatography to give the title compound (573 mg).

¹H-NMR (CDCl₃) δ (ppm): 3.92 (3H, s), 3.94 (3H, s), 5.99 (1H, d), 6.51-6.55 (2H, m), 6.73 (1H, s), 6.85 (1H, s), 7.45-7.49 (2H, m), 7.53-7.56 (1H, m), 7.58-7.61 (2H, m).

### Example B141

### 1-(4-Butylbenzyl)-6,7-dimethoxyisoquinoline

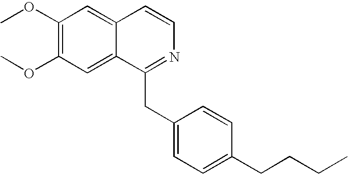

The title compound was obtained by treating the compound of Example B140 and the compound of Example B1 in the same manner as in Example B2.

¹H-NMR (CDCl₃) δ (ppm): 0.90 (3H, t), 1.27-1.36 (2H, m), 1.51-1.58 (2H, m), 2.54 (2H, t), 3.88 (3H, s), 4.01 (3H, s), 4.57 (2H, s), 7.05 (1H, s), 7.07 (2H, d), 7.19 (2H, d), 7.32 (1H, s), 7.43 (1H, d), 8.37 (1H, d).

### Example B142

### 1-(3-Methoxyphenyl)-2-nitro-1-ethanol

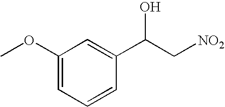

An aqueous sodium hydroxide solution (1.5 g of sodium hydroxide (37 mmol) was dissolved in 15 ml of water) was added dropwise to a solution of m-anisaldehyde (5.0 g, 37 mmol) and nitromethane (4.0 ml, 73 mmol) in methanol (50 ml) keeping the temperature of the solution at not higher than 30° C. The reaction mixture was then stirred at room temperature for 4 hours. Upon cooling on ice, an aqueous acetic acid solution (glacial acetic acid (37 mmol) was dissolved in 250 ml of water) was added, the resulting reaction mixture was extracted with ethyl acetate. The ethyl acetate layer was washed successively with water and a 5% aqueous sodium hydrogencarbonate solution, dried over anhydrous magnesium sulfate, then concentrated under reduced pressure. The residue was purified by silica gel column chromatography to give the title compound (6.09 g).

¹H-NMR (CDCl₃) δ (ppm): 3.83 (3H, s), 4.52 (1H, dd), 4.61 (1H, dd), 4.76-4.78 (1H, m), 5.44-5.48 (1H, m), 6.90 (1H, dd), 6.96-6.98 (2H, m), 7.25-7.34 (1H, m).

### Example B143

### 2-Amino-1-(3-methoxyphenyl)-1-ethanol

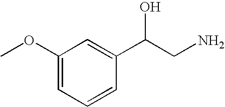

Palladium-carbon (10%, 0.64 g) and ammonium formate (4.8 g) were added to a mixed solution of the compound of Example B142 (3.0 g, 15 mmol) in tetrahydrofuran (43 ml) and methanol (43 ml), and this mixture was stirred at room temperature for 18 hours. The catalyst was removed by filtration, the filtrate was diluted with ether, precipitates were removed by filtration, and the obtained filtrate was concentrated to give the title compound (1.82 g). This compound was used in the following reaction without further purification.

### Example B144

### 2-(4-Butylphenyl)acetic Acid

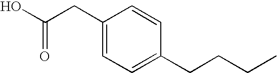

Thionyl chloride (4.7 ml, 66 mmol) was added dropwise to a solution of 4-n-butylbenzyl alcohol (9.6 g, 59 mmol) in ether (120 ml), and this mixture was stirred at room temperature for 2 hours. The solvent was removed under reduced pressure, and excess thionyl chloride was removed by azeotropic distillation with benzene. The residue was dissolved in dimethyl sulfoxide (50 ml), sodium cyanide (86 g, 1.8 mol) and n-tetrabutylammonium iodide (2.2 g, 5.9 mmol) were added to this solution, and the resulting mixture was stirred at room temperature for 16 hours. Water was added, and this mixture was extracted with ethyl acetate. The ethyl acetate layer was washed successively with water and saturated brine, dried over anhydrous magnesium sulfate, and then concentrated under reduced pressure. The residue was purified by silica gel column chromatography to give n-butylphenylacetonitrile (8.2 g) as a yellow oil. Next, concentrated sulfuric acid (48 ml) was added dropwise to water (58 ml), this solution was cooled to 50° C., and n-Butylphenylacetonitrile (8.2 g) obtained above was added dropwise to the solution. The resulting mixture was stirred while heating under reflux for 16 hours. Upon cooling to room temperature, the precipitated crystals were collected by filtration, washed with water, and dissolved in a 0.1 N aqueous sodium hydroxide solution (200 ml). Norit (5 g) was added, and this mixture was stirred and refluxed for 2 hours. After Norit was removed by filtration through celite, the filtrate was cooled to room temperature and acidified with 1 N hydrochloric acid to precipitate crystals. The precipitated crystals were collected by filtration, washed with water, and dried to give the title compound (3.5 g).

¹H-NMR (CDCl₃) δ (ppm): 0.93 (3H, t), 1.30-1.40 (2H, m), 1.53-1.62 (2H, m), 2.59 (2H, t), 3.62 (2H, s), 7.15 (2H, d), 7.20 (2H, d).

The OH of the carboxyl group was not observed in the NMR spectrum.

### Example B145

### N-[2-Hydroxy-2-(3-methoxyphenyl)ethyl]-2-(4-butylphenyl)acetamide

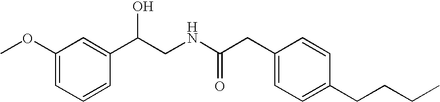

Thionyl chloride (0.76 ml, 10 mmol) was added to a solution of the compound of Example B144 (1.0 g, 5.2 mmol) in benzene (10 ml), and the mixture was stirred under reflux for 2 hours. Upon concentration, excess thionyl chloride was removed by azeotropic distillation with benzene. The obtained residue and the compound of Example B143 (0.87 g, 5.2 mmol) were dissolved in ether (5 ml), an aqueous sodium hydroxide solution (0.21 g of sodium hydroxide was dissolved in 4.2 ml of water) was added thereto, and the mixture was stirred vigorously at room temperature for 30 minutes. The ether layer was separated and concentrated under reduced pressure to give the title compound (600 mg).

¹H-NMR (CDCl₃) δ (ppm): 0.94 (3H, t), 1.31-1.40 (2H, m), 1.57-1.63 (2H, m), 2.60 (2H, m), 3.30-3.37 (1H, m), 3.56 (2H, s), 3.60-3.66 (1H, m), 3.80 (3H, s), 3.81 (1H, d), 4.79-4.81 (1H, m), 6.80-6.89 (3H, m), 7.10 (2H, d), 7.16 (2H, d), 7.20-7.25 (1H, m).

### Example B146

### 1-(4-Butylbenzyl)-6-methoxyisoquinoline

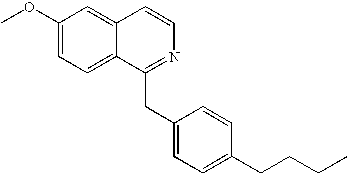

Phosphorus oxychloride (1.6 ml) was added to a solution of the compound of Example B145 (600 mg, 1.7 mmol) in acetonitrile (15 ml), and the mixture was stirred under reflux for 1 hour 30 minutes. The mixture was cooled on ice, made alkaline with a 5% aqueous sodium hydrogencarbonate solution, extracted with ethyl acetate, dried over anhydrous magnesium sulfate, and then concentrated under reduced pressure. The residue was purified by silica gel column chromatography to give the title compound (82 mg).

¹H-NMR (CDCl₃) δ (ppm): 0.89 (3H, t), 1.27-1.36 (2H, m), 1.50-1.58 (2H, m), 2.53 (2H, t), 3.92 (3H, s), 4.57 (2H, s), 7.05-7.07 (3H, m), 7.13-7.18 (3H, m), 7.45 (1H, d), 8.06 (1H, d), 8.41 (1H, d).

### Example 147

### 1-(4-Butylbenzyl)-6-isoquinolinol

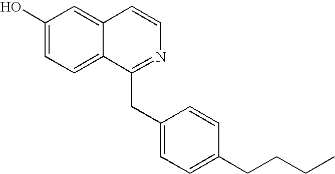

A 47% hydrobromic acid solution was added to the compound of Example B146 (82 mg), and the mixture was stirred under reflux for 19 hours. The mixture was concentrated under reduced pressure, water was added, and the resulting mixture was neutralized with sodium carbonate to precipitate crystals. The obtained crystals were collected by filtration, washed with water, and then dried to give the title compound (74 mg).

¹H-NMR (CD₃OD) δ (ppm): 0.89 (3H, t), 1.25-1.34 (2H, m), 1.49-1.57 (2H, m), 2.52 (2H, t), 4.63 (2H, s), 7.03-7.13 (6H, m), 7.49 (1H, d), 8.10 (1H, d), 8.18 (1H, d).

### Example B148

### 1-(4-Butylbenzyl)-6-propoxyisoquinoline

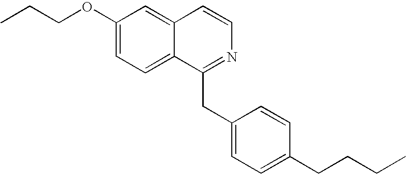

Silver carbonate (40 mg, 0.14 mmol) was added to a solution of the compound of Example B147 (20 mg, 0.069 mmol) and 1-iodopropane (0.4 ml, 4.1 mmol) in toluene (1.0 ml), and the mixture was stirred in the dark at 50° C. for 4 hours. Upon cooling to room temperature, the mixture was filtered through celite and washed with a mixed solution of toluene and methanol (9:1). The obtained filtrate was concentrated under reduced pressure, and the resulting residue was purified by silica gel column chromatography to give the title compound (13 mg).

¹H-NMR (CDCl₃) δ (ppm): 0.90 (3H, t), 1.08 (3H, t), 1.30-1.33 (2H, m), 1.51-1.57 (2H, m), 1.86-1.91 (2H, m), 2.54 (2H, t), 4.05 (2H, t), 4.58 (2H, s), 7.05-7.07 (3H, m), 7.14-7.18 (3H, m), 7.43-7.44 (1H, m), 8.05-8.07 (1H, m), 8.40-8.41 (1H, m).

### Example B149

### 1-(4-Butylbenzyl)-6-(2-piperidinoethoxy)isoquinoline

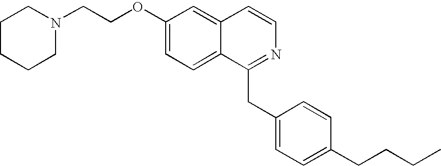

The title compound was obtained in the same manner as in Example 148.

¹H-NMR (CDCl₃) δ (ppm): 0.89 (3H, t), 1.26-1.36 (2H, m), 1.46-1.57 (8H, m), 2.50-2.54 (6H, m), 2.83-2.86 (2H, m), 4.23 (2H, t), 4.56 (2H, s), 7.04-7.06 (3H, m), 7.13-7.17 (3H, m), 7.43 (1H, d), 8.04 (1H, d), 8.40 (1H, d).

### Example B150

### N-({[1-(4-Butylbenzyl)-6-isoquinolyl]oxy}ethyl)-N,N-dimethylamine

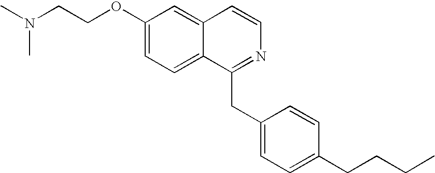

The title compound was obtained in the same manner as in Example 148.

¹H-NMR (CDCl₃) δ (ppm): 0.89 (3H, t), 1.26-1.36(2H, m), 1.49-1.57 (2H, m), 2.37 (6H, s), 2.52 (2H, t), 2.80 (2H, t), 4.19 (2H, t), 4.57 (2H, s), 7.04-7.06 (3H, m), 7.15-7.19 (3H, m), 7.43 (1H, d), 8.05 (1H, d), 8.40 (1H, d).

### Example B151

### 2-Benzoyl-7-methoxy-1,2-dihydro-1-isoquinolinecarbonitrile

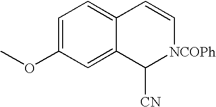

The title compound was obtained by treating 7-methoxyisoquinoline, which was synthesized according to Tetrahedron, 27, 1253 (1971), in the same manner as in Example B140.

¹H-NMR (CDCl₃) δ (ppm): 3.87 (3H, s), 6.03 (1H, brd), 6.56-6.54 (2H, m), 6.90 (1H, s), 6.95 (1H, dd), 7.17 (1H, d), 7.46-7.50 (2H, m), 7.54-7.62 (3H, m).

### Example B152

### 1-(4-Butylbenzyl)-7-methoxyisoquinoline

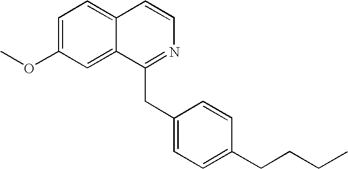

The title compound was obtained by treating the compound of Example B1 and the compound of Example B151 in the same manner as in Example B2.

¹H-NMR (CDCl₃) δ (ppm): 0.89 (3H, t), 1.27-1.36 (2H, m), 1.56-1.58 (2H, m), 2.55 (2H, t), 3.82 (3H, s), 4.59 (2H, s), 7.07 (2H, d), 7.20 (2H, d), 7.26-7.29 (1H, m), 7.35 (1H, d), 7.49 (1H, d), 7.70 (1H, d), 8.38-8.40 (1H, m).

### Example B153

### 1-(4-Bromobenzyl)-7-methoxyisoquinoline

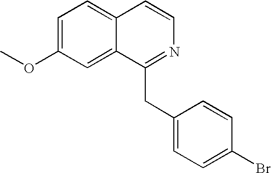

The title compound was obtained by treating the compound of Example B31 and the compound of Example B151 in the same manner as in Example B2.

¹H-NMR (CDCl₃) δ (ppm): 3.84 (3H, s), 4.57 (2H, s), 7.14-7.16 (2H, m), 7.26 (1H, s), 7.29-7.32 (1H, m), 7.37-7.39 (2H, m), 7.51 (1H, d), 7.73 (1H, d), 8.39 (1H, d).

### Example B154

### 1-(4-Butylbenzyl)-7-isoquinolinol

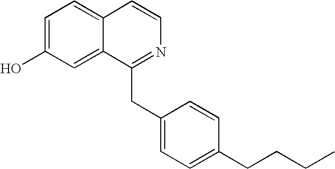

The title compound was obtained by treating the compound of Example B152 in the same manner as in Example B147.

¹H-NMR (DMSO-d₆) δ (ppm): 0.83 (3H, t), 1.21-1.26 (2H, m), 1.44-1.48 (2H, m), 4.68 (2H, s), 7.11 (2H, d), 7.18 (2H, d), 7.59-7.62 (2H, m), 8.10-8.17 (2H, m), 8.38 (1H, d), 10.9 (1H, brs); (The two methylene protons of the butyl group overlapped with the DMSO signal and could not be observed.)

### Example B155

### 1-(4-Butylbenzyl)-7-isoquinolyl Trifluoromethanesulfonate

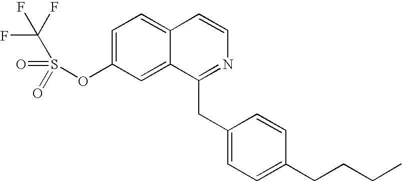

4-Nitrophenol triflate (0.72 g, 2.7 mmol), which was synthesized according to J. Org. Chem., 64,7638 (1999), and potassium carbonate (1.1 g, 8.1 mmol) were added to a solution of the compound of Example B154 (1.0 g, 2.7 mmol) in dimethylformamide (30 ml), and the mixture was stirred at room temperature for 2 hours. After water was added, the resulting mixture was extracted with ethyl acetate. The ethyl acetate layer was washed with 1 N sodium hydroxide and saturated brine, dried over magnesium sulfate, and then concentrated under reduced pressure. The residue was purified by silica gel column chromatography to give the title compound (1.0 g).

¹H-NMR (CDCl₃) δ (ppm): 0.90 (3H, t), 1.27-1.37 (2H, m), 1.51-1.59 (2H, m), 2.54 (2H, t), 5.10 (2H, s), 6.38 (1H, s), 6.95 (2H, d), 7.04 (2H, d), 7.44 (1H, d), 7.55 (1H, d), 7.75 (1H, d), 8.45 (1H, d).

### Example B156

### 1-(4-Butylbenzyl)-7-isoquinolinecarbonitrile

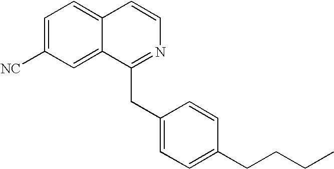

Zinc cyanide (215 mg, 1.8 mmol), tetrakis(triphenylphosphine)palladium (41 mg, 0.035 mmol), and lithium chloride (120 mg, 2.8 mmol) were added to a solution of the compound of Example B155 (400 mg, 0.95 mmol) in dimethylformamide (2 ml) under nitrogen atmosphere, and the mixture was stirred at 120° C. for 2 hours. After cooling to room temperature, saturated sodium hydrogencarbonate was added, and the resulting mixture was extracted with ethyl acetate. The ethyl acetate layer was washed with saturated brine, dried over anhydrous magnesium sulfate, and then concentrated under reduced pressure. The residue was purified by silica gel column chromatography to give the title compound (71 mg).

¹H-NMR (CDCl₃) δ (ppm): 0.89 (3H, t), 1.26-1.35 (2H, m), 1.47-1.55 (2H, m), 2.50 (2H, t), 4.91 (2H, s), 6.97 (2H, d), 7.07 (2H, d), 7.28-7.31 (1H, m), 7.42 (1H, d), 7.51 (1H, d), 7.74 (1H, d), 8.34 (1H, d).

### Example B157

### 1-(4-Butylbenzyl)-7-[2-(1,1,1-trimethylsilyl)-1-ethynyl]isoquinoline

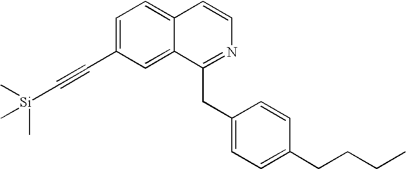

Palladium acetate (11 mg, 0.047 mmol), 1,1′-bis(diphenylphosphino)ferrocene (72 mg, 0.13 mmol), and lithium chloride (25 mg, 0.59 mmol) were added to a solution of the compound of Example B155 (100 mg, 0.24 mmol) and trimethylsilylacetylene (65 μl, 0.47 mmol) in dimethylformamide (3.0 ml), and the reaction system was purged with nitrogen. Triethylamine (59 μl, 0.43 mmol) and copper iodide (2 mg, 0.018 mmol) were added, and the resulting mixture was stirred at 80° C. for 21 hours, then cooled to room temperature. After water and ethyl acetate were added for partition, the ethyl acetate layer was washed with water, dried over anhydrous magnesium sulfate, and then concentrated under reduced pressure. The residue was purified by silica gel column chromatography to give the title compound (7.0 mg).

¹H-NMR (CDCl₃) δ (ppm): 0.28-0.32 (9H, m), 0.92 (3H, t), 1.32-1.38 (2H, m), 1.54-1.57 (2H, m), 2.57 (2H, t), 4.63 (2H, s), 7.10 (2H, d), 7.20 (2H, d), 7.52 (1H, d), 7.67-7.69 (1H, m), 7.75 (1H, d), 8.34 (1H, d), 8.51 (1H, d).

### Example B158

### 1-(4-Butylbenzyl)-7-(1-ethynyl)isoquinoline

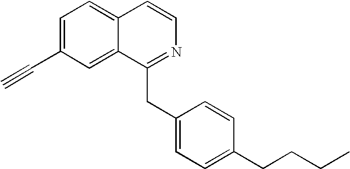

Potassium carbonate (13 mg, 0.094 mmol) was added to a solution of the compound of Example B157 (6 mg, 0.016 mmol) in methanol (1.0 ml), and the mixture was stirred at room temperature for 1 hour. Upon concentration under reduced pressure, the obtained residue was purified by silica gel column chromatography to give the title compound (3.0 mg).

¹H-NMR (CDCl₃) δ (ppm): 0.91 (3H, t), 1.29-1.38 (2H, m), 1.52-1.57 (2H, m), 2.55 (2H, t), 3.19 (1H, s), 4.62 (2H, s), 7.09 (2H, d), 7.20 (2H, d), 7.53 (1H, d), 7.67-7.69 (1H, m), 7.77 (1H, d), 8.36 (1H, s), 8.52 (1H, d).

### Example B159

### 1-(4-Butylbenzyl)-7-ethylisoquinoline

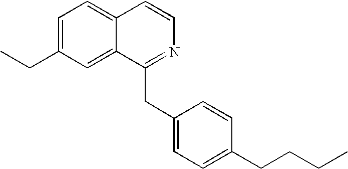

Palladium-carbon (10%, 5.0 mg) was added to a solution of the compound of Example B158 (2.0 mg) in tetrahydrofuran (2.0 ml), and the mixture was stirred at room temperature under nitrogen atmosphere (1 atm) for 1 hour. The catalyst was removed by filtration, and the filtrate was concentrated. The residue was purified by silica gel column chromatography to give the title compound (0.21 mg).

¹H-NMR (CDCl₃) δ (ppm): 0.89 (6H, t), 1.25-1.32 (2H, m), 1.48-1.57 (2H, m), 2.53 (2H, t), 2.80 (2H, q), 4.62 (2H, s), 7.06 (2H, d), 7.20 (2H, d), 7.49-7.52 (2H, m), 7.73 (1H, d), 7.95 (1H, s), 8.43 (1H, d).

### Example B160

### 1-(4-Butylbenzyl)-7-[4-(tetrahydro-2H-2-pyranyloxy)-1-butynyl]-isoquinoline

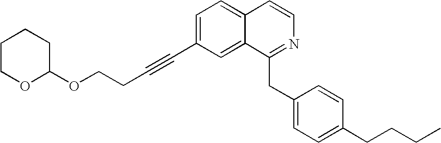

Palladium acetate (11 mg, 0.047 mmol), 1,1′-bis (diphenylphosphino) ferrocene (72 mg, 0.13 mmol), and lithium chloride (25 mg, 0.59 mmol) were added to a solution of the compound of Example B155 (100 mg, 0.24 mmol) and 2-(3-butynyloxy)tetrahydro-2H-pyran (73 mg, 0.47 mmol) in dimethylformamide (3.0 ml), and the system was purged with nitrogen. Furthermore, triethylamine (59 μl, 0.43 mmol) and copper iodide (2 mg, 0.018 mmol) were added, and the resulting mixture was stirred at 80° C. for 24 hours. The mixture was cooled to room temperature, water was added, and the resulting mixture was extracted with ethyl acetate. The ethyl acetate layer was washed with water, dried over anhydrous magnesium sulfate, and then concentrated under reduced pressure. The residue was purified by silica gel column chromatography to give the title compound (25 mg).

¹H-NMR (CDCl₃) δ (ppm): 0.90 (3H, t), 1.28-1.38 (2H, m), 1.52-1.67 (6H, m), 1.72-1.79 (1H, m), 1.79-1.88 (1H, m), 2.54 (2H, t), 2.78 (2H, t), 3.53-3.56 (1H, m), 3.66-3.72 (1H, m), 3.91-3.99 (2H, m), 4.60 (2H, s), 4.71-4.73 (1H, m) 7.08 (2H, d), 7.19 (2H, d), 7.50 (1H, d), 7.59-7.62 (1H, m), 7.72 (1H, d), 8.24 (1H, s), 8.48 (1H, d).

### Example B161

### 4-[1-(4-Butylbenzyl)-7-isoquinolyl]-3-butyn-1-ol

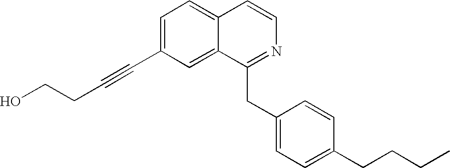

The title compound was obtained by treating the compound of Example B160 in the same manner as in Example B29.

¹H-NMR (CDCl₃) δ (ppm): 0.89 (3H, t), 1.27-1.39 (2H, m), 1.51-1.57 (2H, m), 1.83 (1H, brs), 2.55 (2H, t), 2.75 (2H, t), 3.84-3.89 (2H, m), 4.60 (2H, s), 7.08 (2H, d), 7.18 (2H, d), 7.50 (1H, d), 7.60-7.62 (1H, m), 7.73 (1H, d), 8.25 (1H, s), 8.48 (1H, d).

### Example B162

### 4-[1-(4-Butylbenzyl)-7-isoquinolyl]-1-butanol

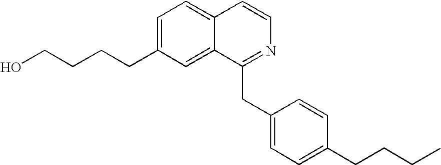

The title compound was obtained by treating the compound of Example B161 in the same manner as in Example B30.

¹H-NMR (CDCl₃) δ (ppm): 0.89 (3H, t), 1.28-1.36 (2H, m), 1.50-1.59 (4H, m), 1.67-1.77 (3H, m), 2.53 (2H, t), 2.79 (2H, t), 3.63 (2H, t), 4.62 (2H, s), 7.06 (2H, d), 7.18 (2H, d), 7.47-7.52 (2H, m), 7.73 (1H, d), 7.92 (1H, s), 8.43 (1H, d).

### Example B163

### 1-(4-Butylbenzyl)-7-propoxyisoquinoline

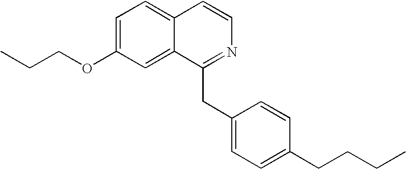

The title compound was obtained by treating the compound of Example B154 in the same manner as in Example B148.

¹H-NMR (CDCl₃) δ (ppm): 0.90 (3H, t), 1.05 (3H, t), 1.27-1.36 (2H, m), 1.50-1.56 (2H, m), 1.76-1.84 (2H, m), 2.53 (2H, t), 3.92 (2H, t), 4.58 (2H, s), 7.06 (2H, d), 7.19 (2H, d), 7.26-7.29 (1H, m), 7.34 (1H, d), 7.48 (1H, d), 7.70 (1H, d), 8.38 (1H, d).

### Example B164

### 1-(4-Butylbenzyl)-7-(2-piperidinoethoxy)isoquinoline

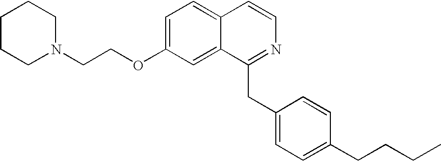

The title compound was obtained in the same manner as in Example B148.

¹H-NMR (CDCl₃) δ (ppm): 0.89 (3H, t), 1.27-1.36 (2H, m), 1.43-1.58 (4H, m), 1.61-1.69 (4H, m), 2.51-2.55 (6H, m), 2.79 (2H, t), 4.11 (2H, t), 4.57 (2H, s), 7.06 (2H, d), 7.18 (2H, d), 7.28-7.30 (1H, m), 7.36 (1H, d), 7.48 (1H, d), 7.70 (1H, d), 8.38 (1H, d).

### Example B165

### N-(2-{[1-(4-Butylbenzyl)-7-isoquinolyl]oxy}ethyl)-N,N-dimethylamine

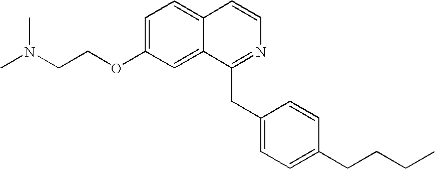

The title compound was obtained in the same manner as in Example B148.

¹H-NMR (CDCl₃) δ (ppm): 0.89 (3H, t), 1.27-1.36 (2H, m), 1.50-1.57 (2H, m), 2.35 (6H, s), 2.53 (2H, t), 2.75 (2H, t), 4.06 (2H, t), 4.58 (2H, s), 7.06 (2H, d), 7.18 (2H, d), 7.30-7.33 (1H, m), 7.36 (1H, d), 7.48 (1H, d), 7.70 (1H, d), 8.39 (1H, d).

### Example B166

### 1-(4-Butylbenzyl)-7-isoquinolyl-(2-morpholinoethyl)ether

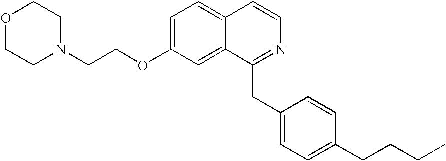

The title compound was obtained in the same manner as in Example B148.

¹H-NMR (CDCl₃) δ (ppm): 0.89 (3H, t), 1.27-1.36 (2H, m), 1.50-1.58 (2H, m), 2.51-2.58 (6H, m), 2.81 (2H, t), 3.75 (4H, t), 4.11 (2H, t), 4.58 (2H, s), 7.06 (2H, d), 7.17 (2H, d), 7.28-7.31 (1H, m), 7.35 (1H, d), 7.49 (1H, d), 7.71 (1H, d), 8.39 (1H, d).

### Example B167

### 7-(Benzyloxy)-1-(4-butylbenzyl)isoquinoline

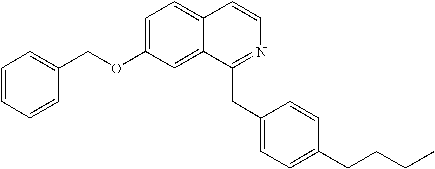

The title compound was obtained in the same manner as in Example B148.

¹H-NMR (CDCl₃) δ (ppm): 0.89 (3H, t), 1.27-1.36 (2H, m), 1.50-1.54 (2H, m), 2.54 (2H, t), 4.54 (2H, s), 5.06 (2H, s), 7.05 (2H, d), 7.14 (2H, d), 7.34-7.43 (7H, m), 7.49 (1H, d), 7.72 (1H, d), 8.39 (1H, d).

### Example B168

### 1-(4-Butylbenzyl)-7-(2-pyridylmethoxy)isoquinoline

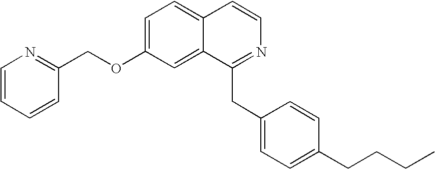

The title compound was obtained in the same manner as in Example B148.

¹H-NMR (CDCl₃) δ (ppm): 0.89 (3H, t), 1.27-1.36 (2H, m), 1.49-1.57 (2H, m), 2.52 (2H, t), 4.51 (2H, s), 5.25 (2H, s), 7.02 (2H, d), 7.14 (2H, d), 7.24-7.27 (1H, m), 7.40 (1H, dd), 7.47-7.50 (3H, m), 7.68-7.72 (1H, d), 7.74 (1H, d), 8.39 (1H, d), 8.64-8.66 (1H, m).

### Example B169

### 1-(4-Butylbenzyl)-7-(3-pyridylmethoxy)isoquinoline

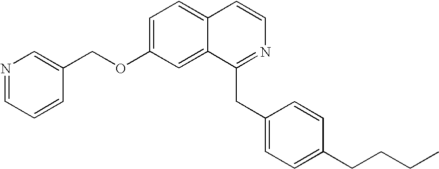

The title compound was obtained in the same manner as in Example B148.

¹H-NMR (CDCl₃) δ (ppm): 0.89 (3H, t), 1.27-1.36 (2H, m), 1.50-1.58 (2H, m), 2.54 (2H, t), 4.57 (2H, s), 5.06 (2H, s), 7.07 (2H, d), 7.15 (2H, d), 7.31-7.36 (2H, m), 7.42 (1H, d), 7.51 (1H, d), 7.74-7.76 (2H, m), 8.42 (1H, d), 8.61-8.62 (1H, m), 8.69-8.70 (1H, m).

### Example B170

### 1-(4-Butylbenzyl)-7-(4-pyridylmethoxy)isoquinoline

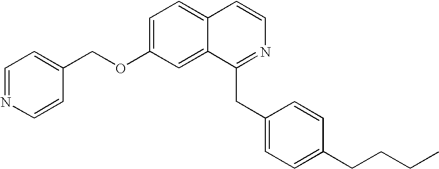

The title compound was obtained in the same manner as in Example B148.

¹H-NMR (CDCl₃) δ (ppm): 0.89 (3H, t), 1.27-1.36 (2H, m), 1.50-1.56 (2H, m), 2.54 (2H, t), 4.53 (2H, s), 5.09 (2H, s), 7.04 (2H, d), 7.09 (2H, d), 7.33-7.39 (4H, m), 7.51 (1H, d), 7.76 (1H, d), 8.41 (1H, d), 8.63-8.64 (2H, m).

### Example B171

### 1-(4-Butylbenzyl)-7-[(2-methoxybenzyl)oxy]isoquinoline

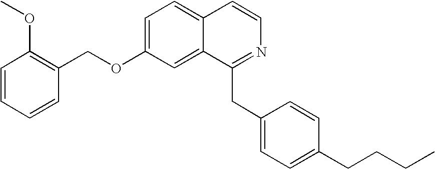

The title compound was obtained in the same manner as in Example B148.

¹H-NMR (CDCl₃) δ (ppm): 0.89 (3H, t), 1.27-1.36 (2H, m), 1.50-1.57 (2H, m), 2.53 (2H, t), 3.82 (3H, s), 4.52 (2H, s), 5.04 (2H, s), 6.88-6.91 (1H, m), 6.99-7.02 (2H, m), 7.05 (2H, d), 7.14 (2H, d), 7.32 (1H, t), 7.36 (1H, dd), 7.43 (1H, d), 7.48 (1H, d), 7.72 (1H, d), 8.39 (1H, d).

### Example B172

### 1-(4-Butylbenzyl)-7-[(3-methoxybenzyl)oxy]isoquinoline

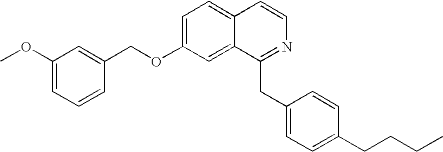

The title compound was obtained in the same manner as in Example B148.

¹H-NMR (CDCl₃) δ (ppm): 0.89 (3H, t), 1.27-1.36 (2H, m), 1.50-1.56 (2H, m), 2.53 (2H, t), 3.90 (3H, s), 4.53 (2H, s), 5.16 (2H, s), 6.93-6.98 (2H, m), 7.03 (2H, d), 7.15 (2H, d), 7.30-7.35 (1H, m), 7.37 (1H, dd), 7.41-7.43 (1H, m), 7.47 (1H, d), 7.51 (1H, d), 7.71 (1H, d), 8.37 (1H, d).

### Example B173

### 1-(4-Butylbenzyl)-7-[(4-methoxybenzyl)oxy]isoquinoline

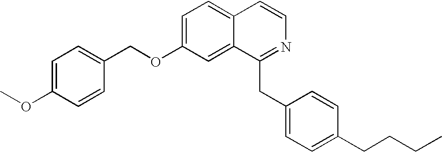

The title compound was obtained in the same manner as in Example B148.

¹H-NMR (CDCl₃) δ (ppm): 0.89 (3H, t), 1.27-1.37 (2H, m), 1.51-1.57 (2H, m), 2.54 (2H, t), 3.83 (3H, s), 4.55 (2H, s), 4.99 (2H, s), 6.93 (2H, d), 7.06 (2H, d), 7.15 (2H, d), 7.32-7.36 (3H, m), 7.44 (1H, d), 7.48 (1H, d), 7.71 (1H, d), 8.38 (1H, d).

### Example B174

### 7-(1,3-Benzodioxol-5-ylmethoxy)-1-(4-butylbenzyl)isoquinoline

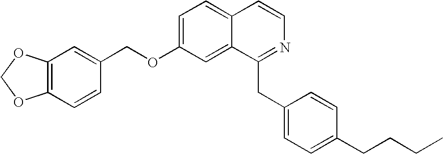

The title compound was obtained in the same manner as in Example B148.

¹H-NMR (CDCl₃) δ (ppm): 0.89 (3H, t), 1.27-1.37 (2H, m), 1.51-1.57 (2H, m), 2.54 (2H, t), 4.55 (2H, s), 4.95 (2H, s), 5.98 (2H, s), 6.82 (1H, d), 6.88 (1H, dd), 6.92 (1H, d), 7.06 (2H, d), 7.15 (2H, d), 7.33 (1H, dd), 7.42 (1H, d), 7.48 (1H, d), 7.72 (1H, d), 8.39 (1H, d).

### Example B175

### 1-(4-Butylbenzyl)-7-[(2-nitrobenzyl)oxy]isoquinoline

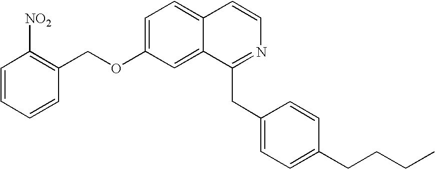

The title compound was obtained in the same manner as in Example B148.

¹H-NMR (CDCl₃) δ (ppm): 0.87 (3H, t), 1.26-1.34 (2H, m), 1.48-1.56 (2H, m), 2.51 (2H, t), 4.53 (2H, s), 5.49 (2H, s), 7.03 (2H, d), 7.14 (2H, d), 7.40 (1H, dd), 7.430-7.434 (1H, m), 7.45-7.49 (1H, m), 7.51 (1H, d), 7.64-7.68 (1H, m), 7.76 (1H, d), 7.85-7.87 (1H, m), 8.22-8.24 (1H, d), 8.41 (1H, d).

### Example B176

### 1-(4-Butylbenzyl)-7-[(3-nitrobenzyl)oxy]isoquinoline

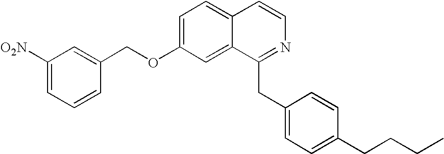

The title compound was obtained in the same manner as in Example B148.

¹H-NMR (CDCl₃) δ (ppm): 0.89 (3H, t), 1.27-1.36 (2H, m), 1.50-1.56 (2H, m), 2.54 (2H, t), 4.55 (2H, s), 5.14 (2H, s), 7.05 (2H, d), 7.11 (2H, d), 7.37-7.40 (2H, m), 7.51 (1H, d), 7.55-7.59 (1H, m), 7.73-7.78 (2H, m), 8.19-8.22 (1H, m), 8.32-8.33 (1H, m), 8.42 (1H, d).

### Example B177

### 1-(4-Butylbenzyl)-7-(phenethyloxy)isoquinoline

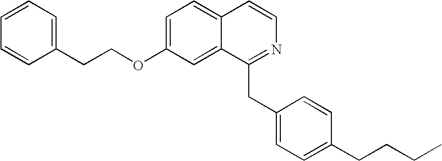

The title compound was obtained in the same manner as in Example B148.

¹H-NMR (CDCl₃) δ (ppm): 0.89 (3H, t), 1.26-1.36 (2H, m), 1.49-1.57 (2H, m), 2.52 (2H, t), 3.10 (2H, t), 4.18 (2H, t), 4.56 (2H, s), 7.04 (2H, d), 7.16 (2H, d), 7.26-7.28 (4H, m), 7.33-7.35 (3H, m), 7.48 (1H, d), 7.70 (1H, d), 8.38-8.39 (1H, m).

### Example B178

### 1-(4-Butylbenzyl)-7-(3-phenylpropoxy)isoquinoline

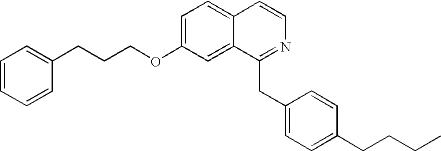

The title compound was obtained in the same manner as in Example B148.

¹H-NMR (CDCl₃) δ (ppm): 0.89 (3H, t), 1.27-1.36 (2H, m), 1.49-1.57 (2H, m), 2.09-2.15 (2H, m), 2.52 (2H, t), 2.82 (2H, t), 3.97 (2H, t), 4.55 (2H, s), 7.04 (2H, d), 7.16 (2H, d), 7.20-7.23 (3H, m), 7.27-7.33 (4H, m), 7.48 (1H, d), 7.70 (1H, d), 8.38 (1H, d).

### Example B179

### 1-(4-Butylbenzyl)-7-(2-cyclohexylethoxy)isoquinoline

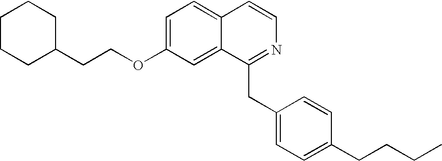

The title compound was obtained in the same manner as in Example B148.

¹H-NMR (CDCl₃) δ (ppm): 0.89 (3H, t), 0.94-1.02 (2H, m), 1.17-1.36 (4H, m), 1.36-1.57 (4H, m), 1.65-1.76 (7H, m), 2.53 (2H, t), 3.98 (2H, t), 4.58 (2H, s), 7.06 (2H, d), 7.19 (2H, d), 7.25-7.28 (1H, m), 7.33 (1H, d), 7.47 (1H, d), 7.69 (1H, d), 8.37 (1H, d).

### Example B180

### 6-Benzoyl-5,6-dihydro[1,3]dioxolo[4,5-g]isoquinoline-5-carbonitrile

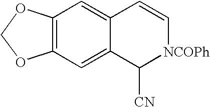

The title compound was obtained by treating [1,3]dioxolo[4,5-g]isoquinoline in the same manner as in Example B140.

¹H-NMR (CDCl₃) δ (ppm): 5.94-5.96 (1H, m), 6.03 (1H, d), 6.04 (1H, d), 6.47-6.54 (2H, m), 6.70 (1H, s), 6.83 (1H, s), 7.45-7.49 (2H, m), 7.54-7.62 (3H, m).

### Example B181

### 5-(4-Butylbenzyl)[1,3]dioxolo[4,5-g]isoquinoline

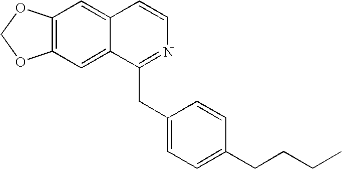

The title compound was obtained by treating the compound of Example B180 and the compound of Example B1 in the same manner as in Example B2.

¹H-NMR (CDCl₃) δ (ppm): 0.90 (3H, t), 1.28-1.37 (2H, m), 1.51-1.57 (2H, m), 2.54 (2H, t), 4.50 (2H, s), 6.05 (2H, s), 7.05-7.07 (3H, m), 7.16 (2H, d), 7.38 (7.40 (2H, m), 8.35 (1H, d).

### Example B182

### 2-Benzoyl-6-bromo-1,2-dihydro-1-isoquinolinecarbonitrile

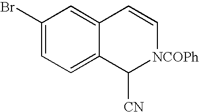

The title compound was obtained by treating 6-bromoisoquinoline, which was synthesized according to J. Am. Chem. Soc., 183 (1942), in the same manner as in Example B140.

¹H-NMR (CDCl₃) δ (ppm): 6.01 (1H, d), 6.53 (1H, brs), 6.70 (1H, brd), 7.24 (1H, d), 7.33 (1H, d), 7.47-7.51 (3H, m), 7.56 (3H, m).

### Example B183

### 6-Bromo-1-(4-butylbenzyl)isoquinoline

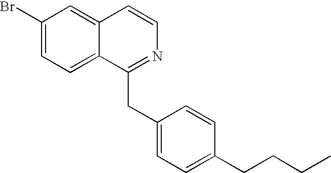

The title compound was obtained by treating the compound of Example B182 and the compound of Example B1 in the same manner as in Example B2.

¹H-NMR (CDCl₃) δ (ppm): 0.89 (3H, t), 1.27-1.36 (2H, m), 1.50-1.58 (2H, m), 2.53 (2H, t), 4.60 (2H, s), 7.06 (2H, d), 7.15 (2H, d), 7.46 (1H, d), 7.59 (1H, q), 7.98 (1H, d), 8.02 (1H, d), 8.51 (1H, d).

### Example B184

### A Mixture of 2-Benzoyl-5-bromo-1,2-dihydro-1-isoquinolinecarbonitrile and 2-Benzoyl-7-bromo-1,2-dihydro-1-isoquinolinecarbonitrile

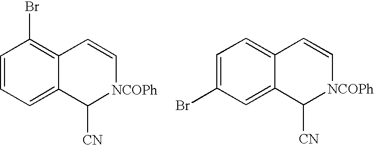

The title compounds were obtained by treating 5- or 7-bromoisoquinoline, which was synthesized according to J. Am. Chem. Soc., 61, 183 (1939), in the same manner as in Example B140. The obtained compounds were used in the following reaction without separation and purification.

### Example B185

### 7-Bromo-1-(4-butylbenzyl)isoquinoline

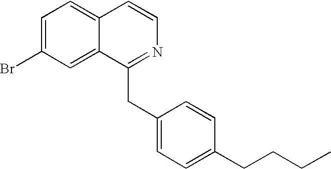

The title compound was obtained by treating the compound of Example B184 and the compound of Example B1 in the same manner as in Example B2.

¹H-NMR (CDCl₃) δ (ppm): 0.90 (3H, t), 1.28-1.37 (2H, m), 1.51-1.58 (2H, m), 2.55 (2H, t), 4.58 (2H, s), 7.09 (2H, d), 7.18 (2H, d), 7.51-7.53 (1H, m), 7.69-7.70 (2H, m), 8.33-8.34 (1H, m), 8.52 (1H, d).

### Example B186

### 5-Benzoyl-4,5-dihydrothieno[3,2-c]pyridine-4-carbonitrile

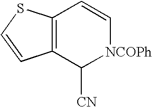

The title compound was obtained by treating thieno[3,2-c]pyridine, synthesized according to J. Heterocycl. Chem., 30, 183 (1993), in the same manner as in Example B140.

¹H-NMR (CDCl₃) δ (ppm): 6.05 (1H, d), 6.57 (1H, brd), 6.66 (1H, s), 7.07 (1H, d), 7.32 (1H, d), 7.46-7.50 (2H, m), 7.54-7.62 (3H, m).

### Example B187

### 4-(4-Butylbenzyl)thieno[3,2-c]pyridine

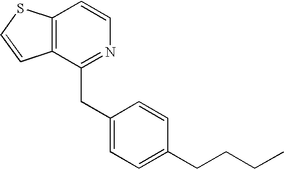

The title compound was obtained by treating the compound of Example B186 and the compound of Example B1 in the same manner as in Example B2.

¹H-NMR (CDCl₃) δ (ppm): 0.90 (3H, t), 1.27-1.37 (2H, m), 1.51-1.59 (2H, 1.5 m), 2.54 (2H, t), 4.47 (2H, s), 7.07 (2H, d), 7.19 (2H, d), 7.42 (1H, d), 7.47 (1H, dd), 7.68 (1H, d), 8.41 (1H, d).

### Example B188

### 4-(4-Methoxybenzyl)thieno[3,2-c]pyridine

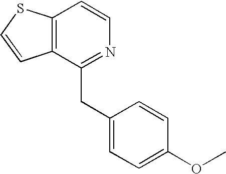

The title compound was obtained by treating the compound of Example B186 and 4-methoxybenzyl chloride in the same manner as in Example B2.

¹H-NMR (CDCl₃) δ (ppm): 3.75 (3H, s), 4.44 (2H, s), 6.79-6.82 (2H, m), 25 7.19-7.22 (2H, m), 7.43 (1H, d), 7.46 (1H, dd), 7.68 (1H, d), 8.41 (1H, d).

### Example B189

### 4-(Thieno[3,2-c]pyridin-4-ylmethyl)phenyl Trifluoromethanesulfonate

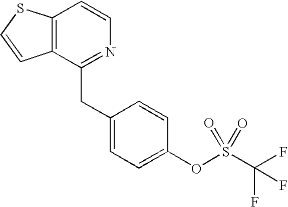

A solution of boron tribromide in methylene chloride (1.0 M, 10 ml, 10 mmol) was added dropwise to a solution of the compound of Example B188 (510 mg, 2.0 mmol) in methylene chloride (10 ml)cooled to 0° C. , and this reaction mixture was stirred at that temperature for 1.5 hours. The reaction mixture was made weakly alkaline by addition of a saturated aqueous sodium hydrogencarbonate solution, extracted with ethyl acetate, dried over anhydrous magnesium sulfate, and then concentrated under reduced pressure. The obtained residue was dissolved in pyridine, and the resulting solution was cooled to 0° C. After trifluoromethanesulfonic anhydride (0.34 ml, 2.1 mmol) was added dropwise thereto, the mixture was stirred at that temperature for 2 hours, poured on ice, extracted with ethyl acetate, dried over anhydrous magnesium sulfate, and then concentrated under reduced pressure. The residue was purified by silica gel chromatography to give the title compound (312 mg).

¹H-NMR (CDCl₃) δ (ppm): 4.52 (2H, s), 7.16-7.18 (2H, m), 7.36 (2H, m), 7.43-7.44 (1H, m), 7.49 (1H, d), 7.73 (1H, d), 8.42 (1H, d).

### Example B190

### 4-(4-Bromobenzyl)thieno[3,2-c]pyridine

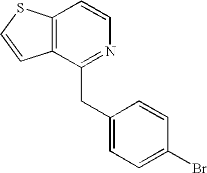

The title compound was obtained by treating the compound of Example B186 and the compound of Example B31 in the same manner as in Example B2.

¹H-NMR (CDCl₃) δ (ppm): 4.45 (2H, s), 7.14-7.16 (2H, m), 7.37-7.39 (2H, m), 7.41-7.43 (1H, m), 7.45 (1H, d), 7.71 (1H, d), 8.41 (1H, d).

### Example B191

### 4-(4-Bromo-2-fluorobenzyl)thieno[3,2-c]pyridine

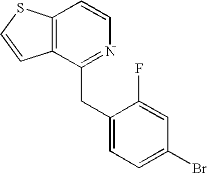

The title compound was obtained by treating the compound of Example B186 and 4-bromo-2-fluorobenzyl bromide in the same manner as in Example B2.

¹H-NMR (CDCl₃) δ (ppm): 4.46 (2H, s), 7.11 (1H, t), 7.15-7.18 (1H, m), 7.22-7.25 (1H, m), 7.47 (1H, d), 7.49 (1H, d), 7.71 (1H, d), 8.41 (1H, d).

### Example B192

### 4-{4-[4-(Tetrahydro-2H-2-pyranyloxy)-1-butynyl]benzyl}thieno[3,2-c]pyridine

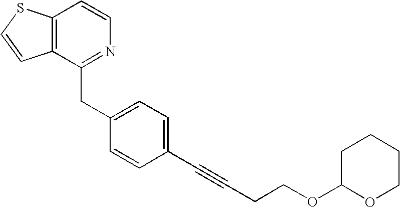

The title compound was obtained by treating the compound of Example B189 and 2-(3-butynyloxy)tetrahydro-2H-pyran in the same manner as in Example B42.

¹H-NMR (CDCl₃) δ (ppm): 1.40-1.90 (6H, m), 2.69 (2H, t), 3.45-3.65 (2H, m), 3.78-3.95 (2H, m), 4.48 (2H, s), 4.66-4.69 (1H, m), 7.18 (2H, d), 7.27 (2H, d), 7.41 (1H, d), 7.44 (1H, d), 7.70 (1H, d), 8.41 (1H, d).

### Example B193

### 4-[4-(Thieno[3,2-c]pyridin-4-ylmethyl)phenyl]-3-butyn-1-ol

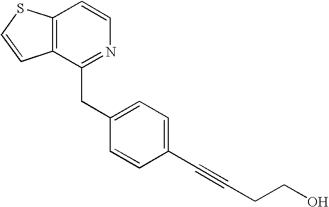

The title compound was obtained by treating the compound of Example B192 in the same manner as in Example B47.

¹H-NMR (CDCl₃) δ (ppm): 2.67 (2H, t), 3.79 (2H, t), 4.50 (2H, s), 7.20 (2H, d), 7.32 (2H, d), 7.41 (1H, d), 7.44 (1H, d), 7.71 (1H, d), 8.42 (1H, d).

The proton of the hydroxyl group was not observed in the NMR spectrum.

### Example B194

### 6-Benzoyl-6,7-dihydrothieno[2,3-c]pyridine-7-carbonitrile

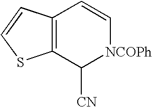

The title compound was obtained by treating thieno[2,3-c]pyridine, which was synthesized according to J. Heterocycl. Chem., 30, 183 (1993), in the same manner as in Example B140.

¹H-NMR (CDCl₃) δ (ppm): 6.07 (1H, d), 6.56 (1H, brd), 6.75 (1H, s), 6.97 (1H, d), 7.37 (1H, d), 7.46-7.51 (2H, m), 7.54-7.64 (3H, m).

### Example B195

### 7-(4-Butylbenzyl)thieno[2,3-c]pyridine

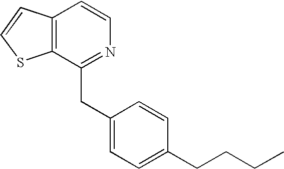

The title compound was obtained by treating the compound of Example B194 and the compound of Example B1 in the same manner as in Example B2.

¹H-NMR (CDCl₃) δ (ppm): 0.90 (3H, t), 1.28-1.37 (2H, m), 1.51-1.59 (2H, m), 2.55 (2H, t), 4.40 (2H, s), 7.09 (2H, d), 7.28 (2H, d), 7.34 (1H, d), 7.57 (1H, d), 7.62 (1H, d), 8.47 (1H, d).

### Example B196

### 7-(4-Methoxybenzyl)thieno[2,3-c]pyridine

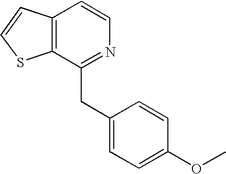

The title compound was obtained by treating the compound of Example B194 and 4-methoxybenzyl chloride in the same manner as in Example B2.

¹H-NMR (CDCl₃) δ (ppm): 3.76 (3H, s), 4.38 (2H, s), 6.81-6.83 (2H, m), 7.28-7.30 (2H, m), 7.35 (1H, d), 7.57 (1H, d), 7.62 (1H, d), 8.47 (1H, d).

### Example B197

### 4-(Thieno[2,3-c]pyridin-7-ylmethyl)phenyl Trifluoromethanesulfonate

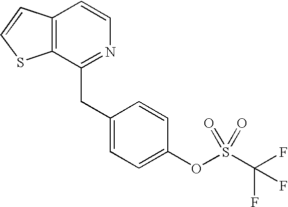

The title compound was obtained by treating the compound of Example B196 in the same manner as in Example B189.

¹H-NMR (CDCl₃) δ (ppm): 4.44 (2H, s), 7.17-7.19 (2H, m), 7.38-7.40 (1H, m), 7.44-7.46 (2H, m), 7.61 (1H, d), 7.65-7.67 (1H, m), 8.47-8.49 (1H, m).

### Example B198

### 7-(4-Bromobenzyl)thieno[2,3-c]pyridine

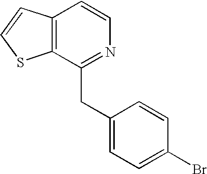

The title compound was obtained by treating the compound of Example B194 and the compound of Example B31 in the same manner as in Example B2.

¹H-NMR (CDCl₃) δ (ppm): 4.37 (2H, s), 7.23-7.25 (2H, m), 7.37 (1H, d), 7.39-7.41 (2H, m), 7.59 (1H, d), 7.63-7.65 (1H, m), 8.47 (1H, d).

### Example B199

### 7-(4-Bromo-2-fluorobenzyl)thieno[2,3-c]pyridine

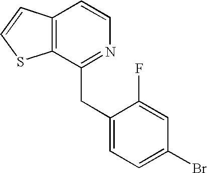

The title compound was obtained by treating the compound of Example B194 and 4-bromo-2-fluorobenzyl bromide in the same manner as in Example B2.

¹H-NMR (CDCl₃) δ (ppm): 4.40-4.41 (2H, m), 7.12-7.20 (2H, m), 7.23-7.26 (1H, m), 7.37-7.39 (1H, m), 7.59-7.62 (1H, m), 7.65-7.67 (1H, m), 8.45-8.47 (1H, m).

### Example B200

### 7-{4-[4-(Tetrahydro-2H-2-pyranyloxy)-1-butynyl]benzyl}thieno[2,3-c]pyridine

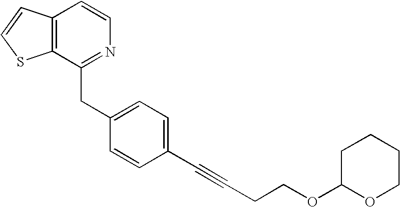

The title compound was obtained by treating the compound of Example B197 and 2-(3-butynyloxy)tetrahydro-2H-pyran in the same manner as in Example B42.

¹H-NMR (CDCl₃) δ (ppm): 1.50-1.90 (6H, m), 2.69 (2H, t), 3.49-3.54 (1H, m), 3.58-3.65 (1H, m), 3.85-3.95 (2H, m), 4.41 (2H, s), 4.68 (1H, t), 7.26-7.31 (4H, m), 7.36 (1H, d), 7.58 (1H, d), 7.63 (1H, d), 8.47 (1H, d).

### Example B201

### 4-[4-(Thieno[2,3-c]pyridin-7-ylmethyl)phenyl]-3-butyn-1-ol

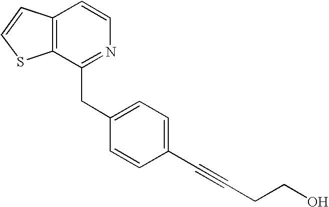

The title compound was obtained by treating the compound of Example B200 in the same manner as in Example B47.

¹H-NMR (CDCl₃) δ (ppm): 1.99 (1H, brs), 2.67 (2H, t), 3.79 (2H, t), 4.42 (2H, s), 7.27-7.34 (4H, m), 7.36 (1H, d), 7.59 (1H, d), 7.64 (1H, d), 8.47 (1H, d).

### Example B202

### 2-Chloro-3-(methoxymethoxy)pyridine

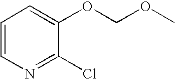

Sodium hydride (66%, 633 mg, 17.4 mmol) was added to an ice-cooled solution of 2-chloro-3-hydroxypyridine (2.05 g, 15.8 mmol) in tetrahydrofuran (30 ml) under nitrogen atmosphere, and this reaction mixture was stirred at that temperature for 15 minutes. Chloromethyl methyl ether (1.32 ml, 17.4 mmol) was added, and the resulting reaction mixture was stirred at that temperature for 30 minutes, then at room temperature for another 2 hours. After water was added, the reaction mixture was extracted with ethyl acetate, washed with saturated brine, and then concentrated under reduced pressure. The residue was purified by silica gel column chromatography to give the title compound (2.44 g).

¹H-NMR (CDCl₃) δ (ppm): 3.53 (3H, s), 5.28 (2H, s), 7.19 (1H, dd), 7.49 (1H, dd), 8.06 (1H, dd).

### Example B203

### 2-Chloro-4-iodo-3-(methoxymethoxy)pyridine

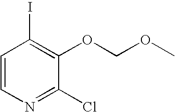

A solution of the compound of Example B202 (1.40 g, 8.06 mmol) in diethyl ether (8 ml) was added dropwise to a solution of 1.51 M t-butyllithium-n-pentane solution (8.01 ml, 12.1 mmol) in diethyl ether (15 ml) cooled to −78° C. under nitrogen atmosphere, and the reaction mixture was stirred at that temperature for 15 minutes. After iodine (3.07 g, 12.1 mmol) was added, the reaction mixture was gradually warmed to room temperature. An aqueous sodium thiosulfate solution was further added, and the diethyl ether layer was separated, washed with saturated brine, and then concentrated under reduced pressure. The residue was purified by silica gel column chromatography to give the title compound (356 mg).

¹H-NMR (CDCl₃) δ (ppm): 3.73 (3H, s), 5.22 (2H, s), 7.69 (1H, d), 7.80 (1H, d).

### Example B204

### 7-Chlorofuro[2,3-c]pyridine

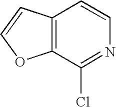

Trimethylsilylacetylene (28.3 μl, 0.201 mmol) and triethylamine (59.8 μl, 0.429 mmol) were added to a solution of the compound of Example B203 (36.6 mg, 0.143 mmol), tetrakis(triphenylphosphine)palladium (16.5 mg, 0.0143 mmol), and copper(I) iodide (2.7 mg, 0.014 mmol) in dimethylformamide (1.5 ml), and this mixture was stirred at 50° C. for 4 hours. After allowing the mixture to cool to room temperature, water was added thereto, and the resulting mixture was extracted with ethyl acetate, washed with saturated brine, and then concentrated under reduced pressure. The residue was dissolved in methanol (5 ml), potassium carbonate (100 mg, 0.724 mmol) was added thereto, and the resulting mixture was stirred at room temperature for 1 hour. After water was added, the mixture was extracted with diethyl ether, washed with saturated brine, and then concentrated under reduced pressure. The residue was purified by silica gel column chromatography to give the title compound (5.5 mg).

¹H-NMR (CDCl₃) δ (ppm): 6.89 (1H, d), 7.51 (1H, d), 7.83 (1H, d), 8.21 (1H, d).

### Example B205

### 4-Butylbenzylmagnesium Chloride

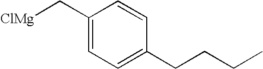

A mixed solution of the compound of Example B1 (1.04 g, 5.69 mmol), magnesium (761 mg, 31.3 mmol), and a catalytic amount of 1,2-dibromoethane in diethyl ether (11 ml) was initiated by heating under reflux. After the heat source was removed, a solution of the compound of Example B1 (4.16 g, 22.8 mmol) in diethyl ether (60 ml) was added dropwise to the reaction mixture at a rate that maintains gentle reflux, and the mixture was heated under reflux for 30 minutes. The mixture was then allowed to cool to room temperature to give the title compound as a 0.4 M solution in diethyl ether. This solution was used in the following reaction as it is.

### Example B206

### 7-(4-Butylbenzyl)furo[2,3-c]pyridine

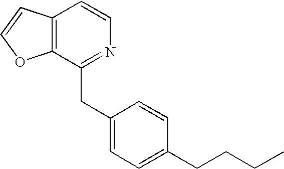

The compound of Example B205 (300 μl, 0.1 mmol) was added to a solution of the compound of Example B204 (5.0 mg, 0.033 mmol) and [1,1′-bis(diphenylphosphino)ferrocene]dichloronickel(II) (4.5 mg, 0.0065 mmol) in tetrahydrofuran (1 ml), and the mixture was stirred at 50° C. for 1 hour. After allowing the mixture to cool to room temperature, ethyl acetate was added thereto. The resulting mixture was washed with a saturated aqueous ammonium chloride solution and saturated brine, then concentrated under reduced pressure. The residue was purified by NH-silica gel column chromatography to give the title compound (2.9 mg).

¹H-NMR (CDCl₃) δ (ppm): 0.89 (3H, t), 1.29-1.35 (2H, m), 1.50-1.58 (2H, m), 2.54 (2H, t), 4.40 (2H, s), 6.78 (1H, d), 7.08 (2H, d), 7.30 (2H, d), 7.40 (1H, d), 7.72 (1H, d), 8.34 (1H, d).

### Example B207

### 7-(4-Butylbenzyl)-1H-pyrrolo[2,3-c]pyridine

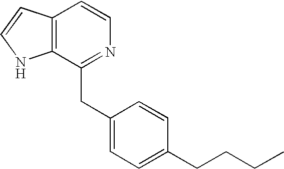

The compound of Example B205 (800 μl, 0.3 mmol) was added to a solution of 1-chloropyrrolopyridine (19.4 mg, 0.127 mmol), which was synthesized from 2-chloro-3-aminopyridine according to the method of H07-165,708A, and dichloro(diphenylphosphinopropane) nickel (6.9 mg, 0.013 mmol) in tetrahydrofuran (1 ml) under ice-cooling, and the mixture was stirred while heating under reflux for 4 hours. After allowing the mixture to cool to room temperature, ethyl acetate was added thereto. The resulting mixture was washed with a saturated aqueous ammonium chloride solution and saturated brine, then concentrated under reduced pressure. The residue was purified by silica gel column chromatography to give the title compound (7.1 mg).

¹H-NMR (CDCl₃) δ (ppm): 0.91 (3H, t), 1.31-1.37 (2H, m), 1.55-1.59 (2H, m), 2.58 (2H, t), 4.44 (2H, s), 6.50 (1H, d), 7.12 (2H, d), 7.18 (1H, d), 7.22 (2H, d), 7.45 (1H, d), 8.21 (1H, d).

The NH proton was not observed in the NMR spectrum.

### Example B208

### 4-(4-Butylbenzyl)-1-imidazo[4,5-c]pyridine

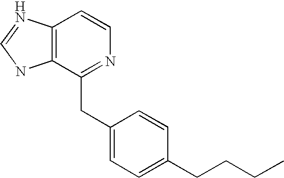

The compound of Example B205 (3.45 ml, 1.38 mmol) was added to a solution of 1-chloroimidazopyridine (88.6 mg, 0.577 mmol), which was synthesized from 4-amino-2-chloropyridine according to the method described in J. Heterocycl. Chem., 2, 196 (1965), and dichloro(diphenylphosphinopropane)nickel (31.3 mg, 0.0577 mmol) in tetrahydrofuran (2 ml), and the mixture was stirred while heating under reflux for 2 hours. After allowing the mixture to cool to room temperature, ethyl acetate was added thereto. The resulting mixture was filtered through silica gel and concentrated under reduced pressure. The residue was purified by silica gel column chromatography to give the title compound (64.2 mg).

¹H-NMR (CDCl₃) δ (ppm): 0.86 (3H, t), 1.23-1.32 (2H, m), 1.44-1.52 (2H, m), 2.47 (2H, t), 4.56 (2H, s), 7.02 (2H, d), 7.19 (2H, d), 7.34 (1H, d), 8.00 (1H, s), 8.25-8.27 (1H, m).

The NH proton was not observed in the NMR spectrum.

### Example B209

### 4-Bromo-1-isoquinolinol

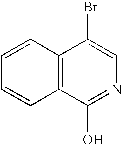

Bromine (1.78 ml, 34.5 mmol) was added to an ice-cooled solution of 1-hydroxyisoquinoline (5.01 g, 34.5 mmol) in acetic acid (50 ml) and this reaction mixture was stirred at room temperature for 2 hours. Water, ethyl acetate, and tetrahydrofuran were added, and the resulting reaction mixture was filtered through filter paper. The organic layer was washed with saturated brine and concentrated under reduced pressure. The residue was recrystallized from ethyl acetate and hexane to give the title compound (6.19 g).

¹H-NMR (DMSO-d6) δ (ppm): 7.56 (1H, s), 7.59-7.63 (1H, m), 7.76-7.78 (1H, m), 7.84-7.89 (1H, m), 8.23-8.26 (1H, m), 11.59 (1H, br s).

### Example B210

### 1,4-Dibromoisoquinoline

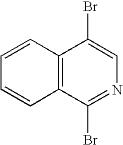

A mixed solution of the compound of Example B209 (1.40 g, 8.06 mmol) and phosphorus tribromide (6 ml) was stirred at 150° C. for 1 hour, and then heated under reflux for another 1 hour. The reaction mixture was allowed to cool to room temperature, poured on ice, then warmed to room temperature. Ethyl acetate was added, and the resulting mixture was washed with saturated brine and concentrated under reduced pressure. The residue was purified by silica gel column chromatography to give the title compound (845 mg).

¹H-NMR (CDCl₃) δ (ppm): 7.76-7.80 (1H, m), 7.86-7.90 (1H, m), 8.19 (1H, d), 8.31-8.34 (1H, m), 8.48 (1H, s).

### Example B211

### 4-Bromo-1-(4-butylbenzyl)isoquinoline

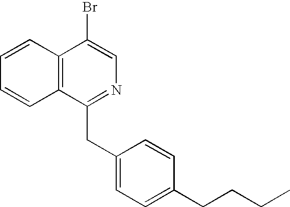

The compound of Example B205 (2.5 ml, 1 mmol) was added to a solution of the compound of Example B210 (200 mg, 0.697 mmol) and [1,1′-bis(diphenylphosphino)ferrocene]dichloronickel(II) (75.6 mg, 0.139 mmol) in tetrahydrofuran (2 ml), and the mixture was stirred at room temperature for 30 minutes. After ethyl acetate was added, the resulting mixture was washed successively with a saturated aqueous ammonium chloride solution, a saturated aqueous sodium hydrogencarbonate solution, and saturated brine, then concentrated under reduced pressure. The residue was purified by silica gel column chromatography to give the title compound (98 mg).

¹H-NMR (CDCl₃) δ (ppm): 0.89 (3H, t), 1.29-1.34 (2H, m), 1.51-1.60 (2H, m), 2.53 (2H, t), 4.59 (2H, s), 7.06 (2H, d), 7.16 (2H, d), 7.57-7.61 (1H, m), 7.73-7.77 (1H, m), 8.15-8.19 (2H, m), 8.69 (1H, s).

### Example B212

### 1-(4-Butylbenzyl)-5,6,7,8-tetrahydroisoquinoline

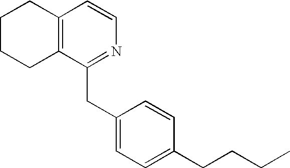

The compound of Example B211 (13.0 mg, 0.0367 mmol) was dissolved in a mixed solution of ethyl acetate and methanol (1:1, 1 ml), 10% palladium-carbon (containing 50% water, 13 mg) was added, and the mixture was stirred at room temperature under hydrogen atmosphere at atmospheric pressure for 12 hours. After purging the reaction system with nitrogen, the catalyst was removed by filtration through celite. The obtained filtrate was concentrated under reduced pressure to give the title compound (8.8 mg).

¹H-NMR (CDCl₃) δ (ppm): 0.90 (3H, t), 1.28-1.38 (2H, m), 1.52-1.59 (2H, m), 1.74-1.82 (4H, m), 2.55 (2H, t), 2.66 (2H, t), 2.81 (2H, t), 4.26 (2H, s), 7.07-7.15 (5H, m), 8.32 (1H, d).

### Example B213

### 1-[2-(Phenyl)benzyl]isoquinoline

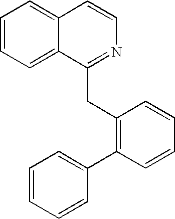

The title compound was obtained by treating 2-phenylbenzyl bromide instead of n-butylbenzyl chloride in the same manner as in Example B2.

¹H-NMR (CDCl₃) δ (ppm): 4.62 (2H, s), 7.05 (1H, d), 7.16 (1H, dd), 7.22-7.50 (8H, m), 7.52 (1H, d), 7.58 (1H, dd), 7.65 (1H, d), 7.76 (1H, d), 8.47 (1H, d).

### Example B214

### 1-[4-Fluoro-2-(trifluoromethyl)benzyl]isoquinoline

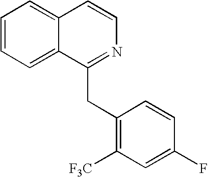

The title compound was obtained by treating 4-fluoro-2-(trifluoromethyl)benzyl methanesulfonate instead of n-butylbenzyl chloride in the same manner as in Example B2.

¹H-NMR (CDCl₃) δ (ppm): 4.83 (2H, s), 6.87 (1H, dd), 7.01 (1H, ddd), 7.43 (1H, dd), 7.54 (1H, dd), 7.61 (1H, d), 7.67 (1H, dd), 7.85 (1H, d), 7.96 (1H, d), 8.49 (1H, d).

### Example B215

### 1,3-Benzodioxoyl-4-yl-(1-isoquinolyl)methanol

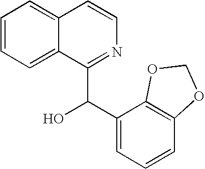

The title compound was obtained by treating2,3-methylenedioxybenzaldehyde in the same manner as in Example B82.

¹H-NMR (CDCl₃) δ (ppm): 5.97-5.99 (1H, m), 6.09 (1H, brs), 6.20-6.40 (1H, m), 6.54-6.60 (2H, m), 6.65-6.70 (2H, m), 7.52 (1H, dd), 7.63 (1H, d), 7.64 (1H, dd), 7.84 (1H, d), 8.04 (1H, d), 8.53 (1H, d).

### Example B216

### 1,3-Benzodioxoyl-4-yl-(1-isoquinolyl)methyl Acetate

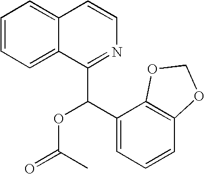

The title compound was obtained by treating the compound of Example B215 in the same manner as in Example B38.

¹H-NMR (CDCl₃) δ (ppm): 2.23 (3H, s), 5.98-6.02 (2H, m), 6.74-6.79 (1H, m), 6.90-6.93 (1H, m), 7.15-7.19 (1H, m), 7.23-7.28 (1H, m), 7.58 (1H, dd), 7.60 (1H, d), 7.66 (1H, dd), 7.83 (1H, d), 8.28 (1H, d), 8.57 (1H, d).

### Example B217

### 1-(1,3-Benzodioxoyl-4-ylmethyl)isoquinoline

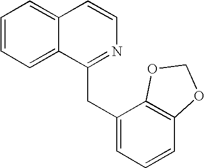

The title compound was obtained by treating the compound of Example B216 in the same manner as in Example B39.

¹H-NMR (CDCl₃) δ (ppm): 4.62 (2H, s), 6.02 (2H, s), 6.64-6.70 (3H, m), 7.57 (1H, dd), 7.58 (1H, d), 7.66 (1H, dd), 7.83 (1H, d), 8.23 (1H, d), 8.50 (1H, d).

### Example B218

### 1-(1-Naphthylmethyl)isoquinoline

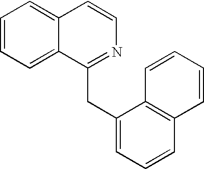

The title compound was obtained by treating 1-(chloromethyl)naphthalene instead of n-butylbenzyl chloride in the same manner as in Example B2.

¹H-NMR (CDCl₃) δ (ppm): 5.13 (2H, s), 6.96 (1H, d), 7.29 (1H, d), 7.45-7.67 (5H, m), 7.72 (1H, d), 7.84-7.90 (2H, m), 8.08 (1H, d), 8.26 (1H, d), 8.52 (1H, d).

### Example B219

### 3-Bromophenylbutyrate

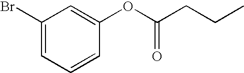

n-Butyryl chloride (7.25 ml) was added to an ice-cooled solution of 3-bromophenol (10.0 g) in pyridine (50 ml), and this reaction mixture was stirred at that temperature for 3 hours, then at room temperature for another 3.5 hours. After ice was added, the reaction mixture was extracted with ethyl acetate, washed with 1 N hydrochloric acid and water, dried over anhydrous magnesium sulfate, and then concentrated under reduced pressure. The residue was purified by silica gel column chromatography to give the title compound (12.77 g).

¹H-NMR (CDCl3) δ (ppm): 1.04 (3H, t), 1.72-1.82 (2H, m), 2.54 (2H, t), 7.04 (1H, dd), 7.22-7.29 (2H, m), 7.36 (1H, d).

### Example B220

### 1-(4-Bromo-2-hydroxyphenyl)-1-butanone

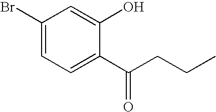

Aluminum chloride (10.51 g) was added to a solution of the compound of Example B219 (12.77 g) in chlorobenzene (70 ml) under nitrogen atmosphere, and this reaction mixture was stirred while heating under reflux for 9 hours. After the reaction mixture was cooled to room temperature, ice was added thereto. The resulting mixture was extracted with ethyl acetate, washed with water, dried over anhydrous magnesium sulfate, and then concentrated under reduced pressure. The compound thus obtained was used in the following reaction without further purification.

¹H-NMR (CDCl3) δ (ppm): 0.91 (3H, t), 1.53-1.65 (2H, m), 3.00 (2H, t), 7.02 (1H, dd), 7.19 (1H, d), 7.78 (1H, d), 12.50 (1H, s).

### Example B221

### 1-(4-Bromo-2-methoxyphenyl)-1-butanone

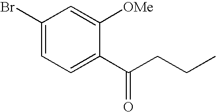

Potassium carbonate (9.07 g) and methyl iodide (3.92 ml) were added to a solution of the compound of Example B220 (13.30 g) in acetone (75 ml), and this reaction mixture was stirred while heating under reflux for 4 hours. The reaction mixture was filtered through celite, ether was added to remove insoluble material by filtration, and the filtrate was concentrated under reduced pressure. The residue was purified by silica gel column chromatography to give the title compound (9.52 g).

¹H-NMR (CDCl3) δ (ppm): 0.95 (3H, t), 1.64-1.74 (2H, m), 2.91 (2H, t), 3.90 (3H, s), 7.10 (1H, d), 7.14 (1H, dd), 7.54 (1H, d).

### Example B222

### 4-Bromo-1-butyl-2-methoxybenzene

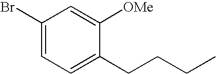

The title compound was obtained by treating the compound of Example B221 in the same manner as in Example B3.

¹H-NMR (CDCl3) δ (ppm): 0.92 (3H, t), 1.29-1.39 (2H, m), 1.48-1.56 (2H, m), 2.54 (2H, t), 3.81 (3H, s), 6.95 (1H, s), 6.96-7.02 (2H, m).

### Example B223

### (4-Butyl-3-methoxyphenyl)(1-isoquinolyl)ketone

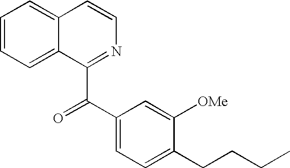

A mixture containing the title compound was obtained by treating the compound of Example B222 in the same manner as in Example B36.

This mixture was used in the following reaction without separation and purification.

### Example B224

### (4-Butyl-3-methoxyphenyl)(1-isoquinolyl)methanol

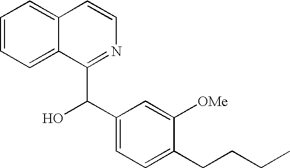

A mixture containing the title compound was obtained by treating the compound of Example B223 in the same manner as in Example B37.

This mixture was used in the following reaction without separation and purification.

### Example B225

### (4-Butyl-3-methoxyphenyl)(1-isoquinolyl)methyl Acetate

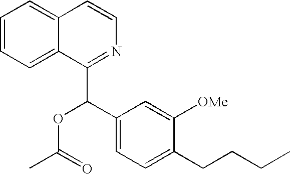

The title compound was obtained by treating the compound of Example B224 in the same manner as in Example B38.

¹H-NMR (CDCl3) δ (ppm): 0.90 (3H, t), 1.24-1.38 (2H, m), 1.46-1.60 (2H, m), 2.24 (3H, s), 2.54 (2H, t), 3.76 (3H, s), 6.97 (1H, s), 6.98 (1H, d), 7.06 (1H, d), 7.53-7.67 (4H, m), 7.83 (1H, d), 8.26 (1H, d), 8.58 (1H, d).

### Example B226

### 1-(4-Butyl-3-methoxybenzyl)isoquinoline

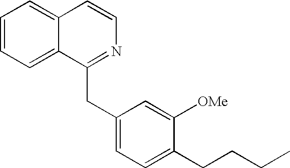

The title compound was obtained by treating the compound of Example B225 in the same manner as in Example B39.

¹H-NMR (CDCl3) δ (ppm): 0.89 (3H, t), 1.27-1.38 (2H, t), 1.45-1.54 (2H, t), 2.52 (2H, t), 3.72 (3H, s), 4.63 (2H, s), 6.78 (1H, d), 6.79 (1H, s), 6.99 (1H, d), 7.53 (1H, dd), 7.55 (1H, d), 7.64 (1H, dd), 7.80 (1H, d), 8.19 (1H, d), 8.49 (1H, d).

### Example B227

### 2-Butyl-5-(1-isoquinolylomethyl)phenol

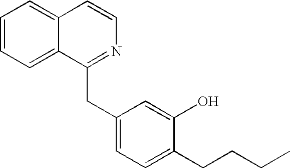

The title compound was obtained by treating the compound of Example B226 in the same manner as in Example B40.

¹H-NMR (CDCl3) δ (ppm): 0.91 (3H, t), 1.30-1.40 (2H, m), 1.52-1.65 (2H, m), 2.55 (2H, t), 4.55 (2H, s), 6.46 (1H, brs), 6.85 (1H, d), 7.03 (1H, d), 7.32-7.40 (1H, m), 7.55 (1H, dd), 7.68 (1H, dd), 7.81 (1H, d), 7.94-8.05 (1H, m), 8.14 (1H, d).

The proton of the phenolic hydroxyl group was not observed in the NMR spectrum.

### Example B228

### 2-Bromo-3-(methoxymethoxy)pyridine

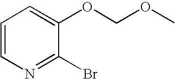

The title compound was synthesized in the same manner as in Example B202 by using 2-bromo-3-hydroxypyridine.

¹H-NMR (CDCl₃) δ (ppm): 3.53 (3H, s), 5.29 (2H, s), 7.19-7.23 (1H, m), 7.42-7.45 (1H, m), 8.04-8.06 (1H, m).

### Example B229

### 2-(4-Butylbenzyl)-3-(methoxymethoxy)pyridine

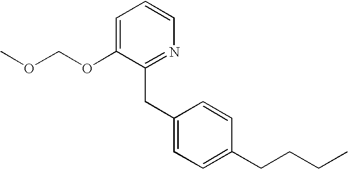

The compound of Example B205 (7 ml, 3 mmol) was added to an ice-cooled mixed solution of the compound of Example B228 (524 mg, 2.40 mmol) and dichloro(diphenylphosphinopropane)nickel (65.0 mg, 0.120 mmol) in tetrahydrofuran (10 ml), and the mixture was stirred while heating under reflux for 5 hours. After allowing the mixture to cool to room temperature, ethyl acetate was added. The resulting mixture was washed successively with a saturated aqueous ammonium chloride solution, a saturated aqueous sodium hydrogencarbonate solution, and saturated brine, then concentrated under reduced pressure. The residue was filtered through NH-silica gel. After concentrating under reduced pressure, the residue was dissolved in methanol (15 ml), triethylamine (500 μl, 3.59 mmol) and 10% palladium-carbon (containing 50% water, 50 mg) were added, and the resulting mixture was stirred at room temperature under hydrogen atmosphere at atmospheric pressure for 3 hours. After purging the reaction system with nitrogen, the catalyst was removed by filtration through celite, and the filtrate was concentrated under reduced pressure. The residue was purified by silica gel column chromatography to give the title compound (280 mg).

¹H-NMR (CDCl₃) δ (ppm): 0.89 (3H, t), 1.28-1.34 (2H, m), 1.52-1.58 (2H, m), 2.53 (2H, t), 3.33 (3H, s), 4.16 (2H, s), 5.16 (2H, s), 7.04-7.10 (3H, m), 7.20 (2H, d), 7.33-7.35 (1H, m), 8.19-8.20 (1H, m).

### Example B230

### 2-(4-Butylbenzyl)-3-pyridinol

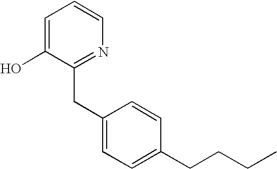

Trifluoroacetic acid (1 ml) was added to a solution of the compound of Example B229 (256 mg, 0.849 mmol) in methylene chloride (5 ml), and this reaction mixture was stirred at room temperature overnight. After a saturated aqueous sodium hydrogencarbonate solution and ethyl acetate were added, the reaction mixture was washed with saturated brine and concentrated under reduced pressure. The residue was purified by silica gel column chromatography to give the title compound (182 mg).

¹H-NMR (CDCl₃) δ (ppm): 0.90 (3H, t), 1.28-1.37 (2H, m), 1.51-1.58 (2H, m), 2.54 (2H, t), 4.20 (2H, s), 7.02-7.08 (4H, m), 7.22 (2H, d), 8.08-8.09 (1H, m).

The proton of the phenolic hydroxyl group was not observed in the NMR spectrum.

### Example B231

### 2-(4-Butylbenzyl)-3-methoxypyridine

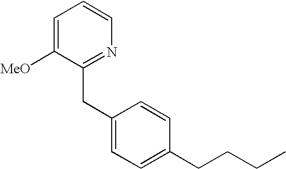

Potassium carbonate (33.0 mg, 0.239 mmol) and methyl iodide (14.9 μl, 0.239 mmol) were added to a solution of the compound of Example B230 (19.2 mg, 0.0796 mmol) in acetone (1 ml), and this reaction mixture was stirred at room temperature for 3 hours. After ethyl acetate was added, the reaction mixture was washed with saturated brine and concentrated under reduced pressure. The residue was purified by silica gel column chromatography to give the title compound (1.47 mg).

¹H-NMR (CDCl₃) δ (ppm): 0.90 (3H, t), 1.32-1.34 (2H, m), 1.53-1.57 (2H, m), 2.54 (2H, t), 3.82 (3H, s), 4.14 (2H, s), 7.06 (2H, d), 7.10-7.11 (2H, m), 7.21 (2H, d), 8.12-8.14 (1H, m).

### Example B232

### 2-(4-Butylbenzyl)-3-chloropyridine

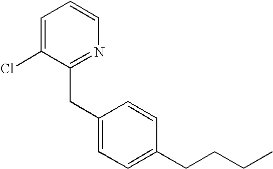

The compound of Example B205 (12 ml, 5 mmol) was added to an ice-cooled mixed solution of 2,3-dichloropyridine (525 mg, 3.55 mmol) and dichloro(diphenylphosphinopropane)nickel (96.2 mg, 0.178 mmol) in tetrahydrofuran (4 ml), and this reaction mixture was stirred at room temperature for 1 hour. After ethyl acetate was added, the reaction mixture was washed successively with a saturated aqueous ammonium chloride solution, a saturated aqueous sodium hydrogencarbonate solution, and saturated brine, and then concentrated under reduced pressure. The residue was purified by silica gel column chromatography to give the title compound (199 mg).

¹H-NMR (CDCl₃) δ (ppm): 0.91 (3H, t), 1.29-1.38 (2H, m), 1.52-1.60 (2H, m), 2.56 (2H, t), 4.28 (2H, s), 7.08-7.13 (3H, m), 7.21 (2H, d), 7.64 (1H, dd), 8.46 (1H, dd).

### Example B233

### 2-(4-Butylbenzyl)-3-ethylpyridine

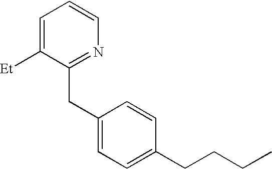

Ethylmagnesium chloride (0.97 M, 102 μl, 0.993 mmol) was added to a mixed solution of the compound of Example B232 (12.9 mg, 0.0496 mmol) and dichloro(diphenylphosphinoferrocene)nickel (3.4 mg, 0.0050 mmol) in tetrahydrofuran (1 ml). The reaction mixture was stirred at 50° C. for 1 hour, then heated under reflux for another 2 hours. After allowing the reaction mixture to reach room temperature, ethyl acetate was added thereto. The reaction mixture was washed with a saturated aqueous ammonium chloride solution and saturated brine, then concentrated under reduced pressure. The residue was purified by silica gel column chromatography to give the title compound (3.29 mg).

¹H-NMR (CDCl₃) δ (ppm): 0.90-0.93 (6H, m), 1.30-1.37 (2H, m), 1.54-1.59 (2H, m), 2.55-2.59 (4H, m), 4.12 (2H, s), 7.05-7.18 (5H, m), 7.55-7.59 (1H, m), 8.53-8.55 (1H, m).

### Example B234

tert-Butyl N-(2-Bromo-3-pyridyl)carbamate

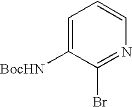

N-bromosuccinimide (7.51 g, 42.2 mmol) was added to an ice-cooled mixed solution of 3-aminopyridine (3.97 g, 42.2 mmol) in dimethylformamide (25 ml), and this reaction mixture was stirred at that temperature for 30 minutes. After ethyl acetate was added, the reaction mixture was washed with saturated brine and concentrated under reduced pressure. A solution of the residue in methylene chloride (20 ml) was cooled on ice, then triethylamine (3.74 ml, 26.8 mmol), a catalytic amount of dimethylaminopyridine, and di-t-butyl dicarbonate (3.08 ml, 13.4 mmol) were added to the solution, and the mixture was stirred at room temperature overnight. After concentration under reduced pressure, the residue was purified by silica gel column chromatography to give the title compound (344 mg).

¹H-NMR (CDCl₃) δ (ppm): 1.55 (9H, s), 7.03 (1H, brs), 7.25 (1H, dd), 8.03 (1H, dd), 8.46 (1H, d).

### Example B235

### 2-Bromo-3-(N-t-butoxycarbonyl-N-methyl)aminopyridine

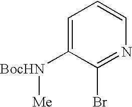

Methyl iodide (157 μl, 2.52 mmol) and 66% sodium hydride (91.6 mg, 2.52 mmol) were added to an ice-cooled solution of the compound of Example B234 (344 mg, 1.26 mmol) in dimethylformamide (5 ml), and this reaction mixture was stirred at that temperature for 40 minutes. After ethyl acetate was added, the reaction mixture was washed with saturated brine and filtered through silica gel. The organic layer was concentrated under reduced pressure to give the title compound (356 mg).

¹H-NMR (CDCl₃) δ (ppm): 1.36 (9H, s), 3.17 (3H, s), 7.30 (1H, dd), 7.55 (1H, d), 8.30 (1H, dd).

### Example B236

### N-[2-(4-Butylbenzyl)-3-pyridyl]-N-methylamine

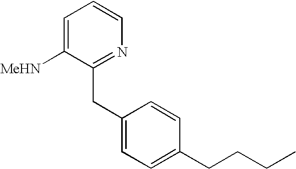

To a methylene chloride solution (2 ml) of a compound, which was obtained by introduction of a 4-butylbenzyl group to the compound of Example B235 (62.8 mg, 0.219 mmol) in the same manner as in Example B211, trifluoroacetic acid (2 ml) was added. The mixture was stirred at room temperature for 1 hour, and then added dropwise to an aqueous solution of sodium hydrogencarbonate. After ethyl acetate was added, the mixture was washed with saturated brine and concentrated under reduced pressure. The residue was purified by silica gel column chromatography to give the title compound (29.7 mg).

¹H-NMR (CDCl₃) δ (ppm): 0.91 (3H, t), 1.29-1.38 (2H, m), 1.53-1.60 (2H, m), 2.56 (2H, t), 2.72 (3H, s), 3.63 (1H, br s), 4.09 (2H, s), 6.86 (1H, d), 7.08-7.12 (5H, m), 7.98 (1H, dd).

### Example B237

### N-[2-(4-Butylbenzyl)-3-pyridyl]-N,N-dimethylamine

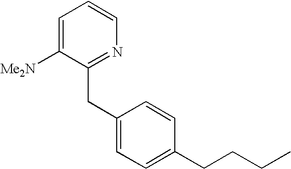

Acetic acid (12.1 μl, 0.211 mmol), 37% formalin (15.8 μl, 0.211 mmol), and sodium triacetoxyborohydride (44.7 mg, 0.211 mmol) were added to an ice-cooled solution of the compound of Example B236 (26.8 mg, 0.105 mmol) in methylene chloride (2 ml), and the mixture was stirred at room temperature for 30 minutes. After ethyl acetate was added, the mixture was washed with a saturated aqueous sodium hydrogencarbonate solution and saturated brine and concentrated under reduced pressure. The residue was purified by silica gel column chromatography to give the title compound (23.3 mg)

¹H-NMR (CDCl₃) δ (ppm): 0.91 (3H, t), 1.30-1.36 (2H, m), 1.52-1.59 (2H, m), 2.55 (2H, t), 2.67 (6H, s), 4.24 (2H, s), 7.06 (2H, d), 7.10 (1H, dd), 7.18 (2H, d), 7.40 (1H, dd), 8.27 (1H, dd).

### Example B238

### 2-(4-Butylbenzyl)-4-methoxypyridine

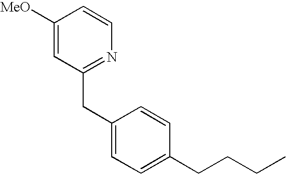

The title compound was obtained in the same manner as in Example B211 using 2-chloro-4-methoxypyridine.

¹H-NMR (CDCl₃) δ (ppm): 0.91 (3H, t), 1.31-1.37 (2H, m), 1.53-1.59 (2H, m), 2.57 (2H, t), 3.78 (3H, s), 4.06 (2H, s), 6.61-6.65 (2H, m), 7.11 (2H, d), 7.17 (2H, d), 8.36 (1H, d).

### Example B239

### 2-(4-Butylbenzyl)-4-chloropyridine

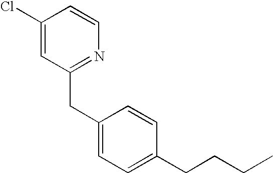

Phosphorus oxychloride (57.0 μl, 0.612 mmol) was added to an ice-cooled solution of the compound of Example B238 (52.0 mg, 0.204 mmol) in dimethylformamide (1 ml), and this reaction mixture was stirred at 100° C. for 8 hours. The reaction mixture was allowed to cool, poured on ice, and warmed to room temperature. After ethyl acetate was added, the mixture was washed with a saturated aqueous sodium hydrogencarbonate solution and saturated brine, then concentrated under reduced pressure. The residue was purified by silica gel column chromatography to give the title compound (2.29 mg).

¹H-NMR (CDCl₃) δ (ppm): 0.92 (3H, t), 1.31-1.38 (2H, m), 1.53-1.61 (2H, m), 2.59 (2H, t), 4.10 (2H, s), 7.12-18 (6H, m), 8.44 (1H, d).

### Example B240

### 2-Chloro-3-methoxypyridine

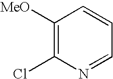

The title compound was obtained in the same manner as in Example B231 using 2-chloro-3-hydroxypyridine.

¹H-NMR (CDCl₃) δ (ppm): 3.93 (3H, s), 7.21-7.22 (2H, m), 7.99-8.01 (1H, m).

### Example B241

### 2-Chloro-3,4-dimethoxypyridine

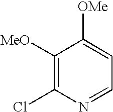

A solution of diisopropylamine (84.0 μl, 0.599 mmol) and the compound of Example B240 (860 mg, 5.99 mmol) in tetrahydrofuran (4 ml) was added to a solution of 1.06 M phenyllithium cyclopentane-diethyl ether solution in tetrahydrofuran (11 ml) cooled to −78° C. under nitrogen atmosphere. This reaction mixture was stirred at −40° C. for 1 hour, then at −18° C. for another 20 minutes. The reaction mixture was cooled again to −78° C., trimethoxyborate (2.04 ml, 18.0 mmol) was added dropwise thereto, and the resulting mixture was stirred at 0° C. for 20 minutes. At that temperature, aqueous ammonia (29%, 30 ml), ammonium chloride (4.5 g,), and an aqueous hydrogen peroxide solution (30%, 12 ml) were added in this order, and the mixture was stirred at room temperature for 2 hours. Saturated sodium thiosulfate, acetic acid and ethyl acetate were added, and the mixture was washed with saturated brine. The ethyl acetate layer obtained upon filtration through silica gel was concentrated under reduced pressure. The resulting residue was treated in the same manner as in Example B231 to obtain the title compound (31.3 mg).

¹H-NMR (CDCl₃) δ (ppm): 3.89 (3H, s), 3.94 (3H, s), 6.82 (1H, d), 8.05 (1H, d).

### Example B242

### 2-(4-Butylbenzyl)-3,4-dimethoxypyridine

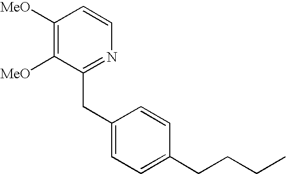

The title compound was obtained in the same manner as in Example B206 using the compound of Example B241.

¹H-NMR (CDCl₃) δ (ppm): 0.90 (3H, t), 1.26-1.35 (2H, m), 1.53-1.57 (2H, m), 2.54 (2H, t), 3.70 (3H, s), 3.89 (3H, s), 4.12 (2H, s), 6.72 (1H, d), 7.06 (2H, d), 7.21 (2H, d), 8.20 (1H, d).

### Example B243

### 2,4-Di-(4-Butylbenzyl)-3-methoxypyridine

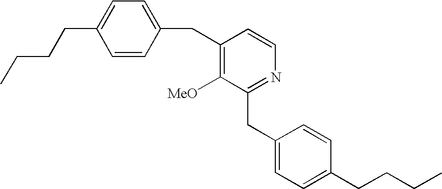

A solution of the compound of Example B240 (436 mg, 3.04 mmol) in diethyl ether (2 ml) was added to a solution of 1.43 M t-butyllithium n-pentane solution (2.76 ml, 3.95 mmol) in diethyl ether (5 ml) cooled to −78° C. under nitrogen atmosphere, and this reaction mixture was stirred at that temperature for 30 minutes. A solution of tetramethylethylenediamine (688 μl, 4.56 mmol) and hexachloroethane (719 mg, 3.04 mmol) in diethyl ether (3 ml) was further added and the reaction mixture was stirred at that temperature for 1 hour. After warming gradually to room temperature, ethyl acetate was added, and the mixture was washed with saturated brine. The ethyl acetate layer obtained upon filtration through silica gel was concentrated under reduced pressure. The resulting residue was treated in the same manner as in Example B206 to obtain the title compound (10.1 mg).

¹H-NMR (CDCl₃) δ (ppm): 0.89-0.94 (6H, m), 1.31-1.37 (4H, m), 1.52-1.62 (4H, m), 2.53-2.59 (4H, m), 3.74 (3H, s), 4.07 (2H, s), 4.13 (2H, s), 6.84 (1H, d), 6.98 (1H, d), 7.04-7.22 (8H, m).

### Example B244

### 2-(4-Bromo-2-fluorobenzyl)-3-(methoxymethoxy)pyridine

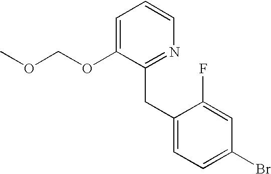

A solution of the compound of Example B228 (422 mg, 1.94 mmol) in tetrahydrofuran (3 ml) was added to a solution of 2.47 M n-butyllithium n-hexane solution (862 μl, 2.13 mmol) in tetrahydrofuran (3 ml) cooled to −78° C. under nitrogen atmosphere, and this reaction mixture was stirred at that temperature for 1 hour. After copper(I) bromide (139 mg, 0.968 mmol) was added, the reaction mixture was stirred at 0° C. for 1 hour and cooled again to −78° C. Next, 4-bromo-2-fluorobenzyl bromide (259 mg, 0.968 mmol) was added, and the resulting mixture was stirred at 0° C. for 1 hour. Tetramethylethylenediamine (584 μl, 3.88 mmol) was further added, and the resulting reaction mixture was stirred at that temperature for 1 hour. After diethyl ether and an aqueous ammonia solution were added to the reaction mixture, the organic layer was washed with saturated brine and concentrated under reduced pressure. The residue was purified by silica gel column chromatography to give the title compound (81.0 mg).

¹H-NMR (CDCl₃) δ (ppm): 3.38 (3H, s), 4.17 (2H, s), 5.18 (2H, s), 7.04 (1H, t), 7.11-7.22 (3H, m), 7.38 (1H, dd), 8.19 (1H, dd).

### Example B245

### 2-(4-Bromo-2-fluorobenzyl)-3-pyridinol

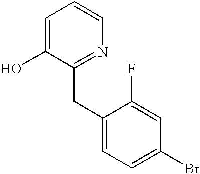

Trifluoroacetic acid (1 ml) was added to the compound of Example B244 (134 mg, 0.411 mmol) in methylene chloride (4 ml), and this reaction mixture was stirred at room temperature overnight. After neutralizing the mixture with saturated aqueous sodium hydrogencarbonate, ethyl acetate was added. The ethyl acetate layer was washed with saturated brine and concentrated under reduced pressure. The residue was purified by silica gel column chromatography to give the title compound (97.5 mg).

¹H-NMR (CDCl₃) δ (ppm): 4.17 (2H, s), 7.10-7.24 (5H, m), 8.15 (1H, t).

The proton of the phenolic hydroxyl group was not observed in the NMR spectrum.

### Example B246

### 2-(4-Bromo-2-fluorobenzyl)-3-methoxypyridine

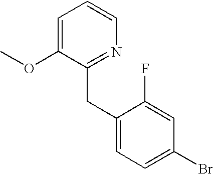

Potassium carbonate (38.7 mg, 0.280 mmol) and methyl iodide (10.5 μl, 0.168 mmol) were added to a solution of the compound of Example B245 (15.8 mg, 0.0560 mmol) in dimethylformamide (1 ml), and this reaction mixture was stirred at room temperature for 2 hours. After ethyl acetate was added, the reaction mixture was washed with saturated brine and concentrated under reduced pressure. The residue was purified by silica gel column chromatography to give the title compound (14.0 mg).

¹H-NMR (CDCl₃) δ (ppm): 3.82 (3H, s), 4.15 (2H, s), 7.03 (1H, t), 7.12-7.22 (4H, m), 8.13 (1H, dd).

The following compounds of Example B were synthesized in the same manner as in Example B246, and purification was performed by LC-MS [eluent: an acetonitrile solution containing 0.1% trifluoroacetic acid: an aqueous solution containing 0.1% trifluoroacetic acid=1:99 to 100:0/20-minute cycle, flow rate: 20 ml/minute, column: YMC Combiprep ODS-AM, 20 mm Φ×50 mm (long)].

### Example B247

### 2-(4-Bromo-2-fluorobenzyl)-3-ethoxypyridine

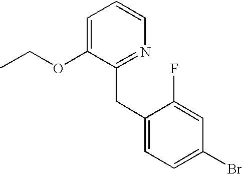

MS m/z (ESI: MH⁺): 310.0.

### Example B248

### 2-(4-Bromo-2-fluorobenzyl)-3-propoxypyridine

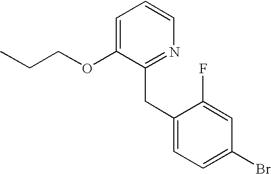

MS m/z (ESI: MH⁺): 324.0.

### Example B249

### 2-(4-Bromo-2-fluorobenzyl)-3-butoxypyridine

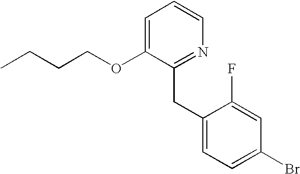

MS m/z (ESI: MH⁺): 338.1.

### Example B250

### 2-(4-Bromo-2-fluorobenzyl)-3-(pentyloxy)pyridine

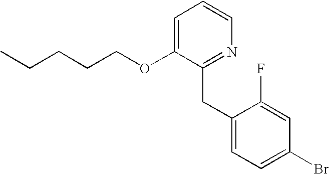

MS m/z (ESI: MH⁺): 352.1

### Example B251

### 2-(4-Bromo-2-fluorobenzyl)-3-(hexyloxy)pyridine

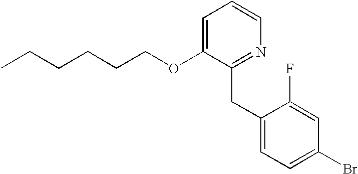

MS m/z (ESI: MH⁺) 366.0

### Example B252

### 2-(4-Bromo-2-fluorobenzyl)-3-(2-fluoroethoxy)pyridine

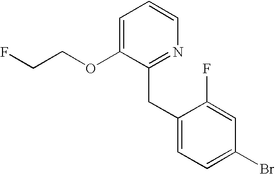

MS m/z (ESI: MH⁺) 328.0

### Example B253

### 2-(4-Bromo-2-fluorobenzyl)-3-(3-fluoropropoxy)pyridine

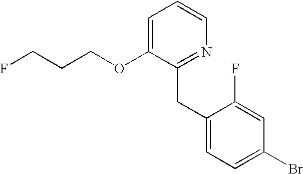

MS m/z (ESI: MH⁺) 342.0

### Example B254

### 2-(4-Bromo-2-fluorobenzyl)-3-isopropoxypyridine

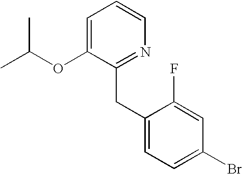

MS m/z (ESI: MH⁺): 324.0.

### Example B255

### 2-(4-Bromo-2-fluorobenzyl)-3-(2,2,2-trifluoroethoxy)pyridine

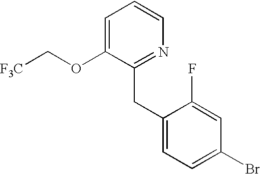

MS m/z (ESI: MH⁺): 364.0.

### Example B256

### 2-(4-Bromo-2-fluorobenzyl)-3-(3,3,3-trifluoropropoxy)pyridine

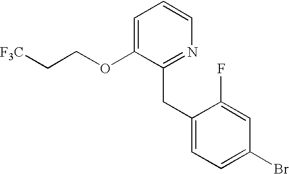

MS m/z (ESI: MH⁺): 378.0.

### Example B257

Compounds were evaluated using the S. cerevisiae reporter system of Example A2. The lowest concentration at which cephalosporinase activity in the cell wall fraction became 50% or less compared to that obtained where the compound was not treated, was defined to be the IC50 value. Effects of the representative compounds are shown in Table 1.

TABLE 1

| | IC50 |
|---|---|
| Compound | (μg/ml) |
| 1- (4-butylbenzyl)isoquinoline (Example B2) | 0.39 |
| N1-{3-[4-(1-isoquinolylmethyl)phenyl]-2-propynyl} | 6.25 |
| acetamide (Example B60) | |
| N1-{3-[4-(1-isoquinolylmethyl)phenyl]propyl}-N1-methyl | 50 |
| acetamide (Example B73) | |
| 5-butyl-2-(1-isoquiriolylmethyl)phenol (Example B85) | 0.20 |
| 4-(4-butylbenzyl)thieno[3,2-c]pyridine (Example B187) | 0.78 |
| 7-(4-butylbenzyl)thieno[2,3-c]pyridine (Example B195) | 0.39 |
| 2-(4-butylbenzyl)-3-methoxypyridine (Example B231) | 0.78 |
| 2-(4-butylbenzyl)-3,4-dimethoxypyridine (Example B242) | 0.78 |

### INDUSTRIAL APPLICABILITY

The present invention revealed genes encoding the proteins participating in the transport process of the GPI-anchored proteins to the cell wall. Furthermore, this invention discloses a method of screening for compounds that inhibit the activity of these proteins, and also discloses representative compounds having the inhibitory activity.

Using novel compounds, the present invention showed that antifungal agents having a novel mechanism of inhibiting the transport process of the GPI-anchored proteins to the cell wall can be provided.

1. A DNA that encodes a protein having an activity to confer resistance of a fungus against the compound shown in formula (Ia) when the DNA is overexpressed in the fungus, wherein the DNA is selected from the group consisting of:
(a) a DNA encoding a protein comprising the amino acid sequence of SEQ ID NO: 2, 4, 6, 28, 40, or 59,
(b) a DNA comprising the nucleotide sequence of SEQ ID NO: 1, 3, 5, 27, 39, 41, 54, or 58,
(c) a DNA that hybridizes under stringent conditions to a DNA comprising the nucleotide sequence of SEQ ID NO: 1, 3, 5, 27, 39, 41, 54, or 58,
(d) a DNA encoding a protein comprising the amino acid sequence of SEQ ID NO: 2, 4, 6, 28, 40, or 59, wherein one or more amino acids have been added, deleted, substituted, and/or inserted, and
(e) a DNA that is amplified using SEQ ID NOS: 29 and 31 or SEQ ID NOS: 29 and 30 as primers

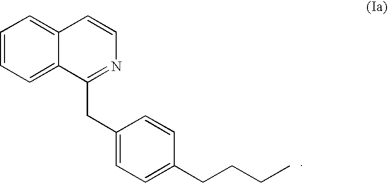

2. A DNA that encodes a protein having an activity to decrease the amount of a GPI-anchored protein in the cell wall of a fungus due to a defect in the function of the DNA, wherein the DNA is selected from the group consisting of:
(a) a DNA encoding a protein comprising the amino acid sequence of SEQ ID NO: 2, 4, 6, 28, 40, or 59,
(b) a DNA comprising the nucleotide sequence of SEQ ID NO: 1, 3, 5, 27, 39, 41, 54, or 58,
(c) a DNA that hybridizes under stringent conditions to a DNA comprising the nucleotide sequence of SEQ ID NO: 1, 3, 5, 27, 39, 41, 54, or 58,
(d) a DNA encoding a protein comprising the amino acid sequence of SEQ ID NO: 2, 4, 6, 28, 40, or 59, wherein one or more amino acids have been added, deleted, substituted, and/or inserted, and
(e) a DNA that is amplified using SEQ ID NOS: 29 and 31 or SEQ ID NOS: 29 and 30 as primers.

3. A protein encoded by the DNA of claim 1 or 2.

4. A vector into which the DNA of claim 1 or 2 has been inserted.

5. A transformant harboring the DNA of claim 1 or 2, or the vector of claim 4.

6. The transformant of claim 5 which is a fungus that overexpresses the protein of claim 3.

7. A fungus, wherein the function of the protein of claim 3 is defective.

8. A method for producing the protein of claim 3, which comprises the steps of culturing the transformant of claim 5, and collecting the expressed protein from the transformant, or from the culture supernatant thereof.

9. An antibody that binds to the protein of claim 3.

10. A method of screening for a compound having an antifungal action, wherein the method comprises the steps of:
(a) contacting a test sample with the protein of claim 3;
(b) detecting the binding activity between the protein and the test sample; and
(c) selecting a compound having an activity to bind to the protein.

11. A method of screening for a compound that has an antifungal action, which comprises the steps of:
(a) contacting a test sample with a fungus that is overexpressing the protein of claim 3;
(b) detecting the amount of transport of a GPI-anchored protein to the cell wall in the fungus; and
(c) selecting a compound that diminishes the amount of transport of the GPI-anchored protein to the cell wall detected in step
(b) as compared to the amount of transport detected when the test sample was contacted with a fungus that is not overexpressing the protein of claim 3.

12. A compound having an antifungal action that is isolated by the screening of claim 10 or 11.

13. An antifungal agent, comprising as an active ingredient a compound that inhibits the transport of GPI-anchored proteins to the cell wall of a fungus.

14. An antifungal agent, comprising as an active ingredient the antibody of claim 9 or the compound of claim 12.

15. The antifungal agent of claim 13, comprising as an active ingredient the compound represented by the general formula (I), a salt thereof, or a hydrate thereof, wherein in formula (I): 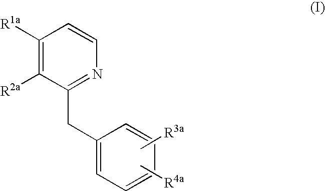
[R^{1a} and R^{2a} are identical to or different from each other and denote individually a hydrogen atom, halogen atom, hydroxyl group, nitro group, cyano group, trifluoromethyl group, trifluoromethoxy group, a substituted or unsubstituted C₁₋₆ alkyl group, C₂₋₆ alkenyl group, C₂₋₆ alkynyl group, a substituted or unsubstituted C₁₋₆ alkoxy group, or a group represented by the formula: 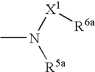
(wherein X¹ stands for a single bond, carbonyl group, or a group represented by the formula —S(O)₂—;
R^{5a} and R^{6a} are identical to or different from each other and denote a hydrogen atom or a substituted or unsubstituted C₁₋₆ alkyl group); R^{1a} and R^{2a} may form together a condensed ring selected from the group consisting of a substituted or unsubstituted benzene ring, a substituted or unsubstituted pyridine ring, a substituted or unsubstituted pyrrole ring, a substituted or unsubstituted thiophene ring, a substituted or unsubstituted furan ring, a substituted or unsubstituted pyridazine ring, a substituted or unsubstituted pyrimidine ring, a substituted or unsubstituted pyrazine ring, a substituted or unsubstituted imidazole ring, a substituted or unsubstituted oxazole ring, a substituted or unsubstituted thiazole ring, a substituted or unsubstituted pyrazole ring, a substituted or unsubstituted isoxazole ring, a substituted or unsubstituted isothiazole ring, a substituted or unsubstituted cyclohexane ring, and a substituted or unsubstituted cyclopentane ring;
R^{3a} and R^{4a} are identical to or different from each other and denote individually a hydrogen atom, halogen atom, hydroxyl group, nitro group, cyano group, carboxyl group, formyl group, hydroxyimino group, trifluoromethyl group, trifluoromethoxy group, C₁₋₆ alkyl group, C₁₋₆ alkoxy group, C₂₋₆ alkenyl group, C₂₋₆ alkynyl group, a group represented by the formula —C(O)NR^{7a}R^{7b} (wherein R^{7a} and R^{7b} are identical to or different from each other and denote individually a hydrogen atom, or a C₁₋₆ alkyl group), the formula —CO₂R^{7a} (wherein R^{7a} has the same meaning as defined above), the formula —S(O)ₙR^{7a} (wherein n stands for an integer of 0 to 2 and R^{7a} has the same meaning as defined above), the formula —S(O)₂NR^{7a}R^{7b} (wherein R^{7a} and R^{7b} have the same meaning as defined above), a group of the formula 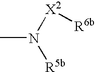
(wherein X² denotes a single bond, carbonyl group, or a group of the formula —S(O)₂—;
R^{5b} and R^{6b} are identical to or different from each other, and denote a hydrogen atom, a substituted or unsubstituted C₁₋₆ alkyl group, or a substituted or unsubstituted C₆₋₁₄ aryl group), or a group of the formula —Z¹—Z²
(wherein Z¹ denotes a single bond, oxygen atom, vinylene group, or ethynylene group;
Z² denotes a single bond, or a C₁₋₆ alkyl group substituted or unsubstituted with 0 to 4 substituents); R^{3a} and R^{4a} may together stand for a methylenedioxy group or 1,2-ethylenedioxy group, alternatively, R^{3a} and R^{4a} may together stand for the formation of a condensed ring selected from a group consisting of a substituted or unsubstituted benzene ring, substituted or unsubstituted pyridine ring, substituted or unsubstituted pyrrole ring, substituted or unsubstituted thiophene ring, substituted or unsubstituted furan ring, substituted or unsubstituted pyridazine ring, substituted or unsubstituted pyrimidine ring, substituted or unsubstituted pyrazine ring, substituted or unsubstituted imidazole ring, substituted or unsubstituted oxazole ring, substituted or unsubstituted thiazole ring, substituted or unsubstituted pyrazole ring, substituted or unsubstituted isoxazole ring, substituted or unsubstituted isothiazole ring, substituted or unsubstituted cyclohexane ring, and substituted or unsubstituted cyclopentane ring, except in cases where both R^{1a} and R^{2a} do not stand for hydrogen atoms].

16. The antifungal agent of claim 13, comprising as the active ingredient compound (Ia) of the formula:

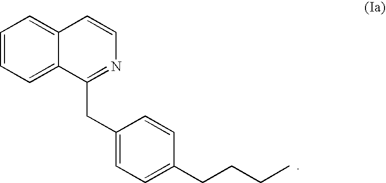

17. A compound represented by the formula (II), a salt or a hydrate thereof, wherein in formula (II), 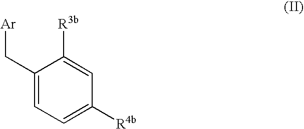
[Ar stands for a substituent selected from a group consisting of the formulae (IIIa) to (IIIf): 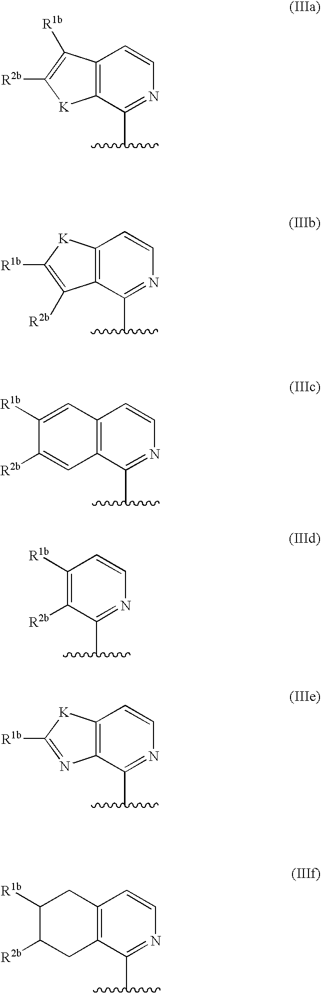
(wherein K denotes a sulfur atom, oxygen atom, or a group represented by the formula —NH—;
R^{1b} and R_{2b} are identical to or different from each other and denote individually a hydrogen atom, halogen atom, hydroxyl group, nitro group, cyano group, trifluoromethyl group, trifluoromethoxy group, a group represented by the formula 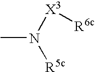
(wherein X³ denotes a single bond, carbonyl group, or a group represented by the formula —S(O)₂—;
R^{5c} and R^{6c} are identical to or different from each other and denote a hydrogen atom, or a substituted or unsubstituted C₁₋₆ alkyl group), or a group represented by the formula —X⁴—R^{8a} (wherein X⁴ denotes a single bond, oxygen atom, or sulfur atom; R^{8a} denotes a C₁₋₆ alkyl group, C₂₋₆ alkenyl group, C₂₋₆ alkynyl group, C₃₋₈ cycloalkyl group, or C₃₋₈ cycloalkenyl group); R^{1b} and R^{2b} together may form a methylenedioxy group, or a 1,2-ethylenedioxy group);
R^{3b} and R^{4b} are identical to or different from each other and denote individually a hydrogen atom, halogen atom, hydroxyl group, nitro group, cyano group, carboxyl group, formyl group, hydroxyimino group, trifluoromethyl group, trifluoromethoxy group, C₁₋₆ alkyl group, C₁₋₆ alkoxy group, C₂₋₆ alkenyl group, C₂₋₆ alkynyl group, or a group represented by the formula —Z^{1b}—Z^{2b}
(wherein Z^{1b} denotes a single bond, vinylene group, or ethynylene group;
Z^{2b} denotes a single bond, or a C₁₋₆ alkyl group that is substituted or unsubstituted with 0 to 4 substituents); except in cases where (1) Ar stands for the aforementioned formula (IIId) wherein R^{1b} and R^{2b} are both hydrogen atoms, (2) at least one of R³b or R⁴b denotes a hydrogen atom and the other is a hydrogen atom, methoxy group, hydroxyl group, methyl group, benzyloxy group, or a halogen atom, and Ar stands for the aforementioned formula (IIIc) wherein R^{1b} and R^{2b} both denote hydrogen atoms or methoxy groups, (3) at least one of R^{3b} or R^{4b} denotes a hydrogen atom and the other is a hydrogen atom, hydroxyl group, methoxy group, or benzyloxy group, and Ar stands for the formula (IIIc) wherein R^{1b} and R^{2b} both denote hydroxyl groups or benzyloxy groups, or (4) Ar stands for the formula (IIId) wherein R^{1b} is a hydrogen atom and R^{2b} is a formyl group, hydroxymethyl group, or methoxycarbonyl group].

18. The compound of claim 17, or a salt or hydrate thereof, wherein Ar stands for the formula: 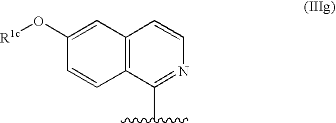
(wherein R^{1c} denotes a hydrogen atom, a substituted or unsubstituted C₁₋₆ alkyl group, or a benzyl group), and excluding the case when R³b denotes a hydrogen atom.

19. A compound represented by the formula (IIIc2), or a salt or hydrate thereof, wherein in formula (IIIc2), 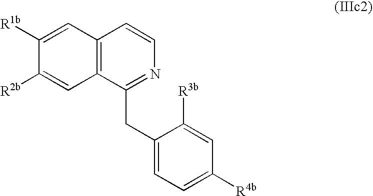
[R^{1b} and R^{2b} have the same meaning as defined above, except in cases wherein (1) R^{1b} denotes a group represented by the formula R^{1c}—O— (wherein R^{1c} has the same meaning as defined above), R^{2b} is a hydrogen atom, and R^{3b} denotes a hydrogen atom, (2) at least one of R^{3b} or R^{4b} denotes a hydrogen atom, and the other is a hydrogen atom, methoxy group, hydroxyl group, methyl group, benzyloxy group, or a halogen atom, and R^{1b} and R^{2b} both denote hydrogen atoms or methoxy groups, or (3) at least one of R^{3b} or R^{4b} denotes a hydrogen atom, and the other is a hydrogen atom, hydroxyl group, methoxy group, or benzyloxy group, and R^{1b} and R^{2b} both denote hydroxyl groups or benzyloxy groups].

20. The antifungal agent of claim 17, having an antifungal action.

21. The antifungal agent of claim 15, wherein at least one of R^{3a} and R^{4a} denotes a group represented by the formula —C(O)NR^{7a}R^{7b} (wherein R^{7a} and R^{7b} have the same meaning as defined above), the formula —CO₂R^{7a} (wherein R^{7a} has the same meaning as defined above), the formula —S(O)ₙR^{7a} (wherein n denotes an integer of 0 to 2 and R^{7a} has the same meaning as defined above), the formula —S(O)₂NR^{7a} R^{7b} (wherein R^{7a} and R^{7b} have the same meaning as defined above), the formula 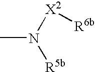
(wherein X², R^{5b}, and R^{6b} have the same meaning as defined above), or a C₁₋₆ alkoxy group substituted or unsubstituted with 0 to 4 substituents, or R^{3a} and R^{4a} together denote a methylenedioxy group, or a 1,2-ethylenedioxy group.

22. The antifungal agent of claim 15, wherein the compound having an antifungal action is (1) 1-benzylisoquinoline, (2) 1-(4-bromobenzyl)isoquinoline, (3) 1-(4-chlorobenzyl)isoquinoline, (4) 1-(4-fluorobenzyl)isoquinoline, (5) 1-(4-iodobenzyl)isoquinoline, (6) 1-(3-methylbenzyl)isoquinoline, (7) 1-(4-methylbenzyl)isoquinoline, (8) 1-(3,4-dimethylbenzyl)isoquinoline, (9) 1-(3-methoxybenzyl)isoquinoline, (10) 1-(4-methoxybenzyl)isoquinoline, (11) 1-(3,4-methylenedioxybenzyl)isoquinoline, (12) 1-(4-benzyloxybenzyl)isoquinoline, (13) 1-(4-cyanobenzyl)isoquinoline, (14) 1-(4-nitrobenzyl)isoquinoline, (15) 1-(4-aminobenzyl)isoquinoline, (16) 1-(4-methoxybenzyl)-6,7-dichloro-isoquinoline, (17) 1-(4-methoxy-2-nitro-benzyl)-isoquinoline, (18) 1-(4-methoxybenzyl)-6,7-methylenedioxy-isoquinoline, (19) 1-(2-amino-4-methoxy-benzyl)isoquinoline, (20) 1-(4-methoxybenzyl)-7-hydroxy-6-methoxy-isoquinoline, (21) 1-(4-benzyloxybenzyl)-6,7-dimethoxy-isoquinoline, (22) 1-(4-methoxybenzyl)-6,7-dimethoxy-isoquinoline, (23) 1(4-methoxy-2-nitro-benzyl)-isoquinoline, (24) 3-(4-(1-isoquinolylmethyl)phenoxylpropylcyanide, (25) 1-[4-(2,2,3,3-tetrafluoropropoxy)benzyl]isoquinoline, (26) 1-[4-(2-piperidinoethoxy)benzyl]isoquinoline, (27) 4-(1-isoquinolylmethyl)phenyl(2-morpholinoethyl)ether, (28) 1-[4-(2-methoxyethoxy)benzyl]isoquinoline, (29) N-{2-[4-(1-isoquinolylmethyl)phenoxy]ethyl}-N,N-dimethylamine, (30) 1-[4-(phenethyloxy)benzyl]isoquinoline, (31) 1-{4-[(2-methylallyl)oxy]benzyl}isoquinoline, (32) 1-(4-isobutoxybenzyl)isoquinoline, (33) 1-[4-(2-phenoxyethoxy)benzyl]isoquinoline, (34) methyl 2-[4-(1-isoquinolylmethyl)phenoxy]acetate, (35) 2-[4-(1-isoquinolylmethyl)phenoxy]-1-ethanol, (36) t-butyl N-{2-[4-(1-isoquinolylmethyl)phenoxy]ethyl}carbamate, (37) 1-{4-[3-(tetrahydro-2H-2-pyranyloxy)propoxy]benzyl}isoquinoline, (38) 2-[4-(1-isoquinolylmethyl)phenoxy]-1-ethaneamine, (39) 1-[4-(3-piperidinopropoxy)benzyl]isoquinoline, (40) 3-[4-(1-isoquinolylmethyl)phenoxy]-1-propanol, (41) 1-[4-(2-ethylbutoxy)benzyl]isoquinoline, (42) 4-[4-(1-isoquinolylmethyl)phenoxy]butanoic acid, (43) 1-(4-{3-[(4-benzylpiperazino)sulfonyl]propoxy}benzyl)isoquinoline, (44) 1-(4-{3-[4-(4-chlorophenyl)piperazino]propoxy}benzyl)isoquinoline, (45) 4-(1-isoquinolylmethyl)aniline, (46) N-(4-(1-isoquinolylmethyl)phenyl]butaneamide, (47) N-[4-(1-isoquinolylmethyl)phenyl]propaneamide, (48) N-[4-(1-isoquinolylmethyl)phenyl]-1-ethanesulfonamide, (49) N-[4-(1-isoquinolylmethyl)phenyl]-N-methyl-ethanesulfonamide, (50) N-[4-(1-isoquinolylmethyl)phenyl]-N-methylamine, (51) N-[4-(1-isoquinolylmethyl)phenyl]-N-propylamine, or (52) N-[4-(1-isoquinolylmethyl)phenyl]-N-methyl-N-propylamine.

23. A method for treating a mycotic infection comprising administering a therapeutically effective dose of any one of the antifungal agents of claims 13 to 22 to a mammal.
